(19) United States
(12) Patent Application Publication
(10) Pub. No.: US 2016/0100579 A1
(43) Pub. Date: Apr. 14, 2016

(54) NEMATICIDAL N-(2-SUBSTITUTED 2-PHENYLETHYL)CARBOXAMIDES AND N-(2-SUBSTITUTED 2-PHENYLETHYL)-THIOCARBOXAMIDES

(71) Applicant: BAYER CROPSCIENCE AG, Monheim am Rhein (DE)

(72) Inventors: Anne DECOR, Langenfeld (DE); Joerg Nico GREUL, Leverkusen (DE); Hans-Georg SCHWARZ, Dorsten (DE); Sebastian HOFFMANN, Neuss (DE); Lothar WILLMS, Hofheim (DE); Alexander SUDAU, Langenfeld (DE); Martin FUESSLEIN, Duesseldorf (DE); Peter LOESEL, Leverkusen (DE); Peter LUEMMEN, Idstein (DE); Ulrich GOERGENS, Ratingen (DE); Daniela PORTZ, Vettweiss (DE); Kerstin ILG, Koeln (DE); Olga MALSAM, Roesrath (DE); Eike Kevin HEILMANN, Duesseldorf (DE); Pierre-Yves COQUERON, Lyon (FR); Norio SASAKI, Ibaraki (JP); Philippe DESBORDES, Lyon (FR); Tomoki TSUCHIYA, Lyon (FR)

(21) Appl. No.: 14/787,533

(22) PCT Filed: Apr. 25, 2014
(86) PCT No.: PCT/EP2014/058504

§ 371 Date: Oct. 28, 2015

(30) Foreign Application Priority Data
Apr. 30, 2013 (EP) 13166000.3

(51) Int. Cl. A01N 43/60; A01N 37/20; C07D 241/24; C07D 333/38; A01N 43/10; A01N 43/08; A01N 43/40; C07D 231/14; A01N 43/56; C07D 311/04; A01N 43/16; C07D 307/68; C07C 233/73; C07D 213/81

(52) U.S. Cl. CPC A01N 43/60; C07C 233/73; A01N 37/20; C07D 241/24; C07D 333/38; A01N 43/10; C07D 213/81; A01N 43/40; C07D 231/14; A01N 43/56; C07D 311/04; A01N 43/16; C07D 307/68; A01N 43/08

(57) ABSTRACT

The present invention relates to the use of known and novel N-(2-substituted 2-phenylethyl)carboxamides and N-(2-substituted 2-phenylethyl)thiocarboxamides, for the control of plant damaging nematodes in agriculture and further relates to novel N-(2-substituted 2-phenylethyl)carboxamides and N-(2-substituted 2-phenylethyl)thiocarboxamides, processes and intermediate compounds for their preparation, their use as nematicides and their use as anthelmintics against endoparasites in animals and humans, compositions containing such compounds and methods for the control of nematodes and helminths.

The present invention relates to the use of known and novel N-(2-substituted 2-phenylethyl)carboxamides and N-(2-substituted 2-phenylethyl)thiocarboxamides, for the control of plant damaging nematodes predominantly in agriculture.

The present invention further relates to novel N-(2-substituted 2-phenylethyl)carboxamides and N-(2-substituted 2-phenylethyl)thiocarboxamides, processes and intermediate compounds for their preparation, their use as nematicides and their use as anthelmintics against endoparasites in animals and humans, and to the use thereof for controlling animal pests, which include arthropods and especially insects, compositions containing such compounds and methods for the control of nematodes, helminths and arthropods.

Nematodes cause a substantial loss in agricultural product including food and industrial crops and are combated with chemical compounds having nematicidal activity. To be useful in agriculture these compounds should have a high activity, a broad spectrum activity against different strains of nematodes and should not be toxic to non-target organisms.

The occurrence of resistances against commercially available anthelmintics seems to be a growing problem in the area of veterinary medicine. Therefore, endoparasiticides with new molecular modes of action are urgently desired. The new active ingredients should perform with excellent efficacy against a broad spectrum of helminths and nematodes without any adverse toxic effects to the treated animal. Endoparasiticides are pharmaceuticals (medicaments) for combat or suppression of endoparasites in animals or humans.

In JP2012201640, certain fungicidal benzene-2-carboxamide derivatives are generically embraced in a broad disclosure of numerous compounds of the following formula:

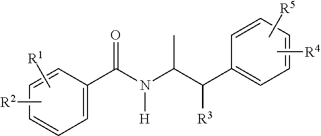

wherein R³ can represent an hydroxyl or an alkoxy group. However, this document is silent about a nematicidal efficacy of the disclosed compounds.

In WO 2011/128989 A1, certain fungicidal pyrazine-2-carboxamide derivatives are generically embraced in a broad disclosure of numerous compounds of the following formula:

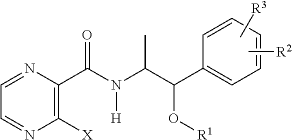

wherein X can represent a halogen atom or a trifluoromethyl group and R¹ can represent an hydrogen atom or an C₁-C₄ alkyl group. However, this document is silent about a nematicidal efficacy of the disclosed compounds.

In WO 2008/148570 A1, certain fungicidal pyrazole carboxylic acid amides are generically embraced in a broad disclosure of numerous compounds of the following formula:

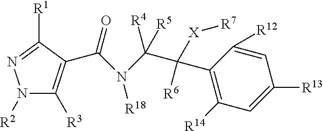

wherein X can represent an oxygen atom, R⁷ can be a C₁-C₈-alkyl, C₂-C₈-alkenyl, C₂-C₈-alkynyl, C₃-C₆-cycloalkyl. However, this document does not disclose compounds with nematicidal activity.

In WO 2012/118139 A1, certain parasiticides are generically embraced in a broad disclosure of numerous compounds of the following formula:

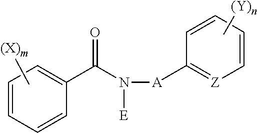

wherein A can represent a C₁-C₈ alkylene chain, possibly interrupted by an heteroatom, possibly substituted by a halogen atom, a C₁-C₆ alkyl or C₃-C₆ cycloalkyl, Z can be a carbon atom. However, the document does not disclose compounds which are encompassed by the present invention.

In WO2011/151369 A1 certain [(het)arylethyl)]pyrazolecarboxamides and thio-carboxamides are generically embraced in a broad disclosure of numerous compounds of the following formula:

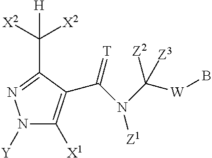

wherein B can represent a substituted or unsubstituted ring and W can, amongst other groups, represent CZ⁴Z⁵, in particular Z⁴ or Z⁵ can represent an alkoxy group or the like. However, this document does not disclose the use of the disclosed compounds against nematodes.

In PCT/EP2012/073431 certain 2-alkoxy-phenethylcarboxamides and their use against endoparasites in animals and humans are disclosed. Nothing is said about any efficacy of the compounds against plant damaging nematodes.

Modern crop protection compositions have to meet many demands, for example in relation to the level, duration and breadth of their action and possible use. Questions of toxicity and of combinability with other active ingredients or formulation auxiliaries play a role, as does the question of the expense that the synthesis of an active ingredient requires. In addition, resistances can occur. For all these reasons, the search for novel crop protection compositions cannot be considered to be complete, and there is a constant need for novel compounds having properties which, compared to the known compounds, are improved at least in relation to individual aspects.

It was an object of the present invention to provide compounds which widen the spectrum of the pesticides in various respects.

This object, and further objects which are not stated explicitly but can be discerned or derived from the connections discussed herein, are achieved by the use of compounds of formula (I)

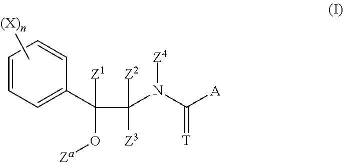

wherein
A represents phenyl of formula A^{a}

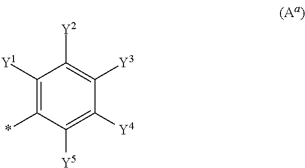

wherein
* indicates the bond which connects A^{a} to the C=T moiety of the compounds of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group optionally substituted by 1 to 5 groups R, wherein
R independently from each other R represents hydrogen, halogen, nitro, cyano, hydroxy, amino, sulfanyl, pentafluoro-λ⁶-sulfanyl, substituted or unsubstituted (C₁-C₈-alkoxyimino)-C₁-C₈-alkyl, substituted or unsubstituted (benzyloxyimino)-C₁-C₈-alkyl, substituted or unsubstituted C₁-C₈-alkyl, C₁-C₈-halogenoalkyl having 1 to 5 halogen atoms, substituted or unsubstituted C₂-C₈-alkenyl, C₂-C₈-halogenoalkenyl having 1 to 5 halogen atoms, substituted or unsubstituted C₂-C₈-alkynyl, C₂-C₈-halogenoalkynyl having 1 to 5 halogen atoms, substituted or unsubstituted C₁-C₈-alkoxy, C₁-C₈-halogenoalkoxy having 1 to 5 halogen atoms, substituted or unsubstituted C₁-C₈-alkylsulfanyl, C₁-C₈-halogenoalkylsulfanyl having 1 to 5 halogen atoms, substituted or unsubstituted C₁-C₈-alkylsulfinyl, C₁-C₈-halogenoalkylsulfinyl having 1 to 5 halogen atoms, substituted or unsubstituted C₁-C₈-alkylsulfonyl, C₁-C₈-halogenoalkylsulfonyl having 1 to 5 halogen atoms, substituted or unsubstituted C₁-C₈-alkylamino, substituted or unsubstituted di-C₁-C₈-alkylamino, substituted or unsubstituted C₂-C₈-alkenyloxy, substituted or unsubstituted C₃-C₈-alkynyloxy, substituted or unsubstituted C₃-C₇-cycloalkyl, C₃-C₇-halogenocycloalkyl having 1 to 5 halogen atoms, substituted or unsubstituted tri-(C₁-C₈-alkyl)-silyl, substituted or unsubstituted C₁-C₈-alkylcarbonyl, C₁-C₈-halogenoalkylcarbonyl having 1 to 5 halogen atoms, substituted or unsubstituted C₁-C₈-alkoxycarbonyl, C₁-C₈-halogenoalkoxycarbonyl having 1 to 5 halogen atoms, substituted or unsubstituted C₁-C₈-alkylcarbamoyl, substituted or unsubstituted di-(C₁-C₈)-alkylcarbamoyl, phenoxy, phenylsulfanyl, phenylamino, benzyloxy, benzylsulfanyl, or benzylamino,
T represents oxygen or sulphur,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkoxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to 9 halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ represents hydrogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl,
Z² and Z³ independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Z⁴ represents hydrogen, cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Z^{a} represents substituted or unsubstituted C₁-C₈-alkyl, substituted or unsubstituted C₃-C₈-alkenyl, substituted or unsubstituted C₃-C₈-alkynyl, substituted or unsubstituted C₃-C₇-cycloalkyl, substituted or unsubstituted C₁-C₆-alkylideneamino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
and N-oxides, metallic complexes, metalloidic complexes and optically active isomers of the compounds of formula (I),
for controlling nematodes which damage plants.

In the above definitions, unless stated otherwise,

halogen means fluorine, bromine, chlorine, iodine,
carboxy means —C(═O)OH,
carbonyl means —C(═O)—,
carbamoyl means —C(═O)NH₂,
alkylcarbamoyl means —C(═O)NHalkyl
dialkylcarbamoyl means —C(═O)N(alkyl)₂
N-hydroxycarbamoyl means —C(═O)NHOH,
SO represents a sulfoxyde group,
SO₂ represents a sulfone group,
an alkyl group, an alkenyl group and an alkynyl group as well as moieties containing these terms, can be linear or branched.

The term “aryl”, also in terms like arylalkyl, arylalkenyl, arylalkynyl, aryloxy means phenyl or naphthyl, wherein phenyl is optionally substituted by 1 to 5 groups Q, and naphtyl is optionally substituted by 1 to 6 groups Q.

The term “heterocyclyl” means a saturated or unsaturated 4-, 5-, 6-, 7-, 8-, 9-, or 10-membered ring comprising 1 to 4 heteroatoms selected from the list consisting of oxygen (O), nitrogen (N), and sulphur (S).

Heteroatom means an atom selected from the group consisting of O, N, and S.

Unless indicated otherwise, if more than one halogen atom is present in a halogenated radical, like e.g. halogenoalkyl, halogenoalkoxy, those halogen atoms can be the same or different.

Unless indicated otherwise, a group or a substituent which is substituted according to the invention can be substituted by one or more of the following groups or atoms: halogen, nitro, hydroxy, cyano, amino, sulfanyl, pentafluoro-λ⁶-sulfanyl, formyl, formyloxy, formylamino, carbamoyl, N-hydroxycarbamoyl, carbamate, hydroxyimino-C₁-C₆-alkyl, C₁-C₈-alkyl, tri-(C₁-C₈-alkyl)-silyl-C₁-C₈-alkyl, C₁-C₈-cycloalkyl, tri-(C₁-C₈-alkyl)-silyl-C₁-C₈-cycloalkyl, C₁-C₈-halogenoalkyl having 1 to 5 halogen atoms, C₃-C₈-halogenocycloalkyl having 1 to 5 halogen atoms, C₂-C₈-alkenyl, C₂-C₈-alkynyl, C₂-C₈-alkenyloxy, C₃-C₈-alkynyloxy, C₁-C₈-alkylamino, di-(C₁-C₈-alkyl)-amino, C₁-C₈-alkoxy, C₁-C₈-halogenoalkoxy having 1 to 5 halogen atoms, C₁-C₈-alkylsulfanyl, C₁-C₈-halogenoalkylsulfanyl having 1 to 5 halogen atoms, C₂-C₈-alkenyloxy, C₂-C₈-halogenoalkenyloxy having 1 to 5 halogen atoms, C₃-C₈-alkynyloxy, C₃-C₈-halogenoalkynyloxy having 1 to 5 halogen atoms, C₁-C₈-alkylcarbonyl, C₁-C₈-halogenoalkylcarbonyl having 1 to 5 halogen atoms, C₁-C₈-alkylcarbamoyl, di-(C₁-C₈-alkyl)-carbamoyl, N—C₁-C₈-alkyloxycarbamoyl, C₁-C₈-alkoxycarbamoyl, N—C₁-C₈-alkyl-C₁-C₈-alkoxycarbamoyl, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylcarbonylamino, C₁-C₈-halogenoalkylcarbonylamino having 1 to 5 halogen atoms, C₁-C₈-alkylaminocarbonyloxy, di-(C₁-C₈-alkyl)-aminocarbonyloxy, C₁-C₈-alkyloxycarbonyloxy, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylaminosulfamoyl, di-(C₁-C₈-alkyl)-aminosulfamoyl, C₁-C₆-alkoxyimino-C₁-C₆-alkyl, C₂-C₆-alkenyloxyimino-C₁-C₆-alkyl, C₃-C₆-alkynyloxyimino-C₁-C₆-alkyl, 2-oxopyrrolidin-1-yl, benzyloxyimino-C₁-C₆-alkyl, C₁-C₈-alkoxy-C₁-C₈-alkyl, C₁-C₈-halogenoalkoxy-C₁-C₈-alkyl having 1 to 5 halogen atoms, benzyloxy, benzylsulfanyl, benzylamino, phenoxy, phenylsulfanyl, or phenylamino.

Compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions) and to the mixtures of all the possible stereoisomers, in all proportions. The diastereoisomers and/or the optical isomers can be separated according to the methods which are known per se by the man ordinary skilled in the art.

Compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Compounds of the present invention can also exist in one or more geometric isomer forms depending on the relative position (syn/anti or cis/trans) of the substituents of ring B. The invention thus relates equally to all syn/anti (or cis/trans) isomers and to all possible syn/anti (or cis/trans) mixtures, in all proportions. The syn/anti (or cis/trans) isomers can be separated according to general methods, which are known per se by the man ordinary skilled in the art.

Any of the compounds of formula (I) wherein X represents a hydroxy, a sulfanyl or an amino may be found in its tautomeric form resulting from the shift of the proton of said hydroxy, sulfanyl or amino group. Such tautomeric forms of such compounds are also part of the present invention. More generally speaking, all tautomeric forms of compounds of formula (I) wherein X represents a hydroxy, a sulfanyl group or an amino group, as well as the tautomeric forms of the compounds which can optionally be used as intermediates in the preparation processes and which will be defined in the description of these processes, are also part of the present invention.

A preferred embodiment of the invention is the use of compounds of formula (I) wherein A is a group of formula A^{a} as defined above.

Another preferred embodiment of the invention is the use of compounds of formula (I) wherein A is a heterocycle of formula (A¹)

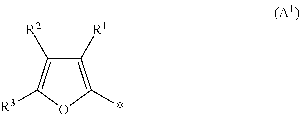

wherein
R¹ to R³ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms.

Another preferred embodiment of the invention is the use of compounds of formula (I) wherein A is a heterocycle of formula (A²)

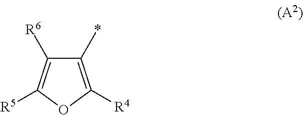

wherein
R⁴ to R⁶ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms.

Another preferred embodiment of the invention is the use of compounds of formula (I) wherein A is a heterocycle of formula (A³)

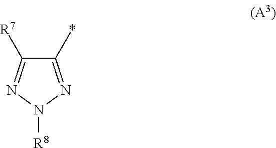

wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl.

Another preferred embodiment of the invention is the use of compounds of formula (I) wherein A is a heterocycle of formula (A⁴)

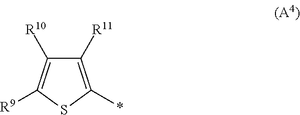

wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms.

Another preferred embodiment of the invention is the use of compounds of formula (I) wherein A is a heterocycle of formula (A⁵)

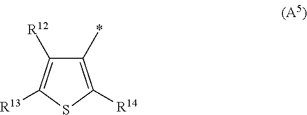

wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms.

Another preferred embodiment of the invention is the use of compounds of formula (I) wherein A is a heterocycle of formula (A⁶)

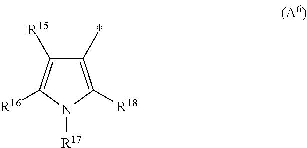

wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl.

Another preferred embodiment of the invention is the use of compounds of formula (I) wherein A is a heterocycle of formula (A⁷)

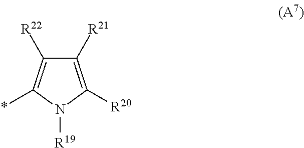

wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A⁸)

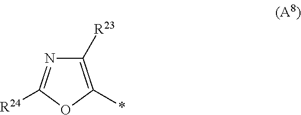

wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A⁹)

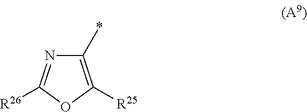

wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A¹⁰)

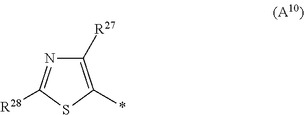

wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino.

Another preferred embodiment of the invention is the use of compounds of formula (I) wherein A is a heterocycle of formula (A¹¹)

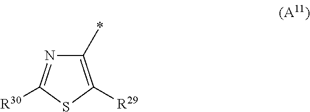

wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino.

Another preferred embodiment of the invention is the use of compounds of formula (I) wherein A is a heterocycle of formula (A¹²)

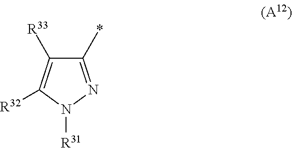

wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) wherein A is a heterocycle of formula (A¹³)

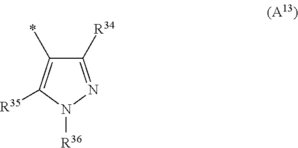

wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl.

Another preferred embodiment of the invention is the use of compounds of formula (I) wherein A is a heterocycle of formula (A¹⁴)

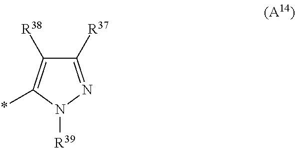

wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl.

Another preferred embodiment of the invention is the use of compounds of formula (I) wherein A is a heterocycle of formula (A¹⁵)

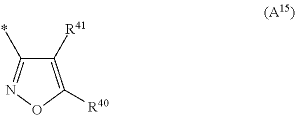

wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A¹⁶)

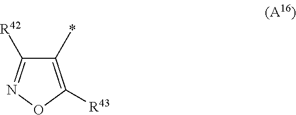

wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino.

Another preferred embodiment of the invention is the use of compounds of formula (I) wherein A is a heterocycle of formula (A¹⁷)

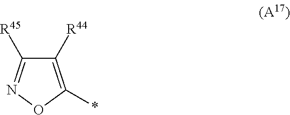

wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A¹⁸)

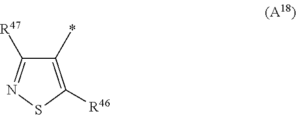

wherein
R⁴⁷ represents hydrogen, halogen, substituted or unsubstituted C₁-C₅-alkyl or C₁-C₅-halogenoalkyl comprising 1 to 9 halogen atoms, and
R⁴⁶ represents hydrogen, halogen, substituted or unsubstituted C₁-C₅-alkyl, C₁-C₅-halogenoalkyl comprising 1 to 9 halogen atoms or substituted or unsubstituted C₁-C₅-alkylsulfanyl.

Another preferred embodiment of the invention is the use of compounds of formula (I) wherein A is a heterocycle of formula (A¹⁹)

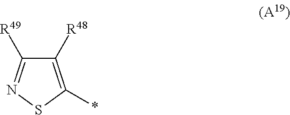

wherein
R⁴⁹ and R⁴⁸ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) wherein A is a heterocycle of formula (A²⁰)

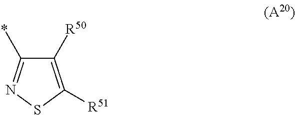

wherein
R⁵⁰ and R⁵¹ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

Another preferred embodiment of the invention is the use of compounds of formula (I) wherein A is a heterocycle of formula (A²¹)

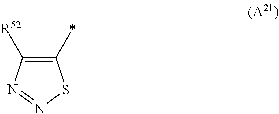

wherein
R⁵² represents hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A²²)

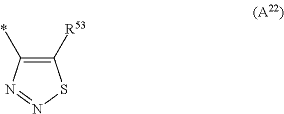

wherein
R⁵³ represents hydrogen, halogen, substituted or unsubstituted C₁-C₅-alkyl or C₁-C₅-halogenoalkyl comprising 1 to 9 halogen atoms.

Another preferred embodiment of the invention is the use of compounds of formula (I) wherein A is a heterocycle of formula (A²³)

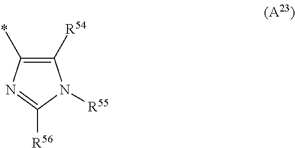

wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl.

Another preferred embodiment of the invention is the use of compounds of formula (I) wherein A is a heterocycle of formula (A²⁴)

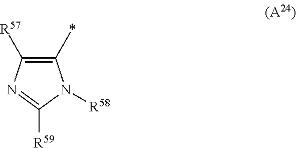

wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl.

Another preferred embodiment of the invention is the use of compounds of formula (I) wherein A is a heterocycle of formula (A²⁵)

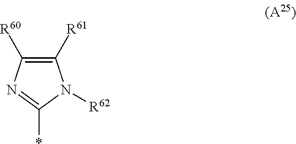

wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl.

Another preferred embodiment of the invention is the use of compounds of formula (I) wherein A is a heterocycle of formula (A²⁶)

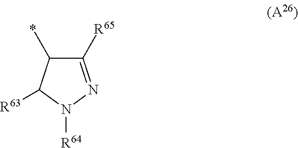

wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms.

Another preferred embodiment of the invention is the use of compounds of formula (I) wherein A is a heterocycle of formula (A²⁷)

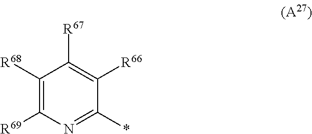

wherein
R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, SC₁-C₄-alkylsulfinyl and C₁-C₄-alkylsulfonyl.

Another preferred embodiment of the invention is the use of compounds of formula (I) wherein A is a heterocycle of formula (A²⁸)

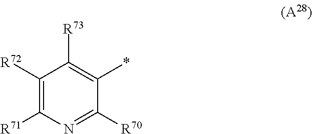

wherein
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl, C₁-C₄-alkylsulfonyl, N-morpholine optionally substituted by halogen or C₁-C₄-alkyl, and thienyl (optionally substituted by halogen or C₁-C₄-alkyl).

Another preferred embodiment of the invention is the use of compounds of formula (I) wherein A is a heterocycle of formula (A²⁹)

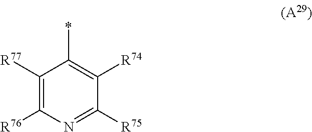

wherein
R⁷⁴, R⁷⁵, R⁷⁶ and R⁷⁷ independently from each other represent hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms, C₁-C₄-halogenoalkoxy having 1 to 5 halogen atoms, C₁-C₄-alkylsulfinyl and —C₄-alkylsulfonyl. Another preferred embodiment of the invention is the use of compounds of formula (I) wherein A is a heterocycle of formula (A³⁰)

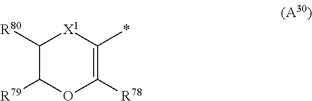

wherein
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl.

Another preferred embodiment of the invention is the use of compounds of formula (I) wherein A is a heterocycle of formula (A³¹)

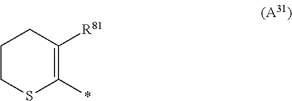

wherein
R⁸¹ represents C₁-C₄-alkyl and C₁-C₄-halogenoalkyl having 1 to 5 halogen atoms.

Another preferred embodiment of the invention is the use of compounds of formula (I) wherein A is a heterocycle of formula (A³²)

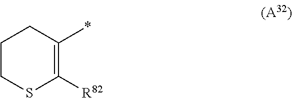

wherein
R⁸² represents C₁-C₄-alkyl and C₁-C₄-halogenoalkyl having 1 to 5 halogen atoms.

Another preferred embodiment of the invention is the use of compounds of formula (I) wherein A is a heterocycle of formula (A³³)

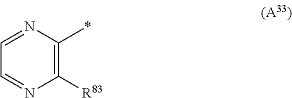

wherein
R⁸³ represents hydrogen, halogen, C₁-C₄-alkyl and C₁-C₄-halogenoalkyl having 1 to 5 halogen atoms.

The * in formulae A¹ to A³³ indicates the bond which connects A¹ to A³³ to the C=T moiety of the compounds of formula (I).

Finally, it has been found that the compounds of the formula (I) have very pronounced biological properties and are suitable in particular for controlling plant damaging nematodes, which are encountered predominantly in agriculture.

Preferred substituents or ranges for the radicals shown in the compounds of the formula (I) are elucidated below, their combination represents a preferred group (W) of compounds of formula (I).

A represents A^{a}.

Y¹ represents halogen, nitro, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₁-C₈-alkylsulfanyl, C₁-C₈-halogenoalkylsulfanyl having 1 to 5 halogen atoms, C₁-C₈-alkoxy, C₁-C₈-halogenoalkoxy having 1 to 5 halogen atoms.

Y², Y³, Y⁴ and Y⁵ independently from each other represent hydrogen, halogen, C₁-C₈-alkyl, C₁-C₈-halogenoalkyl having 1 to 5 halogen atoms.

A also represents A⁴, wherein

R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulfanyl,
C₁-C₅-halogenoalkyl comprising 1 to 9 halogen atoms or C₁-C₅-halogenoalkoxy comprising 1 to 9 halogen atoms.

A also represents A⁵, wherein

R¹² and R¹³ independently from each other represent hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms.

A also represents A¹², wherein

R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms.

A also represents A¹³, wherein

R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di(C₁-C₅-alkyl)amino, and
R³⁶ represents a hydrogen atom or substituted or unsubstituted C₁-C₅-alkyl.

A also represents A¹⁴, wherein

R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl.

A also represents A²⁷, in which

R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfanyl.

A also represents A²⁸, in which

R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkysulfanyl, C₂-C₅-alkenysulfanyll, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfonyl,

A also represents A³³, in which

R⁸³ represents hydrogen, halogen, C₁-C₄-alkyl and C₁-C₄-halogenoalkyl having 1 to 5 halogen atoms.

T represents oxygen.

n represents 1, 2 or 3.

X independently from each other X represents halogen, C₁-C₈-alkyl, C₁-C₈-halogenoalkyl having 1 to 9 halogen atoms, C₁-C₈-alkoxy, C₁-C₈-halogenoalkoxy having 1 to 9 halogen atoms, aryl optionally substituted by 1 to 6 groups Q which can be the same or different, aryloxy optionally substituted by 1 to 6 groups Q which can be the same or different, pyridinyloxy which is optionally substituted by 1 to 4 groups Q.

Q independently from each other Q represents represents halogen, methyl, difluoromethyl, trifluoromethyl, methoxy and trifluoromethoxy.

Z¹ represents hydrogen, substituted or unsubstituted C₁-C₈-alkyl, substituted or unsubstituted C₁-C₈-alkoxy, and preferably represents hydrogen.

Z² and Z³ independently represent hydrogen, fluorine or substituted or unsubstituted C₁-C₈-alkyl, and preferably represent hydrogen, fluorine, methyl and ethyl.

Z² and Z³ can also form together with the carbon atom to which they are attached a cyclopropyl ring.

Z⁴ represents hydrogen, methyl, cyano, cyclopropyl.

Z^{a} represents substituted or unsubstituted C₁-C₈-alkyl, substituted or unsubstituted C₃-C₇-cycloalkyl, substituted or non-substituted C₁-C₆-alkylideneamino, and preferably represents methyl, ethyl, n-propyl and i-propyl.

A preferred embodiment within group (W) are compounds of formula (I) in which A represents A^{a}.

Another preferred embodiment within group (W) are compounds of formula (I) in which A represents A⁴.

Another preferred embodiment within group (W) are compounds of formula (I) in which A represents A⁵.

Another preferred embodiment within group (W) are compounds of formula (I) in which A represents A¹².

Another preferred embodiment within group (W) are compounds of formula (I) in which A represents A¹³.

Another preferred embodiment within group (W) are compounds of formula (I) in which A represents A¹⁴.

Another preferred embodiment within group (W) are compounds of formula (I) in which A represents A²⁷.

Another preferred embodiment within group (W) are compounds of formula (I) in which A represents A²⁸.

Another preferred embodiment within group (W) are compounds of formula (I) in which A represents A³³.

Further preferred embodiments within group (W) are groups (W1) to (W6).

(W1) Compounds of formula (I) wherein A represents A^{a} in which Y¹ represents a radical selected from the group consisting of halogen, methyl, difluoromethyl, trifluoromethyl, methoxy, difluoromethoxy and trifluoromethoxy, Y², Y³ and Y⁴ represent hydrogen and Y⁵ represents hydrogen or fluorine.

(W2) Compounds of formula (I) wherein A represents A⁴ in which wherein R⁹ and R¹⁰ represent hydrogen and R¹¹ represents halogen, methyl, C₁-fluorooalkyl comprising 1 to 3 fluorine atoms.

(W3) Compounds of formula (I) wherein A represents A⁵ in which R¹² and R¹⁴ independently from each other represent hydrogen, halogen, methyl, methoxy, C₁-fluorooalkyl comprising 1 to 3 fluorine atoms that can be the same or different, and

R¹³ represents hydrogen.

(W4) Compounds of formula (I) wherein A represents A¹³ in which R³⁴ represents hydrogen, halogen, C₁-C₂-alkyl, cyclopropyl, C₁-C₂-halogenoalkyl comprising 1 to 9 halogen atoms, C₁-C₂-alkoxy, or C₁-C₂-halogenoalkoxy comprising 1 to 5 halogen atoms, and

R³⁵ represents hydrogen, halogen, methyl, ethyl, C₁-halogenoalkyl comprising 1 to 3 halogen atoms, C₁-halogenoalkoxy comprising 1 to 3 halogen atoms, and
R³⁶ represents methyl.

(W5) Compounds of formula (I) wherein A represents A²⁸ in which R⁷⁰ represents hydrogen, halogen, cyano, methyl, ethyl, difluoromethyl, trifluoromethyl, methoxy, trifluromethylsulfanyl, trifluoromethoxy, and

R⁷¹, R⁷² represent hydrogen and R⁷³ represents hydrogen, halogen, cyano, methyl, ethyl, difluoromethyl, trifluoromethyl, methoxy, trifluromethylsulfanyl, trifluoromethoxy.

(W6) Compounds of formula (I) wherein A represents A³³ in which R⁸³ represents hydrogen, halogen, methyl, ethyl, difluoromethyl and trifluoromethyl.

Particularly preferred substituents or ranges for the radicals shown in the compounds of the formula (I) are elucidated below, their combination represents a particularly preferred group (U) of compounds of formula (I).

A represents A^{a}.

Y¹ represents a radical selected from the group consisting of halogen, methyl, difluoromethyl, trifluoromethyl, methoxy, difluoromethoxy and trifluoromethoxy.

Y⁵ represents hydrogen or fluorine.

A also represents A⁴, wherein

R⁹ to R¹¹ independently from each other represent hydrogen, halogen, substituted or unsubstituted C₁-C₅-alkyl, substituted or unsubstituted C₁-C₅-alkoxy, substituted or unsubstituted C₁-C₅-alkylsulfanyl, C₁-C₅-halogenoalkyl comprising 1 to 9 halogen atoms or C₁-C₅-halogenoalkoxy comprising 1 to 9 halogen atoms.

A also represents A⁵, wherein

R¹² and R¹³ independently from each other represent hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, C₁-C₅-halogenoalkyl comprising 1 to 9 halogen atoms or C₁-C₅-halogenoalkoxy comprising 1 to 9 halogen atoms.

A also represents A¹³, wherein

R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₂-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di(C₁-C₅-alkyl)amino, and
R³⁶ represents a hydrogen atom or substituted or unsubstituted C₁-C₅-alkyl.

A also represents A²⁸, in which

R⁷⁰ represents hydrogen, halogen, hydroxy, cyano, C₁-C₄-alkyl, C₁-C₄-halogenoalkyl having 1 to5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl and C₁-C₄-alkylsulfonyl.

A also represents A³³, in which

R⁸³ represents hydrogen, halogen, C₁-C₄-alkyl and C₁-C₄-halogenoalkyl having 1 to 5 halogen atoms.

T represents oxygen.

n represents 1, 2 or 3.

X independently from each other X represents halogen, C₁-C₃-alkyl, C₁-C₂-halogenoalkyl having 1 to 2 halogen atoms, C₁-C₄-alkoxy, C₁-C₂-halogenoalkoxy having 1 to 5 halogen atoms.

Z¹ represents hydrogen,

Z² and Z³ independently represent hydrogen, methyl or ethyl.

Z² and Z³ can also form together with the carbon atom to which they are attached a cyclopropyl ring.

Z⁴ represents hydrogen. Z^{a} represents methyl, ethyl, n-propyl or i-propyl.

Preferred embodiments within group (U) are groups (U1) to (U6).

(U1) Compounds of formula (I) wherein A represents A^{a} in which Y¹ represents a radical selected from the group consisting of halogen, methyl, difluoromethyl, trifluoromethyl, methoxy, difluoromethoxy and trifluoromethoxy, Y², Y³ and Y⁴ represent hydrogen and Y⁵ represents hydrogen or fluorine.

(U2) Compounds of formula (I) wherein A represents A⁴ in which R⁹ and R¹⁰ represent hydrogen and R¹¹ represents halogen, methyl, C₁-fluoroalkyl comprising 1 to 3 fluorine atoms.

(U3) Compounds of formula (I) wherein A represents A⁵ in which R¹² and R¹⁴ independently from each other represent hydrogen, halogen, methyl, methoxy, C₁-fluoroalkyl comprising 1 to 3 fluorine atoms that can be the same or different, and

R¹³ represents hydrogen.

(U4) Compounds of formula (I) wherein A represents A¹³ in which R³⁴ represents hydrogen, halogen, C₁-C₂-alkyl, cyclopropyl, C₁-C₂-halogenoalkyl comprising 1 to 5 halogen atoms that can be the same or different, C₁-C₂-alkoxy, or C₁-C₂-halogenoalkoxy comprising 1 to 5 halogen atoms that can be the same or different, and

R³⁵ represents hydrogen, halogen, methyl, ethyl, C₁-halogenoalkyl comprising 1 to 3 halogen atoms, C₁-halogenoalkoxy comprising 1 to 3 halogen atoms, and
R³⁶ represents methyl.

(U5) Compounds of formula (I) wherein A represents A²⁸ in which R⁷⁰ represents hydrogen, halogen, cyano, methyl, ethyl, difluoromethyl, trifluoromethyl, methoxy, trifluromethylsulfanyl, trifluoromethoxy, and

R⁷¹, R⁷² represent hydrogen and R⁷³ represents hydrogen, halogen, cyano, methyl, ethyl, difluoromethyl, trifluoromethyl, methoxy, trifluromethylsulfanyl, trifluoromethoxy.

(U6) Compounds of formula (I) wherein A represents A³³ in which R⁸³ represents hydrogen, halogen, methyl, ethyl, difluoromethyl and trifluoromethyl.

The above mentioned preferences with regard to the substituents of the compounds according to the invention can be combined in various manners. These combinations of preferred features thus provide sub-classes of compounds according to the invention.

The present invention is also directed to novel compounds of formula (I-1)

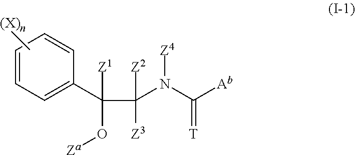

in which T, Z¹, Z^{a}, X and n have the meanings mentioned above for the compounds of formula (I),
Z² and Z³ independently represent halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Q being as defined above,
Z⁴ represents a hydrogen atom and
A^{b} represents A³ to A¹⁷, A¹¹ to A²⁶ and A³⁰ to A³³ as defined above, provided that when A^{b} represents A¹³, R³⁴ does not represent a halogenomethyl group.

Preferred are compounds of formula (I-1) in which Z² and Z³ represent methyl or together with the carbon atom to which they are attached represent cyclopropyl.

The preferred and particularly preferred definitions of T, Z¹, Z^{a}, X, Q, n, A³ to A¹⁷, A¹¹ to A²⁶ and A³⁰ to A³³ for the compounds of formula (I) also apply with regard to the compounds of formula (I-1).

The present invention is further directed to novel compounds of formula (I-2)

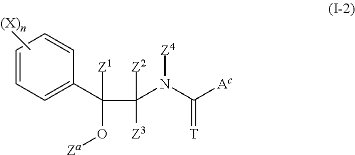

in which T, Z¹, Z², Z³, Z^{a}, X and n have the meanings mentioned above for the compounds of formula (I),
Z⁴ represents cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino, and
A^{c} represents A³ to A¹⁷, A¹¹ to A²⁶ and A³⁰ to A³³ as defined above, provided that when A^{c} represents A¹³, R³⁴ does not represent a halogenomethyl group.

Preferred are compounds of formula (I-2) in which Z⁴ represents cyclopropyl.

The preferred and particularly preferred definitions of T, Z¹, Z², Z³, Z^{a}, X, n, A³ to A¹⁷, A¹¹ to A²⁶ and A³⁰ to A³³ for the compounds of formula (I) also apply with regard to the compounds of formula (I-1).

The present invention also relates to a process for the preparation of the compounds of formula (I-1).

Thus, according to a further aspect of the present invention there is provided a process P1 for the preparation of a compound of formula (I-1) as herein-defined and wherein T represents O and that comprises reaction of an amine of formula (II-1) or one of its salts

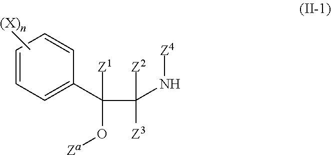

in which T, Z¹, Z^{a}, X and n have the meanings mentioned above for the compounds of formula (I),
Z² and Z³ independently represent halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Q being as defined above,
Z⁴ represents a hydrogen atom, with a carboxylic acid derivative of formula (III-1):

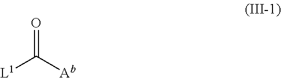

wherein
A^{b} has the meaning given above and L¹ represents a leaving group selected in the list consisting of a halogen atom, a hydroxyl group, —OR^{a}, —OC(═O)R^{a}, R^{a} being a substituted or non-substituted C₁-C₆-alkyl, a substituted or non-substituted C₁-C₆-haloalkyl, a benzyl, 4-methoxybenzyl or pentafluorophenyl group, or a group of formula O—C(═O)A^{b}; in the presence of a catalyst and in the presence of a condensing agent in case L¹ represents a hydroxyl group, and in the presence of an acid binder in case L¹ represents a halogen atom.

Amine derivatives of formula (II-1) are known or can be prepared by known processes such as reductive amination of aldehydes or ketones (Organic Reactions (Hoboken, N.J., United States) (2002), 59,), or reduction of oximesnitro and azido groups, or nucleophilic substitution of a halogen, mesylate or tosylate (Journal of Medicinal Chemistry (2002), 45, 3887).

Carboxylic acid derivatives of formula (III-1) are known or can be prepared by known processes.

In case L¹ represents a hydroxy group, process P1 according to the present invention is conducted in the presence of condensing agent. Suitable condensing agent may be selected in the non-limited list consisting of acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloro-methane, 4-(4,6-dimethoxy[1.3.5]-triazin-2-yl)-4-methylmorpholinium chloride hydrate, bromotripyrrolidinophosphonium-hexafluorophosphate or propanephosphonic anhydride (T3P).

Process P1 according to the present invention is conducted in the presence of a catalyst. Suitable catalyst may be selected in the list consisting of N,N-dimethylpyridin-4-amine, 1-hydroxy-benzotriazole or N,N-dimethylformamide.

In case L¹ represents a halogen atom, process P1 according to the present invention is conducted in the presence of an acid binder. Suitable acid binders for carrying out process P1 according to the invention are in each case all inorganic and organic bases that are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as caesium carbonate, sodium carbonate, potassium carbonate, potassium bicarbonate, sodium bicarbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylpyridin-4-amine, diazabicyclooctane (DABCO), diazabicyclo-nonene (DBN) or diazabicycloundecene (DBU).

It is also possible to work in the absence of an additional condensing agent or to employ an excess of the amine component, so that it simultaneously acts as acid binder agent.

Suitable solvents for carrying out process P1 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone, or hexamethylphosphoric triamide; alcohols such as methanol, ethanol, propanol, iso-propanol; esters, such as methyl acetate or ethyl acetate, sulfoxides, such as dimethyl sulfoxide, or sulfones, such as sulfolane.

When carrying out process P1 according to the invention, the amine derivative of formula (II) can be employed as its salt, such as chlorhydrate or any other convenient salt.

When carrying out process P1 according to the invention, 1 mole or an excess of the amine derivative of formula (II) and from 1 to 3 moles of the acid binder can be employed per mole of the reagent of formula (III).

It is also possible to employ the reaction components in other ratios. Work-up is carried out by known methods.

According to a further aspect according to the invention, there is provided a second process P2 for the preparation of a compound of formula (I-1) wherein T represents S, starting from a compound of formula (I-1) wherein T represents 0 according to the following reaction scheme:

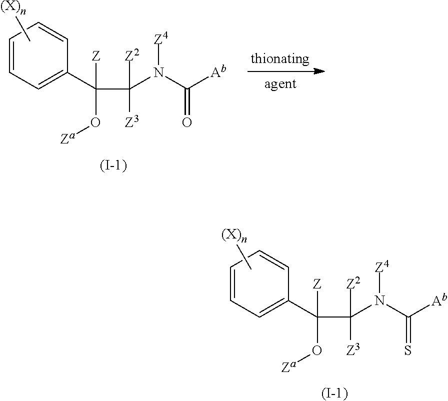

Process P2

in which Z¹, Z^{a}, X and n have the meanings mentioned above for the compounds of formula (I),
Z² and Z³ independently represent halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Q being as defined above,
Z⁴ represents a hydrogen atom and
A^{b} has the meaning given above.

Process P2 according to the invention is performed in the presence of a thionating agent.

Amide derivatives of formula (I-1) wherein T represents O can be prepared according to process P1.

Suitable thionating agents for carrying out process P2 according to the invention can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in Journal of the Chemical Society, Perkin 1 (2001), 358, in the optionally presence of a catalytic or stoichiometric or excess amount, quantity of a base such as an inorganic and organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethylpyridin-4-amine or N-methyl-piperidine.

Suitable solvents for carrying out process P2 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin, chlorobenzene, dichlorobenzene, dichloromethane, chloroform, carbon tetrachloride, dichlorethane or trichlorethane, ethers, such as diethyl ether, diisopropyl ether, methyl t-butyl ether, methyl t-amyl ether, dioxane, tetrahydrofuran, 1,2-dimethoxyethane or 1,2-diethoxyethane, nitriles, such as acetonitrile, propionitrile, n- or i-butyronitrile or benzonitrile, sulfurous solvents, such as sulfolane or carbon disulfide.

When carrying out process P2 according to the invention, 1 mole or an excess of the sulfur equivalent of the thionating agent and from 1 to 3 moles of the base can be employed per mole of the amide reactant (I).

It is also possible to employ the reaction components in other ratios. Work-up is carried out by known methods.

Processes P1 and P2 according to the invention are generally carried out under atmospheric pressure. It is also possible to operate under elevated or reduced pressure.

When carrying out processes P1 and P2 according to the invention, the reaction temperatures can be varied within a relatively wide range. In general, these processes are carried out at temperatures from 0° C. to 200° C., preferably from 10° C. to 160° C. A way to control the temperature for the processes according to the invention is to use micro-wave technology.

In general, the reaction mixture is concentrated under reduced pressure. The residue that remains can be freed by known methods, such as chromatography or crystallization, from any impurities that can still be present.

The present invention also relates to a process for the preparation of the compounds of formula (I-2).

Thus, according to a further aspect of the present invention there is provided a process P′1 for the preparation of a compound of formula (I-2) as herein-defined and wherein T represents 0 and that comprises reaction of an amine of formula (II-2) or one of its salts:

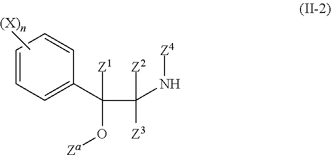

in which Z¹, Z², Z³, Z^{a}, X and n have the meanings mentioned above for the compounds of formula (I),
Z⁴ represents cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino, and
with a carboxylic acid derivative of formula (III-2):

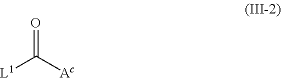

wherein
A^{c} represents A³ to A¹⁷, A¹¹ to A²⁶ and A³⁰ to A³³ as defined above, provided that when A^{c} represents A¹³, R³⁴ does not represent a halogenomethyl group, and
L¹ represents a leaving group selected from the list consisting of halogen, OH, —OR^{a}, —OC(═O)R^{a}, R^{a} being a substituted or non-substituted C₁-C₆-alkyl, a substituted or non-substituted C₁-C₆-haloalkyl, a benzyl, 4-methoxybenzyl or pentafluorophenyl group, or a group of formula O—C(═O)A^{c}; in the presence of a catalyst and in the presence of a condensing agent in case L¹ represents a hydroxyl group, and in the presence of an acid binder in case L¹ represents a halogen atom.

Amine derivatives of formula (II-2) are known or can be prepared by known processes.

Carboxylic acid derivatives of formula (III-2) are known or can be prepared by known processes.

In case L¹ represents OH, process P′1 according to the present invention is conducted in the presence of condensing agent. Suitable condensing agent may be selected in the non-limited list consisting of acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloro-methane, 4-(4,6-dimethoxy[1.3.5]-triazin-2-yl)-4-methylmorpholinium chloride hydrate, bromo-tripyrrolidinophosphoniumhexafluorophosphate or propanephosphonic anhydride (T3P).

Process P′1 according to the present invention is conducted in the presence of a catalyst. Suitable catalyst may be selected in the list consisting of N,N-dimethylpyridin-4-amine, 1-hydroxy-benzotriazole or N,N-dimethylformamide.

In case L¹ represents a halogen atom, process P′1 according to the present invention is conducted in the presence of an acid binder. Suitable acid binders for carrying out process P1 according to the invention are in each case all inorganic and organic bases that are customary for such reactions. Preference is given to using alkaline earth metal, alkali metal hydride, alkali metal hydroxides or alkali metal alkoxides, such as sodium hydroxide, sodium hydride, calcium hydroxide, potassium hydroxide, potassium tert-butoxide or other ammonium hydroxide, alkali metal carbonates, such as caesium carbonate, sodium carbonate, potassium carbonate, potassium bicarbonate, sodium bicarbonate, alkali metal or alkaline earth metal acetates, such as sodium acetate, potassium acetate, calcium acetate and also tertiary amines, such as trimethylamine, triethylamine, diisopropylethylamine, tributylamine, N,N-dimethylaniline, pyridine, N-methylpiperidine, N,N-dimethylpyridin-4-amine, diazabicyclooctane (DABCO), diazabicyclo-nonene (DBN) or diazabicycloundecene (DBU).

It is also possible to work in the absence of an additional condensing agent or to employ an excess of the amine component, so that it simultaneously acts as acid binder agent.

Suitable solvents for carrying out process P′1 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin; chlorobenzene, dichlorobenzene, dichloromethane, chloroform, carbon tetrachloride, dichlorethane or trichlorethane; ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone, or hexamethylphosphoric triamide; alcohols such as methanol, ethanol, propanol, iso-propanol; esters, such as methyl acetate or ethyl acetate, sulfoxides, such as dimethyl sulfoxide, or sulfones, such as sulfolane.

When carrying out process P′1 according to the invention, the amine derivative of formula (II) can be employed as its salt, such as chlorhydrate or any other convenient salt.

When carrying out process P′1 according to the invention, 1 mole or an excess of the amine derivative of formula (II) and from 1 to 3 moles of the acid binder can be employed per mole of the reagent of formula (III).

It is also possible to employ the reaction components in other ratios. Work-up is carried out by known methods.

According to a further aspect according to the invention, there is provided a second process P′2 for the preparation of a compound of formula (I-2) wherein T represents S, starting from a compound of formula (I-2) wherein T represents O and illustrated according to the following reaction scheme:

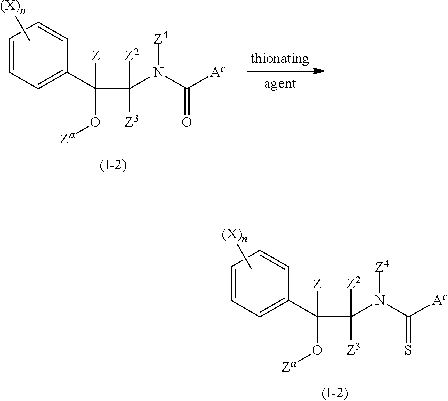

Process P2

in which Z¹, Z², Z³, Z^{a}, X and n have the meanings mentioned above for the compounds of formula (I),
Z⁴ represents cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino, and
A^{c} has the meaning given above.

Process P′2 according to the invention is performed in the presence of a thionating agent.

Starting amide derivatives of formula (I-2) wherein T represents O can be prepared according to processes P′1.

Suitable thionating agents for carrying out process P′2 according to the invention can be sulfur (S), sulfhydric acid (H₂S), sodium sulfide (Na₂S), sodium hydrosulfide (NaHS), boron trisulfide (B₂S₃), bis(diethylaluminium) sulfide ((AlEt₂)₂S), ammonium sulfide ((NH₄)₂S), phosphorous pentasulfide (P₂S₅), Lawesson's reagent (2,4-bis(4-methoxyphenyl)-1,2,3,4-dithiadiphosphetane 2,4-disulfide) or a polymer-supported thionating reagent such as described in Journal of the Chemical Society, Perkin 1 (2001), 358, in the optionally presence of a catalytic or stoichiometric or excess amount, quantity of a base such as an inorganic and organic base. Preference is given to using alkali metal carbonates, such as sodium carbonate, potassium carbonate, potassium bicarbonate, sodium bicarbonate; heterocyclic aromatic bases, such as pyridine, picoline, lutidine, collidine; and also tertiary amines, such as trimethylamine, triethylamine, tributylamine, N,N-dimethylaniline, N,N-dimethylpyridin-4-amine or N-methyl-piperidine.

Suitable solvents for carrying out process P′2 according to the invention can be customary inert organic solvents. Preference is given to using optionally halogenated aliphatic, alicyclic or aromatic hydrocarbons, such as petroleum ether, hexane, heptane, cyclohexane, methylcyclohexane, benzene, toluene, xylene or decalin, chlorobenzene, dichlorobenzene, dichloromethane, chloroform, carbon tetrachloride, dichlorethane or trichlorethane, ethers, such as diethyl ether, diisopropyl ether, methyl t-butyl ether, methyl t-amyl ether, dioxane, tetrahydrofuran, 1,2-dimethoxyethane or 1,2-diethoxyethane, nitriles, such as acetonitrile, propionitrile, n- or i-butyronitrile or benzonitrile, sulfurous solvents, such as sulfolane or carbon disulfide.

When carrying out process P′2 according to the invention, 1 mole or an excess of the sulfur equivalent of the thionating agent and from 1 to 3 moles of the base can be employed per mole of the amide reactant (I).

It is also possible to employ the reaction components in other ratios. Work-up is carried out by known methods.

Processes P′1 and P′2 according to the invention are generally carried out under atmospheric pressure. It is also possible to operate under elevated or reduced pressure.

When carrying out processes P′1 and P′2 according to the invention, the reaction temperatures can be varied within a relatively wide range. In general, these processes are carried out at temperatures from 0° C. to 200° C., preferably from 10° C. to 160° C. A way to control the temperature for the processes according to the invention is to use micro-wave technology.

In general, the reaction mixture is concentrated under reduced pressure. The residue that remains can be freed by known methods, such as chromatography or crystallization, from any impurities that can still be present.

Work-up is carried out by customary methods. Generally, the reaction mixture is treated with water and the organic phase is separated and, after drying, concentrated under reduced pressure. If appropriate, any impurities that may still be present the remaining residue can be removed by customary methods, such as chromatography, crystallization or distillation.

The compound according to the present invention can be prepared according to the processes described above. It will nevertheless be understood that, on the basis of his general knowledge and of available publications, the skilled worker will be able to adapt this method according to the specifics of each of the compounds, which it is desired to synthesize.

“Controlling nematodes” according to the invention shall mean to kill nematodes or to prevent their development or growth. The efficacy of the compositions or combinations according to the invention is assessed by comparing the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil treated with a composition or combination according to the invention and the untreated plant, plant part or soil (100%). Preferred is a reduction by 25-50% in comparison with the untreated plant, plant part or soil, very preferred a reduction by 51-79%, and particularly preferred the complete killing and the complete prevention of the development or growth by a reduction from 80% to 100% in comparison with the untreated plant, plant part or soil.

“Controlling nematodes” according to the invention shall mean the control of the reproduction of the nematodes (e.g. development of cysts or eggs). The compositions according to the invention can be used for keeping the plants healthy and can be used curatively, preventively or systemically for controlling nematodes.

The skilled person knows methods for determining the mortality of nematodes, the development of galls, the formation of cysts, the concentration of nematodes per volume of soil, of cysts, the concentration of nematodes per root, the number of nematode eggs per volume of soil, the motility of the nematodes between a plant, a plant part or the soil. The treatment according to the invention reduces the damages caused by nematodes to the plant and leads to an increase in yield.

“Nematodes” as used herein encompass all species of the phylum Nematoda and in particular species that are parasitic or cause health problems to plant or to fungi (for example species of the orders Aphelenchida, Meloidogyne, Tylenchida and others).

“Nematodes” as used herein, refer to plant nematodes meaning all nematodes that cause damage to plants. Plant nematodes encompass plant parasitic nematodes and nematodes living in the soil. Plant parasitic nematodes include, but are not limited to, ectoparasites such as Xiphinema spp., Longidorus spp., and Trichodorus spp.; semiparasites such as Tylenchulus spp.; migratory endoparasites such as Pratylenchus spp., Radopholus spp., and Scutellonerna spp.; sedentary parasites such as Heterodera spp., Globodera spp., and Meloidogyne spp., and stem and leaf endoparasites such as Ditylenchus spp., Aphelenchoides spp., and Hirshmaniella spp. Especially harmful root parasitic soil nematodes are such as cystforming nematodes of the genera Heterodera or Globodera, and/or root knot nematodes of the genus Meloidogyne. Harmful species of these genera are for example Meloidogyne incognita, Heterodera glycines (soybean cyst nematode), Globodera pallida and Globodera rostochiensis (potato cyst nematode), which species are effectively controlled with the compounds described herein. However, the use of the compounds described herein is in no way restricted to these genera or species, but also extends in the same manner to other nematodes.

Plant nematodes include but are not limited to e.g. Aglenchus agricola, Anguina tritici, Aphelenchoides arachidis, Aphelenchoides fragaria and the stem and leaf endoparasites Aphelenchoides spp. in general, Belonolaimus gracilis, Belonolaimus longicaudatus, Belonolaimus nortoni, Bursaphelenchus cocophilus, Bursaphelenchus eremus, Bursaphelenchus xylophilus and Bursaphelenchus spp. in general, Cacopaurus pestis, Criconemella curvata, Criconemella onoensis, Criconemella ornata, Criconemella rusium, Criconemella xenoplax (=Mesocriconema xenoplax) and Criconemella spp. in general, Criconemoides ferniae, Criconemoides onoense, Criconemoides omaturn and Criconemoides spp. in general, Ditylenchus destructor, Ditylenchus dipsaci, Ditylenchus myceliophagus and the stem and leaf endoparasites Ditylenchus spp. in general, Dolichodorus heterocephalus, Globodera pallida (=Heterodera pallida), Globodera rostochiensis (potato cyst nematode), Globodera solanacearum, Globodera tabacum, Globodera virginia and the sedentary, cyst forming parasites Globodera spp. in general, Helicotylenchus digonicus, Helicotylenchus dihystera, Helicotylenchus erythrine, Helicotylenchus multicinctus, Helicotylenchus nannus, Helicotylenchus pseudorobustus and Helicotylenchus spp. in general, Hemicriconemoides, Hemicycliophora arenaria, Hemicycliophora nudata, Hemicycliophora parvana, Heterodera avenae, Heterodera cruciferae, Heterodera glycines (soybean cyst nematode), Heterodera oryzae, Heterodera schachtii, Heterodera zeae and the sedentary, cyst forming parasites Heterodera spp. in general, Hirschmaniella gracilis, Hirschmaniella oryzae Hirschmaniella spinicaudata and the stem and leaf endoparasites Hirschmaniella spp. in general, Hoplolaimus aegyptii, Hoplolaimus californicus, Hoplolaimus columbus, Hoplolaimus galeatus, Hoplolaimus indicus, Hoplolaimus magnistylus, Hoplolaimus pararobustus, Longidorus africanus, Longidorus breviannulatus, Longidorus elongatus, Longidorus laevicapitatus, Longidorus vineacola and the ectoparasites Longidorus spp. in general, Meloidogyne acronea, Meloidogyne africana, Meloidogyne arenaria, Meloidogyne arenaria thamesi, Meloidogyne artiella, Meloidogyne chitwoodi, Meloidogyne coffeicola, Meloidogyne ethiopica, Meloidogyne exigua, Meloidogyne fallax, Meloidogyne graminicola, Meloidogyne graminis, Meloidogyne hapla, Meloidogyne incognita, Meloidogyne incognita acrita, Meloidogyne javanica, Meloidogyne kikuyensis, Meloidogyne minor, Meloidogyne naasi, Meloidogyne paranaensis, Meloidogyne thamesi and the sedentary parasites Meloidogyne spp. in general, Meloinema spp., Nacobbus aberrans, Neotylenchus vigissi, Paraphelenchus pseudoparietinus, Paratrichodorus allius, Paratrichodorus lobatus, Paratrichodorus minor, Paratrichodorus nanus, Paratrichodorus porosus, Paratrichodorus teres and Paratrichodorus spp. in general, Paratylenchus hamatus, Paratylenchus minutus, Paratylenchus projectus and Paratylenchus spp. in general, Pratylenchus agilis, Pratylenchus alleni, Pratylenchus andinus, Pratylenchus brachyurus, Pratylenchus cerealis, Pratylenchus coffeae, Pratylenchus crenatus, Pratylenchus delattrei, Pratylenchus giibbicaudatus, Pratylenchus goodeyi, Pratylenchus hamatus, Pratylenchus hexincisus, Pratylenchus loosi, Pratylenchus neglectus, Pratylenchus penetrans, Pratylenchus pratensis, Pratylenchus scribneri, Pratylenchus teres, Pratylenchus thornei, Pratylenchus vulnus, Pratylenchus zeae and the migratory endoparasites Pratylenchus spp. in general, Pseudohalenchus minutus, Psilenchus magnidens, Psilenchus tumidus, Punctodera chalcoensis, Quinisulcius acutus, Radopholus citrophilus, Radopholus similis, the migratory endoparasites Radopholus spp. in general, Rotylenchulus borealis, Rotylenchulus parvus, Rotylenchulus reniformis and Rotylenchulus spp. in general, Rotylenchus laurentinus, Rotylenchus macrodoratus, Rotylenchus robustus, Rotylenchus uniformis and Rotylenchus spp. in general, Scutellonema brachyurum, Scutellonema bradys, Scutellonema clathricaudatum and the migratory endoparasites Scutellonema spp. in general, Subanguina radiciola, Tetylenchus nicotianae, Trichodorus cylindricus, Trichodorus minor, Trichodorus primitivus, Trichodorus proximus, Trichodorus similis, Trichodorus sparsus and the ectoparasites Trichodorus spp. in general, Tylenchorhynchus agri, Tylenchorhynchus brassicae, Tylenchorhynchus clarus, Tylenchorhynchus claytoni, Tylenchorhynchus digitatus, Tylenchorhynchus ebriensis, Tylenchorhynchus maximus, Tylenchorhynchus nudus, Tylenchorhynchus vulgaris and Tylenchorhynchus spp. in general, Tylenchulus semipenetrans and the semiparasites Tylenchulus spp. in general, Xiphinema americanum, Xiphinema brevicolle, Xiphinema dimorphicaudatum, Xiphinema index and the ectoparasites Xiphinema spp. in general.

Examples of nematodes to which a nematicide of the present invention is applicable include, but are not limited to, nematodes of the genus Meloidogyne such as the southern root-knot nematode (Meloidogyne incognita), Javanese root-knot nematode (Meloidogyne javanica), northern root-knot nematode (Meloidogyne hapla), and peanut root-knot nematode (Meloidogyne arenaria); nematodes of the genus Ditylenchus such as the potato rot nematode (Ditylenchus destructor) and bulb and stem nematode (Ditylenchus dipsaci); nematodes of the genus Pratylenchus such as the cob root-lesion nematode (Pratylenchus penetrans), chrysanthemum root-lesion nematode (Pratylenchus fallax), coffee root-lesion nematode (Pratylenchus coffeae), tea root-lesion nematode (Pratylenchus loosi), and walnut root-lesion nematode (Pratylenchus vulnus); nematodes of the genus Globodera such as the golden nematode

(Globodera rostochiensis) and potato cyst nematode (Globodera pallida); nematodes of the genus Heterodera such as the soybean cyst nematode (Heterodera glycines) and sugar beet cyst nematode (Heterodera schachtii); nematodes of the genus Aphelenchoides such as the rice white-tip nematode (Aphelenchoides besseyi), chrysanthemum foliar nematode (Aphelenchoides ritzemabosi), and strawberry nematode (Aphelenchoides fragariae); nematodes of the genus Aphelenchus such as the mycophagous nematode (Aphelenchus avenae); nematodes of the genus Radopholus such as the burrowing nematode (Radopholus similis); nematodes of the genus Tylenchulus such as the citrus nematode (Tylenchulus semipenetrans); nematodes of the genus Rotylenchulus such as the reniform nematode (Rotylenchulus reniformis); nematodes that occur in trees, such as the pine wood nematode (Bursaphelenchus xylophilus), and the like.

Plants for which a nematicide of the present invention can be used are not particularly limited; for example, plants such as cereals (for example, rice, barley, wheat, rye, oat, corn, and the like), beans (soybeans, azuki beans, broad beans, peas, peanuts and the like), fruit trees/fruits (apples, citrus species, pears, grapes, peaches, Japanese apricots, cherries, walnuts, almonds, bananas, strawberries and the like), vegetables (cabbage, tomato, spinach, broccoli, lettuce, onion, Welsh onion, pepper and the like), root crops (carrot, potato, sweet potato, radish, lotus root, turnip and the like), industrial crops (cotton, hemp, paper mulberry, mitsumata, rape, beet, hop, sugarcane, sugar beet, olive, rubber, palms, coffee, tobacco, tea and the like), pepos (pumpkin, cucumber, watermelon, melon and the like), pasture plants (orchard grass, sorghum, thimosy, clover, alfalfa and the like), lawn grasses (mascarene grass, bent grass and the like), crops for flavorings etc. (lavender, rosemary, thyme, parsley, pepper, ginger and the like), and flower plants (chrysanthemum, rose, orchids and the like) can be mentioned.

The compound(s) and compositions comprising the compound(s) of the present invention is/are particularly useful in controlling nematodes in coffee belonging to at least one species selected from the group of the phytoparasitic nematodes consisting of Pratylenchus brachyurus, Pratylenchus coffeae, Meloidogyne exigua, Meloidogyne incognita, Meloidogyne coffeicola, Helicotylenchus spp. and also consisting of Meloidogyne paranaensis, Rotylenchus spp., Xiphinema spp., Tylenchorhynchus spp., Scutellonema spp.

Compound(s) and compositions comprising compound(s) of the present invention is/are particularly useful in controlling nematodes in potato belonging to at least one species selected from the group of the phytoparasitic nematodes consisting of Pratylenchus brachyurus, Pratylenchus pratensis, Pratylenchus scribneri, Pratylenchus penetrans, Pratylenchus coffeae, Ditylenchus dipsaci and also consisting of Pratylenchus alleni, Pratylenchus andinus, Pratylenchus cerealis, Pratylenchus crenatus, Pratylenchus hexincisus, Pratylenchus loosi, Pratylenchus neglectus, Pratylenchus teres, Pratylenchus thornei, Pratylenchus vulnus, Belonolaimus longicaudatus, Trichodorus cylindricus, Trichodorus primitivus, Trichodorus proximus, Trichodorus similis, Trichodorus sparsus, Paratrichodorus minor, Paratrichodorus allius, Paratrichodorus nanus, Paratrichodorus teres, Meloidogyne arenaria, Meloidogyne fallax, Meloidogyne hapla, Meloidogyne thamesi, Meloidogyne incognita, Meloidogyne chitwoodi, Meloidogyne javanica, Nacobbus aberrans, Globodera rostochiensis, Globodera pallida, Ditylenchus destructor, Radopholus similis, Rotylenchulus reniformis, Neotylenchus vigissi, Paraphelenchus pseudoparietinus, Aphelenchoides fragariae, Meloinema spp.

Compound(s) and compositions comprising the compound(s) of the present invention is/are particularly useful in controlling nematodes in tomato belonging to at least one species selected from the group of the phytoparasitic nematodes consisting of Meloidogyne arenaria, Meloidogyne hapla, Meloidogyne javanica, Meloidogyne incognita, Pratylenchus penetrans and also consisting of Pratylenchus brachyurus, Pratylenchus coffeae, Pratylenchus scribneri, Pratylenchus vulnus, Paratrichodorus minor, Meloidogyne exigua, Nacobbus aberrans, Globodera solanacearum, Dolichodorus heterocephalus, Rotylenchulus reniformis.

The compound(s) and compositions comprising the compound(s) of the present invention is/are particularly useful in controlling nematodes in cucurbits belonging to at least one species selected from the group of the phytoparasitic nematodes consisting of Meloidogyne arenaria, Meloidogyne hapla, Meloidogyne javanica, Meloidogyne incognita, Rotylenchulus reniformis and also consisting of Pratylenchus thornei.

The compound(s) and compositions comprising the compound(s) of the present invention is/are particularly useful in controlling nematodes in cotton belonging to at least one species selected from the group of the phytoparasitic nematodes consisting of Belonolaimus longicaudatus, Meloidogyne incognita, Hoplolaimus columbus, Hoplolaimus galeatus, Rotylenchulus reniformis.

The compound(s) and compositions comprising the compound(s) of the present invention is/are particularly useful in controlling nematodes in corn belonging to at least one species selected from the group of the phytoparasitic nematodes, especially consisting of Belonolaimus longicaudatus, Paratrichodorus minor and also consisting of Pratylenchus brachyurus, Pratylenchus delattrei, Pratylenchus hexincisus, Pratylenchus penetrans, Pratylenchus zeae, (Belonolaimus gracilis), Belonolaimus nortoni, Longidorus breviannulatus, Meloidogyne arenaria, Meloidogyne arenaria thamesi, Meloidogyne graminis, Meloidogyne incognita, Meloidogyne incognita acrita, Meloidogyne javanica, Meloidogyne naasi, Heterodera avenae, Heterodera oryzae, Heterodera zeae, Punctodera chalcoensis, Ditylenchus dipsaci, Hoplolaimus aegyptii, Hoplolaimus magnistylus, Hoplolaimus galeatus, Hoplolaimus indicus, Helicotylenchus digonicus, Helicotylenchus dihystera, Helicotylenchus pseudorobustus, Xiphinema americanum, Dolichodorus heterocephalus, Criconemella ornata, Criconemella onoensis, Radopholus similis, Rotylenchulus borealis, Rotylenchulus parvus, Tylenchorhynchus agri, Tylenchorhynchus clarus, Tylenchorhynchus claytoni, Tylenchorhynchus maximus, Tylenchorhynchus nudus, Tylenchorhynchus vulgaris, Quinisulcius acutus, Paratylenchus minutus, Hemicycliophora parvana, Aglenchus agricola, Anguina tritici, Aphelenchoides arachidis, Scutellonema brachyurum, Subanguina radiciola.

The compound(s) and compositions comprising the compound(s) of the present invention is/are particularly useful in controlling nematodes in soybean belonging to at least one species selected from the group of the phytoparasitic nematodes, especially consisting of Pratylenchus brachyurus, Pratylenchus pratensis, Pratylenchus penetrans, Pratylenchus scribneri, Belonolaimus longicaudatus, Heterodera glycines, Hoplolaimus columbus and also consisting of Pratylenchus coffeae, Pratylenchus hexincisus, Pratylenchus neglectus, Pratylenchus crenatus, Pratylenchus alleni, Pratylenchus agilis, Pratylenchus zeae, Pratylenchus vulnus, (Belonolaimus gracilis), Meloidogyne arenaria, Meloidogyne incognita, Meloidogyne javanica, Meloidogyne hapla, Hoplolaimus columbus, Hoplolaimus galeatus, Rotylenchulus reniformis.

The compound(s) and compositions comprising the compound(s) of the present invention is/are particularly useful in controlling nematodes in tobacco belonging to at least one species selected from the group of the phytoparasitic nematodes, especially consisting of Meloidogyne incognita, Meloidogyne javanica and also consisting of Pratylenchus brachyurus, Pratylenchus pratensis, Pratylenchus hexincisus, Pratylenchus penetrans, Pratylenchus neglectus, Pratylenchus crenatus, Pratylenchus thornei, Pratylenchus vulnus, Pratylenchus zeae, Longidorus elongatu, Paratrichodorus lobatus, Trichodorus spp., Meloidogyne arenaria, Meloidogyne hapla, Globodera tabacum, Globodera solanacearum, Globodera virginiae, Ditylenchus dipsaci, Rotylenchus spp., Helicotylenchus spp., Xiphinema americanum, Criconemella spp., Rotylenchulus reniformis, Tylenchorhynchus claytoni, Paratylenchus spp., Tetylenchus nicotianae.

The compound(s) and compositions comprising the compound(s) of the present invention is/are particularly useful in controlling nematodes in citrus belonging to at least one species selected from the group of the phytoparasitic nematodes, especially consisting of Pratylenchus coffeae and also consisting of Pratylenchus brachyurus, Pratylenchus vulnus, Belonolaimus longicaudatus, Paratrichodorus minor, Paratrichodorus porosus, Trichodorus, Meloidogyne incognita, Meloidogyne incognita acrita, Meloidogyne javanica, Rotylenchus macrodoratus, Xiphinema americanum, Xiphinema brevicolle, Xiphinema index, Criconemella spp., Hemicriconemoides, Radopholus similisrespectively Radopholus citrophilus, Hemicycliophora arenaria, Hemicycliophora nudata, Tylenchulus semipenetrans.

The compound(s) and compositions comprising the compound(s) of the present invention is/are particularly useful in controlling nematodes in banana belonging to at least one species selected from the group of the phytoparasitic nematodes, especially consisting of Pratylenchus coffeae, Radopholus similis and also consisting of Pratylenchus giibbicaudatus, Pratylenchus loosi, Meloidogyne spp., Helicotylenchus multicinctus, Helicotylenchus dihystera, Rotylenchulus spp.

The compound(s) and compositions comprising the compound(s) of the present invention is/are particularly useful in controlling nematodes in pine apple belonging to at least one species selected from the group of the phytoparasitic nematodes, especially consisting of Pratylenchus zeae, Pratylenchus pratensis, Pratylenchus brachyurus, Pratylenchus goodeyi., Meloidogyne spp., Rotylenchulus reniformis and also consisting of Longidorus elongatus, Longidorus laevicapitatus, Trichodorus primitivus, Trichodorus minor, Heterodera spp., Ditylenchus myceliophagus, Hoplolaimus californicus, Hoplolaimus pararobustus, Hoplolaimus indicus, Helicotylenchus dihystera, Helicotylenchus nannus, Helicotylenchus multicinctus, Helicotylenchus erythrine, Xiphinema dimorphicaudatum, Radopholus similis, Tylenchorhynchus digitatus, Tylenchorhynchus ebriensis, Paratylenchus minutus, Scutellonema clathricaudatum, Scutellonema bradys, Psilenchus tumidus, Psilenchus magnidens, Pseudohalenchus minutus, Criconemoides ferniae, Criconemoides onoense, Criconemoides ornatum.

The compound(s) and compositions comprising the compound(s) of the present invention is/are particularly useful in controlling nematodes in grapes belonging to at least one species selected from the group of the phytoparasitic nematodes, especially consisting of Pratylenchus vulnus, Meloidogyne arenaria, Meloidogyne incognita, Meloidogyne javanica, Xiphinema americanum, Xiphinema index and also consisting of Pratylenchus pratensis, Pratylenchus scribneri, Pratylenchus neglectus, Pratylenchus brachyurus, Pratylenchus thornei, Tylenchulus semipenetrans.

The compound(s) and compositions comprising the compound(s) of the present invention is/are particularly useful in controlling nematodes in tree crops—pome fruits, belonging to at least one species selected from the group of the phytoparasitic nematodes, especially consisting of Pratylenchus penetrans and also consisting of Pratylenchus vulnus, Longidorus elongatus, Meloidogyne incognita, Meloidogyne hapla.

The compound(s) and compositions comprising the compound(s) of the present invention is/are particularly useful in controlling nematodes in tree crops—stone fruits, belonging to at least one species selected from the group of the phytoparasitic nematodes, especially consisting of Pratylenchus penetrans, Pratylenchus vulnus, Meloidogyne arenaria, Meloidogyne hapla, Meloidogyne javanica, Meloidogyne incognita, Criconemella xenoplax and also consisting of Pratylenchus brachyurus, Pratylenchus coffeae, Pratylenchus scribneri, Pratylenchus zeae, Belonolaimus longicaudatus, Helicotylenchus dihystera, Xiphinema americanum, Criconemella curvata, Tylenchorhynchus claytoni, Paratylenchus hamatus, Paratylenchus projectus, Scutellonema brachyurum, Hoplolaimus galeatus.

The compound(s) and compositions comprising the compound(s) of the present invention is/are particularly useful in controlling nematodes in tree crops—nuts, belonging to at least one species selected from the group of the phytoparasitic nematodes, especially consisting of Trichodorus spp., Criconemella rusium and also consisting of Pratylenchus vulnus, Paratrichodorus spp., Meloidogyne incognita, Helicotylenchus spp., Tylenchorhynchus spp., Cacopaurus pestis.

The novel compounds of formula (I-1) and formula (I-2) can furthermore be used to control nematodes which cause damage to humans or animals.

Specific nematode species harmful to humans or animals are:

Trichinellida for example: Trichuris spp., Capillaria spp., Trichomosoides spp., Trichinella spp.

From the order of the Tylenchida for example: Micronema spp., Strongyloides spp.

From the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Many known nematicides are equally active against other parasitic helminths and are therefore used to control human and animal parasitic worms, which do not necessarily belong to the group of nematoda. Therefore, it is envisaged by the present invention that the compounds described herein may also be used as anthelmintic drugs in a more general meaning. Pathogenic endoparasitic helminths include platyhelmintha (e.g. monogenea, cestodes and trematodes), acanthocephala, and pentastoma. The following helminths may be mentioned by way of example and by way of preference—but without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp., Metorchis spp., Heterophyes spp., Metagonimus spp.

Acantocephala: From the order of the Oligacanthorhynchida z.B: Macracanthorhynchus spp., Prosthenorchis spp.; from the order of the Polymorphida for example: Filicollis spp.; from the order of the Moniliformida for example: Moniliformis spp.,

From the order of the Echinorhynchida for example Acanthocephalus spp., Echinorhynchus spp., Leptorhynchoides spp.

Pentastoma: From the order of the Porocephalida for example Linguatula spp.

In the veterinary field and in animal keeping, the administration of the active compounds according to the invention is carried out in the known manner directly or enterally, parenterally, dermally or nasally in the form of suitable preparations. Administration can be carried out prophylactically or therapeutically.

A further aspect of the invention are nematicidal compositions, comprising an effective amount of at least one compound as defined herein and at least one of the following: surfactant, solid or liquid diluent, characterized in that the surfactant or the diluent is normally used in nematicidal compositions. In an embodiment, said composition comprises at least two compounds as defined herein.

A related aspect of the invention is a method for preparing a nematicidal composition as described herein, comprising the step of mixing at least one compound as described herein with a surfactant or diluent normally used in nematicidal compositions. In an embodiment, said method comprises mixing least two compounds as defined herein with a surfactant or diluent normally used in nematicidal compositions.

In particular, the present invention relates to nematicidal composition developed to be used in agriculture or horticulture. These nematicidal compositions may be prepared in a manner known per se.

In the animal health field, i.e. in the field of veterinary medicine, the active compounds according to the present invention are active against animal parasites, in particular ectoparasites or endoparasites. The term endoparasite includes in particular helminths and protozoae, such as coccidia. Ectoparasites are typically and preferably arthropods, in particular insects and acarids. The compounds of formula (I) are preferably active against helminths.

In the field of veterinary medicine the compounds according to the invention are suitable, with favourable warm blood toxicity, for controlling parasites which occur in animal breeding and animal husbandry in livestock, breeding, zoo, laboratory, experimental and domestic animals. They are active against all or specific stages of development of the parasites.

Agricultural livestock include, for example mammals, such as, sheep, goats, horses, donkeys, camels, buffaloes, rabbits, reindeers, fallow deers, and in particular cattle and pigs; or poultry such as turkeys, ducks, geese, and in particular chickens; or fish or crustaceans e.g. in aquaculture; or as the case may be insects such as bees.

Domestic animals include, for example mammals, such as hamsters, guinea pigs, rats, mice, chinchillas, ferrets or in particular dogs, cats; cage birds; reptiles; amphibians or aquarium fish.

According to a preferred embodiment, the compounds according to the invention are administered to mammals.

According to another preferred embodiment, the compounds according to the invention are administered to birds, namely cage birds or in particular poultry.

By using the active compounds according to the invention to control animal parasites, it is intended to reduce or prevent illness, cases of deaths and performance reductions (in the case of meat, milk, wool, hides, eggs, honey and the like), so that more economical and simpler animal keeping is made possible and better animal well-being is achievable.

The term “control” or “controlling” as used herein with regard to the animal health field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

Exemplary arthropods include, without any limitation:

from the order of the Anoplurida, for example Haematopinus spp., Linognathus spp., Pediculus spp., Phtirus spp., Solenopotes spp.; from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; from the order of the Diptera and the suborders Nematocerina and Brachy-icerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; from the order of the Siphonapterida, for example Pulex spp., Ctenocephalides spp., Tunga spp., Xenopsylla spp., Ceratophyllus spp.; from the order of the Heteropterida, for example Cimex spp., Triatoma spp., Rhodnius spp., Panstrongylus spp.; as well as nuisance and hygiene pests from the order of the Blattarida.

Further, among the arthropods, the following acari may be mentioned by way of example, without any limitation:

from the subclass of the Acari (Acarina) and the order of the Metastigmata, for example from the family of argasidae like Argas spp., Ornithodorus spp., Otobius spp., from the family of Ixodidae like Ixodes spp., Amblyomma spp., Rhipicephalus (Boophilus) spp Dermacentor spp., Haemophysalis spp., Hyalomma spp., Rhipicephalus spp. (the original genus of multi host ticks); from the order of mesostigmata like Dermanyssus spp., Ornithonyssus spp., Pneumonyssus spp., Raillietia spp., Pneumonyssus spp., Sternostoma spp., Varroa spp., Acarapis spp.; from the order of the Actinedida (Prostigmata), for example Acarapis spp., Cheyletiella spp., Ornithocheyletia spp., Myobia spp., Psorergates spp., Demodex spp., Trombicula spp., Neotrombiculla spp., Listrophorus spp.; and from the order of the Acaridida (Astigmata), for example Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

Exemplary parasitic protozoa include—, without any limitation:

Mastigophora (Flagellata), such as, for example, Trypanosomatidae, for example, Trypanosoma b. brucei, T.b. gambiense, T.b. rhodesiense, T. congolense, T. cruzi, T. evansi, T. equinum, T. lewisi, T. percae, T. simiae, T. vivax, Leishmania brasiliensis, L. donovani, L. tropica, such as, for example, Trichomonadidae, for example, Giardia lamblia, G. canis.

Sarcomastigophora (Rhizopoda), such as Entamoebidae, for example, Entamoeba histolytica, Hartmanellidae, for example, Acanthamoeba sp., Harmanella sp.

Apicomplexa (Sporozoa), such as Eimeridae, for example, Eimeria acervulina, E. adenoides, E. alabamensis, E. anatis, E. anserina, E. arloingi, E. ashata, E. auburnensis, E. bovis, E. brunetti, E. canis, E. chinchillae, E. clupearum, E. columbae, E. contorta, E. crandalis, E. debliecki, E. dispersa, E. ellipsoidales, E. falciformis, E. faurei, E. flavescens, E. gallopavonis, E. hagani, E. intestinalis, E. iroquoina, E. irresidua, E. labbeana, E. leucarti, E. magna, E. maxima, E. media, E. meleagridis, E. meleagrimitis, E. mitis, E. necatrix, E. ninakohlyakimovae, E. ovis, E. parva, E. pavonis, E. perforans, E. phasani, E. piriformis, E. praecox, E. residua, E. scabra, E. spec., E. stiedai, E. suis, E. tenella, E. truncata, E. truttae, E. zuernii, Globidium spec., Isospora belli, I. canis, I. felis, I. ohioensis, I. rivolta, I. spec., I. suis, Cystisospora spec., Cryptosporidium spec., in particular C. parvum; such as Toxoplasmadidae, for example, Toxoplasma gondii, Hammondia heydornii, Neospora caninum, Besnoitia besnoitii; such as Sarcocystidae, for example, Sarcocystis bovicanis, S. bovihominis, S. ovicanis, S. ovifelis, S. neurona, S. spec., S. suihominis, such as Leucozoidae, for example, Leucozytozoon simondi, such as Plasmodiidae, for example, Plasmodium berghei, P. falciparum, P. malariae, P. ovale, P. vivax, P. spec., such as Piroplasmea, for example, Babesia argentina, B. bovis, B. canis, B. spec., Theileria parva, Theileria spec., such as Adeleina, for example, Hepatozoon canis, H. spec.

Exemplary pathogenic endoparasites, which are helminths, include platyhelmintha (e.g. monogenea, cestodes and trematodes), nematodes, acanthocephala, and pentastoma. Additional exemplary helminths include—, without any limitation:

Monogenea: e.g.: Gyrodactylus spp., Dactylogyrus spp., Polystoma spp.

Cestodes: From the order of the Pseudophyllidea for example: Diphyllobothrium spp., Spirometra spp., Schistocephalus spp., Ligula spp., Bothridium spp., Diplogonoporus spp.

From the order of the Cyclophyllida for example: Mesocestoides spp., Anoplocephala spp., Paranoplocephala spp., Moniezia spp., Thysanosoma spp., Thysaniezia spp., Avitellina spp., Stilesia spp., Cittotaenia spp., Andyra spp., Bertiella spp., Taenia spp., Echinococcus spp., Hydatigera spp., Davainea spp., Raillietina spp., Hymenolepis spp., Echinolepis spp., Echinocotyle spp., Diorchis spp., Dipylidium spp., Joyeuxiella spp., Diplopylidium spp.

Trematodes: From the class of the Digenea for example: Diplostomum spp., Posthodiplostomum spp., Schistosoma spp., Trichobilharzia spp., Ornithobilharzia spp., Austrobilharzia spp., Gigantobilharzia spp., Leucochloridium spp., Brachylaima spp., Echinostoma spp., Echinoparyphium spp., Echinochasmus spp., Hypoderaeum spp., Fasciola spp., Fasciolides spp., Fasciolopsis spp., Cyclocoelum spp., Typhlocoelum spp., Paramphistomum spp., Calicophoron spp., Cotylophoron spp., Gigantocotyle spp., Fischoederius spp., Gastrothylacus spp., Notocotylus spp., Catatropis spp., Plagiorchis spp., Prosthogonimus spp., Dicrocoelium spp., Eurytrema spp., Troglotrema spp., Paragonimus spp., Collyriclum spp., Nanophyetus spp., Opisthorchis spp., Clonorchis spp. Metorchis spp., Heterophyes spp., Metagonimus spp.

Nematodes: Trichinellida for example: Trichuris spp., Capillaria spp., Trichomosoides spp., Trichinella spp.

From the order of the Tylenchida for example: Micronema spp., Strongyloides spp.

From the order of the Rhabditina for example: Strongylus spp., Triodontophorus spp., Oesophagodontus spp., Trichonema spp., Gyalocephalus spp., Cylindropharynx spp., Poteriostomum spp., Cyclococercus spp., Cylicostephanus spp., Oesophagostomum spp., Chabertia spp., Stephanurus spp., Ancylostoma spp., Uncinaria spp., Bunostomum spp., Globocephalus spp., Syngamus spp., Cyathostoma spp., Metastrongylus spp., Dictyocaulus spp., Muellerius spp., Protostrongylus spp., Neostrongylus spp., Cystocaulus spp., Pneumostrongylus spp., Spicocaulus spp., Elaphostrongylus spp. Parelaphostrongylus spp., Crenosoma spp., Paracrenosoma spp., Angiostrongylus spp., Aelurostrongylus spp., Filaroides spp., Parafilaroides spp., Trichostrongylus spp., Haemonchus spp., Ostertagia spp., Marshallagia spp., Cooperia spp., Nematodirus spp., Hyostrongylus spp., Obeliscoides spp., Amidostomum spp., Ollulanus spp.

From the order of the Spirurida for example: Oxyuris spp., Enterobius spp., Passalurus spp., Syphacia spp., Aspiculuris spp., Heterakis spp.; Ascaris spp., Toxascaris spp., Toxocara spp., Baylisascaris spp., Parascaris spp., Anisakis spp., Ascaridia spp.; Gnathostoma spp., Physaloptera spp., Thelazia spp., Gongylonema spp., Habronema spp., Parabronema spp., Draschia spp., Dracunculus spp.; Stephanofilaria spp., Parafilaria spp., Setaria spp., Loa spp., Dirofilaria spp., Litomosoides spp., Brugia spp., Wuchereria spp., Onchocerca spp.

Acantocephala: From the order of the Oligacanthorhynchida z.B: Macracanthorhynchus spp., Prosthenorchis spp.; from the order of the Polymorphida for example: Filicollis spp.; from the order of the Moniliformida for example: Moniliformis spp.,

From the order of the Echinorhynchida for example Acanthocephalus spp., Echinorhynchus spp., Leptorhynchoides spp.

Pentastoma: From the order of the Porocephalida for example Linguatula spp.

In the veterinary field and in animal keeping, the administration of the active compounds according to the invention is carried out by methods generally known in the art, such as enterally, parenterally, dermally or nasally in the form of suitable preparations. Administration can be carried out prophylactically or therapeutically.

Thus, one embodiment of the present invention refers to compounds according to the invention for use as a medicament.

Another aspect refers to compounds according to the invention for use as an antiendoparasitical agent (medicament), in particular a helmithicidal agent (medicament) or antiprotozoaic agent (medicament). For example, compounds according to the invention for use as an antiendoparasitical agent, in particular an helmithicidal agent or antiprotozoaic agent, e.g., in animal husbandry, in animal breeding, in animal housing, in the hygiene sector.

Yet another aspect refers to compounds according to the invention for use as an antiectoparasitical agent, in particular an arthropodicidal agent such as an insecticidal agent or acaricidal agent. For example, compounds according to the invention for use as an antiectoparasitical agent, in particular an arthropodicidal agent such as an insecticidal agent or acaricidal agent, e.g., in animal husbandry, in animal breeding, in animal housing, in the hygiene sector.

Furthermore, the novel compounds of formula (I-1) and formula (I-2) according to the invention, in combination with good plant tolerance and favourable toxicity to warm-blooded animals and being tolerated well by the environment, are suitable for protecting plants and plant organs, for increasing harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in protection of stored products and of materials, and in the hygiene sector. They can be preferably employed as plant protection agents. They are active against normally sensitive and resistant species and against all or some stages of development. The abovementioned pests include:

pests from the phylum Arthropoda, especially from the class Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., Epitrimerus pyri, Eutetranychus spp., Eriophyes spp., Glycyphagus domesticus, Halotydeus destructor, Hemitarsonemus spp., Hyalomma spp., Ixodes spp., Latrodectus spp., Loxosceles spp., Metatetranychus spp., Neutrombicula autumnalis, Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., Tetranychus spp., Trombicula alfreddugesi, Vaejovis spp., Vasates lycopersici;

from the class Chilopoda, for example, Geophilus spp., Scutigera spp.;

from the order or the class Collembola, for example, Onychiurus armatus;

from the class Diplopoda, for example, Blaniulus guttulatus;

from the class Insecta, e.g. from the order Blattodea, for example, Blattella asahinai, Blattella germanica, Blatta orientalis, Leucophaea maderae, Panchlora spp., Parcoblatta spp., Periplaneta spp., Supella longipalpa;

from the order Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptolestes ferrugineus, Cryptorhynchus lapathi, Cylindrocopturus spp., Dermestes spp., Diabrotica spp., Dichocrocis spp., Dicladispa armigera, Diloboderus spp., Epilachna spp., Epitrix spp., Faustinus spp., Gibbium psylloides, Gnathocerus cornutus, Hellula undalis, Heteronychus arator, Heteronyx spp., Hylamorpha elegans, Hylotrupes bajulus, Hypera postica, Hypomeces squamosus, Hypothenemus spp., Lachnosterna consanguinea, Lasioderma serricorne, Latheticus oryzae, Lathridius spp., Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Necrobia spp., Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllophaga helleri, Phyllotreta spp., Popillia japonica, Premnotrypes spp., Prostephanus truncatus, Psylliodes spp., Ptinus spp., Rhizobius ventralis, Rhizopertha dominica, Sitophilus spp., Sitophilus oryzae, Sphenophorus spp., Stegobium paniceum, Sternechus spp., Symphyletes spp., Tanymecus spp., Tenebrio molitor, Tenebrioides mauretanicus, Tribolium spp., Trogoderma spp., Tychius spp., Xylotrechus spp., Zabrus spp.;

from the order Diptera, for example, Aedes spp., Agromyza spp., Anastrepha spp., Anopheles spp., Asphondylia spp., Bactrocera spp., Bibio hortulanus, Calliphora erythrocephala, Calliphora vicina, Ceratitis capitata, Chironomus spp., Chrysomyia spp., Chrysops spp., Chrysozona pluvialis, Cochliomyia spp., Contarinia spp., Cordylobia anthropophaga, Cricotopus sylvestris, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hydrellia griseola, Hylemya spp., Hippobosca spp., Hypoderma spp., Liriomyza spp., Lucilia spp., Lutzomyia spp., Mansonia spp., Musca spp., Oestrus spp., Oscinella frit, Paratanytarsus spp., Paralauterborniella subcincta, Pegomyia spp., Phlebotomus spp., Phorbia spp., Phormia spp., Piophila casei, Prodiplosis spp., Psila rosae, Rhagoletis spp., Sarcophaga spp., Simulium spp., Stomoxys spp., Tabanus spp., Tetanops spp., Tipula spp.;

from the order Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptocorisa varicornis, Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.;

from the order Homoptera, for example, Acizzia acaciaebaileyanae, Acizzia dodonaeae, Acizzia uncatoides, Acrida turrita, Acyrthosipon spp., Acrogonia spp., Aeneolamia spp., Agonoscena spp., Aleyrodes proletella, Aleurolobus barodensis, Aleurothrixus floccosus, Allocaridara malayensis, Amrasca spp., Anuraphis cardui, Aonidiella spp., Aphanostigma piri, Aphis spp., Arboridia apicalis, Arytainilla spp., Aspidiella spp., Aspidiotus spp., Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., Calligypona marginata, Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus ficus, Cicadulina mbila, Coccomytilus halli, Coccus spp., Cryptomyzus ribis, Cryptoneossa spp., Ctenarytaina spp., Dalbulus spp., Dialeurodes citri, Diaphorina citri, Diaspis spp., Drosicha spp., Dysaphis spp., Dysmicoccus spp., Empoasca spp., Eriosoma spp., Erythroneura spp., Eucalyptolyma spp., Euphyllura spp., Euscelis bilobatus, Ferrisia spp., Geococcus coffeae, Glycaspis spp., Heteropsylla cubana, Heteropsylla spinulosa, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Macrosteles facifrons, Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nettigoniclla spectra, Nilaparvata lugens, Oncometopia spp., Orthezia praelonga, Oxya chinensis, Pachypsylla spp., Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Prosopidopsylla flava, Protopulvinaria pyriformis, Pseudaulacaspis pentagona, Pseudococcus spp., Psyllopsis spp., Psylla spp., Pteromalus spp., Pyrilla spp., Quadraspidiotus spp., Quesada gigas, Rastrococcus spp., Rhopalosiphum spp., Saissetia spp., Scaphoideus titanus, Schizaphis graminum, Selenaspidus articulatus, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., Trialeurodes vaporariorum, Trioza spp., Typhlocyba spp., Unaspis spp., Viteus vitifolii, Zygina spp.;

from the order Hymenoptera, for example, Acromyrmex spp., Athalia spp., Atta spp., Diprion spp., Hoplocampa spp., Lasius spp., Monomorium pharaonis, Sirex spp., Solenopsis invicta, Tapinoma spp., Urocerus spp., Vespa spp., Xeris spp.;

from the order Isopoda, for example, Armadillidium vulgare, Oniscus asellus, Porcellio scaber;

from the order Isoptera, for example, Coptotermes spp., Cornitermes cumulans, Cryptotermes spp., Incisitermes spp., Microtermes obesi, Odontotermes spp., Reticulitermes spp.;

from the order Lepidoptera, for example, Achroia grisella, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., Choristoneura spp., Clysia ambiguella, Cnaphalocerus spp., Cnaphalocrocis medinalis, Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamstra brassicae, Melanitis leda, Mocis spp., Monopis obviella, Mythimna separata, Nemapogon cloacellus, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudaletia unipuncta, Pseudoplusia includens, Pyrausta nubilalis, Rachiplusia nu, Schoenobius spp., Scirpophaga spp., Scirpophaga innotata, Scotia segetum, Sesamia spp., Sesamia inferens, Sparganothis spp., Spodoptera spp., Spodoptera praefica, Stathmopoda spp., Stomopteryx subsecivella, Synanthedon spp., Tecia solanivora, Thermesia gemmatalis, Tinea cloacella, Tinea pellionella, Tineola bisselliella, Tortrix spp., Trichophaga tapetzella, Trichoplusia spp., Tryporyza incertulas, Tuta absoluta, Virachola spp.;

from the order Orthoptera or Saltatoria, for example, Acheta domesticus, Dichroplus spp., Gryllotalpa spp., Hieroglyphus spp., Locusta spp., Melanoplus spp., Schistocerca gregaria;

from the order Phthiraptera, for example, Damalinia spp., Haematopinus spp., Linognathus spp., Pediculus spp., Ptirus pubis, Trichodectes spp.;

from the order Psocoptera for example Lepinatus spp., Liposcelis spp.;

from the order Siphonaptera, for example, Ceratophyllus spp., Ctenocephalides spp., Pulex irritans, Tunga penetrans, Xenopsylla cheopsis;

from the order Thysanoptera, for example, Anaphothrips obscurus, Baliothrips biformis, Drepanothrips reuteri, Enneothrips flavens, Frankliniella spp., Heliothrips spp., Hercinothrips femoralis, Rhipiphorothrips cruentatus, Scirtothrips spp., Taeniothrips cardamomi, Thrips spp.;

from the order Zygentoma (=Thysanura), for example, Ctenolepisma spp., Lepisma saccharina, Lepismodes inquilinus, Thermobia domestica;

from the class Symphyla, for example, Scutigerella spp.;

pests from the phylum Mollusca, especially from the class Bivalvia, for example, Dreissena spp., and from the class Gastropoda, for example, Anion spp., Biomphalaria spp., Bulinus spp., Deroceras spp., Galba spp., Lymnaea spp., Oncomelania spp., Pomacea spp., Succinea spp.;

The present invention further provides formulations, and application forms prepared from them, as crop protection agents and/or pesticidal agents, such as drench, drip and spray liquors, comprising at least one of the active compounds of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

Examples of typical formulations include water-soluble liquids (SL), emulsifiable concentrates (EC), emulsions in water (EW), suspension concentrates (SC, SE, FS, OD), water-dispersible granules (WG), granules (GR) and capsule concentrates (CS); these and other possible types of formulation are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations may comprise active agrochemical compounds other than one or more active compounds of the invention.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants. The formulations are prepared either in suitable plants or else before or during the application.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

The formulations preferably comprise between 0.00000001% and 98% by weight of active compound or, with particular preference, between 0.01% and 95% by weight of active compound, more preferably between 0.5% and 90% by weight of active compound, based on the weight of the formulation.

The active compound content of the application forms (crop protection products) prepared from the formulations may vary within wide ranges. The active compound concentration of the application forms may be situated typically between 0.00000001% and 95% by weight of active compound, preferably between 0.00001% and 1% by weight, based on the weight of the application form. Application takes place in a customary manner adapted to the application forms.

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term useful plants as used in the present context refers to crop plants which are employed as plants for obtaining foodstuffs, feedstuffs, fuels or for industrial purposes.

The useful plants which can be improved by applying the compounds of formula (I) include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

Examples of trees which can be improved in accordance with the method according to the invention are: Abies sp., Eucalyptus sp., Picea sp., Pinus sp., Aesculus sp., Platanus sp., Tilia sp., Acer sp., Tsuga sp., Fraxinus sp., Sorbus sp., Betula sp., Crataegus sp., Ulmus sp., Quercus sp., Fagus sp., Salix sp., Populus sp.

Preferred trees which can be improved in accordance with the method according to the invention are: from the tree species Aesculus: A. hippocastanum, A. pariflora, A. carnea; from the tree species Platanus: P. aceriflora, P. occidentalis, P. racemosa; from the tree species Picea: P. abies; from the tree species Pinus: P. radiata, P. ponderosa, P. contorta, P. sylvestre, P. elliottii, P. montecola, P. albicaulis, P. resinosa, P. palustris, P. taeda, P. flexilis, P. jeffregi, P. baksiana, P. strobus; from the tree species Eucalyptus: E. grandis, E. globulus, E. camadentis, E. nitens, E. obliqua, E. regnans, E. pilularus.

Especially preferred trees which can be improved in accordance with the method according to the invention are: from the tree species Pinus: P. radiata, P. ponderosa, P. contorta, P. sylvestre, P. strobus; from the tree species Eucalyptus: E. grandis, E. globulus, E. camadentis.

Very particularly preferred trees which can be improved in accordance with the method according to the invention are: horse chestnut, Platanaceae, linden tree, maple tree.

The present invention can also be applied to any turf grasses, including cool-season turf grasses and warm-season turf grasses. Examples of cold-season turf grasses are bluegrasses (Poa spp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis spp.) such as creeping bentgrass (Agrostis palustris Huds.), colonial bentgrass (Agrostis tenuis Sibth.), velvet bentgrass (Agrostis canina L.), South German mixed bentgrass (Agrostis spp. including Agrostis tenuis Sibth., Agrostis canina L., and Agrostis palustris Huds.), and redtop (Agrostis alba L.);

fescues (Festuca spp.), such as red fescue (Festuca rubra L. spp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium spp.), such as annual ryegrass (Lolium multiflorum Lam.), perennial ryegrass (Lolium perenne L.) and Italian ryegrass (Lolium multiflorum Lam.);

and wheatgrasses (Agropyron spp.), such as fairway wheatgrass (Agropyron cristatum (L.) Gaertn.), crested wheatgrass (Agropyron desertorum (Fisch.) Schult.) and western wheatgrass (Agropyron smithii Rydb.).

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.).

Examples of warm-season turf grasses are Bermuda grass (Cynodon spp. L. C. Rich), zoysia grass (Zoysia spp. Willd.), St. Augustine grass (Stenotaphrum secundatum Walt Kuntze), centipede grass (Eremochloa ophiuroides Munro Hack.), carpetgrass (Axonopus affinis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.). Cool-season turf grasses are generally preferred for the use according to the invention. Especially preferred are bluegrass, benchgrass and redtop, fescues and ryegrasses. Bentgrass is especially preferred.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

The control of animal pests by treating the seed of plants has been known for a long time and is a subject of continual improvements. Nevertheless, the treatment of seed entails a series of problems which cannot always be solved in a satisfactory manner. Thus, it is desirable to develop methods for protecting the seed and the germinating plant that remove the need for, or at least significantly reduce, the additional delivery of crop protection compositions in the course of storage, after sowing or after the emergence of the plants. It is desirable, furthermore, to optimize the amount of active ingredient employed in such a way as to provide the best-possible protection to the seed and the germinating plant from attack by animal pests, but without causing damage to the plant itself by the active ingredient employed. In particular, methods for treating seed ought also to take into consideration the intrinsic insecticidal and/or nematicidal properties of pest-resistant or pest-tolerant transgenic plants, in order to achieve optimum protection of the seed and of the germinating plant with a minimal use of crop protection compositions.

The present invention therefore also relates in particular to a method for protecting seed and germinating plants from attack by pests, by treating the seed with a compound of formula (I).

The invention likewise relates to the use of the compound of formula (I) for treating seed for the purpose of protecting the seed and the resultant plant against animal pests.

The invention relates, furthermore, to seed which for protection against animal pests has been treated with a compound of formula (I).

Furthermore, the invention relates to seed which, following treatment with a compound of formula (I) of the invention, is subjected to a film-coating process in order to prevent dust abrasion of the seed.

One of the advantages of the present invention is that, owing to the particular systemic properties of the compositions of the invention, the treatment of the seed with these compositions provides protection from animal pests not only to the seed itself but also to the plants originating from the seed, after they have emerged. In this way, it may not be necessary to treat the crop directly at the time of sowing or shortly thereafter.

A further advantage is to be seen in the fact that, through the treatment of the seed with a compound of formula (I) of the invention, germination and emergence of the treated seed may be promoted.

It is likewise considered to be advantageous that compound of formula (I) may also be used, in particular, on transgenic seed.

It is also stated that a compound of formula (I) may be used in combination with agents of the signalling technology, as a result of which, for example, colonization with symbionts is improved, such as rhizobia, mycorrhiza and/or endophytic bacteria, for example, is enhanced, and/or nitrogen fixation is optimized.

The compositions of the invention are suitable for protecting seed of any variety of plant which is used in agriculture, in greenhouses, in forestry or in horticulture. More particularly, the seed in question is that of cereals (e.g. wheat, barley, rye, oats and millet), maize, cotton, soybeans, rice, potatoes, sunflower, coffee, tobacco, canola, oilseed rape, beets (e.g. sugar beet and fodder beet), peanuts, vegetables (e.g. tomato, cucumber, bean, brassicas, onions and lettuce), fruit plants, lawns and ornamentals. Particularly important is the treatment of the seed of cereals (such as wheat, barley, rye and oats) maize, soybeans, cotton, canola, oilseed rape and rice.

As already mentioned above, the treatment of transgenic seed with a compound of formula (I) is particularly important. The seed in question here is that of plants which generally contain at least one heterologous gene that controls the expression of a polypeptide having, in particular, insecticidal and/or nematicidal properties. These heterologous genes in transgenic seed may come from microorganisms such as Bacillus, Rhizobium, Pseudomonas, Serratia, Trichoderma, Clavibacter, Glomus or Gliocladium. The present invention is particularly suitable for the treatment of transgenic seed which contains at least one heterologous gene from Bacillus sp. With particular preference, the heterologous gene in question comes from Bacillus thuringiensis.

For the purposes of the present invention, the compound of formula (I) of the invention is applied alone or in a suitable formulation to the seed. The seed is preferably treated in a condition in which its stability is such that no damage occurs in the course of the treatment. Generally speaking, the seed may be treated at any point in time between harvesting and sowing. Typically, seed is used which has been separated from the plant and has had cobs, hulls, stems, husks, hair or pulp removed. Thus, for example, seed may be used that has been harvested, cleaned and dried to a moisture content of less than 15% by weight. Alternatively, seed can also be used that after drying has been treated with water, for example, and then dried again.

When treating seed it is necessary, generally speaking, to ensure that the amount of the composition of the invention, and/or of other additives, that is applied to the seed is selected such that the germination of the seed is not adversely affected, and/or that the plant which emerges from the seed is not damaged. This is the case in particular with active ingredients which may exhibit phytotoxic effects at certain application rates.

The compositions of the invention can be applied directly, in other words without comprising further components and without having been diluted. As a general rule, it is preferable to apply the compositions in the form of a suitable formulation to the seed. Suitable formulations and methods for seed treatment are known to the skilled person and are described in, for example, the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The compound of formula (I) which can be used in accordance with the invention may be converted into the customary seed-dressing formulations, such as solutions, emulsions, suspensions, powders, foams, slurries or other coating compositions for seed, and also ULV formulations.

These formulations are prepared in a known manner, by mixing the compound of formula (I) with customary adjuvants, such as, for example, customary extenders and also solvents or diluents, colorants, wetters, dispersants, emulsifiers, antifoams, preservatives, secondary thickeners, stickers, gibberellins, and also water.

Colorants which may be present in the seed-dressing formulations which can be used in accordance with the invention include all colorants which are customary for such purposes. In this context it is possible to use not only pigments, which are of low solubility in water, but also water-soluble dyes. Examples include the colorants known under the designations Rhodamin B, C.I. Pigment Red 112 and C.I. Solvent Red 1.

Wetters which may be present in the seed-dressing formulations which can be used in accordance with the invention include all of the substances which promote wetting and which are customary in the formulation of active agrochemical ingredients. Use may be made preferably of alkylnaphthalenesulphonates, such as diisopropyl- or diisobutyl-naphthalenesulphonates.

Dispersants and/or emulsifiers which may be present in the seed-dressing formulations which can be used in accordance with the invention include all of the nonionic, anionic and cationic dispersants that are customary in the formulation of active agrochemical ingredients. Use may be made preferably of nonionic or anionic dispersants or of mixtures of nonionic or anionic dispersants. Suitable nonionic dispersants are, in particular, ethylene oxide-propylene oxide block polymers, alkylphenol polyglycol ethers and also tristryrylphenol polyglycol ethers, and the phosphated or sulphated derivatives of these. Suitable anionic dispersants are, in particular, lignosulphonates, salts of polyacrylic acid, and arylsulphonate-formaldehyde condensates.

Antifoams which may be present in the seed-dressing formulations which can be used in accordance with the invention include all of the foam inhibitors that are customary in the formulation of active agrochemical ingredients. Use may be made preferably of silicone antifoams and magnesium stearate.

Preservatives which may be present in the seed-dressing formulations which can be used in accordance with the invention include all of the substances which can be employed for such purposes in agrochemical compositions. Examples include dichlorophen and benzyl alcohol hemiformal.

Secondary thickeners which may be present in the seed-dressing formulations which can be used in accordance with the invention include all substances which can be used for such purposes in agrochemical compositions. Those contemplated with preference include cellulose derivatives, acrylic acid derivatives, xanthan, modified clays and highly disperse silica.

Stickers which may be present in the seed-dressing formulations which can be used in accordance with the invention include all customary binders which can be used in seed-dressing products. Preferred mention may be made of polyvinylpyrrolidone, polyvinyl acetate, polyvinyl alcohol and tylose.

Gibberellins which may be present in the seed-dressing formulations which can be used in accordance with the invention include preferably the gibberellins A1, A3 (=gibberellic acid), A4 and A7, with gibberellic acid being used with particular preference. The gibberellins are known (cf. R. Wegler, “Chemie der Pflanzenschutz- and Schädlingsbekämpfungsmittel”, Volume 2, Springer Verlag, 1970, pp. 401-412).

The seed-dressing formulations which can be used in accordance with the invention may be used, either directly or after prior dilution with water, to treat seed of any of a wide variety of types. Accordingly, the concentrates or the preparations obtainable from them by dilution with water may be employed to dress the seed of cereals, such as wheat, barley, rye, oats and triticale, and also the seed of maize, rice, oilseed rape, peas, beans, cotton, sunflowers and beets, or else the seed of any of a very wide variety of vegetables. The seed-dressing formulations which can be used in accordance with the invention, or their diluted preparations, may also be used to dress seed of transgenic plants. In that case, additional synergistic effects may occur in interaction with the substances formed through expression.

For the treatment of seed with the seed-dressing formulations which can be used in accordance with the invention, or with the preparations produced from them by addition of water, suitable mixing equipment includes all such equipment which can typically be employed for seed dressing. More particularly, the procedure when carrying out seed dressing is to place the seed in a mixer, to add the particular desired amount of seed-dressing formulations, either as such or following dilution with water beforehand, and to carry out mixing until the distribution of the formulation on the seed is uniform. This may be followed by a drying operation.

The application rate of the seed-dressing formulations which can be used in accordance with the invention may be varied within a relatively wide range. It is guided by the particular amount of the compound of formula (I) in the formulations, and by the seed. The application rates with regard to the compound of formula (I) are situated generally at between 0.001 and 50 g per kilogram of seed, preferably between 0.01 and 15 g per kilogram of seed.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The method of treatment according to the invention can be used in the treatment of genetically modified organisms (GMOs), e.g. plants or seeds. Genetically modified plants (or transgenic plants) are plants of which a heterologous gene has been stably integrated into genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example, antisense technology, cosuppression technology, RNA interference—RNAi—technology or microRNA—miRNA—technology). A heterologous gene that is located in the genome is also called a transgene. A transgene that is defined by its particular location in the plant genome is called a transformation or transgenic event.

Plants and plant cultivars which are preferably to be treated according to the invention include all plants which have genetic material which impart particularly advantageous, useful traits to these plants (whether obtained by breeding and/or biotechnological means).

Plants and plant cultivars which are also preferably to be treated according to the invention are resistant against one or more biotic stresses, i.e. said plants show a better defense against animal and microbial pests, such as against nematodes, insects, mites, phytopathogenic fungi, bacteria, viruses and/or viroids.

Examples of nematode or insect resistant plants are described in e.g. U.S. patent application Ser. Nos. 11/765,491, 11/765,494, 10/926,819, 10/782,020, 12/032,479, 10/783,417, 10/782,096, 11/657,964, 12/192,904, 11/396,808, 12/166,253, 12/166,239, 12/166,124, 12/166,209, 11/762,886, 12/364,335, 11/763,947, 12/252,453, 12/209,354, 12/491,396, 12/497,221, 12/644,632, 12/646,004, 12/701,058, 12/718,059, 12/721,595, 12/638,591, and in WO 11/002992, WO 11/014749, WO 11/103247, WO 11/103248, WO 12/135436, WO 12/135501.

Plants and plant cultivars which may also be treated according to the invention are those plants which are resistant to one or more abiotic stresses. Abiotic stress conditions may include, for example, drought, cold temperature exposure, heat exposure, osmotic stress, flooding, increased soil salinity, increased mineral exposure, ozone exposure, high light exposure, limited availability of nitrogen nutrients, limited availability of phosphorus nutrients, shade avoidance.

Plants and plant cultivars which may also be treated according to the invention, are those plants characterized by enhanced yield characteristics. Increased yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including but not limited to, early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristic of heterosis or hybrid vigor which results in generally higher yield, vigor, health and resistance towards biotic and abiotic stresses). Such plants are typically made by crossing an inbred male-sterile parent line (the female parent) with another inbred male-fertile parent line (the male parent). Hybrid seed is typically harvested from the male sterile plants and sold to growers. Male sterile plants can sometimes (e.g. in corn) be produced by detasseling, i.e. the mechanical removal of the male reproductive organs (or males flowers) but, more typically, male sterility is the result of genetic determinants in the plant genome. In that case, and especially when seed is the desired product to be harvested from the hybrid plants it is typically useful to ensure that male fertility in the hybrid plants is fully restored. This can be accomplished by ensuring that the male parents have appropriate fertility restorer genes which are capable of restoring the male fertility in hybrid plants that contain the genetic determinants responsible for male-sterility. Genetic determinants for male sterility may be located in the cytoplasm. Examples of cytoplasmic male sterility (CMS) were for instance described in Brassica species (WO 92/05251, WO 95/09910, WO 98/27806, WO 05/002324, WO 06/021972 and U.S. Pat. No. 6,229,072). However, genetic determinants for male sterility can also be located in the nuclear genome. Male sterile plants can also be obtained by plant biotechnology methods such as genetic engineering. A particularly useful means of obtaining male-sterile plants is described in WO 89/10396 in which, for example, a ribonuclease such as barnase is selectively expressed in the tapetum cells in the stamens. Fertility can then be restored by expression in the tapetum cells of a ribonuclease inhibitor such as barstar (e.g. WO 91/02069).

Plants or plant cultivars (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-resistant plants are for example glyphosate-tolerant plants, i.e. plants made tolerant to the herbicide glyphosate or salts thereof. Plants can be made tolerant to glyphosate through different means. For example, glyphosate-tolerant plants can be obtained by transforming the plant with a gene encoding the enzyme 5-enol-pyruvylshikimate-3-phosphate synthase (EPSPS). Examples of such EPSPS genes are the AroA gene (mutant CT7) of the bacterium Salmonella typhimurium (Science 1983, 221, 370-371), the CP4 gene of the bacterium Agrobacterium sp. (Curr. Topics Plant Physiol. 1992, 7, 139-145), the genes encoding a Petunia EPSPS (Science 1986, 233, 478-481), a Tomato EPSPS (J. Biol. Chem. 1988, 263, 4280-4289), or an Eleusine EPSPS (WO 01/66704). It can also be a mutated EPSPS as described in for example EP 0837944, WO 00/66746, WO 00/66747 or WO 02/26995, WO 11/000498. Glyphosate-tolerant plants can also be obtained by expressing a gene that encodes a glyphosate oxido-reductase enzyme as described in U.S. Pat. No. 5,776,760 and U.S. Pat. No. 5,463,175. Glyphosate-tolerant plants can also be obtained by expressing a gene that encodes a glyphosate acetyl transferase enzyme as described in for example WO 02/036782, WO 03/092360, WO 05/012515 and WO 07/024782. Glyphosate-tolerant plants can also be obtained by selecting plants containing naturally-occurring mutations of the above-mentioned genes, as described in for example WO 01/024615 or WO 03/013226. Plants expressing EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Plants comprising other genes that confer glyphosate tolerance, such as decarboxylase genes, are described in e.g. U.S. patent application Ser. Nos. 11/588,811, 11/185,342, 12/364,724, 11/185,560 or 12/423,926.

Other herbicide resistant plants are for example plants that are made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Plants expressing an exogenous phosphinothricin acetyltransferase are for example described in U.S. Pat. Nos. 5,561,236; 5,648,477; 5,646,024; 5,273,894; 5,637,489; 5,276,268; 5,739,082; 5,908,810 and 7,112,665.

Further herbicide-tolerant plants are also plants that are made tolerant to the herbicides inhibiting the enzyme hydroxyphenylpyruvatedioxygenase (HPPD). HPPD is an enzyme that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, WO 99/24586, WO 09/144079, WO 02/046387, U.S. Pat. No. 6,768,044, WO 11/076877, WO 11/076882, WO 11/076885, WO 11/076889 or WO 11/076892. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 04/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 07/103567 and WO 08/150473.

Still further herbicide resistant plants are plants that are made tolerant to acetolactate synthase (ALS) inhibitors. Known ALS-inhibitors include, for example, sulfonylurea, imidazolinone, triazolopyrimidines, pyrimidinyoxy-(thio)benzoates, and/or sulfonylaminocarbonyltriazolinone herbicides. Different mutations in the ALS enzyme (also known as acetohydroxyacid synthase, AHAS) are known to confer tolerance to different herbicides and groups of herbicides, as described for example in Tranel and Wright (Weed Science 2002, 50, 700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and WO 96/33270. Other imidazolinone-tolerant plants are also described in for example WO 04/040012, WO 04/106529, WO 05/020673, WO 05/093093, WO 06/007373, WO 06/015376, WO 06/024351, and WO 06/060634. Further sulfonylurea- and imidazolinone-tolerant plants are also described in for example WO 07/024782, WO 2011/076345, WO 2012058223, WO 2012150335 and U.S. Patent Application 61/288,958.

Other plants tolerant to imidazolinone and/or sulfonylurea can be obtained by induced mutagenesis, selection in cell cultures in the presence of the herbicide or mutation breeding as described for example for soybeans in U.S. Pat. No. 5,084,082, for rice in WO 97/41218, for sugar beet in U.S. Pat. No. 5,773,702 and WO 99/057965, for lettuce in U.S. Pat. No. 5,198,599, or for sunflower in WO 01/065922.

Plants or plant cultivars (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

An “insect-resistant transgenic plant”, as used herein, includes any plant containing at least one transgene comprising a coding sequence encoding:

1) an insecticidal crystal protein from Bacillus thuringiensis or an insecticidal portion thereof, such as the insecticidal crystal proteins listed by Crickmore et al. (Microbiology and Molecular Biology Reviews 1998, 62, 807-813), updated by Crickmore et al. (2005) at the Bacillus thuringiensis toxin nomenclature, online at: http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP-A 1 999 141 and WO 07/107302), or such proteins encoded by synthetic genes as e.g. described in and U.S. patent application Ser. No. 12/249,016; or
2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Nat. Biotechnol. 2001, 19, 668-72; Applied Environm. Microbiol. 2006, 71, 1765-1774) or the binary toxin made up of the Cry1A or Cry1F proteins and the Cry2Aa or Cry2Ab or Cry2Ae proteins (U.S. patent application Ser. No. 12/214,022 and EP-A 2 300 618); or
3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 07/027777); or
4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or
5) an insecticidal secreted protein from Bacillus thuringiensis or Bacillus cereus, or an insecticidal portion thereof, such as the vegetative insecticidal (VIP) proteins listed at:
http://www.lifesci.sussex.ac.uk/home/Neil_Crickmore/Bt/vip.html, e.g., proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or
9) a secreted protein from Bacillus thuringiensis or Bacillus cereus which is insecticidal in the presence of a crystal protein from Bacillus thuringiensis, such as the binary toxin made up of VIP3 and Cry1A or Cry1F (U.S. Patent Applications 61/126,083 and 61/195019), or the binary toxin made up of the VIP3 protein and the Cry2Aa or Cry2Ab or Cry2Ae proteins (U.S. patent application Ser. No. 12/214,022 and EP-A 2 300 618).
10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein)

Of course, an insect-resistant transgenic plant, as used herein, also includes any plant comprising a combination of genes encoding the proteins of any one of the above classes 1 to 10. In one embodiment, an insect-resistant plant contains more than one transgene encoding a protein of any one of the above classes 1 to 10, to expand the range of target insect species affected when using different proteins directed at different target insect species, or to delay insect resistance development to the plants by using different proteins insecticidal to the same target insect species but having a different mode of action, such as binding to different receptor binding sites in the insect.

An “insect-resistant transgenic plant”, as used herein, further includes any plant containing at least one transgene comprising a sequence producing upon expression a double-stranded RNA which upon ingestion by a plant insect pest inhibits the growth of this insect pest, as described e.g. in WO 07/080126, WO 06/129204, WO 07/074405, WO 07/080127 and WO 07/035650.

Plants or plant cultivars (obtained by plant biotechnology methods such as genetic engineering) which may also be treated according to the invention are tolerant to abiotic stresses. Such plants can be obtained by genetic transformation, or by selection of plants containing a mutation imparting such stress resistance. Particularly useful stress tolerance plants include:

1) plants which contain a transgene capable of reducing the expression and/or the activity of poly(ADP-ribose) polymerase (PARP) gene in the plant cells or plants as described in WO 00/04173, WO 06/045633, EP-A 1 807 519, or EP-A 2 018 431.
2) plants which contain a stress tolerance enhancing transgene capable of reducing the expression and/or the activity of the PARG encoding genes of the plants or plants cells, as described e.g. in WO 04/090140.
3) plants which contain a stress tolerance enhancing transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in EP-A 1 794 306, WO 06/133827, WO 07/107326, EP-A 1 999 263, or WO 07/107326.

Plants or plant cultivars (obtained by plant biotechnology methods such as genetic engineering) which may also be treated according to the invention show altered quantity, quality and/or storage-stability of the harvested product and/or altered properties of specific ingredients of the harvested product such as:

1) transgenic plants which synthesize a modified starch, which in its physical-chemical characteristics, in particular the amylose content or the amylose/amylopectin ratio, the degree of branching, the average chain length, the side chain distribution, the viscosity behaviour, the gelling strength, the starch grain size and/or the starch grain morphology, is changed in comparison with the synthesised starch in wild type plant cells or plants, so that this is better suited for special applications. Said transgenic plants synthesizing a modified starch are disclosed, for example, in EP-A 0 571 427, WO 95/04826, EP-A 0 719 338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO 99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 04/056999, WO 05/030942, WO 05/030941, WO 05/095632, WO 05/095617, WO 05/095619, WO 2005/095618, WO 05/123927, WO 06/018319, WO 06/103107, WO 06/108702, WO 07/009823, WO 00/22140, WO 06/063862, WO 06/072603, WO 02/034923, WO 08/017518, WO 08/080630, WO 08/080631, WO 08/090008, WO 01/14569, WO 02/79410, WO 03/33540, WO 04/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 05/002359, U.S. Pat. No. 5,824,790, U.S. Pat. No. 6,013,861, WO 94/04693, WO 94/09144, WO 94/11520, WO 95/35026, WO 97/20936, WO 10/012796, WO 10/003701,
2) transgenic plants which synthesize non starch carbohydrate polymers or which synthesize non starch carbohydrate polymers with altered properties in comparison to wild type plants without genetic modification. Examples are plants producing polyfructose, especially of the inulin and levan-type, as disclosed in EP-A 0 663 956, WO 96/01904, WO 96/21023, WO 98/39460, and WO 99/24593, plants producing alpha-1,4-glucans as disclosed in WO 95/31553, US 2002031826, U.S. Pat. No. 6,284,479, U.S. Pat. No. 5,712,107, WO 97/47806, WO 97/47807, WO 97/47808 and WO 00/14249, plants producing alpha-1,6 branched alpha-1,4-glucans, as disclosed in WO 00/73422, plants producing alternan, as disclosed in e.g. WO 00/47727, WO 00/73422, U.S. Pat. No. 5,908,975 and EP-A 0 728 213,
3) transgenic plants which produce hyaluronan, as for example disclosed in WO 06/032538, WO 07/039314, WO 07/039315, WO 07/039316, JP-A 2006-304779, and WO 05/012529.
4) transgenic plants or hybrid plants, such as onions with characteristics such as ‘high soluble solids content’, low pungency′ (LP) and/or ‘long storage’ (LS), as described in U.S. patent application Ser. No. 12/020,360.
5) Transgenic plants displaying an increase yield as for example disclosed in WO 11/095528

Plants or plant cultivars (that can be obtained by plant biotechnology methods such as genetic engineering) which may also be treated according to the invention are plants, such as cotton plants, with altered fiber characteristics. Such plants can be obtained by genetic transformation, or by selection of plants contain a mutation imparting such altered fiber characteristics and include:

a) Plants, such as cotton plants, containing an altered form of cellulose synthase genes as described in WO 98/00549.
b) Plants, such as cotton plants, containing an altered form of rsw2 or rsw3 homologous nucleic acids as described in WO 04/053219.
c) Plants, such as cotton plants, with increased expression of sucrose phosphate synthase as described in WO 01/17333.
d) Plants, such as cotton plants, with increased expression of sucrose synthase as described in WO 02/45485.
e) Plants, such as cotton plants, wherein the timing of the plasmodesmatal gating at the basis of the fiber cell is altered, e.g. through downregulation of fiber-selective β-1,3-glucanase as described in WO 05/017157, or as described in WO 09/143995.
f) Plants, such as cotton plants, having fibers with altered reactivity, e.g. through the expression of N-acetylglucosaminetransferase gene including nodC and chitin synthase genes as described in WO 06/136351, WO 11/089021, WO 11/089021, WO 12/074868.

Plants or plant cultivars (that can be obtained by plant biotechnology methods such as genetic engineering) which may also be treated according to the invention are plants, such as oilseed rape or related Brassica plants, with altered oil profile characteristics. Such plants can be obtained by genetic transformation, or by selection of plants contain a mutation imparting such altered oil profile characteristics and include:

a) Plants, such as oilseed rape plants, producing oil having a high oleic acid content as described e.g. in U.S. Pat. No. 5,969,169, U.S. Pat. No. 5,840,946 or U.S. Pat. No. 6,323,392 or U.S. Pat. No. 6,063,947
b) Plants such as oilseed rape plants, producing oil having a low linolenic acid content as described in U.S. Pat. No. 6,270,828, U.S. Pat. No. 6,169,190, U.S. Pat. No. 5,965,755 or WO 11/060946
c) Plant such as oilseed rape plants, producing oil having a low level of saturated fatty acids as described e.g. in U.S. Pat. No. 5,434,283 or U.S. patent application Ser. No. 12/668,303
d) Plants such as oilseed rape plants, producing oil having an alter glucosinolate content as described in WO 2012075426.

Plants or plant cultivars (that can be obtained by plant biotechnology methods such as genetic engineering) which may also be treated according to the invention are plants, such as oilseed rape or related Brassica plants, with altered seed shattering characteristics. Such plants can be obtained by genetic transformation, or by selection of plants contain a mutation imparting such altered seed shattering characteristics and include plants such as oilseed rape plants with delayed or reduced seed shattering as described in WO 2009/068313 and WO 2010/006732, WO 2012090499.

Plants or plant cultivars (that can be obtained by plant biotechnology methods such as genetic engineering) which may also be treated according to the invention are plants, such as Tobacco plants, with altered post-translational protein modification patterns, for example as described in WO 10/121818 and WO 10/145846.

Particularly useful transgenic plants which may be treated according to the invention are plants containing transformation events, or combination of transformation events, that are the subject of petitions for non-regulated status, in the United States of America, to the Animal and Plant Health Inspection Service (APHIS) of the United States Department of Agriculture (USDA) whether such petitions are granted or are still pending. At any time this information is readily available from APHIS (4700 River Road, Riverdale, Md. 20737, USA), for instance on its Internet site (URL http://www.aphis.usda.gov/brs/not_reg.html).

Additional particularly useful plants containing single transformation events or combinations of transformation events are listed for example in the databases from various national or regional regulatory agencies (see for example http://gmoinfo.jrc.it/gmp_browse.aspx and http://www.cera-gmc.org/?action=gm_crop_database).

Particularly useful transgenic plants which may be treated according to the invention are plants containing transformation events, or a combination of transformation events, and that are listed for example in the databases for various national or regional regulatory agencies including Event 531/PV-GHBK04 (cotton, insect control, described in WO 2002/040677), Event 1143-14A (cotton, insect control, not deposited, described in WO 06/128569); Event 1143-51B (cotton, insect control, not deposited, described in WO 06/128570); Event 1445 (cotton, herbicide tolerance, not deposited, described in US-A 2002-120964 or WO 02/034946Event 17053 (rice, herbicide tolerance, deposited as PTA-9843, described in WO 10/117737); Event 17314 (rice, herbicide tolerance, deposited as PTA-9844, described in WO 10/117735); Event 281-24-236 (cotton, insect control—herbicide tolerance, deposited as PTA-6233, described in WO 05/103266 or US-A 2005-216969); Event 3006-210-23 (cotton, insect control—herbicide tolerance, deposited as PTA-6233, described in US-A 2007-143876 or WO 05/103266); Event 3272 (corn, quality trait, deposited as PTA-9972, described in WO 06/098952 or US-A 2006-230473); Event 33391 (wheat, herbicide tolerance, deposited as PTA-2347, described in WO 2002/027004), Event 40416 (corn, insect control—herbicide tolerance, deposited as ATCC PTA-11508, described in WO 11/075593); Event 43A47 (corn, insect control—herbicide tolerance, deposited as ATCC PTA-11509, described in WO 11/075595); Event 5307 (corn, insect control, deposited as ATCC PTA-9561, described in WO 10/077816); Event ASR-368 (bent grass, herbicide tolerance, deposited as ATCC PTA-4816, described in US-A 2006-162007 or WO 04/053062); Event B16 (corn, herbicide tolerance, not deposited, described in US-A 2003-126634); Event BPS-CV127-9 (soybean, herbicide tolerance, deposited as NCIMB No. 41603, described in WO 10/080829); Event BLR1 (oilseed rape, restoration of male sterility, deposited as NCIMB 41193, described in WO 2005/074671), Event CE43-67B (cotton, insect control, deposited as DSM ACC2724, described in US-A 2009-217423 or WO 06/128573); Event CE44-69D (cotton, insect control, not deposited, described in US-A 2010-0024077); Event CE44-69D (cotton, insect control, not deposited, described in WO 06/128571); Event CE46-02A (cotton, insect control, not deposited, described in WO 06/128572); Event COT102 (cotton, insect control, not deposited, described in US-A 2006-130175 or WO 04/039986); Event COT202 (cotton, insect control, not deposited, described in US-A 2007-067868 or WO 05/054479); Event COT203 (cotton, insect control, not deposited, described in WO 05/054480); Event DAS21606-3/1606 (soybean, herbicide tolerance, deposited as PTA-11028, described in WO 012/033794), Event DAS40278 (corn, herbicide tolerance, deposited as ATCC PTA-10244, described in WO 11/022469); Event DAS-44406-6/pDAB8264.44.06.1 (soybean, herbicide tolerance, deposited as PTA-11336, described in WO 2012/075426), Event DAS-14536-7/pDAB8291.45.36.2 (soybean, herbicide tolerance, deposited as PTA-11335, described in WO 2012/075429), Event DAS-59122-7 (corn, insect control—herbicide tolerance, deposited as ATCC PTA 11384, described in US-A 2006-070139); Event DAS-59132 (corn, insect control—herbicide tolerance, not deposited, described in WO 09/100188); Event DAS68416 (soybean, herbicide tolerance, deposited as ATCC PTA-10442, described in WO 11/066384 or WO 11/066360); Event DP-098140-6 (corn, herbicide tolerance, deposited as ATCC PTA-8296, described in US-A 2009-137395 or WO 08/112019); Event DP-305423-1 (soybean, quality trait, not deposited, described in US-A 2008-312082 or WO 08/054747); Event DP-32138-1 (corn, hybridization system, deposited as ATCC PTA-9158, described in US-A 2009-0210970 or WO 09/103049); Event DP-356043-5 (soybean, herbicide tolerance, deposited as ATCC PTA-8287, described in US-A 2010-0184079 or WO 08/002872); Event EE-1 (brinjal, insect control, not deposited, described in WO 07/091277); Event FI117 (corn, herbicide tolerance, deposited as ATCC 209031, described in US-A 2006-059581 or WO 98/044140); Event FG72 (soybean, herbicide tolerance, deposited as PTA-11041, described in WO 2011/063413), Event GA21 (corn, herbicide tolerance, deposited as ATCC 209033, described in US-A 2005-086719 or WO 98/044140); Event GG25 (corn, herbicide tolerance, deposited as ATCC 209032, described in US-A 2005-188434 or WO 98/044140); Event GHB119 (cotton, insect control—herbicide tolerance, deposited as ATCC PTA-8398, described in WO 08/151780); Event GHB614 (cotton, herbicide tolerance, deposited as ATCC PTA-6878, described in US-A 2010-050282 or WO 07/017186); Event GJ11 (corn, herbicide tolerance, deposited as ATCC 209030, described in US-A 2005-188434 or WO 98/044140); Event GM RZ13 (sugar beet, virus resistance, deposited as NCIMB-41601, described in WO 10/076212); Event H7-1 (sugar beet, herbicide tolerance, deposited as NCIMB 41158 or NCIMB 41159, described in US-A 2004-172669 or WO 04/074492); Event JOPLIN1 (wheat, disease tolerance, not deposited, described in US-A 2008-064032); Event LL27 (soybean, herbicide tolerance, deposited as NCIMB41658, described in WO 06/108674 or US-A 2008-320616); Event LL55 (soybean, herbicide tolerance, deposited as NCIMB 41660, described in WO 06/108675 or US-A 2008-196127); Event LLcotton25 (cotton, herbicide tolerance, deposited as ATCC PTA-3343, described in WO 03/013224 or US-A 2003-097687); Event LLRICE06 (rice, herbicide tolerance, deposited as ATCC 203353, described in U.S. Pat. No. 6,468,747 or WO 00/026345); Event LLRice62 (rice, herbicide tolerance, deposited as ATCC 203352, described in WO 2000/026345), Event LLRICE601 (rice, herbicide tolerance, deposited as ATCC PTA-2600, described in US-A 2008-2289060 or WO 00/026356); Event LY038 (corn, quality trait, deposited as ATCC PTA-5623, described in US-A 2007-028322 or WO 05/061720); Event MIR162 (corn, insect control, deposited as PTA-8166, described in US-A 2009-300784 or WO 07/142840); Event MIR604 (corn, insect control, not deposited, described in US-A 2008-167456 or WO 05/103301); Event MON15985 (cotton, insect control, deposited as ATCC PTA-2516, described in US-A 2004-250317 or WO 02/100163); Event MON810 (corn, insect control, not deposited, described in US-A 2002-102582); Event MON863 (corn, insect control, deposited as ATCC PTA-2605, described in WO 04/011601 or US-A 2006-095986); Event MON87427 (corn, pollination control, deposited as ATCC PTA-7899, described in WO 11/062904); Event MON87460 (corn, stress tolerance, deposited as ATCC PTA-8910, described in WO 09/111263 or US-A 2011-0138504); Event MON87701 (soybean, insect control, deposited as ATCC PTA-8194, described in US-A 2009-130071 or WO 09/064652); Event MON87705 (soybean, quality trait—herbicide tolerance, deposited as ATCC PTA-9241, described in US-A 2010-0080887 or WO 10/037016); Event MON87708 (soybean, herbicide tolerance, deposited as ATCC PTA-9670, described in WO 11/034704); Event MON87712 (soybean, yield, deposited as PTA-10296, described in WO 2012/051199), Event MON87754 (soybean, quality trait, deposited as ATCC PTA-9385, described in WO 10/024976); Event MON87769 (soybean, quality trait, deposited as ATCC PTA-8911, described in US-A 2011-0067141 or WO 09/102873); Event MON88017 (corn, insect control—herbicide tolerance, deposited as ATCC PTA-5582, described in US-A 2008-028482 or WO 05/059103); Event MON88913 (cotton, herbicide tolerance, deposited as ATCC PTA-4854, described in WO 04/072235 or US-A 2006-059590); Event MON88302 (oilseed rape, herbicide tolerance, deposited as PTA-10955, described in WO 2011/153186), Event MON88701 (cotton, herbicide tolerance, deposited as PTA-11754, described in WO 2012/134808), Event MON89034 (corn, insect control, deposited as ATCC PTA-7455, described in WO 07/140256 or US-A 2008-260932); Event MON89788 (soybean, herbicide tolerance, deposited as ATCC PTA-6708, described in US-A 2006-282915 or WO 06/130436); Event MS11 (oilseed rape, pollination control—herbicide tolerance, deposited as ATCC PTA-850 or PTA-2485, described in WO 01/031042); Event MS8 (oilseed rape, pollination control—herbicide tolerance, deposited as ATCC PTA-730, described in WO 01/041558 or US-A 2003-188347); Event NK603 (corn, herbicide tolerance, deposited as ATCC PTA-2478, described in US-A 2007-292854); Event PE-7 (rice, insect control, not deposited, described in WO 08/114282); Event RF3 (oilseed rape, pollination control—herbicide tolerance, deposited as ATCC PTA-730, described in WO 01/041558 or US-A 2003-188347); Event RT73 (oilseed rape, herbicide tolerance, not deposited, described in WO 02/036831 or US-A 2008-070260); Event SYHT0H2/SYN-000H2-5 (soybean, herbicide tolerance, deposited as PTA-11226, described in WO 2012/082548), Event T227-1 (sugar beet, herbicide tolerance, not deposited, described in WO 02/44407 or US-A 2009-265817); Event T25 (corn, herbicide tolerance, not deposited, described in US-A 2001-029014 or WO 01/051654); Event T304-40 (cotton, insect control—herbicide tolerance, deposited as ATCC PTA-8171, described in US-A 2010-077501 or WO 08/122406); Event T342-142 (cotton, insect control, not deposited, described in WO 06/128568); Event TC1507 (corn, insect control—herbicide tolerance, not deposited, described in US-A 2005-039226 or WO 04/099447); Event VIP1034 (corn, insect control—herbicide tolerance, deposited as ATCC PTA-3925., described in WO 03/052073), Event 32316 (corn, insect control-herbicide tolerance, deposited as PTA-11507, described in WO 11/084632), Event 4114 (corn, insect control-herbicide tolerance, deposited as PTA-11506, described in WO 11/084621), event EE-GM3/FG72 (soybean, herbicide tolerance, ATCC Accession No PTA-11041, WO 2011/063413A2), event DAS-68416-4 (soybean, herbicide tolerance, ATCC Accession No PTA-10442, WO2011/066360A1), event DAS-68416-4 (soybean, herbicide tolerance, ATCC Accession No PTA-10442, WO 2011/066384A1), event DP-040416-8 (corn, insect control, ATCC Accession No PTA-11508, WO 2011/075593A1), event DP-043A47-3 (corn, insect control, ATCC Accession No PTA-11509, WO 2011/075595A1), event DP-004114-3 (corn, insect control, ATCC Accession No PTA-11506, WO 2011/084621A1), event DP-032316-8 (corn, insect control, ATCC Accession No PTA-11507, WO 2011/084632A1), event MON-88302-9 (oilseed rape, herbicide tolerance, ATCC Accession No PTA-10955, WO 2011/153186A1), event DAS-21606-3 (soybean, herbicide tolerance, ATCC Accession No. PTA-11028, WO 2012/033794A2), event MON-87712-4 (soybean, quality trait, ATCC Accession No. PTA-10296, WO 2012/051199A2), event DAS-44406-6 (soybean, stacked herbicide tolerance, ATCC Accession No. PTA-11336, WO 2012/075426A1), event DAS-14536-7 (soybean, stacked herbicide tolerance, ATCC Accession No. PTA-11335, WO 2012/075429A1), event SYN-000H2-5 (soybean, herbicide tolerance, ATCC Accession No. PTA-11226, WO 2012/082548A2), event DP-061061-7 (oilseed rape, herbicide tolerance, no deposit No available, WO 2012071039A1), event DP-073496-4 (oilseed rape, herbicide tolerance, no deposit No available, US2012131692), event 8264.44.06.1 (soybean, stacked herbicide tolerance, Accession No PTA-11336, WO 2012075426A2), event 8291.45.36.2 (soybean, stacked herbicide tolerance, Accession No. PTA-11335, WO 2012075429A2), event SYHT0H2 (soybean, ATCC Accession No. PTA-11226, WO 2012/082548A2), event MON88701 (cotton, ATCC Accession No PTA-11754, WO 2012/134808A1), event KK179-2 (alfalfa, ATCC Accession No PTA-11833, WO2013003558A1), event pDAB8264.42.32.1 (soybean, stacked herbicide tolerance, ATCC Accession No PTA-11993, WO 2013010094A1), event MZDT09Y (corn, ATCC Accession No PTA-13025, WO 2013012775A1).

The treatment of the plants and plant parts with the compounds of formula (I) is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

A preferred direct treatment of the plants is the leaf application treatment, i.e. compounds of formula (I) or compositions containing them are applied to the foliage, it being possible for the treatment frequency and the application rate to be matched to the infection pressure.

In the case of systemically active compounds, compounds of formula (I) or compositions according to the invention reach the plants via the root system. In this case, the treatment of the plants is effected by allowing the compounds of formula (I) or compositions according to the invention to act on the environment of the plant. This can be done for example by drenching, incorporating in the soil or into the nutrient solution, i.e. the location of the plant (for example the soil or hydroponic systems) is impregnated with a liquid form of compounds of formula (I) or compositions according to the invention, or by soil application, i.e. the compounds of formula (I) or compositions according to the invention are incorporated into the location of the plants in solid form (for example in the form of granules). In the case of paddy rice cultures, this may also be done by metering the compounds of formula (I) or compositions according to the invention into a flooded paddy field in a solid use form (for example in the form of granules).

The following examples illustrate in a non-limiting manner the preparation of compounds of formula (I) according to the invention.

### PREPARATION EXAMPLES

### Example 1

### N-[1-(4-chlorophenyl)-1-methoxypropan-2-yl]-2,6-difluorobenzamide (compound 132)

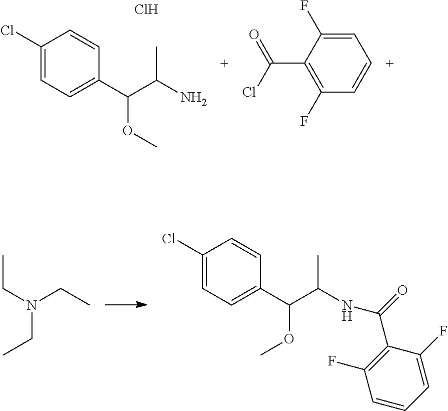

75 mg (0.31 mMol) of 1-(4-chlorophenyl)-1-methoxypropan-2-amine hydrochloride was dissolved in dichloromethane (1 mL) at 0° C. 96 mg of triethylamine (95 mMol) were added to the reaction mixture followed by the slow addition of 67 mg of 2,6-difluorobenzoyl chloride (0.38 mMol). The reaction mixture was then allowed to warm up to room temperature and stirred until completion of the reaction. The reaction mixture was quenched by the addition of 10 mL of dichloromethane and 1 mL of a 1 M HCl aqueous solution. After separation of the phases, the organic phase was washed with 5 mL of a saturated aqueous sodium bicarbonate solution. After separation of the phases, the organic phase was dried over magnesium sulphate, filtered and concentrated in vacuo. The crude product was chromatographed over silica to provide 91 mg of two isomers of N-[1-(4-chlorophenyl)-1-methoxypropan-2-yl]-2,6-difluorobenzamide (yield=83%) as a white solid.

Isomer 1: Log P acid=3.12 Mass (m/z): 340.0 (M+H)⁺

Isomer 2: Log P acid=3.20 Mass (m/z): 340.0 (M+H)⁺

¹H-NMR: 400 MHz, d6-DMSO, δ, 8.8-8.6 (m, 1H), 7.6-7.1 (m, 8H), 4.3-4.0 (m, 2H), 3.21-3.15 (m, 2H), 1.1-0.9 (m, 2H).

### Example 2

### 3-chloro-N-{1-[(2,4-dichlorophenyl)(methoxy)methyl]cyclopropyl}pyrazine-2-carboxamide (compound 210)

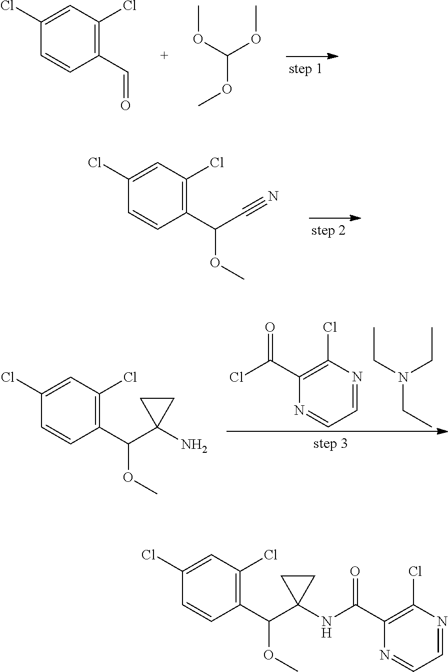

### Step 1: Synthesis of (2,4-dichlorophenyl)(methoxy)acetonitrile

A suspension of 2,4-dichlorobenzaldehyde (17.5 g, 0.1 mol, 1.0 eq.), K-10 (10.4 g, 47.5 mmol, 1.2 eq.), trimethyl orthoformate (32.1 g, 0.15 mmol, 1.5 eq) in hexane (300 mL) was stirred at room temperature for 1 h. After formation of 2,4-dichloro-1-(dimethoxymethyl)benzene, the solvent was removed in vacuo. The obtained oil was dissolved in dichloromethane (150 mL), followed by addition of stannous chloride (1.86 g, 10 mmol, 0.1 eq) and cyanotrimethylsilane (20 g, 0.2 mmol, 2.0 eq). The mixture was stirred at room temperature for 6 h. The mixture was diluted with water (100 mL) and extracted with dichloromethane (150 mL×2). The combined organic layers were washed with brine (150 mL×2), dried over magnesium sulphate, purified by column chromatography on silica gel (eluent: hexane: ethyl acetate=0-100%) to give the title compound (15 g).

### Step 2: Synthesis of 1-[(2,4-dichlorophenyl)(methoxy)methyl]cyclopropanamine

To a degassed mixture of 2-(2,4-dichlorophenyl)-2-methoxyacetonitrile (1.0 g, 4.63 mmol, 1.0 eq.) in diethylether was added Ti(O-i-Pr)4 (1.44 g, 5.1 mmol, 1.1 eq.) and ethylmagnesium bromide (10.1 mL, 1 M in ether). After stirring at 25° C. for 30 min, BF₃.Et₂O (1.2 mL, 9.2 mmol) was added. Then the reaction mixture was stirred for additional 2 h. After the reaction was completed, an aqueous solution of sodium hydroxide (10%, 10 mL) was added and the mixture was extracted with ethyl acetate (150 mL×2). The combined organic layers were washed with brine (150 mL×2), dried over magnesium sulphate and concentrated in vacuo to dryness. The residue was purified by column chromatography on neutral aluminium oxide (eluent: hexane: CH2Cl2=0-100%) to give the title compound (0.41 g).

¹H-NMR: 400 MHz, d6-DMSO, δ, 7.4-7.3 (m, 3H), 4.5 (s, 1H), 3.3 (s, 3H), 1.7 (s, 2H), 0.7-0.6 (m, 2H), 0.5-0.4 (m, 2H).

### Step 3: Synthesis of 3-chloro-N-{1-[(2,4-dichlorophenyl)(methoxy)methyl]cyclopropyl}pyrazine-2-carboxamide (compound 210)

60 mg of 1-[(2,4-dichlorophenyl)(methoxy)methyl]cyclopropanamine (0.24 mmol) were placed in 1 mL of dichloromethane and the reaction mixture was cooled down to 0° C. 0.29 mmol of triethylamine was added and a solution of 0.29 mmol of 3-chloropyrazine-2-carbonyl chloride in 1 mL of dichloromethane was slowly added. The reaction mixture was stirred at room temperature. 10 mL of dichloromethane were added. The organic phase was washed with a saturated solution of NaHCO₃. The organic phase was dried over magnesium sulphate and evaporated leading to the title compound (compound 210).

¹H-NMR: 400 MHz, d6-DMSO: see NMR-Peak list (compound 210)

Log P acid=3.24

### Measurement of log P-values:

Measurement of log P values was performed according EEC directive 79/831 Annex V.A8 by HPLC (High Performance Liquid Chromatography) on reversed phase columns with the following methods:

^{[a]} Measurement was done at pH 2.3 with 0.1% phosphoric acid and acetonitrile as eluent with a linear gradient from 10% acetonitrile to 95% acetonitrile.

^{[b]} measurement of LC-MS was done at pH 2.7 with 0.1% formic acid in water and with acetonitrile (contains 0.1% formic acid) as eluent with a linear gradient from 10% acetonitrile to 95% acetonitrile.

^{[c]} Measurement with LC-MS was done at pH 7.8 with 0.001 molar ammonium hydrogen carbonate solution in water as eluent with a linear gradient from 10% acetonitrile to 95% acetonitrile.

Calibration was done with not branched alkan2-ones (with 3 to 16 carbon atoms) with known log P-values (measurement of log P values using retention times with linear interpolation between successive alkanones).. lambda-maX-values were determined using UV-spectra from 200 nm to 400 nm and the peak values of the chromatographic signals.

TABLE 1
Compounds of formula (Ia)

| | |
|---|---|
| | Formula (Ia) |
| 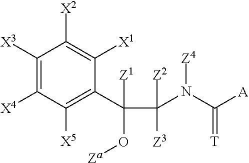 | |

wherein A, T, X¹, X², X³, X⁴, X⁵, Z¹, Z², Z³, Z⁴ and Z^{a} have the meaning given in the table. As to the
indication of stereochemistry, C(Z¹)(OZ^{a}) is C1 and C(Z²)(Z³) is C2.

| Ex- | | | | | | | | | | | | | | |
|---|---|---|---|---|---|---|---|---|---|---|---|---|---|---|
| ample | | | | | | | | | | | | | Stereo- | |
| num- | | | | | | | | | | | | | chem- | |
| ber | A | T | X¹ | X² | X³ | X⁴ | X⁵ | Z¹ | Z² | Z³ | Z⁴ | Z^{a} | istry | logP |
| 1 | 3-methyl-2-thienyl | O | H | H | Cl | H | H | H | H | H | H | prop-2- | | 3.18^{[a]} |
| | | | | | | | | | | | | yn-1-yl | | |
| 2 | 3-methyl-2-thienyl | O | H | H | Cl | H | H | H | H | H | H | allyl | | |
| 3 | 3-methyl-2-thienyl | O | H | H | Cl | Cl | H | H | methyl | methyl | H | prop-2- | | 4.58^{[a]} |
| | | | | | | | | | | | | yn-1-yl | | |
| 4 | 3-methyl-2-thienyl | O | H | H | Cl | H | H | H | methyl | methyl | H | prop-2- | | 4.22^{[a]} |
| | | | | | | | | | | | | yn-1-yl | | |
| 5 | 3-methyl-2-thienyl | O | H | H | Cl | H | H | H | H | H | H | 4-methyl- | | 4.62^{[a]} |
| | | | | | | | | | | | | benzyl | | |
| 6 | 3-methyl-2-thienyl | O | H | H | Cl | H | H | H | H | H | H | cyclo- | | 3.87^{[a]} |
| | | | | | | | | | | | | propyl- | | |
| | | | | | | | | | | | | methyl | | |
| 7 | 3-methyl-2-thienyl | O | H | H | Cl | H | H | H | H | H | H | isobutyl | | 4.49^{[a]} |
| 8 | 2-chloropyridin-3-yl | O | H | H | Cl | H | H | H | H | H | H | allyl | | |
| 9 | 2-chloropyridin-3-yl | O | H | H | Cl | H | H | H | H | H | H | prop-2- | | |
| | | | | | | | | | | | | yn-1-yl | | |
| 10 | 2-chloropyridin-3-yl | O | H | H | Cl | H | H | H | H | H | H | cyclo- | | |
| | | | | | | | | | | | | propyl- | | |
| | | | | | | | | | | | | methyl | | |
| 11 | 2-chloropyridin-3-yl | O | H | H | Cl | H | H | H | H | H | H | isobutyl | | |
| 12 | 3-(difluoromethyl)-1-methyl- | O | Cl | H | Cl | H | H | H | methyl | H | H | methyl | | |
| | 1H-pyrazol-4-yl | | | | | | | | | | | | | |
| 13 | 5-fluoro-1,3-dimethyl-1H- | O | Cl | H | Cl | H | H | H | methyl | H | H | methyl | | 3.13^{[b]} |
| | pyrazol-4-yl | | | | | | | | | | | | | |
| 14 | 3-(difluoromethyl)-5-fluoro-1- | O | Cl | H | Cl | H | H | H | methyl | H | H | methyl | | 3.65^{[b]} |
| | methyl-1H-pyrazol-4-yl | | | | | | | | | | | | | |
| 15 | 2-chlorophenyl | O | H | H | H | H | H | H | H | H | H | methyl | | |
| 16 | 3-(difluoromethyl)-1-methyl- | O | H | H | H | H | H | H | H | H | H | methyl | | 1.93^{[b]} |
| | 1H-pyrazol-4-yl | | | | | | | | | | | | | |
| 17 | 3-(difluoromethyl)-1-methyl- | O | Cl | H | Cl | H | H | H | methyl | H | H | methyl | rel- | 3.25^{[b]} |
| | 1H-pyrazol-4-yl | | | | | | | | | | | | (1R,2S) | |
| 18 | 3-(difluoromethyl)-1-methyl- | O | Cl | H | Cl | H | H | H | methyl | H | H | methyl | rel- | |
| | 1H-pyrazol-4-yl | | | | | | | | | | | | (1R,2R) | |
| 19 | 3-(difluoromethyl)-1-methyl- | O | Cl | H | Cl | H | H | H | methyl | H | H | methyl | (1S,2R) | |
| | 1H-pyrazol-4-yl | | | | | | | | | | | | | |
| 20 | 3-(difluoromethyl)-1-methyl- | O | Cl | H | Cl | H | H | H | methyl | H | H | methyl | (1R,2S) | |
| | 1H-pyrazol-4-yl | | | | | | | | | | | | | |
| 21 | 3-(difluoromethyl)-1-methyl- | O | Cl | H | Cl | H | H | H | methyl | H | H | methyl | (1R,2R) | |
| | 1H-pyrazol-4-yl | | | | | | | | | | | | | |
| 22 | 3-(difluoromethyl)-1-methyl- | O | Cl | H | Cl | H | H | H | methyl | H | H | methyl | (1S,2S) | |
| | 1H-pyrazol-4-yl | | | | | | | | | | | | | |
| 23 | 5-chloro-3-(difluoromethyl)-1- | O | H | H | meth- | prop-2- | H | H | methyl | H | H | methyl | | 2.76^{[b]} |
| | methyl-1H-pyrazol-4-yl | | | | oxy | yn-1- | | | | | | | | |
| | | | | | | yloxy | | | | | | | | |
| 24 | 3,4-dihydro-2H-chromen-2-yl | O | H | H | prop-2- | meth- | H | H | methyl | H | H | methyl | | 3.33^{[b]} |
| | | | | | yn-1- | oxy | | | | | | | | |
| | | | | | yloxy | | | | | | | | | |
| 25 | 3-(difluoromethyl)-1-methyl- | O | H | Cl | H | Cl | H | H | H | H | H | methyl | | |
| | 1H-pyrazol-4-yl | | | | | | | | | | | | | |
| 26 | 3-(difluoromethyl)-1-methyl- | O | Cl | H | Cl | H | H | H | H | H | H | methyl | | |
| | 1H-pyrazol-4-yl | | | | | | | | | | | | | |
| 27 | 3-(difluoromethyl)-1-methyl- | O | H | Cl | H | Cl | H | H | methyl | H | H | methyl | | |
| | 1H-pyrazol-4-yl | | | | | | | | | | | | | |
| 28 | 3-(difluoromethyl)-1-methyl- | O | Cl | H | Cl | H | H | H | ethyl | H | H | methyl | | |
| | 1H-pyrazol-4-yl | | | | | | | | | | | | | |
| 29 | 3-(difluoromethyl)-1-methyl- | O | Cl | H | Cl | H | H | H | methyl | H | H | ethyl | | |
| | 1H-pyrazol-4-yl | | | | | | | | | | | | | |
| 30 | 2-chlorophenyl | O | Cl | H | Cl | H | H | H | methyl | H | H | methyl | | |
| 31 | | | | | | | | | | | | | | |
| 32 | 3-(difluoromethyl)-1-methyl- | O | H | H | Cl | H | Cl | H | ethane-1,2-diyl | | H | methyl | | |
| | 1H-pyrazol-4-yl | | | | | | | | | | | | | |
| 33 | 2-chloro-6-(trifluoromethyl) | O | Cl | H | Cl | H | H | H | methyl | H | H | methyl | | |
| | pyridin-3-yl | | | | | | | | | | | | | |
| 34 | 2-chloropyridin-3-yl | O | Cl | H | Cl | H | H | H | methyl | H | H | methyl | | |
| 35 | 2-(trifluoromethyl)phenyl | O | Cl | H | Cl | H | H | H | methyl | H | H | methyl | | 3.90^{[a]} |
| 36 | 2,6-dichloropyridin-3-yl | O | Cl | H | Cl | H | H | H | methyl | H | H | methyl | | |
| 37 | 2-chloro-6-(trifluoromethyl) | O | Cl | H | Cl | H | H | H | H | H | H | methyl | | |
| | pyridin-3-yl | | | | | | | | | | | | | |
| 38 | 2-chloro-6-methylpyridin-3-yl | O | Cl | H | Cl | H | H | H | methyl | H | H | methyl | | |
| 39 | 2-(trifluoromethyl)pyridin-3-yl | O | Cl | H | Cl | H | H | H | methyl | H | H | methyl | | |
| 40 | 5-chloro-3-(difluoromethyl)-1- | O | H | Cl | H | Cl | H | H | H | H | H | methyl | | 3.55^{[b]} |
| | methyl-1H-pyrazol-4-yl | | | | | | | | | | | | | |
| 41 | 3-(difluoromethyl)-5-fluoro-1- | O | H | Cl | H | Cl | H | H | H | H | H | methyl | | 3.33^{[b]} |
| | methyl-1H-pyrazol-4-yl | | | | | | | | | | | | | |
| 42 | 5-chloro-3-(difluoromethyl)-1- | O | H | Cl | H | Cl | H | H | methyl | H | H | methyl | | 3.92^{[b]} |
| | methyl-1H-pyrazol-4-yl | | | | | | | | | | | | | |
| 43 | 3-(difluoromethyl)-5-fluoro-1- | O | H | Cl | H | Cl | H | H | methyl | H | H | methyl | | 3.70^{[b]} |
| | methyl-1H-pyrazol-4-yl | | | | | | | | | | | | | |
| 44 | 5-chloro-3-(difluoromethyl)-1- | O | Cl | H | Cl | H | H | H | H | H | H | methyl | | 3.48^{[b]} |
| | methyl-1H-pyrazol-4-yl | | | | | | | | | | | | | |
| 45 | 3-(difluoromethyl)-5-fluoro-1- | O | Cl | H | Cl | H | H | H | H | H | H | methyl | | 3.25^{[b]} |
| | methyl-1H-pyrazol-4-yl | | | | | | | | | | | | | |
| 46 | 5-chloro-3-(difluoromethyl)-1- | O | Cl | H | Cl | H | H | H | methyl | H | H | ethyl | | 4.46^{[b]} |
| | methyl-1H-pyrazol-4-yl | | | | | | | | | | | | | |
| 47 | 3-(difluoromethyl)-5-fluoro-1- | O | Cl | H | Cl | H | H | H | methyl | H | H | ethyl | | 4.20^{[b]} |
| | methyl-1H-pyrazol-4-yl | | | | | | | | | | | | | |
| 48 | 5-chloro-3-(difluoromethyl)-1- | O | Cl | H | Cl | H | H | H | ethyl | H | H | methyl | | 4.18^{[b]} |
| | methyl-1H-pyrazol-4-yl | | | | | | | | | | | | | |
| 49 | 3-(difluoromethyl)-5-fluoro-1- | O | Cl | H | Cl | H | H | H | ethyl | H | H | methyl | | 3.94^{[b]} |
| | methyl-1H-pyrazol-4-yl | | | | | | | | | | | | | |
| 50 | 5-chloro-3-(difluoromethyl)-1- | O | Cl | H | Cl | H | H | H | ethane-1,2-diyl | | H | methyl | | 3.70^{[b]} |
| | methyl-1H-pyrazol-4-yl | | | | | | | | | | | | | |
| 51 | 3-(difluoromethyl)-5-fluoro-1- | O | Cl | H | Cl | H | H | H | ethane-1,2-diyl | | H | methyl | | 3.46^{[b]} |
| | methyl-1H-pyrazol-4-yl | | | | | | | | | | | | | |
| 52 | 5-chloro-3-(trifluoromethyl) | O | Cl | H | Cl | H | H | H | methyl | H | H | ethyl | | |
| | pyridin-2-yl | | | | | | | | | | | | | |
| 53 | 5-chloro-3-(trifluoromethyl) | O | Cl | H | Cl | H | H | H | methyl | H | H | methyl | | |
| | pyridin-2-yl | | | | | | | | | | | | | |
| 54 | 5-chloro-3-(trifluoromethyl) | O | H | H | Cl | H | Cl | H | ethane-1,2-diyl | | H | methyl | | |
| | pyridin-2-yl | | | | | | | | | | | | | |
| 55 | 1-ethyl-3,4-dimethyl-1H- | O | H | H | H | H | H | H | methyl | H | H | methyl | | 2.71^{[b]} |
| | pyrazol-5-yl | | | | | | | | | | | | | |
| 56 | 3-(difluoromethyl)-1-methyl- | O | H | H | H | H | H | H | methyl | H | H | methyl | | |
| | 1H-pyrazol-4-yl | | | | | | | | | | | | | |
| 57 | 2-(trifluoromethyl)phenyl | O | Cl | H | Cl | H | H | H | ethane-1,2-diyl | | H | methyl | | |
| 58 | 3-methyl-2-thienyl | O | Cl | H | Cl | H | H | H | methyl | H | H | methyl | | 4.02^{[c]}; |
| | | | | | | | | | | | | | | 4.05^{[b]} |
| 59 | 2-methyl-3-furyl | O | H | H | Cl | H | H | H | methyl | H | H | methyl | | 3.21^{[b]} |
| 60 | 3,4,5-trichloro-2-thienyl | O | H | H | Cl | H | H | H | methyl | H | H | methyl | | |
| 61 | 3,4-dichloro-1,2-thiazol-5-yl | O | H | H | Cl | H | H | H | methyl | H | H | methyl | | 4.34^{[b]} |
| 62 | 3-thienyl | O | H | H | Cl | H | H | H | methyl | H | H | methyl | | 2.84^{[b]} |
| 63 | 2-bromo-3-thienyl | O | H | H | Cl | H | H | H | methyl | H | H | methyl | | 3.68^{[b]} |
| 64 | 2-iodo-3-furyl | O | H | H | Cl | H | H | H | methyl | H | H | methyl | | 3.28^{[b]} |
| 65 | 3-bromo-2-thienyl | O | H | H | Cl | H | H | H | methyl | H | H | methyl | | 3.83^{[c]}; |
| | | | | | | | | | | | | | | 3.89^{[b]} |
| 66 | 3-chloro-2-thienyl | O | H | H | Cl | H | H | H | methyl | H | H | methyl | | 3.83^{[c]}; |
| | | | | | | | | | | | | | | 3.83^{[b]} |
| 67 | | | | | | | | | | | | | | |
| 68 | | | | | | | | | | | | | | |
| 69 | 3-chloro-2-thienyl | O | H | H | Cl | H | H | H | H | H | H | methyl | | 3.39^{[c]}; |
| | | | | | | | | | | | | | | 3.41^{[b]} |
| 70 | 2-chloro-3-thienyl | O | H | H | Cl | H | H | H | H | H | H | methyl | | 3.13^{[c]}; |
| | | | | | | | | | | | | | | 3.15^{[b]} |
| 71 | 2-iodo-3-furyl | O | H | H | Cl | H | H | H | H | H | H | methyl | | 2.90^{[c]}; |
| | | | | | | | | | | | | | | 2.92^{[b]} |
| 72 | 2,6-difluorophenyl | O | H | H | Cl | H | H | H | H | H | H | methyl | | 2.79^{[c]}; |
| | | | | | | | | | | | | | | 2.81^{[b]} |
| 73 | 3-(trifluoromethyl)pyrazin-2-yl | O | H | H | Cl | H | Cl | H | methyl | H | H | methyl | | |
| 74 | 2-fluorophenyl | O | Cl | H | Cl | H | H | H | methyl | methyl | H | methyl | | 4.77^{[c]}; |
| | | | | | | | | | | | | | | 4.80^{[b]} |
| 75 | 2-chlorophenyl | O | Cl | H | Cl | H | H | H | methyl | methyl | H | methyl | | 4.54^{[c]}; |
| | | | | | | | | | | | | | | 4.59^{[b]} |
| 76 | 2-(trifluoromethyl)phenyl | O | Cl | H | Cl | H | H | H | methyl | methyl | H | methyl | | 4.62^{[c]}; |
| | | | | | | | | | | | | | | 4.68^{[b]} |
| 77 | 2,6-difluorophenyl | O | Cl | H | Cl | H | H | H | methyl | methyl | H | methyl | | 4.24^{[c]}; |
| | | | | | | | | | | | | | | 4.29^{[b]} |
| 78 | 3-chloro-2-thienyl | O | Cl | H | Cl | H | H | H | methyl | methyl | H | methyl | | 5.31^{[c]}; |
| | | | | | | | | | | | | | | 5.34^{[b]} |
| 79 | 3-iodo-2-thienyl | O | Cl | H | Cl | H | H | H | methyl | methyl | H | methyl | | |
| 80 | 3-bromo-2-furyl | O | Cl | H | Cl | H | H | H | methyl | methyl | H | methyl | | 4.37^{[c]}; |
| | | | | | | | | | | | | | | 4.59^{[b]} |
| 81 | 2-chloropyridin-3-yl | O | H | H | Cl | H | H | H | methyl | methyl | H | methyl | | 3.23^{[c]}; |
| | | | | | | | | | | | | | | 3.24^{[b]} |
| 82 | 2,6-difluorophenyl | O | H | H | Cl | H | H | H | methyl | methyl | H | methyl | | 3.79^{[c]}; |
| | | | | | | | | | | | | | | 3.78^{[b]} |
| 83 | 2-fluorophenyl | O | H | H | Cl | H | H | H | methyl | methyl | H | methyl | | 3.99^{[c]}; |
| | | | | | | | | | | | | | | 4.24^{[b]} |
| 84 | 2-chlorophenyl | O | H | H | Cl | H | H | H | methyl | methyl | H | methyl | | 3.87^{[c]}; |
| | | | | | | | | | | | | | | 4.12^{[b]} |
| 85 | 2-(trifluoromethyl)phenyl | O | H | H | Cl | H | H | H | methyl | methyl | H | methyl | | 3.99^{[c]}; |
| | | | | | | | | | | | | | | 4.25^{[b]} |
| 86 | 3-chloro-2-thienyl | O | H | H | Cl | H | H | H | methyl | methyl | H | methyl | | 4.43^{[c]}; |
| | | | | | | | | | | | | | | 4.72^{[b]} |
| 87 | 3-iodo-2-thienyl | O | H | H | Cl | H | H | H | methyl | methyl | H | methyl | | 4.47^{[c]}; |
| | | | | | | | | | | | | | | 4.77^{[b]} |
| 88 | 3-bromo-2-furyl | O | H | H | Cl | H | H | H | methyl | methyl | H | methyl | | 3.88^{[c]}; |
| | | | | | | | | | | | | | | 4.09^{[b]} |
| 89 | 2-chloropyridin-3-yl | O | Cl | H | Cl | H | H | H | ethyl | H | H | methyl | | 3.33^{[b]} |
| 90 | 2-(difluoromethyl)pyridin-3-yl | O | Cl | H | Cl | H | H | H | ethyl | H | H | methyl | | 3.35^{[b]} |
| 91 | 2-(trifluoromethyl)pyridin-3-yl | O | Cl | H | Cl | H | H | H | ethyl | H | H | methyl | | 3.70^{[b]} |
| 92 | 3-(trifluoromethyl)pyrazin-2-yl | O | Cl | H | Cl | H | H | H | ethyl | H | H | methyl | | 3.99^{[b]} |
| 93 | 2-(difluoromethyl)pyridin-3-yl | O | H | Cl | H | Cl | H | H | methyl | H | H | methyl | | 2.03^{[b]} |
| 94 | 2-chloropyridin-3-yl | O | H | Cl | H | Cl | H | H | methyl | H | H | methyl | | 3.17^{[b]} |
| 95 | 3-(trifluoromethyl)pyrazin-2-yl | O | H | Cl | H | Cl | H | H | methyl | H | H | methyl | | 3.74^{[b]} |
| 96 | 2-(trifluoromethyl)pyridin-3-yl | O | H | Cl | H | Cl | H | H | methyl | H | H | methyl | | 3.52^{[b]} |
| 97 | 2-(difluoromethyl)pyridin-3-yl | O | Cl | H | Cl | H | H | H | ethane-1,2-diyl | | H | methyl | | 3.06^{[b]} |
| 98 | 2-chloropyridin-3-yl | O | Cl | H | Cl | H | H | H | ethane-1,2-diyl | | H | methyl | | 3.00^{[b]} |
| 99 | 3-(trifluoromethyl)pyrazin-2-yl | O | Cl | H | Cl | H | H | H | ethane-1,2-diyl | | H | methyl | | 3.61^{[b]} |
| 100 | 2-(trifluoromethyl)pyridin-3-yl | O | Cl | H | Cl | H | H | H | ethane-1,2-diyl | | H | methyl | | 3.37^{[b]} |
| 101 | 2-(difluoromethyl)pyridin-3-yl | O | Cl | H | Cl | H | H | H | H | H | H | methyl | | 2.80^{[b]} |
| 102 | 2-chloropyridin-3-yl | O | Cl | H | Cl | H | H | H | H | H | H | methyl | | 2.75^{[b]} |
| 103 | 3-(trifluoromethyl)pyrazin-2-yl | O | Cl | H | Cl | H | H | H | H | H | H | methyl | | 3.35^{[b]} |
| 104 | 2-(trifluoromethyl)pyridin-3-yl | O | Cl | H | Cl | H | H | H | H | H | H | methyl | | 3.11^{[b]} |
| 105 | 2-(difluoromethyl)pyridin-3-yl | O | Cl | H | Cl | H | H | H | methyl | H | H | ethyl | | 3.57^{[b]} |
| 106 | 2-chloropyridin-3-yl | O | Cl | H | Cl | H | H | H | methyl | H | H | ethyl | | 3.59^{[b]} |
| 107 | 3-(trifluoromethyl)pyrazin-2-yl | O | Cl | H | Cl | H | H | H | methyl | H | H | ethyl | | 4.16^{[b]} |
| 108 | 2-(trifluoromethyl)pyridin-3-yl | O | Cl | H | Cl | H | H | H | methyl | H | H | ethyl | | 3.92^{[b]} |
| 109 | 2-chloropyridin-3-yl | O | H | H | H | H | H | methyl | H | H | H | methyl | | 2.11^{[b]} |
| 110 | 2-(difluoromethyl)pyridin-3-yl | O | H | H | H | H | H | methyl | H | H | H | methyl | | 2.14^{[b]} |
| 111 | 2-methyl-5,6-dihydro-1,4- | O | H | H | H | H | H | methyl | H | H | H | methyl | | 2.68^{[b]} |
| | oxathiin-3-yl | | | | | | | | | | | | | |
| 112 | 3-(trifluoromethyl)pyrazin-2-yl | O | H | H | H | H | H | methyl | H | H | H | methyl | | 2.70^{[b]} |
| 113 | 2-(trifluoromethyl)pyridin-3-yl | O | H | H | H | H | H | methyl | H | H | H | methyl | | 2.44^{[b]} |
| 114 | 1-(difluoromethyl)-4-fluoro-3- | O | Cl | H | Cl | H | H | H | methyl | H | H | methyl | | 4.57^{[b]} |
| | methyl-1H-pyrazol-5-yl | | | | | | | | | | | | | |
| 115 | 1-(difluoromethyl)-3-methyl- | O | Cl | H | Cl | H | H | H | methyl | H | H | methyl | | 3.73^{[b]} |
| | 1H-pyrazol-5-yl | | | | | | | | | | | | | |
| 116 | 1-(difluoromethyl)-3,4- | O | Cl | H | Cl | H | H | H | methyl | H | H | methyl | | 3.84^{[c]}; |
| | dimethyl-1H-pyrazol-5-yl | | | | | | | | | | | | | 3.97^{[b]} |
| 117 | 2-(difluoromethyl)pyridin-3-yl | O | H | Cl | H | Cl | H | H | H | H | H | methyl | | 2.88^{[b]} |
| 118 | 2-chloropyridin-3-yl | O | H | Cl | H | Cl | H | H | H | H | H | methyl | | 2.82^{[b]} |
| 119 | 2-(trifluoromethyl)pyridin-3-yl | O | H | Cl | H | Cl | H | H | H | H | H | methyl | | 3.19^{[b]} |
| 120 | 3-(trifluoromethyl)pyrazin-2-yl | O | H | Cl | H | Cl | H | H | H | H | H | methyl | | 3.42^{[b]} |
| 121 | 1-(difluoromethyl)-1H-pyrazol- | O | Cl | H | Cl | H | H | H | ethane-1,2-diyl | | H | methyl | | 3.46^{[b]} |
| | 5-yl | | | | | | | | | | | | | |
| 122 | 1-(difluoromethyl)-3-nitro-1H- | O | Cl | H | Cl | H | H | H | ethane-1,2-diyl | | H | methyl | | 3.94^{[b]} |
| | pyrazol-5-yl | | | | | | | | | | | | | |
| 123 | 1-(difluoromethyl)-1H-pyrazol- | O | Cl | H | Cl | H | H | H | methyl | H | H | methyl | | 3.09^{[b]} |
| | 5-yl | | | | | | | | | | | | | |
| 124 | 1,3-dimethyl-1H-pyrazol-5-yl | O | Cl | H | Cl | H | H | H | methyl | H | H | methyl | | 3.08^{[b]} |
| 125 | 1,3-dimethyl-1H-pyrazol-5-yl | O | Cl | H | Cl | H | H | H | ethane-1,2-diyl | | H | methyl | | 3.05^{[b]} |
| 126 | 1-(difluoromethyl)-3-nitro-1H- | O | Cl | H | Cl | H | H | H | methyl | H | H | methyl | | |
| | pyrazol-5-yl | | | | | | | | | | | | | |
| 127 | 2-iodophenyl | O | H | H | Cl | H | Cl | H | methyl | H | H | methyl | | |
| 128 | 2-(trifluoromethyl)phenyl | O | H | H | Cl | H | H | H | methyl | H | H | methyl | | 3.35^{[c]}; |
| | | | | | | | | | | | | | | 3.45^{[b]} |
| 129 | 2-methylphenyl | O | H | H | Cl | H | H | H | methyl | H | H | methyl | | 3.20^{[c]}; |
| | | | | | | | | | | | | | | 3.30^{[b]} |
| 130 | 2-chlorophenyl | O | H | H | Cl | H | H | H | methyl | H | H | methyl | | 3.21^{[c]} |
| 131 | 2-bromophenyl | O | H | H | Cl | H | H | H | methyl | H | H | methyl | | 3.30^{[c]}; |
| | | | | | | | | | | | | | | 3.29^{[b]} |
| 132 | 2,6-difluorophenyl | O | H | H | Cl | H | H | H | methyl | H | H | methyl | | 3.12^{[b]} |
| 133 | 2-fluorophenyl | O | H | H | Cl | H | H | H | methyl | H | H | methyl | | 3.44^{[c]}; |
| | | | | | | | | | | | | | | 3.40^{[b]} |
| 134 | 2-fluoro-6-(trifluoromethyl) | O | H | H | Cl | H | H | H | methyl | H | H | methyl | | 3.61^{[c]}; |
| | phenyl | | | | | | | | | | | | | 3.65^{[b]} |
| 135 | 2-chloropyridin-3-yl | O | H | H | Cl | H | H | H | methyl | H | H | methyl | | 2.62^{[c]}; |
| | | | | | | | | | | | | | | 2.65^{[b]} |
| 136 | 2-(difluoromethyl)phenyl | O | H | H | Cl | H | H | H | methyl | H | H | methyl | | 3.42^{[c]}; |
| | | | | | | | | | | | | | | 3.46^{[b]} |
| 137 | 2-iodophenyl | O | H | H | Cl | H | H | H | methyl | H | H | methyl | | 3.42^{[c]}; |
| | | | | | | | | | | | | | | 3.44^{[b]} |
| 138 | 2-nitrophenyl | O | Cl | H | H | H | H | H | methyl | H | H | methyl | | 2.78^{[c]}; |
| | | | | | | | | | | | | | | 2.82^{[b]} |
| 139 | 2-nitrophenyl | O | H | H | Cl | H | H | H | methyl | H | H | methyl | | 2.93^{[c]}; |
| | | | | | | | | | | | | | | 2.83^{[b]} |
| 140 | 2-(difluoromethyl)phenyl | O | Cl | H | H | H | H | H | methyl | H | H | methyl | | 3.39^{[c]}; |
| | | | | | | | | | | | | | | 3.42^{[b]} |
| 141 | 2-fluoro-6-(trifluoromethyl) | O | Cl | H | H | H | H | H | methyl | H | H | methyl | | 3.50^{[c]}; |
| | phenyl | | | | | | | | | | | | | 3.52^{[b]} |
| 142 | 2-bromophenyl | O | Cl | H | H | H | H | H | methyl | H | H | methyl | | 3.31^{[c]}; |
| | | | | | | | | | | | | | | 3.34^{[b]} |
| 143 | 2-chloropyridin-3-yl | O | Cl | H | H | H | H | H | methyl | H | H | methyl | | 2.53^{[c]}; |
| | | | | | | | | | | | | | | 2.55^{[b]} |
| 144 | 2,6-difluorophenyl | O | Cl | H | H | H | H | H | methyl | H | H | methyl | | 3.08^{[c]}; |
| | | | | | | | | | | | | | | 3.11^{[b]} |
| 145 | 2-(trifluoromethyl)phenyl | O | Cl | H | H | H | H | H | methyl | H | H | methyl | | 3.41^{[c]}; |
| | | | | | | | | | | | | | | 3.45^{[b]} |
| 146 | 2-iodophenyl | O | Cl | H | H | H | H | H | methyl | H | H | methyl | | 3.40^{[c]}; |
| | | | | | | | | | | | | | | 3.44^{[b]} |
| 147 | 2-fluorophenyl | O | Cl | H | H | H | H | H | methyl | H | H | methyl | | 3.42^{[c]}; |
| | | | | | | | | | | | | | | 3.44^{[b]} |
| 148 | 2-methylphenyl | O | Cl | H | H | H | H | H | methyl | H | H | methyl | | 3.23^{[c]}; |
| | | | | | | | | | | | | | | 3.26^{[b]} |
| 149 | 2-fluoro-6-(trifluoromethyl) | O | Cl | H | Cl | H | H | H | ethane-1,2-diyl | | H | methyl | | 3.95^{[c]}; |
| | phenyl | | | | | | | | | | | | | 4.00^{[b]} |
| 150 | 2,6-difluorophenyl | O | Cl | H | Cl | H | H | H | ethane-1,2-diyl | | H | methyl | | 3.53^{[c]}; |
| | | | | | | | | | | | | | | 3.57^{[b]} |
| 151 | 2-bromophenyl | O | Cl | H | Cl | H | H | H | ethane-1,2-diyl | | H | methyl | | 3.76^{[c]}; |
| | | | | | | | | | | | | | | 3.82^{[b]} |
| 152 | 2-iodophenyl | O | Cl | H | Cl | H | H | H | ethane-1,2-diyl | | H | methyl | | 3.88^{[c]}; |
| | | | | | | | | | | | | | | 3.92^{[b]} |
| 153 | 2-methylphenyl | O | Cl | H | Cl | H | H | H | ethane-1,2-diyl | | H | methyl | | 3.72^{[c]}; |
| | | | | | | | | | | | | | | 3.75^{[b]} |
| 154 | 2-chlorophenyl | O | Cl | H | Cl | H | H | H | ethane-1,2-diyl | | H | methyl | | 3.71^{[c]}; |
| | | | | | | | | | | | | | | 3.76^{[b]} |
| 155 | 2-fluorophenyl | O | Cl | H | Cl | H | H | H | ethane-1,2-diyl | | H | methyl | | 3.75^{[c]}; |
| | | | | | | | | | | | | | | 3.78^{[b]} |
| 156 | 2-chlorophenyl | O | Cl | H | H | H | H | H | methyl | H | H | methyl | | 3.26^{[c]}; |
| | | | | | | | | | | | | | | 3.74^{[b]} |
| 157 | 3-(difluoromethyl)-1-methyl- | O | Cl | H | Cl | H | H | H | methyl | H | cyclo- | methyl | | |
| | 1H-pyrazol-4-yl | | | | | | | | | | propyl | | | |
| 158 | 3-(difluoromethyl)-1-methyl- | O | Cl | H | Cl | H | H | H | H | H | cyclo- | methyl | | |
| | 1H-pyrazol-4-yl | | | | | | | | | | propyl | | | |
| 159 | 5-chloro-3-(trifluoromethyl) | O | Cl | H | Cl | H | H | H | methyl | H | cyclo- | methyl | | 5.39^{[b]} |
| | pyridin-2-yl | | | | | | | | | | propyl | | | |
| 160 | 2-(trifluoromethyl)phenyl | O | Cl | H | Cl | H | H | H | methyl | H | cyclo- | methyl | | |
| | | | | | | | | | | | propyl | | | |
| 161 | 3-(difluoromethyl)-5-fluoro-1- | O | Cl | H | Cl | H | H | H | H | H | cyclo- | methyl | | |
| | methyl-1H-pyrazol-4-yl | | | | | | | | | | propyl | | | |
| 162 | 5-chloro-3-(trifluoromethyl) | O | Cl | H | Cl | H | H | H | H | H | cyclo- | methyl | | |
| | pyridin-2-yl | | | | | | | | | | propyl | | | |
| 163 | 2-(trifluoromethyl)phenyl | O | Cl | H | Cl | H | H | H | H | H | cyclo- | methyl | | |
| | | | | | | | | | | | propyl | | | |
| 164 | 2-(difluoromethyl)pyridin-3-yl | O | H | H | Cl | H | Cl | H | H | H | cyclo- | methyl | | |
| | | | | | | | | | | | propyl | | | |
| 165 | 3-(difluoromethyl)-5-fluoro-1- | O | Cl | H | Cl | H | H | H | methyl | H | cyclo- | methyl | | |
| | methyl-1H-pyrazol-4-yl | | | | | | | | | | propyl | | | |
| 166 | 2-(trifluoromethyl)pyridin-3-yl | O | Cl | H | Cl | H | H | H | H | H | cyclo- | methyl | | 4.16^{[b]} |
| | | | | | | | | | | | propyl | | | |
| 167 | 2-chloropyridin-3-yl | O | Cl | H | Cl | H | H | H | H | H | cyclo- | methyl | | 3.76^{[b]} |
| | | | | | | | | | | | propyl | | | |
| 168 | 3-(trifluoromethyl)pyrazin-2-yl | O | Cl | H | Cl | H | H | H | H | H | cyclo- | methyl | | 4.37^{[b]} |
| | | | | | | | | | | | propyl | | | |
| 169 | 2-methyl-5,6-dihydro-1,4- | O | Cl | H | Cl | H | H | H | H | H | cyclo- | methyl | | 4.11^{[b]} |
| | oxathiin-3-yl | | | | | | | | | | propyl | | | |
| 170 | 2-chloropyridin-3-yl | O | Cl | H | Cl | H | H | H | methyl | H | cyclo- | methyl | | 4.03^{[b]} |
| | | | | | | | | | | | propyl | | | |
| 171 | 2-(difluoromethyl)pyridin-3-yl | O | Cl | H | Cl | H | H | H | methyl | H | cyclo- | methyl | | 3.92^{[b]} |
| | | | | | | | | | | | propyl | | | |
| 172 | 2-methyl-5,6-dihydro-1,4- | O | Cl | H | Cl | H | H | H | methyl | H | cyclo- | methyl | | 4.30^{[b]} |
| | oxathiin-3-yl | | | | | | | | | | propyl | | | |
| 173 | 2-(trifluoromethyl)pyridin-3-yl | O | Cl | H | Cl | H | H | H | methyl | H | cyclo- | methyl | | 4.44^{[b]} |
| | | | | | | | | | | | propyl | | | |
| 174 | 3-(trifluoromethyl)pyrazin-2-yl | O | Cl | H | Cl | H | H | H | methyl | H | cyclo- | methyl | | 4.69^{[b]} |
| | | | | | | | | | | | propyl | | | |
| 175 | 2-chlorophenyl | O | H | H | H | H | H | H | H | H | methyl | methyl | | |
| 176 | | | | | | | | | | | | | | 3.55^{[b]} |
| 177 | 2-chloropyridin-3-yl | O | H | H | H | Cl | H | H | H | H | methyl | methyl | | 2.70^{[b]} |
| 178 | 2-methyl-5,6-dihydro-1,4- | O | H | H | H | Cl | H | H | H | H | methyl | methyl | | 2.98^{[b]} |
| | oxathiin-3-yl | | | | | | | | | | | | | |
| 179 | 3-(trifluoromethyl)pyrazin-2-yl | O | H | H | H | Cl | H | H | H | H | methyl | methyl | | 3.25^{[b]} |
| 180 | 2-(trifluoromethyl)pyridin-3-yl | O | H | H | H | Cl | H | H | H | H | methyl | methyl | | 3.13^{[b]} |
| 181 | 2-(difluoromethyl)pyridin-3-yl | O | H | H | H | Cl | H | H | H | H | methyl | methyl | | 2.68^{[b]} |
| 182 | 2-(trifluoromethyl)phenyl | O | Cl | H | Cl | H | H | H | methyl | methyl | methyl | methyl | | 5.34^{[c]}; |
| | | | | | | | | | | | | | | 5.42^{[b]} |
| 183 | 2-fluorophenyl | O | H | H | fluoro | H | H | H | methyl | H | H | methyl | | 3.02^{[c]}; |
| | | | | | | | | | | | | | | 3.04^{[b]} |
| 184 | 2-chloropyridin-3-yl | O | H | H | fluoro | H | H | H | methyl | H | H | methyl | | 2.23^{[c]}; |
| | | | | | | | | | | | | | | 2.25^{[b]} |
| 185 | 2,6-difluorophenyl | O | H | H | fluoro | H | H | H | methyl | H | H | methyl | | 2.76^{[c]}; |
| | | | | | | | | | | | | | | 2.80^{[b]} |
| 186 | 2-bromophenyl | O | H | H | fluoro | H | H | H | methyl | H | H | methyl | | 2.95^{[c]}; |
| | | | | | | | | | | | | | | 2.99^{[b]} |
| 188 | 2-methylphenyl | O | H | H | fluoro | H | H | H | methyl | H | H | methyl | | 2.86^{[c]}; |
| | | | | | | | | | | | | | | 2.91^{[b]} |
| 189 | 2-fluoro-6-(trifluoromethyl) | O | H | H | fluoro | H | H | H | methyl | H | H | methyl | | 3.19^{[c]}; |
| | phenyl | | | | | | | | | | | | | 3.24^{[b]} |
| 190 | 2-(trifluoromethyl)phenyl | O | H | H | fluoro | H | H | H | methyl | H | H | methyl | | 3.16^{[c]} |
| 191 | 2-chlorophenyl | O | H | H | fluoro | H | H | H | methyl | H | H | methyl | | 2.95^{[c]}; |
| | | | | | | | | | | | | | | 3.02^{[b]} |
| 193 | 2-fluoro-6-(trifluoromethyl) | O | Cl | H | Cl | H | H | H | methyl | H | H | ethyl | | 4.53^{[c]}; |
| | phenyl | | | | | | | | | | | | | 4.55^{[b]} |
| 194 | 2-(trifluoromethyl)phenyl | O | Cl | H | Cl | H | H | H | methyl | H | H | ethyl | | 4.44^{[c]}; |
| | | | | | | | | | | | | | | 4.50[b] |
| 195 | 2-fluorophenyl | O | Cl | H | Cl | H | H | H | methyl | H | H | ethyl | | 4.65^{[c]}; |
| | | | | | | | | | | | | | | 4.66^{[b]} |
| 196 | 2,6-difluorophenyl | O | Cl | H | Cl | H | H | H | methyl | H | H | ethyl | | 4.13^{[c]}; |
| | | | | | | | | | | | | | | 4.14^{[b]} |
| 197 | 2-iodophenyl | O | Cl | H | Cl | H | H | H | methyl | H | H | ethyl | | 4.56^{[c]}; |
| | | | | | | | | | | | | | | 4.51^{[b]} |
| 198 | 2-methylphenyl | O | Cl | H | Cl | H | H | H | methyl | H | H | ethyl | | 4.33^{[c]}; |
| | | | | | | | | | | | | | | 4.35^{[b]} |
| 199 | 2-(difluoromethyl)phenyl | O | Cl | H | Cl | H | H | H | methyl | H | H | ethyl | | 4.40^{[c]}; |
| | | | | | | | | | | | | | | 4.45^{[b]} |
| 200 | 2-bromophenyl | O | Cl | H | Cl | H | H | H | methyl | H | H | ethyl | | 4.39^{[c]}; |
| | | | | | | | | | | | | | | 4.43^{[b]} |
| 201 | 2-chlorophenyl | O | Cl | H | Cl | H | H | H | methyl | H | H | ethyl | | 4.39^{[c]}; |
| | | | | | | | | | | | | | | 4.35^{[b]} |
| 202 | 2-nitrophenyl | O | Cl | H | Cl | H | H | H | methyl | H | H | ethyl | | 3.80^{[c]}; |
| | | | | | | | | | | | | | | 3.83^{[b]} |
| 203 | 2-iodo-3-thienyl | O | Cl | H | H | H | H | H | methyl | H | H | methyl | | 3.53^{[c]}; |
| | | | | | | | | | | | | | | 3.53^{[b]} |
| 204 | 2-methyl-4-(trifluoromethyl)- | O | Cl | H | Cl | H | H | H | methyl | H | H | ethyl | | 4.42^{[c]}; |
| | 1,3-thiazol-5-yl | | | | | | | | | | | | | 4.37^{[b]} |
| 205 | 2-iodo-3-thienyl | O | Cl | H | Cl | H | H | H | methyl | H | H | ethyl | | 4.60^{[c]}; |
| | | | | | | | | | | | | | | 4.63^{[b]} |
| 206 | pyridin-3-yl | O | Cl | H | Cl | H | H | H | methyl | H | H | ethyl | | 3.03^{[c]}; |
| | | | | | | | | | | | | | | 2.89^{[b]} |
| 207 | pyridin-3-yl | O | Cl | H | H | H | H | H | methyl | H | H | methyl | | 2.09^{[c]}; |
| | | | | | | | | | | | | | | 1.94^{[b]} |
| 208 | 3-(trifluoromethyl)pyrazin-2-yl | O | Cl | H | H | H | H | H | methyl | H | H | methyl | | 3.10^{[c]}; |
| | | | | | | | | | | | | | | 3.11^{[b]} |
| 209 | 2-(trifluoromethyl)pyridin-3-yl | O | Cl | H | H | H | H | H | methyl | H | H | methyl | | 3.24^{[c]}; |
| | | | | | | | | | | | | | | 3.27^{[b]} |
| 210 | 3-chloropyrazin-2-yl | O | Cl | H | Cl | H | H | H | ethane-1,2-diyl | | H | methyl | | 3.19^{[c]}; |
| | | | | | | | | | | | | | | 3.24^{[b]} |
| 211 | 2-nitrophenyl | O | Cl | H | Cl | H | H | H | ethane-1,2-diyl | | H | methyl | | 3.26^{[c]}; |
| | | | | | | | | | | | | | | 3.33^{[b]} |
| 212 | 2-methyl-4-(trifluoromethyl)- | O | Cl | H | H | H | H | H | methyl | H | H | methyl | | 3.31^{[c]}; |
| | 1,3-thiazol-5-yl | | | | | | | | | | | | | 3.34^{[b]} |
| 213 | 3-chloropyrazin-2-yl | O | Cl | H | H | H | H | H | methyl | H | H | methyl | | 2.73^{[c]}; |
| | | | | | | | | | | | | | | 2.76^{[b]} |
| 214 | 3-chloropyrazin-2-yl | O | Cl | H | Cl | H | H | H | methyl | H | H | ethyl | | 3.85^{[c]}; |
| | | | | | | | | | | | | | | 3.89^{[b]} |

### NMR-Peak Lists

1H-NMR data of selected examples are written in form of 1H-NMR-peak lists. To each signal peak are listed the δ-value in ppm and the signal intensity in round brackets. Between the δ-value—signal intensity pairs are semicolons as delimiters.

The peak list of an example has therefore the form:

δ₁ (intensity₁); δ₂ (intensity₂); . . . ; δᵢ (intensityᵢ); . . . ; δₙ (intensityₙ)

Intensity of sharp signals correlates with the height of the signals in a printed example of a NMR spectrum in cm and shows the real relations of signal intensities. From broad signals several peaks or the middle of the signal and their relative intensity in comparison to the most intensive signal in the spectrum can be shown.

For calibrating chemical shift for 1H spectra, we use tetramethylsilane and/or the chemical shift of the solvent used, especially in the case of spectra measured in DMSO. Therefore in NMR peak lists, tetramethylsilane peak can occur but not necessarily.

The 1H-NMR peak lists are similar to classical 1H-NMR prints and contain therefore usually all peaks, which are listed at classical NMR-interpretation.

Additionally they can show like classical 1H-NMR prints signals of solvents, stereoisomers of the target compounds, which are also object of the invention, and/or peaks of impurities.

To show compound signals in the delta-range of solvents and/or water the usual peaks of solvents, for example peaks of DMSO in DMSO-D₆ and the peak of water are shown in our 1H-NMR peak lists and have usually on average a high intensity.

The peaks of stereoisomers of the target compounds and/or peaks of impurities have usually on average a lower intensity than the peaks of target compounds (for example with a purity >90%).

Such stereoisomers and/or impurities can be typical for the specific preparation process. Therefore their peaks can help to recognize the reproduction of our preparation process via “side-products-fingerprints”.

An expert, who calculates the peaks of the target compounds with known methods (MestreC, ACD-simulation, but also with empirically evaluated expectation values) can isolate the peaks of the target compounds as needed optionally using additional intensity filters. This isolation would be similar to relevant peak picking at classical 1H-NMR interpretation.

Further details of NMR-data description with peak lists you find in the publication “Citation of NMR Peaklist Data within Patent Applications” of the Research Disclosure Database Number 564025.

### Example 12

¹H-NMR (400.1 MHz. CDCl3): δ=7.922(2.4); 7.796(1.9); 7.519(0.3); 7.407(1.4); 7.401(1.6); 7.396(3.0); 7.391(3.1); 7.386(2.0); 7.372(1.3); 7.367(2.3); 7.362(1.5); 7.333(1.1); 7.312(2.3); 7.308(1.9); 7.303(1.1); 7.287(1.1); 7.282(0.8); 7.270(7.2); 7.265(45.6); 7.260(61.9); 7.191(1.2); 7.186(0.8); 7.174(0.6); 7.170(0.9); 7.165(0.6); 6.996(0.3); 6.965(0.7); 6.933(0.6); 6.833(1.5); 6.829(1.6); 6.801(1.1); 6.797(1.2); 6.694(0.7); 6.662(0.6); 6.613(0.4); 4.695(1.5); 4.690(1.5); 4.687(1.5); 4.602(1.7); 4.598(1.3); 4.539(0.6); 4.451(0.4); 4.430(0.5); 4.413(0.4); 3.929(7.5); 3.892(6.5); 3.318(13.7); 3.316(16.0); 3.314(11.7); 2.009(2.0); 2.004(2.7); 1.569(27.0); 1.565(29.9); 1.376(3.2); 1.371(3.8); 1.359(3.2); 1.354(3.7); 1.031(3.6); 1.027(4.3); 1.014(3.7); 1.010(4.2); 0.010(2.8); 0.005(16.5); 0.000(22.6)

### Example 16

¹H-NMR (400.1 MHz. CDCl3): δ=7.914(2.9); 7.518(0.4); 7.398(0.4); 7.395(0.5); 7.381(1.2); 7.378(1.7); 7.374(1.4); 7.367(0.6); 7.360(3.6); 7.354(2.3); 7.350(4.1); 7.338(1.7); 7.334(1.8); 7.324(0.5); 7.317(1.2); 7.309(0.4); 7.300(0.4); 7.270(0.4); 7.259(70.7); 7.252(0.5); 6.995(0.4); 6.971(1.0); 6.835(2.3); 6.700(1.0); 4.341(0.9); 4.331(0.9); 4.318(1.0); 4.309(0.9); 3.989(0.4); 3.930(8.6); 3.913(0.5); 3.903(0.4); 3.894(0.5); 3.884(0.5); 3.879(0.5); 3.869(0.5); 3.860(0.5); 3.850(0.5); 3.371(0.6); 3.362(0.6); 3.349(0.6); 3.340(0.7); 3.337(0.6); 3.328(0.5); 3.314(0.5); 3.305(0.6); 3.294(16.0); 2.027(0.3); 2.004(0.4); 0.008(1.2)

### Example 18

¹H-NMR (601.6 MHz. CDCl3): δ=7.798(3.2); 7.367(2.6); 7.364(2.7); 7.328(1.9); 7.314(2.2); 7.265(4.1); 7.188(1.3); 7.185(1.2); 7.174(1.0); 7.171(1.0); 6.895(0.9); 6.805(1.8); 6.715(0.9); 6.628(0.4); 6.622(0.4); 6.615(0.4); 4.600(1.9); 4.596(1.9); 4.439(0.4); 4.428(0.6); 4.425(0.4); 4.417(0.4); 3.931(0.4); 3.893(9.0); 3.317(16.0); 2.012(4.9); 1.625(3.6); 1.371(6.3); 1.359(6.2); 0.000(3.0)

### Example 19

¹H-NMR (601.6 MHz. DMSO): δ=8.312(2.2); 8.066(1.0); 8.052(1.1); 7.581(2.9); 7.577(3.1); 7.486(0.9); 7.482(0.9); 7.472(1.6); 7.468(1.6); 7.435(2.8); 7.421(1.7); 7.317(0.6); 7.226(1.6); 7.136(0.7); 4.588(1.8); 4.579(2.0); 4.301(0.4); 4.292(0.4); 4.290(0.5); 4.287(0.5); 4.281(0.5); 4.278(0.5); 4.276(0.5); 4.267(0.4); 3.911(9.7); 3.344(53.8); 3.321(0.4); 3.186(16.0); 2.510(7.3); 2.507(16.1); 2.504(22.5); 2.501(16.4); 2.498(7.4); 2.078(0.4); 1.068(5.2); 1.057(5.2); 0.000(1.8)

### Example 20

¹H-NMR (601.6 MHz. DMSO): δ=8.311(2.2); 8.066(1.0); 8.051(1.1); 7.581(2.9); 7.577(3.0); 7.486(0.9); 7.482(0.9); 7.472(1.6); 7.468(1.6); 7.435(2.8); 7.421(1.7); 7.316(0.6); 7.226(1.6); 7.136(0.7); 4.588(1.9); 4.579(2.0); 4.301(0.4); 4.292(0.4); 4.289(0.6); 4.287(0.5); 4.280(0.5); 4.278(0.5); 4.275(0.5); 4.266(0.4); 3.911(9.8); 3.345(73.9); 3.321(0.5); 3.186(16.0); 3.173(0.6); 3.164(0.6); 2.525(0.3); 2.522(0.4); 2.519(0.4); 2.510(8.7); 2.507(19.3); 2.504(26.5); 2.501(19.3); 2.498(8.7); 2.078(0.5); 1.068(5.2); 1.057(5.2); 0.000(2.1)

### Example 21

¹H-NMR (601.6 MHz. DMSO): δ=8.303(2.2); 7.997(1.0); 7.982(1.0); 7.613(2.9); 7.609(2.9); 7.413(0.9); 7.410(0.8); 7.399(1.6); 7.396(1.6); 7.363(3.0); 7.349(1.8); 7.246(0.6); 7.156(1.9); 7.066(0.7); 4.704(1.8); 4.696(1.9); 4.331(0.4); 4.322(0.4); 4.319(0.5); 4.316(0.4); 4.311(0.4); 4.308(0.5); 4.305(0.4); 4.296(0.4); 4.117(0.4); 4.108(0.4); 3.902(9.8); 3.343(86.4); 3.320(0.5); 3.172(2.6); 3.169(16.0); 3.164(2.0); 2.524(0.4); 2.521(0.5); 2.518(0.4); 2.509(10.6); 2.506(23.6); 2.503(32.5); 2.500(23.8); 2.497(10.7); 2.077(0.6); 1.114(4.8); 1.103(4.8); 0.000(5.3)

### Example 22

¹H-NMR (601.6 MHz. DMSO): δ=8.302(2.2); 7.996(1.0); 7.981(1.0); 7.613(2.9); 7.609(2.9); 7.413(0.9); 7.409(0.8); 7.399(1.6); 7.395(1.6); 7.363(2.9); 7.349(1.7); 7.245(0.6); 7.155(1.8); 7.065(0.7); 4.703(1.8); 4.695(1.9); 4.330(0.4); 4.322(0.4); 4.318(0.5); 4.315(0.4); 4.310(0.4); 4.307(0.5); 4.304(0.4); 4.296(0.4); 3.901(9.8); 3.342(181.9); 3.318(1.3); 3.171(2.2); 3.169(16.0); 3.163(1.5); 2.615(0.5); 2.612(0.3); 2.524(0.9); 2.521(1.1); 2.518(1.1); 2.509(23.6); 2.506(52.2); 2.503(71.9); 2.500(52.4); 2.497(23.6); 2.390(0.3); 2.387(0.5); 2.384(0.3); 2.077(2.0); 1.114(4.8); 1.102(4.8); 0.000(5.5)

### Example 25

¹H-NMR (400.1 MHz. CDCl3): δ=7.908(3.2); 7.316(1.1); 7.312(2.5); 7.307(1.6); 7.260(40.7); 7.250(5.1); 7.245(4.5); 6.959(1.0); 6.823(2.0); 6.757(0.4); 6.754(0.4); 6.688(1.0); 5.298(1.9); 4.297(0.8); 4.288(0.9); 4.276(0.9); 4.266(0.9); 3.930(8.6); 3.875(0.5); 3.866(0.4); 3.857(0.5); 3.847(0.5); 3.840(0.5); 3.831(0.5); 3.822(0.5); 3.812(0.5); 3.316(0.8); 3.310(16.0); 3.300(0.7); 3.295(0.8); 3.285(0.7); 3.282(0.7); 3.277(0.4); 3.272(0.7); 3.260(0.6); 3.250(0.5); 2.169(3.0); 1.558(9.5); 0.000(9.2)

### Example 26

¹H-NMR (400.1 MHz. CDCl3): δ=7.901(3.6); 7.517(1.1); 7.423(2.0); 7.402(3.1); 7.395(2.9); 7.390(2.9); 7.294(2.0); 7.289(1.8); 7.265(24.3); 7.260(196.7); 7.259(192.6); 7.208(0.4); 6.996(1.1); 6.961(1.1); 6.826(2.2); 6.771(0.6); 6.690(1.1); 5.297(0.4); 4.764(1.1); 4.756(1.1); 4.744(1.2); 4.735(1.1); 4.067(0.4); 3.967(0.6); 3.958(0.7); 3.950(0.6); 3.940(0.7); 3.922(10.9); 3.905(0.6); 3.351(0.7); 3.340(0.7); 3.331(0.7); 3.306(16.0); 3.305(16.0); 3.286(0.6); 2.170(0.8); 2.168(0.8); 2.004(0.6); 1.546(137.2); 0.006(3.0); 0.000(23.7)

### Example 27

¹H-NMR (400.1 MHz. CDCl3): δ=11.874(0.9); 7.926(4.2); 7.865(0.9); 7.853(3.5); 7.720(0.9); 7.644(1.0); 7.519(9.0); 7.411(1.1); 7.387(1.0); 7.362(1.1); 7.360(1.0); 7.326(0.9); 7.308(4.3); 7.289(4.5); 7.284(4.4); 7.261(1446.3); 7.260(1575.7); 7.244(10.5); 7.209(3.5); 7.181(5.1); 7.176(5.0); 7.162(1.0); 7.156(1.0); 7.049(1.0); 6.996(8.8); 6.963(1.6); 6.931(1.3); 6.827(2.5); 6.794(2.2); 6.755(0.9); 6.717(0.9); 6.691(1.5); 6.659(1.2); 6.443(1.1); 4.367(2.0); 4.359(2.6); 4.327(1.3); 4.310(1.3); 4.203(2.3); 4.193(1.9); 3.936(10.6); 3.911(10.1); 3.661(3.6); 3.353(16.0); 3.333(15.1); 2.689(0.8); 2.624(1.0); 2.013(0.9); 2.004(1.7); 1.642(1.0); 1.627(1.2); 1.601(1.3); 1.592(3.5); 1.544(928.4); 1.493(1.7); 1.464(0.9); 1.440(1.0); 1.250(6.8); 1.232(6.6); 1.032(7.0); 1.015(6.7); 0.813(0.9); 0.147(3.1); 0.048(1.4); 0.031(1.1); 0.010(15.6); 0.008(17.3); 0.002(511.6)

### Example 28

¹H-NMR (400.1 MHz. CDCl3): δ=7.912(3.4); 7.776(2.8); 7.518(2.6); 7.416(1.8); 7.409(0.4); 7.395(2.6); 7.381(2.5); 7.376(2.8); 7.358(2.1); 7.352(2.3); 7.310(5.6); 7.306(1.9); 7.298(1.7); 7.293(1.8); 7.285(2.4); 7.277(2.1); 7.260(451.2); 7.210(2.2); 7.159(1.4); 7.153(1.1); 7.138(0.8); 7.133(0.8); 6.996(2.5); 6.954(1.0); 6.929(0.8); 6.818(2.0); 6.793(1.7); 6.683(1.0); 6.658(1.2); 6.630(0.5); 6.560(0.5); 4.718(1.5); 4.712(1.7); 4.687(1.8); 4.677(2.0); 4.446(0.5); 4.436(0.5); 4.422(0.5); 4.269(0.5); 4.251(0.5); 3.924(8.9); 3.904(0.3); 3.888(7.1); 3.301(13.5); 3.280(16.0); 2.004(2.7); 1.834(0.4); 1.816(0.6); 1.797(0.7); 1.781(0.5); 1.764(0.6); 1.745(0.9); 1.726(0.7); 1.711(0.4); 1.689(0.3); 1.641(0.3); 1.590(3.1); 1.540(262.7); 1.491(1.6); 1.472(1.4); 1.453(2.4); 1.435(1.9); 1.417(0.6); 1.061(2.6); 1.042(5.4); 1.023(2.4); 0.871(3.2); 0.852(6.5); 0.834(3.0); 0.146(0.7); 0.050(1.9); 0.008(5.3)

### Example 29

¹H-NMR (400.1 MHz. CDCl3): δ=7.930(5.6); 7.801(1.8); 7.518(1.2); 7.515(1.2); 7.426(2.7); 7.406(3.8); 7.382(3.4); 7.377(4.2); 7.374(3.6); 7.359(1.2); 7.345(1.6); 7.342(1.5); 7.338(1.5); 7.291(2.8); 7.269(24.1); 7.260(207.7); 7.256(196.4); 7.206(0.5); 7.203(0.4); 7.193(0.3); 7.169(0.9); 7.152(0.7); 6.996(1.2); 6.993(1.1); 6.951(1.6); 6.922(1.3); 6.816(3.0); 6.789(1.1); 6.752(0.4); 6.681(1.5); 6.654(0.5); 4.788(3.3); 4.779(3.4); 4.680(1.3); 4.545(0.9); 4.532(1.2); 4.414(0.4); 4.404(0.4); 4.397(0.5); 4.376(0.4); 3.925(16.0); 3.896(0.9); 3.886(5.5); 3.488(0.6); 3.471(1.5); 3.449(2.2); 3.432(1.8); 3.414(1.3); 3.398(2.1); 3.379(2.0); 3.359(1.0); 3.342(0.3); 2.004(1.4); 2.001(1.3); 1.549(108.5); 1.546(104.1); 1.491(0.4); 1.382(3.1); 1.368(3.1); 1.244(6.5); 1.226(12.9); 1.209(6.1); 1.025(9.0); 1.023(8.9); 1.008(9.0); 0.145(0.5); 0.008(9.1); 0.000(80.7)

### Example 30

¹H-NMR (400.0 MHz. DMSO): δ=8.475(1.2); 8.453(1.2); 8.396(1.1); 8.374(1.1); 8.319(0.4); 7.625(3.9); 7.622(4.4); 7.506(0.8); 7.501(1.3); 7.496(0.7); 7.484(2.3); 7.480(4.6); 7.475(3.1); 7.466(4.6); 7.462(4.7); 7.453(1.2); 7.438(4.3); 7.433(2.4); 7.427(1.9); 7.423(1.9); 7.409(3.0); 7.405(2.9); 7.389(1.5); 7.385(1.6); 7.380(1.1); 7.375(1.9); 7.371(1.2); 7.361(1.6); 7.357(2.9); 7.353(1.8); 7.343(0.8); 7.339(1.2); 7.335(0.8); 7.274(1.5); 7.270(1.4); 7.256(1.2); 7.252(1.0); 7.226(1.7); 7.222(1.6); 7.207(1.4); 7.203(1.3); 4.688(1.9); 4.676(2.0); 4.619(2.2); 4.604(2.4); 4.342(0.5); 4.321(1.0); 4.304(1.3); 4.289(0.9); 4.284(0.9); 4.267(0.5); 3.904(9.4); 3.334(135.5); 3.184(16.0); 3.177(14.3); 3.161(0.8); 2.676(0.6); 2.671(0.8); 2.667(0.6); 2.542(0.5); 2.524(2.9); 2.507(100.6); 2.502(130.9); 2.498(100.6); 2.333(0.6); 2.329(0.8); 2.325(0.6); 1.135(5.1); 1.114(7.5); 1.097(6.0); 0.008(0.4); 0.000(11.2); −0.008(0.5)

### Example 32

¹H-NMR (400.1 MHz. CDCl3): δ=14.892(0.4); 7.823(4.4); 7.518(2.0); 7.460(0.4); 7.407(2.5); 7.386(3.0); 7.371(0.5); 7.356(3.3); 7.311(2.2); 7.259(335.1); 7.257(313.8); 7.234(1.8); 7.210(1.7); 7.207(3.3); 7.204(0.6); 6.995(2.0); 6.896(1.1); 6.761(2.1); 6.625(1.2); 6.487(1.1); 5.298(3.6); 5.296(3.4); 5.070(4.2); 4.050(0.5); 4.003(0.7); 3.903(12.6); 3.226(16.0); 3.224(15.5); 1.584(0.7); 1.532(132.7); 1.530(127.7); 1.480(1.4); 1.476(0.3); 1.255(0.3); 1.101(1.2); 1.081(1.3); 0.902(0.7); 0.892(1.2); 0.867(2.2); 0.853(5.3); 0.841(1.4); 0.821(0.4); 0.809(0.3); 0.145(0.9); 0.052(0.9); 0.008(4.5)

### Example 33

¹H-NMR (400.1 MHz. CDCl3): δ=8.274(1.9); 8.254(2.1); 8.077(1.5); 8.057(1.6); 7.741(2.7); 7.721(2.3); 7.683(1.9); 7.664(1.7); 7.637(0.3); 7.520(2.9); 7.517(2.6); 7.467(0.4); 7.458(0.4); 7.429(2.8); 7.424(4.1); 7.419(2.0); 7.408(1.9); 7.386(3.0); 7.377(0.5); 7.358(0.7); 7.337(2.1); 7.316(2.6); 7.307(1.1); 7.305(1.0); 7.303(1.0); 7.300(1.1); 7.295(2.9); 7.289(1.3); 7.287(1.5); 7.287(1.5); 7.286(1.4); 7.285(1.3); 7.284(1.3); 7.283(1.5); 7.282(1.5); 7.282(1.4); 7.281(1.7); 7.280(1.7); 7.279(1.9); 7.278(2.0); 7.278(2.3); 7.277(2.6); 7.276(2.8); 7.275(3.1); 7.274(3.4); 7.273(3.5); 7.273(4.2); 7.272(4.4); 7.271(4.9); 7.270(5.5); 7.264(84.9); 7.261(515.0); 7.258(416.9); 7.253(5.3); 7.252(4.8); 7.251(3.9); 7.250(3.1); 7.249(2.5); 7.249(2.6); 7.248(2.3); 7.247(2.4); 7.246(2.0); 7.245(2.0); 7.245(1.6); 7.244(1.7); 7.242(1.8); 7.241(1.6); 7.234(0.4); 7.225(1.0); 7.207(0.5); 6.997(2.7); 6.994(2.4); 6.893(0.7); 6.875(0.8); 6.732(0.5); 6.720(0.6); 6.702(0.7); 4.879(0.3); 4.775(2.1); 4.766(2.3); 4.640(1.8); 4.634(2.0); 4.619(0.6); 4.605(0.8); 4.597(0.7); 4.589(0.6); 4.582(0.6); 4.541(0.7); 4.522(0.8); 4.507(0.6); 3.740(1.3); 3.423(0.4); 3.404(0.3); 3.395(0.3); 3.365(0.4); 3.341(11.7); 3.338(10.1); 3.327(16.0); 3.324(13.9); 2.819(0.9); 2.809(0.9); 2.307(1.2); 2.063(1.2); 1.934(0.4); 1.645(0.5); 1.611(0.4); 1.545(128.9); 1.493(0.6); 1.461(5.0); 1.444(4.8); 1.255(0.4); 1.146(0.5); 1.130(0.6); 1.104(6.3); 1.087(6.4); 0.842(0.4); 0.145(1.0); 0.020(0.5); 0.000(231.6)

### Example 34

¹H-NMR (400.0 MHz. DMSO): δ=8.683(1.2); 8.662(1.2); 8.598(0.7); 8.576(0.7); 8.452(1.6); 8.447(1.7); 8.439(2.4); 8.435(2.4); 8.427(1.1); 8.422(1.0); 7.723(1.6); 7.718(1.6); 7.704(1.9); 7.699(1.8); 7.685(1.0); 7.680(1.0); 7.666(1.2); 7.661(1.1); 7.639(2.6); 7.634(4.0); 7.628(1.9); 7.511(1.3); 7.506(1.2); 7.490(3.9); 7.488(3.8); 7.485(3.8); 7.476(2.3); 7.474(2.1); 7.468(4.6); 7.458(3.4); 7.446(1.5); 7.443(1.3); 7.438(1.1); 4.677(1.3); 4.665(1.4); 4.637(2.2); 4.624(2.3); 4.329(0.4); 4.323(0.4); 4.314(0.7); 4.307(0.5); 4.301(0.9); 4.297(0.8); 4.294(0.8); 4.284(0.7); 4.280(0.7); 4.263(0.5); 4.111(0.3); 4.098(0.3); 3.904(10.1); 3.334(105.5); 3.200(16.0); 3.189(9.8); 3.174(1.6); 3.161(1.4); 3.156(0.5); 2.676(0.5); 2.672(0.7); 2.667(0.5); 2.542(0.5); 2.525(2.0); 2.511(40.3); 2.507(80.1); 2.503(106.4); 2.498(81.3); 2.494(42.4); 2.334(0.5); 2.329(0.7); 2.325(0.5); 1.172(3.4); 1.155(3.4); 1.090(5.9); 1.073(5.8); 0.985(0.3); 0.008(0.5); 0.000(16.3); −0.008(0.7)

### Example 35

¹H-NMR (400.0 MHz. DMSO): δ=8.566(0.8); 8.544(0.8); 8.501(0.6); 8.478(0.6); 7.756(0.9); 7.738(1.8); 7.719(1.3); 7.697(1.3); 7.678(0.9); 7.649(1.9); 7.644(2.1); 7.639(1.8); 7.635(2.2); 7.615(1.1); 7.596(0.4); 7.529(0.4); 7.524(0.3); 7.508(1.7); 7.503(1.6); 7.489(2.4); 7.483(1.5); 7.469(0.7); 7.461(2.4); 7.440(1.0); 7.346(0.7); 7.327(0.7); 7.288(1.0); 7.270(0.9); 4.675(1.0); 4.662(1.1); 4.623(1.4); 4.609(1.5); 4.309(0.5); 4.306(0.4); 4.302(0.4); 4.288(0.7); 4.279(0.6); 4.274(0.6); 4.257(0.3); 3.904(6.4); 3.335(62.9); 3.182(16.0); 3.161(0.6); 2.723(0.4); 2.676(0.4); 2.672(0.6); 2.667(0.4); 2.542(0.4); 2.507(68.1); 2.503(88.0); 2.498(67.3); 2.334(0.4); 2.329(0.5); 2.325(0.4); 1.114(2.6); 1.096(2.7); 1.080(3.9); 1.063(3.8); 0.985(0.3); 0.008(0.4); 0.000(11.0); −0.008(0.5)

### Example 36

¹H-NMR (400.1 MHz. CDCl3): δ=18.777(0.6); 17.997(0.6); 8.121(2.8); 8.100(3.1); 7.939(2.3); 7.919(2.4); 7.518(6.6); 7.516(5.2); 7.456(0.7); 7.420(2.7); 7.415(2.8); 7.403(3.4); 7.392(3.2); 7.383(3.2); 7.372(3.0); 7.331(2.9); 7.324(2.0); 7.317(2.1); 7.314(2.3); 7.306(3.3); 7.296(3.3); 7.285(2.5); 7.282(2.7); 7.280(3.1); 7.278(3.3); 7.275(3.9); 7.274(3.9); 7.273(5.2); 7.272(5.7); 7.271(5.6); 7.270(6.3); 7.269(7.2); 7.262(183.0); 7.259(1071.0); 7.257(902.5); 7.253(17.6); 7.252(15.0); 7.251(13.1); 7.250(11.6); 7.249(10.2); 7.248(6.9); 7.247(5.6); 7.246(4.1); 7.245(3.6); 7.245(3.0); 7.244(2.9); 7.243(2.7); 7.242(2.3); 7.241(2.4); 7.240(2.0); 7.240(2.2); 7.238(1.6); 7.235(1.4); 7.232(2.1); 7.227(1.7); 7.227(1.8); 7.204(0.8); 6.995(6.4); 6.993(5.2); 6.980(0.7); 6.955(0.8); 6.948(0.7); 6.811(0.7); 5.298(6.3); 5.296(4.8); 4.761(2.1); 4.751(2.2); 4.633(1.8); 4.627(1.9); 4.600(0.9); 4.593(0.9); 4.562(0.7); 4.527(0.7); 4.503(0.9); 3.399(0.7); 3.388(0.8); 3.337(11.7); 3.324(16.0); 3.322(13.3); 2.823(1.4); 2.812(1.5); 2.049(0.7); 1.695(0.6); 1.623(0.7); 1.588(0.8); 1.532(272.1); 1.438(5.1); 1.421(4.9); 1.143(0.7); 1.127(1.1); 1.110(0.7); 1.087(6.4); 1.070(6.2); 0.857(0.9); 0.821(0.7); 0.145(2.0); 0.045(1.1); 0.033(1.0); 0.000(504.8)

### Example 37

¹H-NMR (400.1 MHz. CDCl3): δ=8.269(1.1); 8.267(1.1); 8.249(1.2); 8.248(1.2); 7.729(2.1); 7.710(1.9); 7.432(2.2); 7.426(3.5); 7.405(2.0); 7.328(1.1); 7.327(1.1); 7.323(1.1); 7.322(1.1); 7.307(0.8); 7.306(0.8); 7.302(0.7); 7.301(0.7); 7.267(0.3); 7.261(26.4); 7.255(0.4); 4.846(0.6); 4.837(0.7); 4.826(0.7); 4.817(0.7); 4.047(0.5); 4.038(0.5); 4.029(0.5); 4.020(0.5); 4.012(0.6); 4.003(0.6); 3.994(0.6); 3.985(0.6); 3.462(0.6); 3.451(0.6); 3.441(0.5); 3.431(0.6); 3.427(0.6); 3.416(0.5); 3.407(0.5); 3.396(0.5); 3.320(16.0); 1.554(9.1)

### Example 38

¹H-NMR (400.1 MHz. CDCl3): δ=18.986(0.5); 16.568(0.6); 13.354(0.5); 8.060(1.9); 8.040(2.1); 7.889(1.3); 7.869(1.3); 7.518(7.5); 7.414(2.3); 7.411(2.7); 7.398(1.4); 7.392(2.3); 7.361(1.0); 7.358(2.5); 7.338(1.0); 7.325(1.2); 7.318(1.8); 7.310(16.0); 7.303(1.5); 7.298(1.6); 7.287(1.3); 7.259(1300.9); 7.223(2.7); 7.218(2.6); 7.211(5.9); 7.196(0.6); 7.185(1.4); 7.162(1.3); 7.148(1.0); 7.129(1.1); 7.020(0.5); 6.996(7.5); 6.842(0.5); 4.768(1.5); 4.758(1.6); 4.639(1.1); 4.632(1.2); 4.619(0.5); 4.608(0.5); 4.592(0.5); 4.585(0.5); 4.510(0.5); 3.491(3.0); 3.331(9.4); 3.325(13.9); 2.581(9.9); 2.557(7.1); 2.004(5.5); 1.634(0.7); 1.585(5.6); 1.560(2.0); 1.535(438.0); 1.487(2.2); 1.434(3.5); 1.417(3.6); 1.408(0.7); 1.087(5.0); 1.070(5.3); 0.813(0.7); 0.147(0.9); 0.050(3.3); 0.008(8.3)

### Example 39

¹H-NMR (400.1 MHz. CDCl3): δ=8.785(0.8); 8.783(0.9); 8.774(0.8); 8.771(0.8); 8.751(0.6); 8.748(0.7); 8.739(0.7); 7.920(0.8); 7.917(0.8); 7.900(0.9); 7.898(0.9); 7.896(0.8); 7.658(0.6); 7.655(0.6); 7.639(0.8); 7.636(0.8); 7.587(0.8); 7.576(0.9); 7.569(0.8); 7.557(0.8); 7.534(0.7); 7.522(0.8); 7.518(1.8); 7.503(0.6); 7.437(1.5); 7.434(3.6); 7.432(2.0); 7.428(2.4); 7.390(1.3); 7.369(2.4); 7.329(1.4); 7.328(1.5); 7.324(1.3); 7.323(1.3); 7.310(0.5); 7.308(1.1); 7.307(1.2); 7.303(0.8); 7.302(0.9); 7.297(0.3); 7.296(0.4); 7.291(0.4); 7.291(0.4); 7.290(0.4); 7.289(0.4); 7.288(0.4); 7.287(0.4); 7.286(0.5); 7.286(0.6); 7.285(0.6); 7.284(0.7); 7.283(0.7); 7.280(3.4); 7.278(3.6); 7.276(5.4); 7.273(1.4); 7.273(1.3); 7.272(1.4); 7.271(1.5); 7.270(1.7); 7.269(1.8); 7.269(1.9); 7.268(2.5); 7.267(2.9); 7.266(3.6); 7.265(4.6); 7.264(5.7); 7.259(303.5); 7.254(7.9); 7.254(6.2); 7.253(5.1); 7.252(4.5); 7.251(3.8); 7.250(3.5); 7.249(3.0); 7.249(2.6); 7.248(2.3); 7.247(2.0); 7.246(1.9); 7.245(1.7); 7.245(1.6); 7.244(1.4); 7.243(1.3); 7.242(1.2); 7.241(1.0); 7.240(1.0); 7.240(0.9); 7.239(0.9); 7.238(0.8); 7.237(0.8); 7.236(0.7); 7.236(0.6); 7.235(0.6); 7.234(0.5); 7.233(0.5); 7.232(0.5); 7.231(0.5); 7.231(0.4); 7.230(0.4); 7.229(0.4); 7.225(0.3); 6.995(1.7); 6.253(0.4); 6.228(0.4); 6.048(0.4); 6.025(0.3); 4.754(1.5); 4.745(1.7); 4.621(1.8); 4.614(2.0); 4.610(0.6); 4.601(0.4); 4.593(0.4); 4.587(0.5); 4.578(0.4); 4.570(0.4); 4.561(0.4); 4.507(0.3); 4.500(0.3); 4.490(0.3); 4.484(0.6); 4.467(0.4); 3.308(14.2); 3.296(16.0); 2.004(3.5); 1.533(85.2); 1.427(4.3); 1.410(4.3); 1.073(5.4); 1.056(5.3); 0.146(0.4); 0.008(4.5); 0.000(139.1)

### Example 40

¹H-NMR (400.0 MHz. DMSO): δ=8.072(0.5); 8.057(1.0); 8.043(0.5); 7.560(1.5); 7.556(3.0); 7.551(1.7); 7.375(5.4); 7.371(5.3); 7.161(1.1); 7.027(2.4); 6.892(1.2); 4.434(0.8); 4.419(1.7); 4.403(0.8); 3.863(10.0); 3.528(0.4); 3.509(0.7); 3.494(1.1); 3.485(0.8); 3.479(0.8); 3.470(1.3); 3.454(0.7); 3.436(0.4); 3.331(25.0); 3.221(16.0); 2.891(0.4); 2.507(14.0); 2.503(19.0); 2.499(13.7)

### Example 41

¹H-NMR (400.0 MHz. DMSO): δ=7.960(0.6); 7.946(1.2); 7.932(0.6); 7.570(1.9); 7.565(3.2); 7.561(1.8); 7.362(5.9); 7.357(5.6); 7.210(1.0); 7.075(2.4); 6.941(1.1); 4.443(0.9); 4.429(1.7); 4.413(0.9); 3.876(0.3); 3.788(8.6); 3.507(0.5); 3.504(0.5); 3.484(0.9); 3.469(1.3); 3.461(1.0); 3.454(1.1); 3.446(1.4); 3.431(0.8); 3.412(0.5); 3.397(0.4); 3.346(15.5); 3.234(1.1); 3.223(16.0); 3.202(0.4); 2.904(0.4); 2.745(0.3); 2.520(11.3); 2.516(14.4)

### Example 42

¹H-NMR (400.0 MHz. DMSO): δ=7.999(1.3); 7.977(1.3); 7.835(0.7); 7.814(0.7); 7.543(1.6); 7.538(3.2); 7.533(2.7); 7.529(2.0); 7.524(1.2); 7.362(3.2); 7.358(3.3); 7.336(6.0); 7.332(6.1); 7.138(0.6); 7.047(1.1); 7.003(1.3); 6.912(2.4); 6.868(0.7); 6.777(1.2); 4.384(1.1); 4.373(1.2); 4.232(2.1); 4.215(2.7); 4.204(0.4); 4.200(0.4); 4.194(0.3); 4.120(0.6); 4.103(0.9); 4.099(0.7); 4.082(0.9); 4.065(0.5); 3.863(6.7); 3.848(11.7); 3.333(27.9); 3.313(0.3); 3.237(8.8); 3.206(16.0); 2.507(17.7); 2.503(24.4); 2.499(18.1); 1.231(0.4); 1.153(6.0); 1.137(6.0); 1.105(3.3); 1.088(3.2)

### Example 43

¹H-NMR (400.0 MHz. DMSO): δ=7.801(1.2); 7.779(1.1); 7.691(1.0); 7.670(1.0); 7.530(1.5); 7.526(4.1); 7.521(4.6); 7.517(2.0); 7.332(4.8); 7.327(4.6); 7.310(5.6); 7.305(5.7); 7.140(0.9); 7.093(1.1); 7.005(1.7); 6.958(2.2); 6.871(1.0); 6.824(1.2); 4.370(1.5); 4.359(1.7); 4.252(1.8); 4.235(2.1); 4.207(0.4); 4.191(0.5); 4.187(0.5); 4.179(0.5); 4.158(0.4); 4.132(0.4); 4.102(0.5); 4.084(0.8); 4.063(0.8); 4.046(0.5); 3.848(0.4); 3.776(7.2); 3.764(8.4); 3.476(0.4); 3.381(0.3); 3.331(99.4); 3.312(0.6); 3.236(0.6); 3.223(13.4); 3.214(16.0); 2.891(0.9); 2.732(0.7); 2.671(0.4); 2.525(0.6); 2.506(46.9); 2.502(62.6); 2.329(0.4); 2.147(0.3); 1.152(0.3); 1.132(5.7); 1.115(5.6); 1.090(4.7); 1.073(4.5); 0.000(0.5)

### Example 44

¹H-NMR (400.0 MHz. DMSO): δ=8.108(0.5); 8.093(1.1); 8.079(0.5); 7.636(1.7); 7.633(3.2); 7.630(2.0); 7.494(6.5); 7.491(6.2); 7.190(1.0); 7.055(2.4); 6.920(1.1); 4.788(0.9); 4.775(1.1); 4.771(1.4); 4.759(1.0); 3.879(10.3); 3.530(0.8); 3.515(1.4); 3.503(0.9); 3.497(1.3); 3.481(0.8); 3.463(0.3); 3.344(15.4); 3.222(16.0); 2.519(8.8); 2.515(12.2); 2.511(8.9)

### Example 45

¹H-NMR (400.0 MHz. DMSO): δ=7.965(0.4); 7.950(0.9); 7.936(0.4); 7.620(2.4); 7.616(2.6); 7.496(0.6); 7.491(0.5); 7.475(2.2); 7.470(2.4); 7.461(3.5); 7.440(0.8); 7.204(0.9); 7.070(2.3); 6.936(1.0); 4.783(0.8); 4.769(1.7); 4.754(0.8); 3.778(6.7); 3.482(1.0); 3.466(2.2); 3.451(1.3); 3.334(12.9); 3.211(0.5); 3.205(0.4); 3.196(16.0); 2.892(0.4); 2.508(6.9); 2.504(9.5); 2.500(6.7)

### Example 46

¹H-NMR (400.0 MHz. DMSO): δ=7.857(1.4); 7.835(1.5); 7.770(0.5); 7.748(0.5); 7.610(3.1); 7.607(4.4); 7.602(1.6); 7.498(0.5); 7.494(0.3); 7.477(3.9); 7.472(7.2); 7.452(1.5); 7.429(0.9); 7.424(0.8); 7.408(0.3); 7.403(0.3); 7.172(1.3); 7.125(0.4); 7.037(2.9); 6.990(0.9); 6.903(1.5); 6.855(0.5); 4.763(0.8); 4.752(0.8); 4.727(2.9); 4.715(3.0); 4.340(0.6); 4.327(0.6); 4.323(0.8); 4.319(0.7); 4.310(0.7); 4.305(0.8); 4.301(0.7); 4.288(0.6); 3.913(0.4); 3.867(16.0); 3.386(0.4); 3.379(1.1); 3.368(1.2); 3.362(3.4); 3.344(3.9); 3.333(29.8); 2.522(0.4); 2.508(17.5); 2.504(24.2); 2.500(17.4); 1.966(0.4); 1.514(0.4); 1.232(0.4); 1.172(2.2); 1.157(5.6); 1.146(2.1); 1.140(9.9); 1.128(3.5); 1.122(4.6); 1.111(1.5); 1.045(7.0); 1.028(6.9)

### Example 47

¹H-NMR (400.0 MHz. DMSO): δ=7.624(1.8); 7.600(6.8); 7.597(6.3); 7.492(0.5); 7.488(0.3); 7.471(5.3); 7.467(10.3); 7.446(0.6); 7.416(3.2); 7.413(3.5); 7.184(1.6); 7.118(0.5); 7.049(3.5); 6.983(1.1); 6.915(1.9); 6.849(0.6); 4.775(1.2); 4.764(1.3); 4.711(3.7); 4.698(3.9); 4.325(0.7); 4.307(0.9); 4.304(0.9); 4.294(0.8); 4.290(1.0); 4.286(0.9); 4.273(0.8); 4.260(0.4); 3.867(0.6); 3.800(0.4); 3.777(16.0); 3.506(0.4); 3.388(0.6); 3.381(1.1); 3.371(1.2); 3.363(3.6); 3.355(2.8); 3.346(3.9); 3.333(46.3); 3.321(1.3); 3.314(0.8); 2.892(0.5); 2.732(0.4); 2.508(20.3); 2.504(28.1); 2.500(19.9); 1.926(0.6); 1.232(0.3); 1.202(0.4); 1.165(2.8); 1.156(6.4); 1.148(3.3); 1.139(13.5); 1.122(7.0); 1.106(2.0); 1.038(8.9); 1.021(8.9)

### Example 48

¹H-NMR (400.0 MHz. DMSO): δ=7.877(1.1); 7.854(1.1); 7.746(0.8); 7.723(0.9); 7.603(2.1); 7.600(4.5); 7.595(3.1); 7.483(0.7); 7.478(0.5); 7.462(2.4); 7.457(2.6); 7.447(3.9); 7.426(1.2); 7.405(4.5); 7.400(2.3); 7.065(1.1); 7.040(0.8); 6.930(2.3); 6.905(1.8); 6.795(1.2); 6.770(0.9); 4.714(1.7); 4.705(1.8); 4.598(2.0); 4.583(2.2); 4.196(0.5); 4.190(0.4); 4.187(0.4); 4.180(0.4); 4.172(0.5); 4.163(0.4); 4.156(0.4); 4.149(0.4); 4.140(0.5); 4.131(0.4); 4.127(0.4); 4.118(0.5); 3.899(0.3); 3.869(0.5); 3.852(12.5); 3.333(40.0); 3.210(11.8); 3.173(16.0); 2.892(1.1); 2.732(0.9); 2.503(23.3); 1.648(0.5); 1.633(0.5); 1.629(0.5); 1.619(0.6); 1.615(0.6); 1.610(0.4); 1.600(0.7); 1.578(0.7); 1.574(0.6); 1.566(0.6); 1.556(0.5); 1.547(0.5); 1.523(0.5); 1.513(0.6); 1.505(0.5); 1.496(0.4); 1.488(0.4); 1.478(0.5); 1.461(0.3); 1.231(0.4); 0.936(2.0); 0.917(4.5); 0.899(1.8); 0.835(2.5); 0.817(5.3); 0.798(2.2)

### Example 49

¹H-NMR (400.0 MHz. DMSO): δ=7.623(1.4); 7.616(1.2); 7.603(3.3); 7.598(3.7); 7.588(3.5); 7.583(3.2); 7.475(0.8); 7.470(0.6); 7.454(2.6); 7.449(2.7); 7.438(3.9); 7.417(1.1); 7.408(0.8); 7.404(0.7); 7.388(1.7); 7.383(1.6); 7.362(2.5); 7.341(0.9); 7.113(1.0); 7.045(0.7); 6.978(2.2); 6.911(1.6); 6.844(1.2); 6.776(0.8); 4.725(1.6); 4.716(1.6); 4.589(2.1); 4.574(2.2); 4.176(0.6); 4.152(0.7); 4.142(0.3); 4.135(0.4); 4.126(0.3); 4.100(0.5); 4.091(0.5); 4.086(0.4); 4.077(0.5); 3.852(0.4); 3.768(13.1); 3.333(53.6); 3.204(11.2); 3.171(16.0); 2.892(0.7); 2.733(0.6); 2.508(21.6); 2.503(27.9); 2.499(19.5); 1.648(0.6); 1.629(0.6); 1.609(0.8); 1.600(0.5); 1.591(0.9); 1.581(0.6); 1.574(0.9); 1.568(0.8); 1.555(0.8); 1.547(0.6); 1.536(0.4); 1.518(0.5); 1.500(0.6); 1.491(0.5); 1.483(0.4); 1.473(0.6); 1.465(0.3); 1.456(0.4); 0.913(2.1); 0.894(4.4); 0.876(1.9); 0.806(2.7); 0.788(5.7); 0.769(2.4)

### Example 50

¹H-NMR (400.1 MHz. DMSO): δ=8.189(2.7); 7.576(3.0); 7.571(3.1); 7.505(1.7); 7.484(3.7); 7.453(2.2); 7.448(2.0); 7.432(1.0); 7.427(1.0); 7.068(1.1); 6.933(2.4); 6.798(1.2); 5.762(1.8); 5.007(4.6); 3.863(0.4); 3.834(11.3); 3.324(2.5); 3.139(16.0); 2.514(10.6); 2.510(19.9); 2.506(26.0); 2.501(19.1); 1.077(0.7); 1.072(0.7); 1.062(0.7); 1.047(0.8); 1.038(0.6); 0.953(0.5); 0.942(0.7); 0.939(0.8); 0.928(0.7); 0.918(0.7); 0.913(0.7); 0.748(0.3); 0.733(0.3); 0.723(0.9); 0.705(2.8); 0.687(0.9); 0.677(0.4); 0.672(0.4); 0.662(0.3)

### Example 51

¹H-NMR (400.1 MHz. DMSO): δ=8.061(2.6); 7.572(2.8); 7.568(2.9); 7.483(1.1); 7.462(4.2); 7.451(2.8); 7.447(2.4); 7.430(0.7); 7.425(0.7); 7.113(1.0); 6.978(2.4); 6.844(1.2); 5.762(1.2); 4.968(4.7); 3.777(0.3); 3.745(8.6); 3.474(0.3); 3.324(3.1); 3.175(0.4); 3.130(16.0); 3.096(0.4); 2.545(0.4); 2.515(13.3); 2.510(25.7); 2.506(34.3); 2.502(25.5); 1.097(0.6); 1.093(0.6); 1.085(0.5); 1.072(1.1); 1.068(1.1); 0.941(1.0); 0.936(1.1); 0.923(0.5); 0.911(0.7); 0.702(1.2); 0.694(2.9); 0.689(2.9); 0.663(0.4)

### Example 52

¹H-NMR (400.1 MHz. DMSO): δ=8.949(5.9); 8.943(4.8); 8.624(2.5); 8.602(2.5); 8.528(0.8); 8.501(5.4); 8.496(5.0); 8.472(1.7); 8.466(1.6); 7.605(7.3); 7.598(2.6); 7.498(1.5); 7.477(15.5); 7.424(1.3); 7.419(1.2); 7.403(0.7); 7.398(0.7); 4.775(1.4); 4.763(1.5); 4.749(5.0); 4.738(5.2); 4.330(0.9); 4.318(1.0); 4.313(1.2); 4.309(1.1); 4.301(1.1); 4.297(1.3); 4.291(1.1); 4.280(1.1); 4.264(0.5); 4.253(0.3); 4.248(0.4); 4.243(0.4); 4.231(0.3); 3.384(0.7); 3.378(1.1); 3.367(2.1); 3.361(3.3); 3.351(5.1); 3.343(3.7); 3.334(5.4); 3.326(2.6); 3.311(115.2); 3.260(0.3); 2.671(0.5); 2.667(0.3); 2.524(0.8); 2.511(24.1); 2.506(52.2); 2.502(73.5); 2.497(53.7); 2.493(26.0); 2.456(0.3); 2.452(0.4); 2.329(0.4); 2.324(0.3); 1.711(0.3); 1.680(0.8); 1.263(0.5); 1.236(2.1); 1.203(0.4); 1.187(0.6); 1.164(4.2); 1.147(11.4); 1.130(16.0); 1.123(3.9); 1.112(7.7); 1.106(6.0); 1.088(2.8); 1.049(12.0); 1.032(12.0); 0.853(0.4)

### Example 53

¹H-NMR (400.1 MHz. DMSO): δ=8.952(1.6); 8.948(3.0); 8.944(1.9); 8.943(1.8); 8.689(0.9); 8.668(0.9); 8.595(0.8); 8.573(0.7); 8.503(1.9); 8.502(1.9); 8.498(1.8); 8.497(1.8); 8.474(1.5); 8.469(1.4); 8.468(1.4); 7.629(2.8); 7.624(3.4); 7.617(2.1); 7.507(0.8); 7.502(0.7); 7.486(1.8); 7.481(1.8); 7.469(0.8); 7.455(3.0); 7.448(2.7); 7.434(2.8); 7.430(1.6); 7.414(0.5); 7.409(0.5); 5.753(1.7); 4.697(1.4); 4.685(1.5); 4.659(1.8); 4.648(1.9); 4.332(0.4); 4.321(0.4); 4.315(0.5); 4.310(0.5); 4.303(0.5); 4.299(0.5); 4.293(0.5); 4.282(0.5); 4.279(0.4); 4.266(0.4); 4.262(0.4); 4.257(0.3); 4.249(0.3); 4.244(0.4); 3.310(66.8); 3.287(1.3); 3.201(16.0); 3.181(12.1); 3.164(0.3); 2.900(0.5); 2.524(0.5); 2.520(0.7); 2.511(11.8); 2.506(26.2); 2.502(37.2); 2.497(26.6); 2.492(12.4); 1.357(0.4); 1.235(1.1); 1.157(3.8); 1.140(3.7); 1.040(5.0); 1.023(5.0)

### Example 54

¹H-NMR (400.1 MHz. CDCl3): δ=8.609(1.6); 8.603(1.7); 8.112(1.7); 8.107(1.7); 7.696(0.9); 7.518(1.4); 7.436(1.9); 7.415(2.4); 7.346(2.4); 7.341(2.6); 7.310(0.4); 7.280(0.3); 7.279(0.3); 7.278(0.4); 7.276(0.4); 7.273(0.7); 7.273(0.8); 7.272(0.9); 7.271(1.0); 7.270(1.1); 7.269(1.2); 7.268(1.8); 7.267(2.1); 7.266(2.6); 7.259(244.5); 7.252(4.7); 7.251(4.6); 7.250(4.4); 7.247(2.3); 7.246(2.5); 7.245(2.6); 7.245(2.6); 7.242(1.4); 7.241(1.2); 7.241(1.1); 7.240(1.0); 7.239(0.9); 7.238(0.8); 7.237(0.8); 7.236(0.7); 7.236(0.7); 7.235(0.7); 7.234(0.7); 7.233(0.6); 7.232(0.6); 7.229(1.4); 7.224(1.1); 7.209(0.5); 6.995(1.3); 5.298(0.5); 5.007(3.4); 3.240(16.0); 3.040(0.9); 3.027(0.8); 2.809(0.5); 2.004(1.3); 1.530(23.8); 1.225(0.4); 1.220(0.5); 1.215(0.5); 1.204(0.5); 1.199(0.7); 1.190(0.7); 1.006(0.7); 1.000(0.8); 0.975(0.6); 0.971(1.0); 0.963(0.8); 0.957(0.4); 0.943(0.9); 0.932(1.2); 0.923(1.7); 0.911(0.6); 0.907(0.7); 0.008(1.3)

### Example 55

¹H-NMR (601.6 MHz. CD3CN): δ=7.391(0.8); 7.378(2.7); 7.367(3.2); 7.357(4.2); 7.346(1.6); 7.321(0.7); 7.319(0.9); 7.307(1.4); 7.298(0.4); 7.296(0.5); 6.575(0.5); 6.563(0.5); 4.308(1.4); 4.298(3.0); 4.289(0.8); 4.278(0.9); 4.274(0.7); 4.268(0.6); 4.264(0.8); 4.253(0.5); 4.061(0.8); 4.059(0.8); 4.049(2.1); 4.047(2.1); 4.037(2.2); 4.035(2.2); 4.025(0.8); 4.023(0.8); 3.238(16.0); 2.160(119.9); 2.083(14.5); 2.057(0.4); 2.053(0.5); 2.049(0.4); 1.967(10.9); 1.959(3.7); 1.951(37.1); 1.947(67.0); 1.943(94.8); 1.938(69.2); 1.934(50.7); 1.832(0.4); 1.828(0.6); 1.824(0.4); 1.201(3.8); 1.189(7.8); 1.177(3.8); 1.157(6.9); 1.146(7.0); 0.000(35.1)

### Example 56

¹H-NMR (400.1 MHz. CDCl3): δ=7.906(2.6); 7.825(1.1); 7.518(0.9); 7.393(0.4); 7.390(0.7); 7.386(0.3); 7.374(1.0); 7.372(1.8); 7.369(1.5); 7.359(0.7); 7.354(2.4); 7.348(0.5); 7.341(2.1); 7.337(3.1); 7.331(0.7); 7.329(0.8); 7.323(1.8); 7.320(1.4); 7.316(0.8); 7.309(2.2); 7.304(1.1); 7.299(0.6); 7.296(0.4); 7.290(2.1); 7.287(3.2); 7.280(1.0); 7.272(1.3); 7.268(1.9); 7.260(167.5); 7.250(1.6); 7.250(1.4); 7.249(1.2); 7.248(1.1); 7.247(1.0); 7.246(0.9); 7.245(0.9); 7.245(0.8); 7.244(0.7); 7.243(0.7); 7.242(0.6); 7.241(0.5); 7.241(0.5); 7.240(0.5); 7.239(0.4); 7.238(0.4); 7.237(0.4); 7.236(0.4); 7.236(0.3); 7.234(0.3); 6.996(0.9); 6.977(0.9); 6.950(0.6); 6.841(1.9); 6.815(1.3); 6.706(0.9); 6.679(0.6); 5.298(3.2); 4.410(1.3); 4.401(1.6); 4.360(0.5); 4.352(0.5); 4.339(0.6); 4.322(0.5); 4.227(1.0); 4.216(0.8); 3.931(7.3); 3.908(4.3); 3.352(16.0); 3.308(10.6); 1.560(3.2); 1.257(0.4); 1.219(3.8); 1.202(3.8); 1.034(6.1); 1.017(6.2); 0.010(0.4); 0.008(1.6); 0.006(0.6); 0.005(0.8); 0.004(1.1)

### Example 57

¹H-NMR (400.1 MHz. CDCl3): δ=7.665(0.8); 7.664(0.7); 7.646(0.9); 7.644(0.9); 7.575(0.8); 7.573(0.8); 7.557(0.7); 7.555(0.6); 7.529(0.4); 7.527(0.6); 7.525(0.7); 7.523(0.5); 7.519(0.5); 7.508(0.7); 7.506(0.8); 7.504(0.6); 7.473(0.9); 7.471(0.9); 7.454(0.7); 7.452(0.7); 7.430(1.6); 7.412(2.3); 7.407(3.9); 7.292(1.1); 7.291(1.2); 7.286(1.0); 7.275(0.3); 7.274(0.4); 7.273(0.5); 7.272(1.0); 7.270(1.2); 7.268(0.9); 7.267(1.1); 7.266(2.0); 7.265(2.3); 7.264(2.6); 7.260(71.6); 7.254(1.7); 7.253(1.4); 7.253(1.1); 7.252(1.0); 7.251(0.9); 7.250(0.8); 7.249(0.7); 7.248(0.6); 7.248(0.6); 7.247(0.5); 7.246(0.4); 7.245(0.4); 7.244(0.4); 7.243(0.4); 7.243(0.3); 6.996(0.4); 6.000(0.7); 5.156(2.9); 3.252(16.0); 1.541(2.8); 1.106(0.4); 1.096(0.5); 1.092(0.6); 1.076(0.7); 1.071(0.5); 0.947(0.3); 0.932(0.5); 0.926(0.7); 0.921(0.9); 0.916(0.7); 0.912(1.2); 0.910(1.3); 0.903(2.7); 0.893(1.0); 0.887(0.6); 0.877(0.3); 0.008(0.6); 0.006(0.3); 0.005(0.4)

### Example 58

¹H-NMR (400.0 MHz. DMSO): δ=7.747(1.1); 7.725(1.2); 7.706(0.9); 7.684(0.9); 7.618(2.0); 7.613(2.1); 7.590(2.3); 7.585(2.5); 7.529(3.0); 7.517(3.1); 7.484(0.7); 7.480(0.6); 7.463(3.0); 7.459(3.0); 7.448(3.9); 7.443(2.1); 7.438(1.8); 7.427(1.1); 7.418(2.9); 7.397(1.1); 6.923(2.1); 6.911(4.4); 6.899(2.5); 4.738(1.6); 4.724(1.7); 4.626(2.1); 4.610(2.3); 4.339(0.7); 4.322(1.1); 4.318(1.1); 4.305(1.0); 4.301(1.2); 4.284(0.8); 3.324(7.9); 3.184(16.0); 3.178(13.5); 2.512(4.2); 2.507(8.6); 2.503(11.5); 2.498(8.5); 2.494(4.2); 2.329(10.2); 2.273(13.3); 1.397(4.5); 1.141(5.9); 1.123(7.3); 1.103(4.6); 0.008(0.4); 0.000(11.4); −0.008(0.4)

### Example 59

¹H-NMR (400.0 MHz. DMSO): δ=7.785(1.0); 7.764(1.0); 7.622(0.8); 7.602(0.8); 7.494(2.1); 7.489(2.2); 7.471(2.6); 7.466(2.7); 7.432(2.7); 7.427(0.9); 7.415(1.0); 7.411(3.9); 7.404(3.2); 7.400(1.0); 7.388(1.2); 7.383(5.0); 7.378(0.7); 7.329(0.7); 7.323(4.4); 7.318(1.2); 7.307(0.9); 7.302(2.6); 7.273(0.4); 7.267(3.2); 7.262(1.0); 7.250(0.8); 7.246(2.5); 6.870(2.0); 6.865(1.9); 6.793(2.4); 6.788(2.4); 4.305(1.2); 4.289(1.7); 4.235(2.0); 4.218(2.5); 4.198(0.6); 4.182(0.3); 4.090(0.5); 4.073(0.8); 4.068(0.6); 4.056(0.5); 4.052(0.8); 4.035(0.5); 3.333(70.8); 3.173(16.0); 3.148(13.4); 2.712(0.3); 2.542(81.2); 2.526(0.6); 2.521(0.8); 2.512(11.0); 2.507(22.8); 2.503(30.6); 2.498(22.6); 2.494(10.9); 2.469(10.8); 2.395(13.4); 2.368(0.4); 1.136(5.6); 1.119(5.6); 0.943(4.4); 0.926(4.4); 0.008(0.5); 0.000(16.9); −0.009(0.5)

### Example 60

¹H-NMR (400.0 MHz. DMSO): δ=8.115(0.6); 8.093(0.6); 7.983(0.9); 7.962(1.0); 7.447(3.2); 7.443(2.7); 7.430(1.4); 7.425(5.4); 7.422(3.6); 7.353(4.0); 7.343(2.7); 7.337(1.3); 7.331(2.6); 7.327(1.0); 7.322(1.8); 4.356(1.5); 4.343(1.7); 4.296(1.0); 4.280(1.2); 4.190(0.4); 4.173(0.6); 4.155(0.5); 4.152(0.5); 4.138(0.4); 4.133(0.4); 4.116(0.5); 4.111(0.4); 4.099(0.3); 4.094(0.4); 3.375(0.4); 3.368(0.4); 3.333(280.8); 3.303(0.4); 3.199(10.7); 3.195(16.0); 2.676(0.4); 2.672(0.6); 2.667(0.5); 2.542(63.2); 2.525(1.5); 2.520(2.2); 2.512(31.5); 2.507(64.6); 2.502(87.0); 2.498(64.4); 2.493(31.3); 2.334(0.4); 2.329(0.6); 2.325(0.4); 1.153(3.4); 1.136(3.4); 1.090(5.4); 1.073(5.4); 0.008(1.0); 0.000(31.9); −0.009(1.0)

### Example 61

¹H-NMR (400.0 MHz. DMSO): δ=8.670(1.1); 8.648(1.2); 8.583(1.0); 8.562(1.1); 7.455(3.5); 7.451(4.6); 7.434(6.2); 7.430(6.0); 7.360(4.3); 7.340(6.8); 7.319(3.2); 5.760(0.4); 4.337(1.7); 4.323(2.1); 4.254(1.9); 4.238(2.4); 4.220(0.5); 4.203(0.7); 4.185(0.7); 4.168(0.4); 4.130(0.6); 4.113(0.9); 4.109(0.7); 4.097(0.7); 4.092(0.9); 4.075(0.5); 3.336(54.0); 3.199(16.0); 3.184(14.2); 2.673(0.4); 2.508(40.9); 2.503(53.4); 2.499(40.6); 2.330(0.4); 1.233(0.4); 1.169(6.4); 1.152(6.3); 1.074(5.5); 1.057(5.4); 0.008(0.4); 0.000(9.1)

### Example 62

¹H-NMR (400.0 MHz. DMSO): δ=8.138(2.5); 8.135(2.9); 8.130(2.3); 8.127(2.0); 8.114(1.3); 8.050(1.9); 8.047(2.0); 8.043(2.1); 8.040(2.0); 7.984(1.0); 7.963(1.1); 7.571(1.3); 7.564(1.4); 7.558(1.9); 7.551(2.1); 7.549(2.1); 7.541(1.7); 7.536(2.1); 7.529(1.9); 7.490(1.9); 7.487(1.9); 7.478(1.4); 7.475(1.4); 7.438(0.4); 7.432(3.2); 7.428(1.2); 7.418(2.7); 7.415(3.4); 7.411(4.9); 7.405(5.2); 7.389(1.4); 7.384(5.6); 7.379(1.0); 7.336(5.0); 7.315(2.7); 7.282(3.8); 7.261(2.9); 4.326(1.5); 4.311(2.1); 4.257(2.3); 4.241(3.0); 4.222(0.7); 4.206(0.4); 4.114(0.5); 4.097(0.8); 4.093(0.7); 4.080(0.6); 4.076(0.9); 4.059(0.5); 3.340(35.3); 3.177(16.0); 3.154(14.0); 2.543(21.6); 2.526(0.7); 2.521(1.0); 2.512(11.9); 2.508(24.0); 2.503(32.0); 2.499(24.1); 2.495(12.2); 1.158(6.2); 1.142(6.1); 0.972(5.1); 0.955(5.1); 0.000(8.0); −0.008(0.3)

### Example 63

¹H-NMR (400.0 MHz. DMSO): δ=8.168(1.1); 8.146(1.2); 8.032(0.9); 8.012(0.9); 7.620(2.7); 7.606(2.9); 7.597(3.5); 7.583(3.6); 7.445(2.7); 7.440(1.1); 7.427(4.2); 7.424(5.0); 7.411(1.5); 7.406(5.3); 7.345(4.9); 7.324(6.2); 7.303(2.6); 7.174(2.8); 7.160(2.7); 7.019(3.6); 7.005(3.5); 4.315(1.4); 4.300(1.8); 4.231(2.0); 4.214(2.7); 4.197(0.7); 4.178(0.6); 4.161(0.4); 4.094(0.6); 4.077(0.9); 4.073(0.7); 4.060(0.6); 4.056(0.9); 4.039(0.5); 3.343(15.0); 3.183(16.0); 3.164(12.9); 2.544(4.5); 2.513(5.6); 2.509(11.4); 2.504(15.2); 2.500(11.5); 2.495(5.9); 1.152(6.2); 1.136(6.2); 0.976(4.7); 0.959(4.7); 0.000(4.3)

### Example 64

¹H-NMR (400.0 MHz. DMSO): δ=7.935(1.3); 7.913(1.3); 7.899(3.2); 7.894(3.2); 7.879(3.8); 7.874(3.8); 7.775(1.1); 7.755(1.1); 7.440(3.1); 7.418(5.4); 7.395(5.5); 7.334(4.9); 7.313(3.0); 7.291(3.7); 7.270(2.8); 6.913(3.0); 6.907(3.0); 6.834(3.7); 6.829(3.7); 4.305(1.5); 4.290(2.0); 4.251(1.9); 4.235(2.3); 4.221(0.5); 4.204(0.7); 4.185(0.7); 4.169(0.4); 4.086(0.5); 4.069(0.8); 4.065(0.7); 4.053(0.6); 4.048(0.8); 4.032(0.5); 3.365(214.1); 3.188(16.0); 3.157(13.3); 2.545(24.0); 2.527(0.8); 2.514(16.6); 2.510(32.7); 2.505(42.9); 2.501(32.2); 2.497(16.1); 1.126(6.1); 1.109(6.1); 0.962(4.8); 0.944(4.8); 0.000(5.3)

### Example 65

¹H-NMR (400.0 MHz. DMSO): δ=8.016(0.7); 7.995(0.7); 7.856(0.5); 7.836(0.5); 7.808(1.2); 7.795(1.2); 7.777(1.6); 7.764(1.6); 7.437(2.8); 7.416(4.8); 7.360(2.4); 7.354(3.1); 7.339(1.7); 7.333(1.8); 7.187(1.2); 7.174(1.2); 7.149(1.7); 7.136(1.6); 6.574(0.4); 4.362(0.8); 4.350(0.9); 4.305(1.1); 4.290(1.2); 4.180(0.3); 4.131(0.4); 4.115(0.5); 4.094(0.5); 3.323(9.2); 3.213(9.8); 3.211(9.4); 2.507(21.7); 2.502(28.1); 2.498(21.9); 1.989(0.4); 1.397(16.0); 1.131(3.6); 1.114(3.6); 1.090(2.7); 1.073(2.6); 0.000(4.4)

### Example 66

¹H-NMR (400.0 MHz. DMSO): δ=7.897(0.6); 7.876(0.7); 7.840(1.1); 7.826(1.2); 7.805(1.6); 7.792(1.6); 7.743(0.5); 7.722(0.5); 7.434(2.3); 7.413(3.7); 7.350(4.2); 7.329(2.7); 7.160(1.1); 7.147(1.1); 7.117(1.6); 7.103(1.6); 4.369(0.7); 4.356(0.8); 4.314(1.1); 4.298(1.2); 4.162(0.3); 4.145(0.4); 4.128(0.5); 4.123(0.4); 4.111(0.4); 4.107(0.5); 3.323(9.5); 3.206(13.2); 2.507(21.2); 2.502(27.4); 2.498(20.8); 1.397(16.0); 1.137(3.6); 1.120(3.5); 1.094(2.5); 1.077(2.4); 0.000(4.2)

### Example 69

¹H-NMR (400.0 MHz. DMSO): =8.041(0.5); 8.027(1.0); 8.013(0.6); 7.839(2.9); 7.826(3.1); 7.456(3.0); 7.451(1.2); 7.434(5.1); 7.373(4.5); 7.352(2.8); 7.148(3.1); 7.135(3.0); 4.451(0.9); 4.435(1.8); 4.419(0.9); 3.493(1.9); 3.478(3.2); 3.463(1.7); 3.323(6.6); 3.193(16.0); 2.525(0.4); 2.511(7.1); 2.507(13.9); 2.503(18.3); 2.498(13.7); 2.494(7.0); 0.000(1.1)

### Example 70

¹H-NMR (400.0 MHz. DMSO): δ=8.346(0.5); 8.332(1.0); 8.317(0.5); 7.478(2.9); 7.463(3.3); 7.452(2.9); 7.448(1.1); 7.436(1.3); 7.431(4.7); 7.426(0.7); 7.360(4.4); 7.339(2.8); 7.192(3.3); 7.177(3.0); 4.405(0.8); 4.389(1.5); 4.373(0.9); 3.421(1.0); 3.412(1.1); 3.407(1.2); 3.404(1.3); 3.398(1.8); 3.390(1.0); 3.383(1.0); 3.321(14.1); 3.171(16.0); 2.524(0.8); 2.510(12.7); 2.506(25.3); 2.502(33.7); 2.497(25.3); 2.493(12.7); 0.000(1.9)

### Example 71

¹H-NMR (400.0 MHz. DMSO): δ=8.242(0.6); 8.228(1.2); 8.213(0.6); 7.898(3.2); 7.892(3.4); 7.456(3.2); 7.452(1.3); 7.440(1.4); 7.435(4.9); 7.353(4.6); 7.332(3.1); 6.894(3.3); 6.888(3.3); 4.389(0.9); 4.375(1.1); 4.371(1.3); 4.357(0.9); 3.381(1.0); 3.367(1.7); 3.358(1.3); 3.354(1.6); 3.340(1.0); 3.323(7.3); 3.165(16.0); 2.525(0.5); 2.511(8.2); 2.507(16.2); 2.502(21.7); 2.498(16.6); 2.494(8.6); 0.000(1.2)

### Example 72

¹H-NMR (400.0 MHz. DMSO): δ=8.872(0.6); 8.858(1.1); 8.844(0.6); 7.509(0.7); 7.505(0.7); 7.488(1.3); 7.472(0.7); 7.467(0.8); 7.452(3.1); 7.431(5.1); 7.374(4.8); 7.353(2.9); 7.163(0.4); 7.155(2.1); 7.135(2.9); 7.115(1.8); 7.107(0.4); 4.381(0.9); 4.365(1.6); 4.349(1.0); 3.454(1.0); 3.439(1.4); 3.437(1.4); 3.429(1.9); 3.423(1.2); 3.414(1.0); 3.322(6.0); 3.180(16.0); 2.506(15.9); 2.502(21.2); 2.497(16.2); 0.000(1.1)

### Example 73

¹H-NMR (400.1 MHz. DMSO): δ=9.016(5.4); 9.010(6.5); 9.010(6.5); 9.004(4.0); 9.004(4.1); 8.998(4.3); 8.998(4.3); 8.981(0.4); 8.951(6.3); 8.945(5.5); 8.930(4.3); 8.924(3.7); 8.877(3.0); 8.856(3.1); 8.781(1.8); 8.759(1.9); 7.658(8.0); 7.652(8.7); 7.642(4.8); 7.638(4.8); 7.603(0.3); 7.526(2.6); 7.521(2.5); 7.505(5.4); 7.500(5.5); 7.489(1.6); 7.483(0.6); 7.469(15.6); 7.462(5.9); 7.457(5.2); 7.448(4.9); 7.441(1.4); 7.436(1.4); 4.699(3.9); 4.687(4.3); 4.679(5.9); 4.669(6.3); 4.383(0.3); 4.372(0.4); 4.366(1.3); 4.355(1.4); 4.349(1.7); 4.345(1.6); 4.338(1.6); 4.334(1.8); 4.327(1.6); 4.317(2.0); 4.311(0.9); 4.304(1.1); 4.299(1.4); 4.294(1.1); 4.287(1.0); 4.282(1.1); 4.277(1.0); 4.265(0.9); 3.949(0.6); 3.872(0.4); 3.387(1.6); 3.336(121.5); 3.308(1.1); 3.295(0.5); 3.287(0.9); 3.271(0.8); 3.252(0.5); 3.221(46.7); 3.201(30.1); 3.172(0.8); 3.154(0.6); 2.566(0.5); 2.561(0.6); 2.557(0.4); 2.533(0.6); 2.528(1.1); 2.520(13.9); 2.515(29.6); 2.511(41.5); 2.506(30.8); 2.502(15.8); 2.466(0.7); 2.461(0.6); 2.457(0.4); 2.199(1.2); 1.197(9.7); 1.180(9.8); 1.044(15.8); 1.027(16.0); 0.999(0.5); 0.883(0.6); 0.868(0.4); 0.864(0.4)

### Example 74

¹H-NMR (400.0 MHz. DMSO): δ=7.836(1.2); 7.829(1.2); 7.630(1.8); 7.627(3.2); 7.624(1.8); 7.570(0.5); 7.565(0.8); 7.551(1.1); 7.546(1.4); 7.532(0.7); 7.528(1.1); 7.523(0.4); 7.514(0.5); 7.508(0.9); 7.504(0.6); 7.502(0.6); 7.495(0.8); 7.493(0.8); 7.485(7.2); 7.482(6.8); 7.475(0.8); 7.470(0.5); 7.278(2.5); 7.260(1.8); 7.258(2.6); 7.251(1.2); 7.242(0.9); 7.239(0.9); 7.230(0.8); 5.305(5.7); 3.322(12.6); 3.124(16.0); 2.525(0.5); 2.511(8.2); 2.507(16.2); 2.502(21.4); 2.498(15.6); 2.493(7.5); 1.335(9.4); 1.278(9.1); 0.000(2.4)

### Example 75

¹H-NMR (400.0 MHz. DMSO): δ=8.042(2.1); 7.641(1.8); 7.638(3.2); 7.635(1.8); 7.486(6.9); 7.483(6.8); 7.462(1.3); 7.459(2.5); 7.457(1.5); 7.437(0.8); 7.425(1.4); 7.414(1.6); 7.406(0.9); 7.395(1.3); 7.381(4.3); 7.371(2.5); 5.400(5.7); 3.322(25.1); 3.123(16.0); 2.524(0.8); 2.519(1.2); 2.511(13.9); 2.506(27.7); 2.502(36.9); 2.497(27.1); 2.492(13.1); 1.336(1.0); 1.298(0.6); 1.282(9.4); 1.274(9.8); 1.259(0.7); 1.249(1.3); 0.000(3.9)

### Example 76

¹H-NMR (400.0 MHz. DMSO): δ=8.125(2.2); 7.764(1.3); 7.745(1.7); 7.736(0.7); 7.718(1.4); 7.699(1.0); 7.650(2.9); 7.648(1.9); 7.640(1.0); 7.621(1.3); 7.602(0.5); 7.490(6.4); 7.487(7.0); 7.462(1.3); 5.409(5.5); 3.321(17.0); 3.107(16.0); 2.524(0.9); 2.511(14.8); 2.506(28.9); 2.502(38.0); 2.497(27.7); 2.493(13.2); 1.336(1.7); 1.298(0.4); 1.264(9.8); 1.247(9.7); 0.000(4.1)

### Example 77

¹H-NMR (400.0 MHz. DMSO): δ=8.397(1.8); 7.639(2.3); 7.635(2.3); 7.515(0.4); 7.510(0.7); 7.498(0.8); 7.489(4.0); 7.484(3.0); 7.479(2.9); 7.473(0.6); 7.461(0.8); 7.456(0.9); 7.439(0.3); 7.151(2.0); 7.132(2.3); 7.111(1.7); 5.757(0.8); 5.365(4.9); 3.322(28.5); 3.115(16.0); 3.019(0.4); 2.695(0.7); 2.524(0.9); 2.520(1.3); 2.511(17.0); 2.506(34.6); 2.502(46.3); 2.497(33.4); 2.492(15.7); 2.328(0.3); 1.281(9.1); 1.226(8.5); 0.008(0.7); 0.000(23.9); −0.009(0.7)

### Example 78

¹H-NMR (400.0 MHz. DMSO): δ=8.163(0.5); 7.844(3.1); 7.831(3.2); 7.648(3.0); 7.647(3.0); 7.632(2.7); 7.497(6.9); 7.165(3.3); 7.152(3.2); 5.141(5.4); 3.322(10.2); 3.286(0.7); 3.139(16.0); 2.696(1.9); 2.675(0.4); 2.671(0.4); 2.506(50.9); 2.502(62.5); 2.498(46.2); 2.333(0.3); 2.329(0.4); 2.177(0.5); 1.903(0.3); 1.339(10.5); 1.317(10.6); 0.000(21.7); −0.008(1.2)

### Example 79

¹H-NMR (400.0 MHz. DMSO): δ=7.704(4.4); 7.700(2.3); 7.691(4.1); 7.640(1.7); 7.637(3.1); 7.634(1.9); 7.495(6.7); 7.492(6.1); 7.215(0.5); 7.211(3.8); 7.198(3.7); 5.757(0.7); 5.229(5.7); 3.323(34.2); 3.145(16.0); 2.667(0.8); 2.524(0.9); 2.520(1.4); 2.511(16.9); 2.507(33.7); 2.502(44.6); 2.497(32.3); 2.493(15.4); 2.130(0.5); 1.358(8.6); 1.286(8.6); 0.008(0.5); 0.000(15.2); −0.009(0.5)

### Example 80

¹H-NMR (400.0 MHz. DMSO): δ=8.162(0.4); 7.874(3.7); 7.869(3.7); 7.633(2.4); 7.629(2.5); 7.506(0.6); 7.502(0.5); 7.485(2.4); 7.480(2.5); 7.472(3.7); 7.451(0.9); 7.426(2.1); 6.843(3.8); 6.838(3.7); 5.229(4.9); 3.323(19.2); 3.107(16.0); 2.671(0.4); 2.524(1.1); 2.520(1.8); 2.511(22.0); 2.507(44.2); 2.502(58.5); 2.497(42.1); 2.493(19.8); 2.329(0.4); 1.340(8.5); 1.281(8.6); 0.008(0.8); 0.000(24.7); −0.009(0.7)

### Example 81

¹H-NMR (400.0 MHz. DMSO): δ=8.440(1.7); 8.435(1.8); 8.428(1.9); 8.423(1.8); 8.095(1.9); 7.746(1.6); 7.742(1.7); 7.728(2.0); 7.723(1.9); 7.480(2.0); 7.467(2.4); 7.465(3.3); 7.461(2.6); 7.449(2.9); 7.444(4.8); 7.438(0.6); 7.361(4.2); 7.356(1.2); 7.344(0.9); 7.340(2.8); 5.757(2.1); 4.927(3.7); 3.322(23.6); 3.181(16.0); 2.524(0.7); 2.519(1.0); 2.511(12.8); 2.506(25.8); 2.501(34.2); 2.497(24.6); 2.492(11.6); 1.441(9.7); 1.101(8.8); 0.008(0.4); 0.000(10.8)

### Example 82

¹H-NMR (400.0 MHz. DMSO): δ=8.242(1.9); 7.483(0.7); 7.478(0.6); 7.466(0.5); 7.462(1.3); 7.457(0.5); 7.445(0.7); 7.441(0.8); 7.424(0.7); 7.418(2.8); 7.413(1.0); 7.402(1.3); 7.397(5.3); 7.391(0.8); 7.351(4.6); 7.346(1.2); 7.334(0.9); 7.329(2.4); 7.141(0.3); 7.133(2.1); 7.114(2.6); 7.093(1.8); 5.757(1.1); 4.916(4.0); 3.323(18.7); 3.175(16.0); 2.524(0.6); 2.519(0.9); 2.510(10.7); 2.506(21.2); 2.501(27.8); 2.497(20.0); 2.492(9.4); 1.414(10.3); 1.169(1.5); 1.083(9.4); 0.000(8.1)

### Example 83

¹H-NMR (400.0 MHz. DMSO): δ=7.759(1.2); 7.754(1.2); 7.506(0.5); 7.500(0.7); 7.495(0.9); 7.493(0.9); 7.488(0.9); 7.482(1.4); 7.477(2.1); 7.471(0.9); 7.463(0.8); 7.458(1.2); 7.438(0.4); 7.432(3.1); 7.427(1.1); 7.415(1.3); 7.411(4.8); 7.405(0.6); 7.338(0.7); 7.332(4.3); 7.328(1.2); 7.316(1.0); 7.311(2.8); 7.306(0.3); 7.260(2.5); 7.240(2.9); 7.235(1.3); 7.223(0.9); 7.221(0.9); 7.214(0.8); 7.211(0.6); 5.757(0.5); 4.908(3.9); 3.324(14.4); 3.183(16.0); 2.519(0.5); 2.511(6.1); 2.506(12.2); 2.502(16.1); 2.497(11.6); 2.493(5.5); 1.444(10.2); 1.336(0.7); 1.249(0.5); 1.170(0.8); 1.126(9.4); 0.000(4.0)

### Example 84

¹H-NMR (400.0 MHz. DMSO): δ=7.905(2.0); 7.465(1.0); 7.461(1.5); 7.455(3.2); 7.450(1.2); 7.445(2.0); 7.441(2.5); 7.434(5.2); 7.428(0.8); 7.422(1.0); 7.417(1.0); 7.404(1.6); 7.399(1.6); 7.384(1.0); 7.379(1.1); 7.375(1.2); 7.370(1.2); 7.362(4.5); 7.356(2.8); 7.352(2.0); 7.345(1.1); 7.341(2.9); 7.334(1.1); 7.302(1.8); 7.298(1.6); 7.284(1.2); 7.279(1.0); 5.756(1.2); 4.973(4.0); 3.322(27.2); 3.181(16.0); 2.524(0.7); 2.519(1.1); 2.510(13.1); 2.506(26.2); 2.501(34.5); 2.497(24.8); 2.492(11.8); 1.451(10.5); 1.336(0.3); 1.249(0.4); 1.088(9.3); 0.000(7.7)

### Example 85

¹H-NMR (400.0 MHz. DMSO): δ=7.981(2.1); 7.752(1.2); 7.732(1.6); 7.712(0.5); 7.694(1.3); 7.675(0.9); 7.625(0.9); 7.606(1.2); 7.587(0.5); 7.486(0.4); 7.479(3.5); 7.475(1.1); 7.463(1.3); 7.458(4.9); 7.452(0.6); 7.356(0.7); 7.350(4.3); 7.345(2.1); 7.333(1.2); 7.329(3.3); 7.323(1.6); 5.756(1.5); 4.949(3.9); 3.322(18.1); 3.169(16.0); 3.159(0.5); 2.524(0.5); 2.519(0.8); 2.510(8.9); 2.506(17.8); 2.501(23.5); 2.497(16.8); 2.492(7.8); 1.989(0.5); 1.388(9.8); 1.336(0.7); 1.249(0.8); 1.098(9.2); 0.000(6.0)

### Example 86

¹H-NMR (400.0 MHz. DMSO): δ=7.832(3.5); 7.819(3.6); 7.592(2.0); 7.451(0.4); 7.445(3.1); 7.441(1.1); 7.429(1.3); 7.424(4.7); 7.418(0.6); 7.343(4.2); 7.339(1.3); 7.326(1.1); 7.322(2.8); 7.316(0.4); 7.153(3.7); 7.140(3.6); 5.757(0.4); 4.697(3.8); 3.324(12.4); 3.195(16.0); 3.179(0.5); 2.578(0.4); 2.525(0.4); 2.520(0.6); 2.512(7.2); 2.507(14.4); 2.503(19.0); 2.498(13.7); 2.494(6.5); 1.373(0.4); 1.355(9.9); 1.336(0.7); 1.249(0.5); 1.230(9.6); 1.170(0.5); 0.000(4.6)

### Example 87

¹H-NMR (400.0 MHz. DMSO): δ=7.687(3.5); 7.674(3.7); 7.653(0.4); 7.644(2.2); 7.439(0.4); 7.433(3.0); 7.429(1.0); 7.417(1.3); 7.412(5.0); 7.407(0.7); 7.350(4.5); 7.345(1.3); 7.329(2.7); 7.323(0.4); 7.199(3.8); 7.187(3.6); 5.756(2.0); 4.818(4.0); 3.323(11.2); 3.200(16.0); 2.525(0.4); 2.511(7.1); 2.507(14.0); 2.502(18.3); 2.498(13.2); 2.493(6.3); 1.410(10.2); 1.336(1.0); 1.249(0.6); 1.175(9.6); 0.000(4.3)

### Example 88

¹H-NMR (400.0 MHz. DMSO): δ=7.855(4.2); 7.850(4.2); 7.433(0.4); 7.427(3.3); 7.423(1.1); 7.411(1.3); 7.406(4.7); 7.400(0.6); 7.349(2.2); 7.304(0.6); 7.298(4.1); 7.294(1.2); 7.282(1.0); 7.277(3.0); 7.271(0.3); 6.838(4.2); 6.834(4.2); 5.757(2.1); 4.787(3.9); 3.324(10.6); 3.173(16.0); 2.526(0.3); 2.521(0.5); 2.512(6.0); 2.508(12.0); 2.503(15.8); 2.499(11.3); 2.494(5.3); 1.363(9.8); 1.337(0.4); 1.249(0.5); 1.193(9.3); 0.000(4.1)

### Example 89

¹H-NMR (400.0 MHz. DMSO): δ=8.515(1.1); 8.493(1.8); 8.470(0.8); 8.443(1.7); 8.438(1.8); 8.431(2.5); 8.426(2.5); 8.418(1.3); 8.413(1.3); 7.952(0.5); 7.624(2.0); 7.621(2.8); 7.617(3.0); 7.611(3.4); 7.606(1.3); 7.592(1.7); 7.589(1.9); 7.588(1.9); 7.585(1.6); 7.571(2.1); 7.566(2.0); 7.516(0.8); 7.511(0.7); 7.495(1.5); 7.490(1.5); 7.482(2.5); 7.478(4.2); 7.474(6.9); 7.471(2.9); 7.464(1.7); 7.456(1.6); 7.452(2.0); 7.450(1.7); 7.445(2.5); 7.438(1.2); 7.424(1.3); 4.712(1.5); 4.703(1.5); 4.594(2.3); 4.578(2.5); 4.167(0.4); 4.156(0.5); 4.146(1.0); 4.136(0.8); 4.123(1.0); 4.114(0.4); 4.108(0.4); 4.100(0.4); 3.411(0.5); 3.357(238.9); 3.200(11.7); 3.172(16.0); 2.892(3.8); 2.732(3.0); 2.527(0.8); 2.522(1.3); 2.513(15.7); 2.509(31.7); 2.504(41.6); 2.500(29.8); 2.495(14.2); 1.694(0.4); 1.687(0.4); 1.676(0.6); 1.669(0.5); 1.660(0.9); 1.653(0.6); 1.641(0.8); 1.634(0.5); 1.627(0.8); 1.609(0.6); 1.605(0.4); 1.586(0.4); 1.529(0.4); 1.510(0.5); 1.501(0.4); 1.494(0.4); 1.483(0.5); 1.476(0.3); 1.466(0.4); 1.235(0.4); 0.979(2.0); 0.961(4.4); 0.942(1.8); 0.903(2.5); 0.884(5.5); 0.866(2.3); 0.008(0.4); 0.000(12.4); −0.009(0.4)

### Example 90

¹H-NMR (400.0 MHz. DMSO): δ=8.752(2.5); 8.748(2.7); 8.740(2.7); 8.736(2.6); 8.670(0.9); 8.645(1.7); 8.620(1.2); 7.953(0.5); 7.872(0.9); 7.853(1.0); 7.776(1.1); 7.757(1.5); 7.657(1.2); 7.639(3.5); 7.634(2.9); 7.628(1.4); 7.610(0.8); 7.598(2.7); 7.484(6.8); 7.464(0.9); 7.459(0.7); 7.443(1.7); 7.438(1.6); 7.411(2.7); 7.390(1.2); 6.853(1.1); 6.847(0.8); 6.717(2.4); 6.711(1.7); 6.582(1.2); 6.575(0.9); 4.731(1.6); 4.722(1.7); 4.593(2.3); 4.575(2.5); 4.210(0.6); 4.200(0.7); 4.196(0.7); 4.186(0.7); 4.177(0.8); 4.163(0.5); 4.152(0.6); 3.325(34.2); 3.209(12.0); 3.170(16.0); 3.155(0.5); 3.137(0.4); 2.891(3.5); 2.732(2.9); 2.525(1.0); 2.511(18.4); 2.507(35.8); 2.502(46.2); 2.498(33.3); 2.493(16.1); 1.749(0.3); 1.738(0.4); 1.731(0.5); 1.722(0.5); 1.715(0.5); 1.704(0.6); 1.696(0.5); 1.689(0.4); 1.670(0.7); 1.656(0.9); 1.637(1.0); 1.616(0.6); 1.575(0.4); 1.556(0.5); 1.547(0.5); 1.539(0.5); 1.529(0.5); 1.522(0.4); 1.512(0.4); 1.235(0.5); 0.953(2.2); 0.935(4.7); 0.916(2.0); 0.889(2.7); 0.871(5.8); 0.852(2.6); 0.008(0.5); 0.000(13.2); −0.009(0.5)

### Example 91

¹H-NMR (400.0 MHz. DMSO): δ=8.778(1.4); 8.768(1.4); 8.766(1.4); 8.758(1.0); 8.755(1.0); 8.747(1.0); 8.744(1.0); 8.625(1.2); 8.602(1.4); 8.596(1.1); 8.572(0.9); 7.954(0.6); 7.784(1.0); 7.772(1.0); 7.765(1.8); 7.753(1.7); 7.746(1.4); 7.735(1.4); 7.724(1.4); 7.720(1.5); 7.704(0.5); 7.700(0.5); 7.679(1.6); 7.662(1.1); 7.660(1.1); 7.647(2.7); 7.643(2.9); 7.626(2.1); 7.621(2.3); 7.533(0.7); 7.528(0.6); 7.512(1.6); 7.507(1.6); 7.502(0.8); 7.498(0.5); 7.481(5.3); 7.477(3.2); 7.470(4.3); 7.460(1.3); 7.449(0.9); 4.707(1.6); 4.698(1.7); 4.609(2.2); 4.595(2.4); 4.175(0.3); 4.169(0.4); 4.160(0.8); 4.156(0.8); 4.147(1.0); 4.138(1.0); 4.134(1.0); 4.125(0.8); 4.118(0.5); 4.111(0.5); 3.325(18.0); 3.212(11.7); 3.180(16.0); 2.891(4.4); 2.732(3.7); 2.525(0.8); 2.511(15.0); 2.507(29.1); 2.503(37.6); 2.498(27.4); 2.494(13.5); 1.646(0.6); 1.631(0.6); 1.627(0.7); 1.622(0.8); 1.613(0.8); 1.603(1.0); 1.586(0.9); 1.580(0.9); 1.568(0.7); 1.561(0.6); 1.519(0.4); 1.501(0.6); 1.492(0.5); 1.484(0.4); 1.474(0.6); 1.466(0.3); 1.457(0.4); 1.235(0.5); 0.953(2.2); 0.935(4.6); 0.916(1.9); 0.866(2.7); 0.848(5.7); 0.829(2.4); 0.008(0.5); 0.000(11.5); −0.008(0.4)

### Example 92

¹H-NMR (400.0 MHz. DMSO): δ=9.014(2.5); 9.008(3.0); 8.997(2.2); 8.939(2.8); 8.933(2.5); 8.915(2.2); 8.910(1.9); 8.712(1.3); 8.689(1.4); 8.628(1.0); 8.605(1.1); 7.715(0.3); 7.622(3.1); 7.616(2.6); 7.611(2.4); 7.499(0.5); 7.495(0.5); 7.478(3.9); 7.471(5.9); 7.460(3.2); 7.450(1.0); 7.428(1.9); 7.423(1.8); 7.407(0.9); 7.402(0.9); 4.737(1.9); 4.729(1.9); 4.647(2.4); 4.635(2.6); 4.223(0.5); 4.211(0.8); 4.199(0.9); 4.188(0.8); 4.176(0.5); 4.113(0.6); 4.093(0.6); 3.375(0.5); 3.334(4.4); 3.291(0.4); 3.259(1.8); 3.208(12.4); 3.193(16.0); 3.163(0.7); 3.145(0.4); 3.136(0.3); 3.046(0.5); 2.892(0.9); 2.815(0.4); 2.732(0.8); 2.507(34.0); 2.503(42.1); 2.330(0.3); 1.684(0.4); 1.666(1.2); 1.647(1.6); 1.627(1.0); 1.610(0.4); 1.543(0.4); 1.527(0.9); 1.516(1.4); 1.509(1.1); 1.498(1.5); 1.491(1.1); 1.480(0.8); 1.473(0.9); 1.456(0.5); 1.251(0.4); 1.233(1.0); 0.964(2.6); 0.946(5.2); 0.927(2.3); 0.850(3.4); 0.832(6.5); 0.814(2.9); 0.785(0.9); 0.766(0.5); 0.000(9.9)

### Example 93

¹H-NMR (400.0 MHz. DMSO): δ=8.761(0.8); 8.758(2.0); 8.754(1.6); 8.750(0.9); 8.746(2.1); 8.742(1.6); 8.733(1.0); 8.711(1.0); 8.673(0.5); 8.651(0.5); 7.853(0.5); 7.833(0.6); 7.747(0.9); 7.729(1.3); 7.727(1.3); 7.653(1.1); 7.641(1.3); 7.633(0.8); 7.622(0.8); 7.567(1.6); 7.563(3.1); 7.558(2.0); 7.551(0.8); 7.373(2.6); 7.368(2.4); 7.342(5.7); 7.337(5.4); 7.091(0.4); 6.955(1.0); 6.912(1.0); 6.820(0.5); 6.776(2.2); 6.641(1.1); 4.385(0.8); 4.373(0.9); 4.222(1.5); 4.204(2.3); 4.154(0.5); 4.137(0.8); 4.133(0.6); 4.120(0.5); 4.116(0.8); 4.099(0.4); 3.325(24.2); 3.313(0.4); 3.228(8.4); 3.223(16.0); 2.891(0.5); 2.732(0.4); 2.525(0.7); 2.520(1.0); 2.511(13.3); 2.507(26.8); 2.502(35.2); 2.498(25.1); 2.493(11.9); 1.235(0.3); 1.204(5.6); 1.188(5.5); 1.106(2.5); 1.089(2.4); 0.008(0.3); 0.000(10.5); −0.009(0.3)

### Example 94

¹H-NMR (400.0 MHz. DMSO): δ=8.618(1.1); 8.596(1.1); 8.542(0.6); 8.521(0.6); 8.450(1.7); 8.446(2.0); 8.438(1.9); 8.434(2.0); 7.705(0.7); 7.700(0.8); 7.686(0.9); 7.681(0.9); 7.609(1.4); 7.604(1.5); 7.590(2.0); 7.585(1.9); 7.565(1.5); 7.560(2.8); 7.556(1.7); 7.552(0.9); 7.547(1.4); 7.542(0.8); 7.487(1.0); 7.482(2.0); 7.475(1.1); 7.470(2.1); 7.463(1.5); 7.456(0.9); 7.451(1.5); 7.388(2.8); 7.383(2.7); 7.336(5.8); 7.331(5.6); 4.390(0.9); 4.379(1.1); 4.218(0.3); 4.209(1.9); 4.192(2.4); 4.112(0.6); 4.095(0.8); 4.090(0.7); 4.078(0.6); 4.073(0.8); 4.056(0.5); 3.325(30.6); 3.228(8.5); 3.221(16.0); 2.891(0.7); 2.732(0.6); 2.524(0.6); 2.511(14.9); 2.507(29.4); 2.502(38.3); 2.498(27.9); 2.493(13.7); 1.235(0.3); 1.181(5.9); 1.165(5.9); 1.106(2.8); 1.089(2.8); 0.008(0.3); 0.000(9.8); −0.009(0.4)

### Example 95

¹H-NMR (400.0 MHz. DMSO): δ=9.000(2.4); 8.994(2.9); 8.940(2.0); 8.935(1.7); 8.927(1.0); 8.921(0.9); 8.827(0.9); 8.806(0.9); 8.699(0.4); 8.677(0.5); 7.547(1.3); 7.542(2.6); 7.537(1.9); 7.532(1.3); 7.527(0.7); 7.412(2.4); 7.408(2.2); 7.343(4.9); 7.338(4.7); 4.387(0.8); 4.376(0.9); 4.286(1.5); 3.229(16.0); 2.892(0.7); 2.733(0.6); 2.547(0.3); 2.525(0.5); 2.521(0.7); 2.512(9.2); 2.508(18.6); 2.503(24.4); 2.498(17.6); 2.494(8.4); 1.147(5.1); 1.137(3.0); 1.130(5.2); 1.120(2.5); 0.000(8.7)

### Example 96

¹H-NMR (400.0 MHz. DMSO): δ=8.786(0.8); 8.784(0.9); 8.775(1.1); 8.772(1.3); 8.762(0.4); 8.689(0.7); 8.668(0.8); 8.625(0.4); 8.603(0.4); 7.781(0.7); 7.767(1.3); 7.761(1.8); 7.756(1.7); 7.750(1.0); 7.652(1.0); 7.649(1.0); 7.633(0.7); 7.630(0.7); 7.585(1.1); 7.581(2.1); 7.576(1.1); 7.564(0.5); 7.559(1.0); 7.555(0.6); 7.390(2.1); 7.385(2.0); 7.335(4.2); 7.330(4.0); 4.362(0.6); 4.350(0.8); 4.225(1.4); 4.209(1.7); 4.111(0.4); 4.094(0.6); 4.090(0.4); 4.078(0.4); 4.073(0.6); 4.056(0.4); 3.325(15.5); 3.225(16.0); 2.891(0.4); 2.732(0.3); 2.525(0.5); 2.520(0.7); 2.511(9.6); 2.507(19.2); 2.502(25.2); 2.498(18.1); 2.493(8.5); 1.149(4.2); 1.132(4.2); 1.100(2.0); 1.083(2.0); 0.000(9.1)

### Example 97

¹H-NMR (400.0 MHz. DMSO): δ=8.897(2.5); 8.741(1.4); 8.738(1.6); 8.729(1.6); 8.726(1.5); 7.953(0.5); 7.811(1.2); 7.791(1.5); 7.624(1.2); 7.613(3.7); 7.608(3.6); 7.592(0.9); 7.518(1.3); 7.497(4.0); 7.480(2.4); 7.475(2.2); 7.459(0.8); 7.454(0.8); 7.131(1.0); 6.995(2.3); 6.860(1.2); 5.007(4.8); 3.325(10.3); 3.169(1.0); 3.152(16.0); 2.891(3.4); 2.731(2.8); 2.525(0.4); 2.511(7.9); 2.507(15.6); 2.502(20.6); 2.498(15.4); 2.494(7.9); 1.132(0.4); 1.120(0.5); 1.116(0.5); 1.105(1.0); 1.095(0.7); 1.091(0.7); 1.081(0.6); 0.989(0.5); 0.979(0.7); 0.975(0.7); 0.966(1.0); 0.954(0.5); 0.950(0.5); 0.939(0.5); 0.822(0.4); 0.811(0.4); 0.806(0.4); 0.797(0.9); 0.785(0.8); 0.782(0.9); 0.772(0.9); 0.765(0.9); 0.755(0.9); 0.751(0.8); 0.739(0.8); 0.730(0.4); 0.725(0.4); 0.000(6.0)

### Example 98

¹H-NMR (400.0 MHz. DMSO): δ=8.742(2.5); 8.429(1.6); 8.424(1.7); 8.417(1.7); 8.412(1.6); 7.953(0.6); 7.715(1.5); 7.711(1.6); 7.697(1.8); 7.692(1.7); 7.609(2.8); 7.604(3.0); 7.528(1.5); 7.506(4.0); 7.485(2.3); 7.480(2.2); 7.470(1.9); 7.464(1.0); 7.458(2.5); 7.451(1.6); 7.439(1.5); 5.041(4.9); 3.325(19.9); 3.161(16.0); 2.891(4.5); 2.732(3.9); 2.524(0.6); 2.506(21.7); 2.502(28.2); 2.498(20.7); 1.081(0.4); 1.071(0.5); 1.067(0.5); 1.057(0.5); 1.047(1.0); 1.042(1.2); 0.943(1.2); 0.939(1.2); 0.927(0.4); 0.913(0.6); 0.905(0.4); 0.778(0.3); 0.753(0.9); 0.738(3.4); 0.722(0.8); 0.000(6.8)

### Example 99

¹H-NMR (400.0 MHz. DMSO): δ=9.020(2.4); 8.992(2.1); 8.986(2.4); 8.924(2.5); 8.918(2.1); 7.613(2.9); 7.608(3.1); 7.543(2.1); 7.522(3.3); 7.455(1.9); 7.449(1.8); 7.434(1.2); 7.428(1.2); 4.980(4.6); 3.329(2.5); 3.189(16.0); 2.892(0.4); 2.526(0.4); 2.512(6.5); 2.508(12.7); 2.503(16.5); 2.499(12.0); 2.494(5.9); 1.008(0.4); 0.997(0.5); 0.993(0.6); 0.983(0.8); 0.974(0.6); 0.968(0.7); 0.957(0.5); 0.860(0.4); 0.847(0.7); 0.843(0.7); 0.830(0.8); 0.824(0.5); 0.818(0.5); 0.806(0.9); 0.789(0.5); 0.782(0.9); 0.766(0.9); 0.754(0.7); 0.743(0.8); 0.732(0.8); 0.727(0.6); 0.716(0.7); 0.708(0.4); 0.703(0.3); 0.000(5.2)

### Example 100

¹H-NMR (400.0 MHz. DMSO): δ=8.850(2.3); 8.763(1.1); 8.760(1.2); 8.751(1.2); 8.749(1.2); 7.826(0.8); 7.823(0.8); 7.807(1.6); 7.803(1.6); 7.772(1.5); 7.761(1.4); 7.753(0.8); 7.741(0.8); 7.632(2.7); 7.627(2.8); 7.532(1.2); 7.510(3.9); 7.496(2.4); 7.491(2.1); 7.475(0.7); 7.470(0.7); 5.019(4.6); 3.326(15.8); 3.169(16.0); 2.891(0.4); 2.525(0.4); 2.511(7.3); 2.507(14.5); 2.502(18.9); 2.498(13.7); 2.493(6.5); 1.035(0.4); 1.030(0.5); 1.025(0.5); 1.021(0.4); 1.005(0.7); 1.000(0.8); 0.896(0.5); 0.891(0.7); 0.887(0.7); 0.882(0.6); 0.866(0.5); 0.861(0.6); 0.858(0.5); 0.740(0.4); 0.715(2.1); 0.711(2.3); 0.706(2.2); 0.702(1.9); 0.000(5.9)

### Example 101

¹H-NMR (400.0 MHz. DMSO): δ=8.954(0.5); 8.940(1.0); 8.925(0.5); 8.774(1.4); 8.770(1.4); 8.762(1.4); 8.758(1.4); 7.927(1.1); 7.907(1.3); 7.665(1.0); 7.653(1.2); 7.644(2.9); 7.642(3.0); 7.633(1.0); 7.490(6.5); 7.128(1.0); 6.992(2.3); 6.857(1.1); 4.820(1.0); 4.808(1.4); 4.804(1.3); 4.792(1.1); 3.621(0.4); 3.605(0.7); 3.587(0.9); 3.571(1.2); 3.554(0.6); 3.517(0.6); 3.504(1.1); 3.491(0.7); 3.483(0.5); 3.470(0.7); 3.457(0.4); 3.326(12.6); 3.219(16.0); 3.209(1.0); 2.891(0.5); 2.732(0.5); 2.525(0.4); 2.512(8.2); 2.507(16.0); 2.503(20.7); 2.498(14.9); 2.494(7.1); 0.000(6.1)

### Example 102

¹H-NMR (400.0 MHz. DMSO): δ=8.798(0.5); 8.783(1.0); 8.769(0.5); 8.460(1.5); 8.455(1.6); 8.448(1.6); 8.443(1.5); 7.790(1.4); 7.785(1.5); 7.771(1.7); 7.766(1.6); 7.642(1.6); 7.639(2.9); 7.636(1.8); 7.505(6.4); 7.502(6.3); 7.494(2.1); 7.482(1.7); 7.475(1.6); 7.463(1.6); 4.796(1.0); 4.780(1.7); 4.766(1.0); 3.513(1.3); 3.500(1.9); 3.496(2.0); 3.486(1.0); 3.481(1.0); 3.325(16.1); 3.217(16.0); 3.208(0.9); 2.891(0.8); 2.731(0.7); 2.525(0.5); 2.511(8.6); 2.507(17.1); 2.502(22.5); 2.498(16.3); 2.493(7.8); 0.000(6.3)

### Example 103

¹H-NMR (400.0 MHz. DMSO): δ=9.014(1.9); 9.014(1.9); 9.007(2.6); 8.991(0.5); 8.953(2.1); 8.947(1.7); 7.652(1.7); 7.650(2.1); 7.648(1.8); 7.647(1.9); 7.501(6.2); 7.497(3.1); 7.476(0.3); 4.789(0.9); 4.779(1.1); 4.770(1.1); 4.760(0.9); 3.649(0.4); 3.639(0.4); 3.634(0.5); 3.624(0.5); 3.614(0.6); 3.604(0.6); 3.599(0.6); 3.589(0.6); 3.457(0.5); 3.442(0.6); 3.438(0.6); 3.423(0.9); 3.408(0.4); 3.403(0.5); 3.389(0.4); 3.329(1.8); 3.219(0.7); 3.211(16.0); 2.892(0.5); 2.733(0.3); 2.732(0.4); 2.521(0.4); 2.513(5.5); 2.508(11.2); 2.504(14.8); 2.499(10.6); 2.495(5.0); 0.000(6.2)

### Example 104

¹H-NMR (400.0 MHz. DMSO): δ=8.889(0.6); 8.875(1.1); 8.860(0.5); 8.788(1.3); 8.779(1.3); 8.777(1.3); 7.904(1.1); 7.886(1.4); 7.787(1.3); 7.775(1.2); 7.767(1.0); 7.755(0.9); 7.653(2.8); 7.509(5.7); 7.506(6.5); 7.496(0.4); 7.493(0.4); 4.772(1.0); 4.761(1.2); 4.753(1.3); 4.743(1.0); 3.583(0.4); 3.573(0.5); 3.569(0.5); 3.558(0.4); 3.549(0.7); 3.538(0.8); 3.535(0.8); 3.524(0.6); 3.450(0.6); 3.435(0.8); 3.432(0.7); 3.416(1.0); 3.401(0.5); 3.397(0.5); 3.382(0.4); 3.325(9.5); 3.211(16.0); 2.891(0.4); 2.511(8.1); 2.507(15.5); 2.503(19.9); 2.498(14.3); 2.494(7.0); 0.000(6.1)

### Example 105

¹H-NMR (400.0 MHz. DMSO): δ=8.763(1.1); 8.757(3.1); 8.753(3.3); 8.745(3.7); 8.741(4.4); 8.718(2.6); 8.698(0.7); 7.882(0.7); 7.862(0.8); 7.838(2.1); 7.818(2.5); 7.661(2.0); 7.649(2.1); 7.641(2.3); 7.630(3.3); 7.592(4.6); 7.589(4.3); 7.516(0.7); 7.495(12.1); 7.491(5.7); 7.478(0.5); 7.474(0.7); 7.469(0.9); 7.464(4.4); 7.462(3.7); 6.982(0.6); 6.958(1.8); 6.846(1.3); 6.823(4.2); 6.711(0.7); 6.687(2.1); 4.788(1.5); 4.775(1.6); 4.720(4.3); 4.704(4.6); 4.382(0.4); 4.377(0.3); 4.364(0.4); 4.359(0.4); 4.340(1.0); 4.323(1.4); 4.319(1.2); 4.307(1.1); 4.302(1.4); 4.286(0.9); 3.381(1.0); 3.375(1.1); 3.364(1.5); 3.358(3.6); 3.351(1.7); 3.347(1.9); 3.340(5.3); 3.326(35.5); 3.316(2.1); 3.305(1.1); 3.298(1.1); 3.280(0.4); 2.891(2.4); 2.732(2.0); 2.672(0.4); 2.525(1.0); 2.520(1.6); 2.512(21.1); 2.507(42.3); 2.503(55.5); 2.498(40.3); 2.494(19.5); 2.329(0.4); 1.235(0.6); 1.173(3.6); 1.158(12.4); 1.141(15.3); 1.126(16.0); 1.108(7.6); 1.092(0.5); 1.083(0.4); 0.008(0.6); 0.000(18.7); −0.009(0.6)

### Example 106

¹H-NMR (400.0 MHz. DMSO): δ=8.626(2.1); 8.605(2.1); 8.558(0.6); 8.536(0.6); 8.451(2.9); 8.446(3.1); 8.439(3.7); 8.434(3.7); 8.428(1.1); 8.423(1.0); 7.953(0.4); 7.705(2.7); 7.700(2.8); 7.686(3.4); 7.681(3.3); 7.673(1.0); 7.659(1.1); 7.654(1.0); 7.612(5.3); 7.611(5.3); 7.507(0.6); 7.487(16.0); 7.478(5.0); 7.471(3.2); 7.466(1.6); 7.459(3.7); 7.447(0.9); 4.764(1.4); 4.751(1.5); 4.734(4.4); 4.720(4.7); 4.322(0.4); 4.317(0.4); 4.308(1.1); 4.300(0.6); 4.294(1.3); 4.291(1.4); 4.287(1.2); 4.277(1.1); 4.273(1.3); 4.256(0.9); 3.397(1.1); 3.391(0.8); 3.379(1.3); 3.373(2.6); 3.356(2.9); 3.347(1.6); 3.342(3.0); 3.334(2.2); 3.325(42.4); 3.307(1.2); 3.302(1.3); 3.284(0.4); 2.891(2.7); 2.732(2.2); 2.671(0.4); 2.525(1.0); 2.511(22.7); 2.507(45.9); 2.502(60.7); 2.498(44.4); 2.494(21.8); 2.329(0.4); 1.235(0.7); 1.164(3.3); 1.148(9.6); 1.137(3.2); 1.131(14.6); 1.120(5.5); 1.113(7.1); 1.101(12.5); 1.084(11.0); 0.008(0.5); 0.000(17.5); −0.009(0.6)

### Example 107

¹H-NMR (400.0 MHz. DMSO): δ=9.010(4.0); 9.004(4.7); 8.997(1.6); 8.991(1.6); 8.944(4.8); 8.938(3.9); 8.921(1.6); 8.915(1.4); 8.804(2.2); 8.782(2.2); 8.714(0.7); 8.692(0.8); 7.632(4.3); 7.631(4.5); 7.629(5.2); 7.627(4.1); 7.619(2.2); 7.614(2.2); 7.512(1.9); 7.490(9.7); 7.486(13.6); 7.466(0.7); 7.447(1.4); 7.442(1.4); 7.426(0.8); 7.421(0.7); 4.767(1.9); 4.761(5.1); 4.756(2.4); 4.751(5.1); 4.355(1.0); 4.344(1.0); 4.337(1.2); 4.333(1.2); 4.327(1.2); 4.322(1.2); 4.316(1.1); 4.311(0.8); 4.305(1.1); 4.299(0.6); 4.294(0.5); 4.289(0.6); 4.282(0.4); 4.277(0.4); 4.272(0.3); 3.409(0.5); 3.392(1.1); 3.386(1.2); 3.377(2.2); 3.369(3.6); 3.359(6.0); 3.351(4.4); 3.342(8.4); 3.330(29.9); 3.046(0.5); 2.892(1.0); 2.814(0.5); 2.733(0.8); 2.677(0.3); 2.672(0.5); 2.668(0.3); 2.547(0.4); 2.525(1.5); 2.512(28.3); 2.508(55.3); 2.503(71.5); 2.499(51.7); 2.494(25.1); 2.334(0.4); 2.330(0.5); 2.325(0.4); 1.235(0.7); 1.208(0.5); 1.192(4.1); 1.175(4.0); 1.156(7.4); 1.139(16.0); 1.134(4.4); 1.121(7.9); 1.116(6.5); 1.099(2.8); 1.080(0.4); 1.062(0.4); 1.043(11.4); 1.026(11.3); 0.968(0.6); 0.951(0.6); 0.008(0.9); 0.000(23.1); −0.009(0.9)

### Example 108

¹H-NMR (400.0 MHz. DMSO): δ=8.785(1.8); 8.777(3.3); 8.769(2.5); 8.757(0.8); 8.754(0.7); 8.703(2.0); 8.682(2.0); 8.654(0.7); 8.632(0.7); 7.954(0.4); 7.789(0.4); 7.776(10.8); 7.768(6.7); 7.756(1.2); 7.745(1.3); 7.739(1.1); 7.734(1.2); 7.719(0.4); 7.633(4.8); 7.625(1.2); 7.622(1.9); 7.619(1.2); 7.510(4.3); 7.507(4.0); 7.484(12.1); 7.465(0.5); 4.751(1.4); 4.739(1.5); 4.728(4.3); 4.715(4.5); 4.321(0.4); 4.311(1.0); 4.298(1.2); 4.294(1.3); 4.291(1.3); 4.281(1.2); 4.277(1.2); 4.273(1.1); 4.260(0.8); 3.383(1.0); 3.377(0.9); 3.366(1.6); 3.360(3.0); 3.350(2.7); 3.340(5.3); 3.330(89.5); 3.316(2.2); 3.305(1.1); 3.299(1.2); 3.281(0.4); 2.892(2.8); 2.732(2.3); 2.525(0.9); 2.512(18.5); 2.508(37.0); 2.503(48.6); 2.499(35.2); 2.494(17.0); 1.235(0.6); 1.149(9.2); 1.141(3.4); 1.131(16.0); 1.124(5.8); 1.114(6.6); 1.106(2.7); 1.092(0.5); 1.069(10.2); 1.052(10.1); 0.000(7.3)

### Example 109

¹H-NMR (400.0 MHz. DMSO): δ=8.580(0.5); 8.566(0.8); 8.551(0.5); 8.440(1.5); 8.436(1.7); 8.428(1.7); 8.424(1.6); 7.719(1.5); 7.714(1.6); 7.700(1.8); 7.695(1.8); 7.468(1.8); 7.456(1.8); 7.449(1.6); 7.437(2.4); 7.430(0.6); 7.418(3.7); 7.415(3.3); 7.405(2.7); 7.400(0.6); 7.387(2.9); 7.384(1.2); 7.367(1.2); 7.315(0.6); 7.312(1.1); 7.307(0.6); 7.300(0.5); 7.294(1.2); 7.288(0.4); 7.277(0.5); 3.658(0.9); 3.641(0.9); 3.624(1.2); 3.607(1.1); 3.454(1.0); 3.441(1.1); 3.420(0.9); 3.407(0.8); 3.351(116.5); 3.021(16.0); 2.525(0.5); 2.512(8.7); 2.508(17.3); 2.503(22.6); 2.499(16.5); 2.494(8.0); 1.566(9.8)

### Example 110

¹H-NMR (400.0 MHz. DMSO): δ=8.745(1.6); 8.742(1.7); 8.734(1.8); 8.730(1.9); 8.710(1.2); 8.695(0.7); 7.872(1.3); 7.852(1.5); 7.632(1.1); 7.620(1.2); 7.612(1.1); 7.600(1.0); 7.433(1.2); 7.429(1.7); 7.411(4.3); 7.398(3.0); 7.380(3.5); 7.360(1.5); 7.311(1.2); 7.307(0.8); 7.294(1.5); 7.276(0.6); 6.915(1.0); 6.780(2.3); 6.644(1.1); 3.644(0.8); 3.628(0.9); 3.611(1.4); 3.594(1.3); 3.515(1.2); 3.501(1.3); 3.482(0.8); 3.467(0.8); 3.328(39.5); 3.041(16.0); 2.890(0.4); 2.731(0.4); 2.506(21.3); 2.502(27.3); 2.497(20.6); 1.558(11.3); 0.000(3.1)

### Example 111

¹H-NMR (400.0 MHz. DMSO): δ=7.388(0.5); 7.386(0.5); 7.374(9.0); 7.367(4.2); 7.360(3.7); 7.345(0.4); 7.339(0.6); 7.296(0.6); 7.289(0.9); 7.281(0.8); 7.274(0.9); 7.266(0.5); 7.260(0.4); 7.196(0.5); 7.182(0.8); 7.167(0.5); 4.229(1.9); 4.218(1.9); 4.206(2.0); 3.509(0.8); 3.493(0.8); 3.475(1.2); 3.459(1.2); 3.361(1.1); 3.347(1.3); 3.327(34.0); 3.313(0.9); 3.015(16.0); 2.983(2.1); 2.975(1.5); 2.972(2.1); 2.961(2.0); 2.524(0.5); 2.511(9.1); 2.506(17.9); 2.502(23.3); 2.497(16.7); 2.493(7.9); 1.918(14.9); 1.478(0.3); 1.462(10.0); 0.000(3.0)

### Example 112

¹H-NMR (400.0 MHz. DMSO): δ=9.001(1.7); 8.995(1.9); 8.933(2.0); 8.927(1.7); 8.666(0.4); 8.652(0.6); 8.637(0.4); 7.448(1.1); 7.444(1.6); 7.439(0.6); 7.427(3.3); 7.424(2.9); 7.408(2.2); 7.402(0.5); 7.390(2.8); 7.386(1.1); 7.374(0.7); 7.370(1.3); 7.323(0.6); 7.319(1.1); 7.315(0.6); 7.307(0.5); 7.301(1.3); 7.296(0.4); 7.287(0.3); 7.284(0.5); 3.707(0.9); 3.690(0.9); 3.673(1.2); 3.656(1.1); 3.482(0.9); 3.470(0.9); 3.448(0.7); 3.436(0.7); 3.329(16.4); 3.018(16.0); 2.891(0.4); 2.525(0.4); 2.520(0.7); 2.511(7.8); 2.507(15.7); 2.502(20.7); 2.498(14.8); 2.493(6.9); 1.558(0.4); 1.542(9.2); 1.231(0.3); 0.000(5.7)

### Example 113

¹H-NMR (400.0 MHz. DMSO): δ=8.763(1.3); 8.754(1.3); 8.752(1.3); 8.640(0.5); 8.625(0.9); 8.610(0.5); 7.803(0.8); 7.801(0.9); 7.784(1.7); 7.781(1.7); 7.751(1.5); 7.739(1.5); 7.731(0.8); 7.720(0.8); 7.440(0.9); 7.436(1.4); 7.418(4.2); 7.415(3.7); 7.410(3.5); 7.391(3.1); 7.371(1.2); 7.320(0.7); 7.316(1.1); 7.311(0.6); 7.299(1.3); 7.285(0.4); 7.281(0.5); 3.646(0.9); 3.629(0.9); 3.612(1.2); 3.595(1.2); 3.466(1.1); 3.452(1.1); 3.432(0.8); 3.419(0.8); 3.328(31.8); 3.020(16.0); 2.890(0.5); 2.731(0.4); 2.506(18.4); 2.502(23.4); 2.497(17.1); 1.547(10.4); 0.000(3.5)

### Example 116

¹H-NMR (400.0 MHz. CD3CN): δ=7.653(0.7); 7.507(3.8); 7.502(2.9); 7.496(0.9); 7.488(1.1); 7.467(1.8); 7.417(1.0); 7.413(1.2); 7.408(1.0); 7.397(1.6); 7.392(0.8); 7.387(0.6); 7.359(0.9); 7.349(1.6); 7.342(0.9); 7.326(0.5); 7.321(0.5); 7.202(0.6); 4.728(1.3); 4.715(1.6); 4.711(1.3); 4.701(1.2); 4.471(0.5); 4.464(0.4); 4.456(0.6); 4.454(0.6); 4.448(0.6); 4.441(0.6); 4.436(0.6); 4.434(0.6); 4.424(0.4); 4.419(0.4); 3.265(13.9); 3.264(14.4); 2.512(3.1); 2.204(7.7); 2.194(6.5); 2.151(10.1); 2.106(5.5); 2.091(6.7); 1.964(1.5); 1.958(2.5); 1.952(9.8); 1.946(17.1); 1.940(22.0); 1.934(15.4); 1.928(8.1); 1.437(16.0); 1.302(3.7); 1.284(3.8); 1.277(0.5); 1.116(4.6); 1.099(4.6); 0.000(2.6)

### Example 117

¹H-NMR (601.6 MHz. DMSO): δ=8.896(0.4); 8.886(0.8); 8.876(0.4); 8.767(1.2); 8.764(1.2); 8.759(1.2); 8.756(1.2); 7.878(0.8); 7.876(0.8); 7.875(0.8); 7.865(0.9); 7.863(0.9); 7.656(0.8); 7.648(0.8); 7.643(0.7); 7.635(0.7); 7.572(1.3); 7.569(2.6); 7.566(1.4); 7.396(4.5); 7.393(4.4); 7.094(0.7); 7.004(1.8); 6.914(0.8); 4.468(0.7); 4.458(1.2); 4.448(0.7); 3.569(0.5); 3.558(0.5); 3.556(0.6); 3.546(0.7); 3.536(0.5); 3.493(0.5); 3.483(0.9); 3.473(0.5); 3.470(0.4); 3.461(0.6); 3.316(22.1); 3.229(16.0); 2.522(0.5); 2.519(0.6); 2.516(0.7); 2.507(11.2); 2.504(24.1); 2.501(33.0); 2.498(24.1); 2.495(11.0); 0.000(0.4)

### Example 118

¹H-NMR (601.6 MHz. DMSO): δ=8.736(0.4); 8.726(0.8); 8.717(0.4); 8.454(1.5); 8.451(1.6); 8.446(1.6); 8.443(1.6); 7.751(1.5); 7.748(1.6); 7.738(1.7); 7.735(1.6); 7.567(1.4); 7.564(2.7); 7.561(1.4); 7.486(1.7); 7.478(1.7); 7.474(1.6); 7.466(1.6); 7.403(4.6); 7.400(4.6); 4.448(0.7); 4.438(1.4); 4.428(0.7); 3.562(0.3); 3.552(0.5); 3.542(0.5); 3.539(0.6); 3.529(0.8); 3.519(0.5); 3.474(0.5); 3.464(1.0); 3.454(0.6); 3.452(0.4); 3.442(0.6); 3.432(0.3); 3.316(21.8); 3.228(16.0); 2.890(0.5); 2.731(0.4); 2.731(0.4); 2.522(0.6); 2.519(0.8); 2.516(0.9); 2.507(11.6); 2.504(24.2); 2.501(32.9); 2.498(23.8); 2.495(10.8); 0.000(0.4)

### Example 119

¹H-NMR (601.6 MHz. DMSO): δ=8.814(0.5); 8.805(1.0); 8.795(0.5); 8.786(1.1); 8.784(1.1); 8.778(1.1); 8.776(1.1); 7.859(0.9); 7.857(0.9); 7.846(1.3); 7.844(1.2); 7.779(1.2); 7.771(1.2); 7.766(0.9); 7.758(0.9); 7.576(1.4); 7.573(2.7); 7.570(1.4); 7.399(5.3); 7.395(5.1); 4.416(0.8); 4.406(1.4); 4.396(0.8); 3.525(0.3); 3.512(0.8); 3.502(1.1); 3.496(0.8); 3.492(0.8); 3.486(1.3); 3.477(0.7); 3.464(0.4); 3.316(23.2); 3.221(16.0); 2.890(0.7); 2.731(0.5); 2.522(0.5); 2.519(0.6); 2.516(0.7); 2.507(13.9); 2.504(29.0); 2.501(39.4); 2.498(28.5); 2.495(13.2); 0.000(0.4)

### Example 120

¹H-NMR (601.6 MHz. DMSO): δ=9.001(1.7); 8.998(1.9); 8.943(2.0); 8.939(1.8); 8.922(0.4); 8.913(0.9); 8.903(0.4); 7.562(1.3); 7.559(2.6); 7.555(1.4); 7.409(5.0); 7.406(4.8); 4.449(0.8); 4.439(1.6); 4.429(0.8); 3.551(1.6); 3.541(3.0); 3.531(1.5); 3.317(26.6); 3.221(16.0); 2.891(0.4); 2.731(0.3); 2.522(0.4); 2.519(0.6); 2.516(0.6); 2.507(13.2); 2.504(28.0); 2.501(38.3); 2.498(27.9); 2.495(12.9); 0.000(0.3)

### Example 128

¹H-NMR (400.0 MHz. DMSO): δ=8.498(1.1); 8.477(1.1); 8.391(0.7); 8.370(0.7); 7.763(0.8); 7.744(2.3); 7.734(0.6); 7.725(1.7); 7.716(1.0); 7.696(0.7); 7.688(0.6); 7.670(1.4); 7.652(1.1); 7.643(0.7); 7.623(1.8); 7.604(1.5); 7.585(0.4); 7.467(3.6); 7.461(2.9); 7.446(5.5); 7.440(3.7); 7.419(1.0); 7.400(0.9); 7.355(5.0); 7.334(3.8); 7.119(1.4); 7.101(1.3); 4.273(1.0); 4.257(1.5); 4.222(1.8); 4.206(2.4); 4.191(0.5); 4.171(0.5); 4.092(0.6); 4.076(0.9); 4.071(0.7); 4.059(0.6); 4.054(0.9); 4.038(0.6); 3.323(16.4); 3.181(16.0); 3.169(10.4); 2.510(15.2); 2.506(29.6); 2.502(38.0); 2.497(27.0); 2.493(12.8); 1.989(0.4); 1.398(2.7); 1.250(0.4); 1.128(6.1); 1.111(6.0); 0.964(3.7); 0.947(3.6); 0.008(1.4); 0.000(32.5); −0.008(1.1)

### Example 129

¹H-NMR (400.0 MHz. DMSO): δ=8.157(0.7); 8.137(0.7); 8.052(0.7); 8.032(0.6); 7.453(2.1); 7.448(0.9); 7.436(3.4); 7.431(3.7); 7.425(0.7); 7.419(1.4); 7.415(4.2); 7.409(0.7); 7.348(3.9); 7.331(3.5); 7.327(3.2); 7.319(0.7); 7.314(1.1); 7.310(2.0); 7.303(0.7); 7.297(0.9); 7.283(0.8); 7.278(1.2); 7.261(1.2); 7.249(0.7); 7.243(1.0); 7.239(1.2); 7.234(1.4); 7.230(1.2); 7.221(2.3); 7.203(1.5); 7.176(0.8); 7.164(1.6); 7.159(1.5); 7.146(1.0); 7.023(1.1); 7.019(1.2); 7.002(1.0); 4.283(0.9); 4.268(1.5); 4.239(0.4); 4.223(0.5); 4.203(0.5); 4.161(0.7); 4.143(2.6); 4.130(0.6); 4.125(0.6); 4.109(0.6); 3.323(13.4); 3.158(16.0); 2.607(1.1); 2.524(0.8); 2.510(15.5); 2.506(31.1); 2.501(40.7); 2.497(29.1); 2.492(13.8); 2.320(0.3); 2.282(6.9); 2.039(9.1); 1.989(0.4); 1.398(1.6); 1.336(0.4); 1.249(0.5); 1.190(4.1); 1.174(4.2); 0.964(3.4); 0.948(3.4); 0.008(1.4); 0.000(38.0); −0.009(1.3)

### Example 130

¹H-NMR (400.0 MHz. DMSO): δ=8.415(0.9); 8.393(0.9); 8.298(0.4); 8.278(0.4); 7.481(0.4); 7.478(0.5); 7.461(0.9); 7.458(1.1); 7.449(4.4); 7.444(2.3); 7.428(7.5); 7.419(1.1); 7.414(1.2); 7.410(1.2); 7.404(0.6); 7.396(2.0); 7.392(1.8); 7.377(1.3); 7.372(1.1); 7.360(0.7); 7.348(5.1); 7.342(1.9); 7.335(2.8); 7.331(3.3); 7.326(3.2); 7.317(1.2); 7.313(0.8); 7.097(1.4); 7.093(1.4); 7.079(1.3); 7.074(1.2); 4.306(0.7); 4.291(0.9); 4.212(1.6); 4.196(2.1); 4.104(0.5); 4.088(0.8); 4.083(0.6); 4.071(0.5); 4.066(0.8); 4.049(0.5); 3.321(11.5); 3.181(16.0); 3.169(7.2); 2.523(0.9); 2.510(15.3); 2.506(30.8); 2.501(40.7); 2.497(29.2); 2.492(14.1); 1.398(1.3); 1.155(5.8); 1.138(5.7); 0.976(2.4); 0.959(2.4); 0.008(1.5); 0.000(39.6); −0.009(1.5)

### Example 131

¹H-NMR (400.0 MHz. DMSO): δ=8.416(1.0); 8.394(1.0); 8.293(0.8); 8.273(0.8); 7.637(1.3); 7.635(1.3); 7.617(1.5); 7.615(1.5); 7.606(1.6); 7.604(1.6); 7.587(1.8); 7.584(1.8); 7.453(4.3); 7.433(6.8); 7.421(1.6); 7.419(1.5); 7.403(1.4); 7.400(1.3); 7.395(0.9); 7.379(1.7); 7.376(1.8); 7.358(5.3); 7.350(5.0); 7.344(2.0); 7.337(3.8); 7.332(4.0); 7.328(4.4); 7.318(0.9); 7.313(2.1); 7.307(2.6); 7.302(1.4); 7.294(0.8); 7.288(1.6); 7.284(1.0); 7.043(1.5); 7.039(1.6); 7.025(1.4); 7.020(1.4); 5.756(4.6); 4.303(1.4); 4.288(1.8); 4.224(1.8); 4.208(2.3); 4.195(0.7); 4.176(0.6); 4.160(0.4); 4.089(0.6); 4.072(0.9); 4.068(0.7); 4.056(0.8); 4.051(0.9); 4.038(0.8); 4.020(0.7); 3.322(17.4); 3.187(16.0); 3.169(13.4); 2.670(0.4); 2.523(1.2); 2.510(21.6); 2.506(43.1); 2.501(56.7); 2.497(40.9); 2.492(20.0); 2.328(0.4); 1.988(2.6); 1.398(4.5); 1.249(0.4); 1.193(0.7); 1.175(1.6); 1.155(6.3); 1.138(6.1); 1.009(0.4); 0.991(0.9); 0.975(4.9); 0.958(4.7); 0.008(1.9); 0.000(48.8); −0.009(2.0)

### Example 132

¹H-NMR (400.0 MHz. DMSO): δ=8.740(1.0); 8.719(0.9); 8.622(0.7); 8.602(0.7); 8.316(0.3); 7.508(0.4); 7.503(0.6); 7.498(0.6); 7.491(0.8); 7.486(0.9); 7.482(1.1); 7.475(0.7); 7.470(1.3); 7.465(1.0); 7.460(0.8); 7.454(0.7); 7.449(0.9); 7.434(5.1); 7.413(7.5); 7.335(4.1); 7.332(5.3); 7.314(3.2); 7.311(3.3); 7.158(0.4); 7.151(1.6); 7.133(3.6); 7.112(3.4); 7.094(1.8); 7.086(0.4); 4.295(1.3); 4.281(1.7); 4.241(1.8); 4.226(2.2); 4.205(0.6); 4.187(0.5); 4.170(0.3); 4.112(0.5); 4.095(0.7); 4.075(0.7); 4.059(0.5); 3.322(62.7); 3.210(0.3); 3.195(16.0); 3.169(12.2); 2.675(0.7); 2.671(0.9); 2.666(0.7); 2.541(0.6); 2.524(2.8); 2.510(58.1); 2.506(116.5); 2.502(152.3); 2.497(109.2); 2.493(52.5); 2.333(0.7); 2.328(1.0); 2.324(0.7); 1.398(1.3); 1.100(6.0); 1.083(5.9); 0.984(4.4); 0.967(4.3); 0.146(0.5); 0.008(4.5); 0.000(120.3); −0.008(4.3); −0.150(0.5)

### Example 133

¹H-NMR (400.0 MHz. DMSO): δ=8.215(0.7); 8.193(0.7); 8.063(0.5); 8.047(0.5); 7.550(0.4); 7.546(0.6); 7.536(0.4); 7.532(0.9); 7.527(1.2); 7.518(0.7); 7.510(1.0); 7.504(0.9); 7.497(1.0); 7.492(1.1); 7.487(0.7); 7.484(0.9); 7.478(0.8); 7.474(0.7); 7.471(0.9); 7.466(0.6); 7.458(0.5); 7.453(0.5); 7.446(0.4); 7.440(2.6); 7.433(3.3); 7.428(1.3); 7.423(1.2); 7.418(4.2); 7.412(5.4); 7.406(0.8); 7.373(0.7); 7.369(0.7); 7.354(1.9); 7.349(5.5); 7.344(1.9); 7.336(4.0); 7.332(2.6); 7.328(3.1); 7.321(1.1); 7.316(2.2); 7.288(0.8); 7.278(0.9); 7.275(0.9); 7.267(0.8); 7.259(2.5); 7.240(2.7); 7.232(1.2); 7.230(1.1); 7.224(1.9); 7.211(0.8); 7.209(0.9); 7.205(1.0); 5.757(4.3); 4.341(1.2); 4.327(1.6); 4.252(2.0); 4.236(2.6); 4.216(0.5); 4.146(0.5); 4.129(0.8); 4.125(0.6); 4.112(0.5); 4.108(0.7); 4.091(0.4); 3.325(4.6); 3.194(16.0); 3.181(11.7); 2.525(0.5); 2.512(9.8); 2.507(19.7); 2.503(25.7); 2.498(18.2); 2.494(8.6); 1.989(0.8); 1.397(1.6); 1.337(0.3); 1.250(0.4); 1.175(0.5); 1.157(0.4); 1.146(6.0); 1.129(6.0); 1.013(4.2); 0.995(4.2); 0.008(0.7); 0.000(17.5); −0.008(0.6)

### Example 134

¹H-NMR (400.0 MHz. DMSO): δ=8.783(1.1); 8.762(1.1); 8.662(0.8); 8.642(0.8); 7.749(0.4); 7.730(0.8); 7.717(1.2); 7.697(2.3); 7.681(1.0); 7.673(0.7); 7.663(1.1); 7.644(1.7); 7.628(2.0); 7.605(4.9); 7.588(2.3); 7.570(0.5); 7.445(3.5); 7.440(1.5); 7.434(2.7); 7.429(2.3); 7.424(5.0); 7.418(1.8); 7.413(4.0); 7.350(3.6); 7.334(5.3); 7.329(3.5); 7.313(3.1); 5.756(2.1); 4.252(1.8); 4.239(2.9); 4.226(2.1); 4.207(0.5); 4.190(0.7); 4.173(0.6); 4.156(0.3); 4.122(0.5); 4.105(0.8); 4.087(0.7); 4.070(0.5); 3.369(9.1); 3.186(16.0); 3.160(12.4); 3.117(0.4); 2.675(0.5); 2.671(0.7); 2.666(0.5); 2.541(0.5); 2.511(45.5); 2.506(86.1); 2.502(108.5); 2.497(75.6); 2.493(35.0); 2.333(0.5); 2.329(0.7); 2.324(0.5); 1.123(0.4); 1.066(6.1); 1.049(6.0); 1.001(0.4); 0.970(4.4); 0.953(4.4); 0.008(0.8); 0.000(18.3); −0.009(0.6)

### Example 135

¹H-NMR (400.0 MHz. DMSO): δ=10.927(0.4); 8.615(0.9); 8.594(1.0); 8.493(0.7); 8.472(0.8); 8.452(1.2); 8.448(1.4); 8.441(2.8); 8.436(2.9); 8.429(1.8); 8.424(1.7); 7.766(1.1); 7.761(1.2); 7.747(1.3); 7.742(1.3); 7.604(1.4); 7.599(1.5); 7.585(2.0); 7.580(1.9); 7.490(1.5); 7.478(1.4); 7.470(2.7); 7.457(5.5); 7.451(3.3); 7.436(6.2); 7.415(0.6); 7.410(0.4); 7.399(0.4); 7.362(3.3); 7.347(4.8); 7.341(3.2); 7.326(3.0); 7.300(0.3); 5.756(3.5); 4.306(1.2); 4.292(1.5); 4.242(1.7); 4.226(2.1); 4.203(0.5); 4.185(0.5); 4.104(0.5); 4.087(0.8); 4.083(0.6); 4.071(0.6); 4.066(0.7); 4.050(0.5); 3.323(41.6); 3.280(1.5); 3.197(16.0); 3.175(11.8); 3.164(0.7); 3.114(0.3); 3.096(0.3); 3.012(0.4); 2.671(0.4); 2.541(0.4); 2.524(1.5); 2.511(27.2); 2.506(55.5); 2.502(73.8); 2.497(53.1); 2.493(25.5); 2.333(0.3); 2.328(0.5); 2.324(0.4); 1.550(1.5); 1.234(0.5); 1.136(5.8); 1.119(5.8); 1.013(4.1); 0.996(4.0); 0.008(0.5); 0.000(14.5); −0.009(0.5)

### Example 136

¹H-NMR (400.0 MHz. DMSO): δ=8.561(0.5); 8.539(0.6); 8.467(0.6); 8.447(0.5); 8.103(1.4); 8.087(1.3); 7.705(0.4); 7.695(0.6); 7.682(0.7); 7.656(0.4); 7.650(0.5); 7.634(1.0); 7.621(1.2); 7.617(1.0); 7.609(1.1); 7.598(1.2); 7.589(1.0); 7.584(1.2); 7.574(1.2); 7.566(0.9); 7.519(0.6); 7.507(0.5); 7.498(0.4); 7.450(1.6); 7.445(1.0); 7.438(2.3); 7.429(2.5); 7.417(3.1); 7.351(2.4); 7.336(2.5); 7.330(2.1); 7.315(2.3); 7.308(1.3); 7.287(0.7); 7.281(0.6); 7.141(1.0); 7.002(0.6); 6.939(0.5); 6.800(1.0); 6.662(0.5); 6.587(1.5); 6.583(1.1); 6.574(1.3); 6.570(1.5); 5.757(1.4); 5.756(1.4); 4.307(0.8); 4.292(1.2); 4.242(0.4); 4.223(0.4); 4.188(0.7); 4.170(1.4); 4.148(0.3); 4.131(0.4); 4.110(0.4); 3.327(4.1); 3.184(0.7); 3.172(10.0); 3.147(2.8); 3.113(0.4); 3.106(0.9); 3.002(0.4); 2.986(0.4); 2.943(16.0); 2.507(20.0); 2.502(25.1); 2.498(18.3); 2.109(1.1); 2.104(0.5); 2.088(0.4); 2.072(0.9); 1.203(3.0); 1.187(2.9); 1.002(2.8); 0.985(2.8); 0.964(3.1); 0.948(3.3); 0.932(0.6); 0.925(1.4); 0.909(1.3); 0.754(0.4); 0.738(0.3); 0.000(4.3)

### Example 137

¹H-NMR (400.0 MHz. DMSO): δ=8.386(1.0); 8.364(1.0); 7.867(0.3); 7.865(0.4); 7.847(0.4); 7.845(0.4); 7.839(1.6); 7.837(1.7); 7.819(1.7); 7.817(1.7); 7.466(0.4); 7.460(3.2); 7.455(1.5); 7.449(0.6); 7.444(1.4); 7.439(5.0); 7.433(2.0); 7.406(0.8); 7.404(0.9); 7.388(1.8); 7.385(1.8); 7.368(1.8); 7.360(0.8); 7.354(4.3); 7.349(1.5); 7.337(1.1); 7.333(2.9); 7.240(0.4); 7.146(1.2); 7.142(1.0); 7.127(1.5); 7.123(1.4); 7.108(0.8); 7.104(0.8); 6.949(1.5); 6.945(1.5); 6.930(1.4); 6.926(1.3); 5.756(0.4); 4.308(0.3); 4.292(0.4); 4.253(1.7); 4.237(2.0); 4.069(0.5); 4.052(0.8); 4.047(0.6); 4.035(0.6); 4.031(0.8); 4.014(0.5); 3.324(16.2); 3.201(16.0); 3.173(3.4); 2.524(0.4); 2.511(8.7); 2.506(17.7); 2.502(23.2); 2.497(16.4); 2.492(7.7); 1.989(0.7); 1.397(2.2); 1.175(0.5); 1.161(5.8); 1.144(5.7); 0.980(1.1); 0.963(1.1); 0.008(0.8); 0.000(21.1); −0.009(0.7)

### Example 138

¹H-NMR (400.0 MHz. DMSO): δ=8.717(0.9); 8.696(1.0); 8.051(1.4); 8.048(1.4); 8.030(1.7); 8.027(1.6); 7.785(0.7); 7.782(0.8); 7.766(1.7); 7.763(1.7); 7.747(1.1); 7.744(1.1); 7.684(1.0); 7.680(1.1); 7.664(1.3); 7.660(1.3); 7.645(0.7); 7.641(0.7); 7.485(1.2); 7.482(1.2); 7.465(1.8); 7.462(2.0); 7.455(0.8); 7.441(1.6); 7.436(1.7); 7.425(0.8); 7.422(0.9); 7.407(1.5); 7.404(1.3); 7.391(1.7); 7.388(2.3); 7.372(1.6); 7.368(2.4); 7.363(1.2); 7.349(1.1); 7.344(1.1); 7.331(0.7); 7.326(0.6); 5.756(0.7); 4.706(2.1); 4.694(2.1); 4.308(0.5); 4.296(0.6); 4.291(0.6); 4.287(0.5); 4.279(0.5); 4.275(0.6); 4.270(0.5); 4.258(0.5); 3.322(17.7); 3.224(1.5); 3.218(16.0); 3.183(0.6); 2.524(0.4); 2.511(10.1); 2.506(20.5); 2.502(26.8); 2.497(19.1); 2.492(8.9); 1.989(0.4); 1.398(0.7); 1.113(0.5); 1.061(5.8); 1.044(5.7); 0.008(1.3); 0.000(34.8); −0.009(1.1)

### Example 139

¹H-NMR (400.0 MHz. DMSO): δ=8.702(1.2); 8.681(1.2); 8.550(0.9); 8.530(0.9); 8.029(2.7); 8.009(3.1); 7.800(0.6); 7.798(0.6); 7.782(1.4); 7.779(1.5); 7.773(0.9); 7.770(0.9); 7.763(1.1); 7.760(1.1); 7.754(1.8); 7.752(1.9); 7.735(1.2); 7.733(1.2); 7.691(0.9); 7.688(1.0); 7.680(1.2); 7.677(1.4); 7.671(1.4); 7.668(1.4); 7.660(1.6); 7.657(1.6); 7.653(0.8); 7.649(0.7); 7.641(0.8); 7.638(0.7); 7.483(1.4); 7.480(1.5); 7.464(4.5); 7.453(2.9); 7.448(2.6); 7.443(5.1); 7.437(2.1); 7.432(3.9); 7.359(3.7); 7.348(4.7); 7.344(2.6); 7.338(2.7); 7.327(3.3); 7.288(1.7); 7.285(1.8); 7.269(1.6); 7.266(1.6); 5.756(0.8); 4.314(1.4); 4.300(2.1); 4.295(2.3); 4.281(2.2); 4.200(0.4); 4.183(0.6); 4.166(0.6); 4.149(0.4); 4.056(0.9); 4.038(2.0); 4.020(2.0); 4.003(0.8); 3.321(28.2); 3.218(16.0); 3.183(12.2); 2.675(0.3); 2.670(0.5); 2.666(0.3); 2.541(0.4); 2.506(55.1); 2.502(70.3); 2.497(52.3); 2.328(0.5); 2.324(0.3); 1.989(5.5); 1.398(9.0); 1.193(1.5); 1.175(2.9); 1.157(1.5); 1.116(6.3); 1.099(6.2); 1.003(4.5); 0.986(4.5); 0.008(4.0); 0.000(66.6); −0.008(3.4)

### Example 140

¹H-NMR (400.0 MHz. DMSO): δ=8.584(1.5); 8.563(1.6); 7.662(1.2); 7.656(1.4); 7.642(1.9); 7.608(1.1); 7.592(3.3); 7.582(2.6); 7.572(2.8); 7.556(0.5); 7.473(1.8); 7.454(3.1); 7.427(3.5); 7.412(2.4); 7.396(3.0); 7.375(1.4); 7.348(1.9); 7.331(2.1); 7.313(0.9); 7.063(1.1); 6.924(2.4); 6.785(1.2); 5.755(4.2); 4.659(2.5); 4.644(2.6); 4.374(0.8); 4.357(1.2); 4.337(1.1); 4.320(0.6); 3.325(11.2); 3.322(13.3); 3.186(16.0); 3.177(3.0); 2.890(1.4); 2.732(1.3); 2.731(1.3); 2.502(35.2); 1.201(0.5); 1.185(0.7); 1.158(7.3); 1.141(7.1); 1.115(0.8); 0.000(28.4)

### Example 141

¹H-NMR (400.0 MHz. DMSO): δ=8.851(1.0); 8.830(1.0); 7.669(0.5); 7.655(0.8); 7.650(0.9); 7.636(0.9); 7.615(2.4); 7.598(1.8); 7.580(0.5); 7.487(1.3); 7.484(1.3); 7.467(1.8); 7.465(1.8); 7.450(0.5); 7.444(0.7); 7.430(1.9); 7.425(2.9); 7.407(1.6); 7.405(1.5); 7.388(0.7); 7.385(0.7); 7.373(1.3); 7.368(1.1); 7.354(1.1); 7.349(1.0); 7.337(0.6); 7.331(0.5); 7.317(0.4); 4.676(2.2); 4.666(2.3); 4.350(0.5); 4.340(0.5); 4.333(0.6); 4.330(0.6); 4.323(0.6); 4.320(0.6); 4.312(0.5); 4.302(0.5); 3.321(41.4); 3.195(16.0); 3.159(1.0); 2.670(0.4); 2.524(0.9); 2.510(23.0); 2.506(45.5); 2.502(59.0); 2.497(42.3); 2.493(20.1); 2.328(0.4); 1.088(0.4); 1.071(0.3); 1.041(0.4); 1.024(0.4); 0.996(6.1); 0.978(6.0); 0.008(2.5); 0.000(61.1); −0.009(2.0)

### Example 142

¹H-NMR (400.0 MHz. DMSO): δ=8.443(0.9); 8.422(0.9); 7.743(0.3); 7.725(0.5); 7.707(0.4); 7.703(0.3); 7.618(1.5); 7.616(1.5); 7.599(1.8); 7.596(1.7); 7.475(1.3); 7.472(1.7); 7.468(1.2); 7.462(1.1); 7.456(2.0); 7.453(2.3); 7.448(2.1); 7.443(1.8); 7.434(0.4); 7.429(0.4); 7.418(0.8); 7.415(1.0); 7.409(0.9); 7.406(0.9); 7.400(1.6); 7.397(1.4); 7.391(1.9); 7.388(1.9); 7.381(0.9); 7.378(0.8); 7.372(1.4); 7.369(1.4); 7.360(1.3); 7.355(1.3); 7.340(2.1); 7.335(2.2); 7.320(1.7); 7.316(1.7); 7.301(0.8); 7.297(0.7); 7.170(1.6); 7.166(1.6); 7.152(1.4); 7.147(1.3); 5.755(3.9); 4.691(2.0); 4.677(2.2); 4.334(0.5); 4.320(0.5); 4.317(0.7); 4.313(0.6); 4.303(0.6); 4.299(0.7); 4.295(0.6); 4.282(0.5); 3.323(3.2); 3.201(16.0); 3.195(1.5); 3.164(0.8); 2.511(5.4); 2.506(10.9); 2.502(14.3); 2.497(10.1); 2.493(4.7); 2.086(2.1); 1.145(0.3); 1.128(0.3); 1.093(6.0); 1.084(0.8); 1.076(5.9); 0.008(0.8); 0.000(20.2); −0.009(0.7)

### Example 143

¹H-NMR (400.0 MHz. DMSO): δ=8.663(1.0); 8.642(1.0); 8.448(1.5); 8.443(1.6); 8.436(1.7); 8.431(1.6); 7.705(1.5); 7.700(1.5); 7.686(1.8); 7.681(1.7); 7.479(2.8); 7.467(2.6); 7.461(3.6); 7.448(3.2); 7.443(1.9); 7.425(0.8); 7.422(0.9); 7.407(1.7); 7.404(1.4); 7.388(0.9); 7.385(0.8); 7.368(1.2); 7.363(1.1); 7.349(1.2); 7.344(1.2); 7.330(0.7); 7.325(0.6); 5.755(4.5); 4.690(2.1); 4.678(2.2); 4.338(0.5); 4.325(0.5); 4.321(0.7); 4.317(0.6); 4.308(0.6); 4.304(0.6); 4.300(0.5); 4.287(0.5); 3.321(22.4); 3.210(16.0); 3.204(1.6); 3.185(0.4); 3.178(0.7); 2.524(0.7); 2.510(17.0); 2.506(34.1); 2.502(44.4); 2.497(31.7); 2.493(15.0); 1.127(0.4); 1.122(0.3); 1.110(0.4); 1.073(6.1); 1.056(6.0); 0.008(1.8); 0.000(47.6); −0.009(1.5)

### Example 144

¹H-NMR (400.0 MHz. DMSO): δ=8.796(0.8); 8.775(0.9); 7.500(0.7); 7.496(0.6); 7.483(0.5); 7.479(1.3); 7.474(1.6); 7.470(1.4); 7.462(0.8); 7.454(2.3); 7.451(2.6); 7.437(1.8); 7.432(1.7); 7.417(0.7); 7.414(0.8); 7.399(1.5); 7.396(1.3); 7.380(0.8); 7.377(0.8); 7.363(1.2); 7.358(1.3); 7.344(1.2); 7.339(1.1); 7.326(0.6); 7.321(0.6); 7.203(0.4); 7.152(0.3); 7.144(2.0); 7.131(0.6); 7.125(2.4); 7.123(2.4); 7.114(0.4); 7.104(1.8); 7.096(0.3); 4.683(2.1); 4.671(2.2); 4.345(0.5); 4.334(0.5); 4.328(0.6); 4.325(0.5); 4.317(0.5); 4.313(0.6); 4.307(0.5); 4.296(0.5); 3.323(4.9); 3.209(16.0); 3.165(1.4); 2.511(7.5); 2.507(15.2); 2.502(20.0); 2.497(14.2); 2.493(6.7); 1.110(0.5); 1.093(0.5); 1.074(0.3); 1.057(0.3); 1.029(5.9); 1.012(5.8); 0.008(0.9); 0.000(25.4); −0.009(0.8)

### Example 145

¹H-NMR (400.0 MHz. DMSO): δ=8.533(1.0); 8.512(1.0); 7.830(0.4); 7.827(0.4); 7.799(0.4); 7.765(0.4); 7.762(0.4); 7.752(1.3); 7.742(0.6); 7.733(1.7); 7.721(0.5); 7.700(0.6); 7.681(1.4); 7.662(1.0); 7.628(1.0); 7.609(1.3); 7.590(0.5); 7.490(1.2); 7.486(1.3); 7.470(1.8); 7.467(1.9); 7.461(0.9); 7.456(1.0); 7.442(1.8); 7.437(1.9); 7.422(0.8); 7.419(0.9); 7.404(1.6); 7.401(1.4); 7.386(0.9); 7.382(0.7); 7.370(1.2); 7.365(1.2); 7.351(1.2); 7.346(1.3); 7.333(0.6); 7.328(0.6); 7.267(1.4); 7.249(1.3); 4.673(2.1); 4.660(2.3); 4.332(0.5); 4.319(0.5); 4.315(0.7); 4.311(0.6); 4.302(0.6); 4.298(0.7); 4.294(0.6); 4.281(0.5); 3.322(5.6); 3.190(16.0); 3.172(0.8); 2.524(0.5); 2.511(11.0); 2.506(21.8); 2.502(28.5); 2.497(20.7); 2.493(10.3); 1.125(0.4); 1.121(0.5); 1.108(0.4); 1.081(0.4); 1.069(6.1); 1.052(6.1); 0.982(0.5); 0.008(1.4); 0.000(30.4); −0.009(1.4)

### Example 146

¹H-NMR (400.0 MHz. DMSO): δ=8.402(0.9); 8.381(0.9); 7.848(1.6); 7.846(1.6); 7.829(1.7); 7.826(1.7); 7.479(1.3); 7.476(1.3); 7.465(1.0); 7.460(2.8); 7.456(1.9); 7.446(1.7); 7.441(1.7); 7.420(1.0); 7.417(1.7); 7.401(1.7); 7.398(2.9); 7.395(2.0); 7.383(0.9); 7.379(1.6); 7.377(1.1); 7.361(1.2); 7.356(1.1); 7.342(1.2); 7.337(1.3); 7.323(0.6); 7.319(0.6); 7.152(0.9); 7.147(1.0); 7.132(1.4); 7.128(1.5); 7.113(0.8); 7.109(0.8); 7.089(1.5); 7.085(1.4); 7.070(1.4); 7.066(1.2); 5.755(0.5); 4.713(2.0); 4.700(2.1); 4.317(0.5); 4.304(0.5); 4.300(0.6); 4.296(0.5); 4.286(0.5); 4.283(0.6); 4.279(0.6); 4.265(0.5); 3.320(22.0); 3.212(16.0); 3.164(1.2); 2.670(0.4); 2.524(0.9); 2.519(1.5); 2.510(21.8); 2.506(44.6); 2.501(58.9); 2.497(41.7); 2.492(19.4); 2.328(0.4); 1.151(0.3); 1.134(0.3); 1.106(0.6); 1.095(5.8); 1.078(5.7); 0.008(2.1); 0.000(62.5); −0.009(2.0)

### Example 147

¹H-NMR (400.0 MHz. DMSO): δ=8.200(0.5); 8.194(0.6); 8.179(0.6); 8.173(0.5); 7.523(0.4); 7.514(0.4); 7.509(0.8); 7.504(0.7); 7.502(0.6); 7.494(1.2); 7.489(1.2); 7.484(0.7); 7.475(1.6); 7.471(1.7); 7.466(2.2); 7.463(1.9); 7.456(1.1); 7.452(0.8); 7.447(3.2); 7.443(3.1); 7.416(0.7); 7.413(0.8); 7.398(1.5); 7.379(0.7); 7.376(0.7); 7.355(1.2); 7.350(1.3); 7.336(1.3); 7.331(1.3); 7.317(0.5); 7.313(0.6); 7.274(1.0); 7.264(1.1); 7.261(1.1); 7.253(1.0); 7.249(1.0); 7.245(2.1); 7.226(1.9); 4.688(2.0); 4.675(2.1); 4.389(0.4); 4.372(0.6); 4.355(0.5); 4.338(0.4); 3.322(6.2); 3.216(16.0); 3.203(0.9); 3.189(0.6); 2.520(0.4); 2.511(6.1); 2.507(12.4); 2.502(16.3); 2.498(11.5); 2.493(5.3); 1.130(0.3); 1.114(0.3); 1.079(6.0); 1.062(5.9); 0.008(0.7); 0.000(19.1); −0.009(0.6)

### Example 148

¹H-NMR (400.0 MHz. DMSO): δ=8.180(1.0); 8.158(1.0); 7.473(1.0); 7.468(1.2); 7.463(0.5); 7.453(2.9); 7.444(0.7); 7.436(2.0); 7.432(1.9); 7.407(0.8); 7.404(0.8); 7.398(0.4); 7.389(1.7); 7.386(1.4); 7.370(1.0); 7.367(0.9); 7.347(1.5); 7.342(1.2); 7.328(1.6); 7.323(1.3); 7.309(0.8); 7.305(0.7); 7.289(0.8); 7.286(0.8); 7.271(1.6); 7.268(1.4); 7.252(1.2); 7.249(1.2); 7.211(0.5); 7.206(0.6); 7.200(0.3); 7.188(1.2); 7.179(2.0); 7.170(1.8); 7.160(1.3); 7.152(1.1); 7.113(1.6); 7.109(1.6); 7.092(1.1); 5.755(1.0); 4.649(2.1); 4.634(2.3); 4.379(0.6); 4.362(0.9); 4.357(0.7); 4.346(0.7); 4.341(0.9); 4.324(0.5); 3.331(70.0); 3.173(16.0); 3.160(1.3); 3.156(2.4); 2.524(0.4); 2.511(10.0); 2.506(19.3); 2.502(24.5); 2.497(17.4); 2.493(8.3); 2.223(1.7); 2.129(11.8); 2.048(0.7); 1.989(1.2); 1.398(2.0); 1.193(0.4); 1.182(0.4); 1.175(0.7); 1.166(0.4); 1.157(0.4); 1.141(6.3); 1.124(6.3); 1.086(0.9); 1.069(0.8); 0.000(0.3)

### Example 149

¹H-NMR (400.0 MHz. DMSO): δ=8.954(2.2); 7.647(0.6); 7.637(0.4); 7.628(1.1); 7.612(4.1); 7.606(4.2); 7.584(3.2); 7.566(1.0); 7.562(1.1); 7.519(1.8); 7.498(3.2); 7.448(1.9); 7.443(1.7); 7.427(1.1); 7.422(1.0); 5.755(1.9); 5.054(4.5); 3.333(107.5); 3.183(16.0); 2.525(0.8); 2.511(15.9); 2.507(31.7); 2.502(41.3); 2.498(29.2); 2.493(13.6); 2.074(0.3); 0.893(0.4); 0.887(0.5); 0.881(0.6); 0.873(0.8); 0.863(0.7); 0.860(0.6); 0.852(0.6); 0.782(0.7); 0.778(0.6); 0.765(1.1); 0.746(1.1); 0.733(0.6); 0.729(0.8); 0.716(0.3); 0.678(0.6); 0.667(0.6); 0.657(0.8); 0.649(0.5); 0.643(0.5); 0.637(0.4); 0.000(0.4)

### Example 150

¹H-NMR (400.0 MHz. DMSO): δ=8.857(2.2); 7.592(2.9); 7.587(3.0); 7.516(1.9); 7.495(3.4); 7.475(0.7); 7.470(0.6); 7.458(0.6); 7.453(1.4); 7.448(2.2); 7.442(1.8); 7.437(0.8); 7.432(0.8); 7.426(1.1); 7.421(1.0); 7.416(0.4); 7.116(0.4); 7.108(2.1); 7.089(2.8); 7.068(1.7); 5.015(4.4); 3.336(159.0); 3.164(16.0); 2.525(0.7); 2.511(15.3); 2.507(29.9); 2.502(38.4); 2.498(27.3); 2.494(12.9); 1.039(0.7); 1.034(0.7); 1.023(0.6); 1.013(0.6); 1.009(0.8); 1.000(0.6); 0.939(0.5); 0.928(0.7); 0.925(0.7); 0.914(0.6); 0.904(0.6); 0.899(0.7); 0.715(0.8); 0.699(3.1); 0.683(0.7); 0.000(0.5)

### Example 151

¹H-NMR (400.0 MHz. DMSO): δ=8.549(2.1); 7.608(2.6); 7.603(2.7); 7.585(1.5); 7.582(1.5); 7.565(1.8); 7.562(1.7); 7.528(1.5); 7.506(3.6); 7.480(2.0); 7.475(1.8); 7.459(0.8); 7.454(0.8); 7.407(0.6); 7.405(0.7); 7.389(1.7); 7.386(1.6); 7.370(1.2); 7.367(1.1); 7.324(1.0); 7.319(1.2); 7.304(1.2); 7.300(1.4); 7.285(0.6); 7.280(0.6); 7.234(1.6); 7.230(1.5); 7.216(1.4); 7.211(1.2); 5.756(5.5); 5.074(4.4); 3.322(34.1); 3.159(16.0); 2.524(0.8); 2.510(18.7); 2.506(37.7); 2.501(49.3); 2.497(34.9); 2.492(16.4); 1.159(0.5); 1.056(0.4); 1.051(0.5); 1.037(0.6); 1.032(0.6); 1.026(0.7); 1.021(0.6); 0.991(0.6); 0.938(0.7); 0.933(0.7); 0.928(0.6); 0.913(0.4); 0.908(0.5); 0.899(0.4); 0.786(0.3); 0.761(0.8); 0.751(0.9); 0.746(1.1); 0.741(1.9); 0.730(0.8); 0.720(0.7); 0.000(2.2)

### Example 152

¹H-NMR (400.0 MHz. DMSO): δ=8.497(2.6); 7.812(1.9); 7.792(2.0); 7.608(2.8); 7.603(3.0); 7.525(1.5); 7.503(3.8); 7.481(2.2); 7.476(2.0); 7.459(0.9); 7.454(0.9); 7.418(0.9); 7.416(0.8); 7.399(1.9); 7.397(1.8); 7.380(1.2); 7.378(1.0); 7.172(1.5); 7.168(1.8); 7.153(1.4); 7.149(1.5); 7.134(1.0); 7.130(0.9); 7.115(1.6); 7.111(1.4); 7.096(0.8); 7.092(0.7); 5.756(8.7); 5.059(4.7); 3.321(37.2); 3.162(16.0); 2.670(0.4); 2.506(50.4); 2.501(64.8); 2.497(46.5); 2.328(0.4); 1.075(0.4); 1.063(0.5); 1.059(0.5); 1.049(0.9); 1.039(0.6); 1.034(0.7); 1.023(0.6); 0.945(0.5); 0.930(0.8); 0.920(0.9); 0.905(0.5); 0.895(0.5); 0.843(0.5); 0.832(0.5); 0.827(0.4); 0.819(0.9); 0.807(0.8); 0.803(0.8); 0.793(0.7); 0.773(0.7); 0.763(0.8); 0.758(0.7); 0.747(0.8); 0.738(0.4); 0.734(0.4); 0.000(2.0)

### Example 153

¹H-NMR (400.0 MHz. DMSO): δ=8.330(2.2); 7.601(2.8); 7.596(2.9); 7.527(1.6); 7.506(3.6); 7.476(2.0); 7.471(1.9); 7.455(0.9); 7.450(0.9); 7.282(0.5); 7.277(0.6); 7.264(1.2); 7.260(0.9); 7.258(0.9); 7.246(0.9); 7.241(1.0); 7.170(1.7); 7.158(1.6); 7.139(3.0); 7.134(2.1); 7.119(0.6); 7.115(0.4); 5.119(4.4); 3.321(13.7); 3.144(16.0); 2.524(0.5); 2.510(10.1); 2.506(20.3); 2.501(26.5); 2.497(18.8); 2.492(8.9); 2.202(11.7); 1.079(0.5); 1.074(0.5); 1.069(0.4); 1.055(1.0); 1.049(1.0); 0.975(0.9); 0.969(0.9); 0.954(0.4); 0.949(0.5); 0.944(0.6); 0.708(2.6); 0.702(2.6); 0.000(0.9)

### Example 154

¹H-NMR (400.0 MHz. DMSO): δ=8.558(2.1); 7.778(0.4); 7.758(0.4); 7.604(2.7); 7.599(2.8); 7.537(0.9); 7.535(0.9); 7.528(2.1); 7.523(0.6); 7.519(0.5); 7.507(3.6); 7.476(2.0); 7.471(1.8); 7.455(1.0); 7.450(0.9); 7.442(0.4); 7.435(0.4); 7.429(0.7); 7.425(1.0); 7.417(0.6); 7.410(2.2); 7.406(3.2); 7.401(1.5); 7.388(1.7); 7.384(1.6); 7.369(0.8); 7.364(0.9); 7.359(1.1); 7.354(1.0); 7.341(1.5); 7.336(1.4); 7.323(0.8); 7.319(0.7); 7.261(1.6); 7.258(1.4); 7.257(1.3); 7.243(1.2); 7.239(1.0); 5.756(2.2); 5.082(4.3); 3.321(11.9); 3.155(16.0); 2.524(0.8); 2.511(17.6); 2.506(34.4); 2.502(44.2); 2.497(31.1); 2.492(14.5); 1.154(0.5); 1.063(0.5); 1.058(0.5); 1.049(0.4); 1.037(1.0); 1.034(1.1); 1.005(0.3); 0.987(0.5); 0.948(1.1); 0.933(0.4); 0.924(0.5); 0.919(0.6); 0.912(0.4); 0.734(0.9); 0.721(3.1); 0.706(0.7); 0.008(0.7); 0.000(18.4); −0.009(0.6)

### Example 155

¹H-NMR (400.0 MHz. DMSO): δ=8.400(1.8); 7.581(2.6); 7.576(2.7); 7.514(1.6); 7.493(3.5); 7.484(0.5); 7.475(0.4); 7.470(0.9); 7.462(2.3); 7.457(2.3); 7.452(0.9); 7.450(0.9); 7.444(0.7); 7.441(1.0); 7.436(1.2); 7.432(0.5); 7.416(0.6); 7.412(0.7); 7.396(1.2); 7.393(1.1); 7.379(0.8); 7.374(0.7); 7.224(1.7); 7.205(2.0); 7.199(1.0); 7.196(1.2); 7.189(0.9); 7.186(0.9); 7.175(0.8); 5.756(0.3); 5.053(4.3); 3.321(26.7); 3.141(16.0); 2.671(0.4); 2.524(1.0); 2.510(22.2); 2.506(44.3); 2.502(57.5); 2.497(41.2); 2.493(19.7); 2.328(0.4); 1.989(0.9); 1.398(4.5); 1.175(0.5); 1.120(0.3); 1.110(0.5); 1.105(0.5); 1.095(0.5); 1.086(0.8); 1.080(0.9); 0.970(0.9); 0.965(0.9); 0.954(0.4); 0.944(0.5); 0.940(0.5); 0.931(0.4); 0.723(0.8); 0.706(2.7); 0.689(0.7); 0.000(2.2)

### Example 156

¹H-NMR (400.0 MHz. DMSO): δ=13.377(8.9); 7.794(4.7); 7.791(8.1); 7.789(4.8); 7.773(6.4); 7.771(6.2); 7.770(5.9); 7.564(0.4); 7.558(1.5); 7.547(15.5); 7.544(16.0); 7.539(10.6); 7.538(10.0); 7.533(9.2); 7.529(7.9); 7.513(1.5); 7.509(1.5); 7.462(1.0); 7.450(5.7); 7.442(4.9); 7.436(4.0); 7.431(5.4); 7.428(4.4); 7.424(4.3); 7.416(3.8); 7.409(3.4); 7.402(0.3); 7.396(0.4); 3.327(1.7); 3.196(2.1); 3.190(0.6); 2.672(0.4); 2.525(1.0); 2.512(22.1); 2.508(44.5); 2.503(58.1); 2.499(41.1); 2.494(19.2); 2.330(0.4); 1.989(0.6); 1.398(0.8); 1.234(0.3); 1.175(0.3); 1.093(0.8); 1.076(0.7); 0.000(2.8)

### Example 158

¹H-NMR (400.1 MHz. CDCl3): δ=7.596(0.7); 7.518(1.4); 7.435(0.4); 7.388(1.9); 7.383(2.1); 7.312(0.7); 7.307(1.2); 7.301(0.4); 7.291(0.9); 7.286(0.9); 7.260(238.2); 7.251(3.2); 7.251(2.8); 7.250(2.5); 7.249(2.2); 7.248(1.9); 7.247(1.7); 7.246(1.4); 7.246(1.4); 7.245(1.2); 7.244(1.1); 7.243(1.0); 7.242(0.9); 7.241(0.8); 7.241(0.8); 7.240(0.7); 7.239(0.6); 7.238(0.6); 7.237(0.6); 7.237(0.5); 7.236(0.5); 7.235(0.4); 7.234(0.4); 7.233(0.4); 7.232(0.3); 6.996(1.5); 5.299(2.8); 5.023(0.4); 5.016(0.3); 3.965(6.6); 3.257(16.0); 2.778(0.3); 2.771(0.4); 2.762(0.7); 2.752(0.4); 2.745(0.4); 1.534(21.8); 0.789(0.5); 0.773(0.5); 0.704(0.3); 0.691(0.5); 0.681(0.5); 0.676(0.4); 0.669(0.4); 0.665(0.4); 0.605(0.4); 0.589(0.3); 0.013(0.3); 0.013(0.4); 0.012(0.5); 0.011(0.5); 0.010(0.7); 0.008(2.5); 0.007(1.4); 0.006(1.5); 0.005(1.8); 0.000(80.7)

### Example 159

¹H-NMR (400.1 MHz. CDCl3): δ=8.723(0.3); 8.722(0.4); 8.718(0.4); 8.717(0.4); 8.588(1.3); 8.583(1.4); 8.013(0.4); 8.012(0.4); 8.008(0.4); 8.007(0.4); 7.949(1.7); 7.948(1.7); 7.943(1.8); 7.942(1.7); 7.449(1.2); 7.428(1.5); 7.411(0.8); 7.407(2.9); 7.402(2.9); 7.274(1.2); 7.271(0.6); 7.269(1.2); 7.268(1.2); 7.266(0.7); 7.265(0.7); 7.265(0.8); 7.264(1.0); 7.259(39.9); 7.254(1.7); 7.254(1.7); 7.253(1.7); 7.248(1.2); 5.111(1.4); 5.093(1.5); 3.242(16.0); 3.176(2.2); 2.433(0.3); 2.426(0.4); 2.415(0.6); 2.405(0.4); 2.398(0.4); 1.578(0.5); 1.479(3.5); 1.462(3.5); 1.212(0.4); 1.195(0.4); 0.437(0.4); 0.070(0.4); 0.008(0.4); 0.000(13.5)

### Example 160

¹H-NMR (400.1 MHz. CDCl3): δ=7.635(1.0); 7.624(1.0); 7.613(1.2); 7.552(0.3); 7.545(0.4); 7.519(2.7); 7.460(1.6); 7.453(1.3); 7.413(1.9); 7.403(1.7); 7.394(1.5); 7.381(0.4); 7.359(0.7); 7.358(0.4); 7.357(0.3); 7.356(0.3); 7.356(0.4); 7.354(0.7); 7.351(0.5); 7.351(0.5); 7.350(0.4); 7.334(0.3); 7.332(0.3); 7.331(0.3); 7.330(0.3); 7.329(0.3); 7.329(0.3); 7.328(0.4); 7.327(0.4); 7.326(0.4); 7.325(0.5); 7.325(0.5); 7.324(0.5); 7.323(0.5); 7.322(0.6); 7.321(0.5); 7.321(0.5); 7.320(0.5); 7.319(0.5); 7.318(0.5); 7.317(0.5); 7.316(0.5); 7.316(0.5); 7.315(0.5); 7.314(0.4); 7.313(0.4); 7.312(0.4); 7.312(0.4); 7.309(4.0); 7.306(1.7); 7.304(1.6); 7.303(1.5); 7.303(1.5); 7.302(1.4); 7.301(1.4); 7.300(1.4); 7.299(1.4); 7.299(1.4); 7.298(1.2); 7.297(1.3); 7.296(1.2); 7.295(1.2); 7.294(1.2); 7.294(1.1); 7.293(1.1); 7.292(1.1); 7.291(1.0); 7.290(1.1); 7.290(1.1); 7.289(1.2); 7.288(1.2); 7.287(1.2); 7.286(1.2); 7.285(1.3); 7.285(1.4); 7.284(1.4); 7.283(1.5); 7.282(1.5); 7.281(1.5); 7.281(1.6); 7.280(1.6); 7.279(1.6); 7.278(1.6); 7.277(1.7); 7.276(1.9); 7.276(2.0); 7.275(2.1); 7.274(2.2); 7.273(2.4); 7.272(2.6); 7.272(3.1); 7.271(3.3); 7.270(3.7); 7.269(4.4); 7.268(5.1); 7.260(428.0); 7.247(1.6); 7.244(1.1); 7.242(0.9); 7.240(0.7); 7.238(0.6); 7.237(0.5); 7.236(0.4); 7.235(0.4); 7.234(0.4); 7.233(0.4); 7.233(0.3); 6.996(2.4); 6.529(0.3); 5.299(1.9); 5.278(0.3); 5.269(0.3); 4.982(0.3); 4.134(0.3); 3.491(0.6); 3.420(0.9); 3.250(16.0); 3.187(0.5); 3.028(0.5); 3.016(0.5); 2.163(0.4); 2.155(0.4); 2.005(0.6); 1.587(2.3); 1.570(2.5); 1.536(24.4); 1.470(1.0); 1.453(0.9); 1.268(0.5); 1.256(0.8); 0.628(0.4); 0.599(0.6); 0.575(0.4); 0.571(0.4); 0.548(0.4); 0.478(0.4); 0.306(0.4); 0.292(0.5); 0.282(0.4); 0.168(0.3); 0.146(0.7); 0.050(0.9); 0.017(0.3); 0.017(0.4); 0.016(0.5); 0.015(0.5); 0.014(0.5); 0.013(0.6); 0.013(0.7); 0.012(0.8); 0.011(1.0); 0.010(1.3); 0.008(3.8); 0.007(2.0); 0.006(2.4); 0.005(3.1); 0.000(124.0)

### Example 161

¹H-NMR (400.1 MHz. CDCl3): =7.518(1.1); 7.515(0.8); 7.457(0.5); 7.411(0.3); 7.390(2.5); 7.385(3.2); 7.359(0.3); 7.309(1.7); 7.304(1.5); 7.284(1.5); 7.274(0.7); 7.260(166.4); 7.256(127.0); 7.210(0.5); 6.996(0.9); 6.992(0.7); 6.936(0.4); 6.801(0.7); 6.666(0.4); 5.002(0.5); 4.988(0.4); 3.893(0.5); 3.889(0.4); 3.803(10.3); 3.752(0.4); 3.747(0.4); 3.703(0.4); 3.698(0.4); 3.660(0.6); 3.626(0.4); 3.491(0.5); 3.487(0.4); 3.483(0.5); 3.480(0.4); 3.270(16.0); 3.267(12.2); 2.867(0.4); 2.857(0.7); 2.844(1.0); 2.832(0.7); 2.005(1.5); 2.001(1.2); 1.537(13.7); 0.729(0.9); 0.647(0.8); 0.634(0.9); 0.623(0.9); 0.548(0.7); 0.008(0.8); 0.005(0.9); 0.000(29.0)

### Example 162

¹H-NMR (400.1 MHz. CDCl3): δ=8.728(1.4); 8.723(1.4); 8.011(1.6); 8.006(1.6); 7.532(1.8); 7.518(2.0); 7.511(2.2); 7.404(2.3); 7.399(2.6); 7.359(0.4); 7.318(1.3); 7.313(1.5); 7.310(1.1); 7.297(1.0); 7.292(1.0); 7.276(0.5); 7.272(0.9); 7.259(348.7); 7.244(1.7); 7.239(1.0); 7.233(0.9); 7.228(0.8); 7.224(0.8); 7.209(1.1); 7.160(0.4); 6.995(2.0); 5.298(0.5); 5.138(0.7); 5.129(0.9); 5.116(0.8); 5.107(0.8); 3.849(0.7); 3.840(0.8); 3.815(1.0); 3.806(1.0); 3.622(1.1); 3.599(1.0); 3.587(0.8); 3.564(0.8); 3.491(0.3); 3.295(16.0); 3.261(0.5); 3.189(2.1); 2.842(0.4); 2.836(0.4); 2.825(0.8); 2.814(0.5); 2.808(0.5); 1.531(29.2); 0.699(0.5); 0.504(0.3); 0.491(1.3); 0.482(1.0); 0.473(1.4); 0.469(1.6); 0.458(0.8); 0.451(0.6); 0.444(0.4); 0.146(0.4); 0.008(3.0); 0.000(107.5)

### Example 163

¹H-NMR (400.0 MHz. CDCl3): δ=7.736(1.9); 7.717(2.2); 7.661(0.4); 7.641(1.0); 7.618(1.5); 7.598(2.8); 7.597(2.6); 7.579(2.2); 7.577(2.1); 7.572(1.6); 7.551(3.0); 7.550(2.7); 7.548(2.6); 7.527(2.1); 7.511(0.7); 7.509(0.9); 7.508(0.9); 7.505(0.9); 7.503(0.9); 7.450(4.5); 7.445(5.0); 7.368(3.1); 7.367(3.2); 7.363(3.1); 7.362(3.1); 7.360(2.0); 7.354(1.9); 7.347(2.7); 7.346(2.6); 7.342(2.7); 7.332(0.6); 7.330(0.6); 7.323(0.4); 7.313(0.4); 7.311(0.4); 7.309(0.4); 7.308(0.4); 7.308(0.4); 7.306(0.5); 7.305(0.6); 7.304(0.7); 7.303(0.7); 7.302(0.8); 7.292(154.4); 7.287(1.4); 7.287(1.1); 7.286(0.8); 7.285(0.6); 7.284(0.5); 7.283(0.5); 7.283(0.4); 7.269(0.5); 7.251(0.5); 7.250(0.5); 7.249(0.5); 7.248(0.4); 7.242(0.6); 7.230(0.6); 7.225(0.6); 7.209(0.8); 7.204(0.8); 7.193(0.4); 7.107(1.2); 7.086(0.8); 7.028(0.9); 5.204(0.9); 5.194(1.0); 5.182(1.0); 5.172(1.0); 4.980(0.4); 4.973(0.5); 4.955(0.5); 4.948(0.5); 3.522(0.3); 3.331(16.0); 3.320(7.9); 3.240(0.9); 3.200(0.4); 3.193(0.4); 3.164(0.6); 3.157(0.6); 3.033(0.5); 3.008(0.5); 2.996(0.4); 2.971(0.4); 2.876(0.4); 1.738(0.4); 1.171(0.3); 0.943(0.5); 0.937(0.5); 0.932(0.7); 0.927(1.0); 0.915(0.8); 0.764(0.4); 0.759(0.3); 0.652(0.6); 0.503(1.1); 0.041(1.4); 0.039(0.4); 0.038(0.4); 0.038(0.5); 0.037(0.7); 0.033(45.8); 0.028(0.6); 0.027(0.5); 0.026(0.4); 0.024(1.3)

### Example 164

¹H-NMR (400.0 MHz. CDCl3): δ=8.758(1.7); 8.749(1.7); 7.710(1.5); 7.690(1.7); 7.549(2.2); 7.528(2.7); 7.518(4.0); 7.469(1.4); 7.457(1.6); 7.450(1.6); 7.437(1.6); 7.428(3.1); 7.423(3.2); 7.355(1.9); 7.351(1.8); 7.335(2.0); 7.312(0.5); 7.311(1.3); 7.309(2.7); 7.303(0.8); 7.297(0.6); 7.296(0.5); 7.296(0.6); 7.295(0.6); 7.294(0.6); 7.293(0.7); 7.292(0.6); 7.292(0.6); 7.291(0.6); 7.290(0.6); 7.288(0.9); 7.287(0.6); 7.285(1.3); 7.283(2.5); 7.281(1.3); 7.281(1.4); 7.280(1.2); 7.279(1.3); 7.278(1.3); 7.277(1.4); 7.277(1.5); 7.276(1.7); 7.275(1.9); 7.274(2.0); 7.273(2.2); 7.273(2.4); 7.272(2.7); 7.271(2.9); 7.270(3.2); 7.269(3.3); 7.269(3.8); 7.268(4.5); 7.267(5.3); 7.266(6.3); 7.265(7.6); 7.264(9.5); 7.260(666.2); 7.254(4.2); 7.253(3.1); 7.252(2.1); 7.252(1.6); 7.251(1.3); 7.250(1.1); 7.249(0.7); 7.159(1.0); 7.060(0.5); 7.049(1.2); 6.996(3.9); 6.913(2.2); 6.777(1.1); 5.168(1.1); 5.158(1.2); 5.146(1.2); 5.135(1.2); 3.851(0.9); 3.828(0.9); 3.816(1.5); 3.793(1.4); 3.697(1.5); 3.687(1.5); 3.663(1.0); 3.652(0.9); 3.491(0.9); 3.321(16.0); 3.278(2.2); 2.689(0.7); 2.682(0.8); 2.673(1.2); 2.664(0.9); 2.656(0.7); 2.005(7.3); 1.557(10.5); 0.647(0.8); 0.636(1.0); 0.610(1.1); 0.588(1.1); 0.575(0.8); 0.558(0.8); 0.539(0.9); 0.526(1.1); 0.504(0.8); 0.488(0.8); 0.480(1.0); 0.464(1.0); 0.452(0.8); 0.146(0.6); 0.049(0.7); 0.023(0.7); 0.012(0.6); 0.011(0.6); 0.010(0.7); 0.010(0.9); 0.008(5.8); 0.007(1.7); 0.006(1.8); 0.005(2.0); 0.004(2.7); 0.000(195.5); −0.005(2.7); −0.006(2.0); −0.007(1.7); −0.009(5.4); −0.011(0.7); −0.149(0.6)

### Example 166

¹H-NMR (400.0 MHz. DMSO): δ=8.827(1.8); 8.815(1.9); 8.801(0.4); 8.788(0.3); 8.000(0.8); 7.979(0.7); 7.827(1.6); 7.815(1.7); 7.807(1.3); 7.795(1.2); 7.674(2.9); 7.670(3.1); 7.596(0.7); 7.590(0.7); 7.584(1.4); 7.563(4.5); 7.548(2.7); 7.544(2.5); 7.527(0.8); 7.522(0.8); 7.439(0.4); 7.435(0.4); 7.418(0.4); 7.413(0.4); 5.296(0.4); 5.037(1.0); 5.026(0.9); 5.017(0.9); 5.006(1.0); 3.686(0.5); 3.664(0.4); 3.603(0.5); 3.596(0.4); 3.456(0.3); 3.436(0.5); 3.339(929.6); 3.264(0.5); 3.223(16.0); 3.190(0.5); 3.163(0.4); 3.147(0.4); 2.671(1.6); 2.667(1.4); 2.649(0.8); 2.638(1.1); 2.612(0.4); 2.507(179.1); 2.502(233.4); 2.498(176.2); 2.329(1.5); 1.280(0.4); 1.235(3.2); 1.211(0.5); 0.870(0.4); 0.854(0.6); 0.755(0.3); 0.743(0.4); 0.530(0.9); 0.433(0.5); 0.428(0.5); 0.376(0.4); 0.146(0.4); 0.008(3.4); 0.000(81.6); −0.009(3.4); −0.150(0.4)

### Example 167

¹H-NMR (400.0 MHz. DMSO): δ=8.480(1.4); 8.476(1.5); 8.468(1.7); 8.464(1.7); 7.849(0.9); 7.845(0.9); 7.830(1.0); 7.826(1.0); 7.671(2.2); 7.667(2.3); 7.588(1.4); 7.567(3.3); 7.547(2.1); 7.541(2.2); 7.539(2.1); 7.527(2.2); 7.520(2.1); 7.508(1.5); 5.054(0.7); 5.038(0.7); 5.022(0.7); 3.676(0.4); 3.657(0.4); 3.464(0.4); 3.411(0.9); 3.342(1187.3); 3.275(0.6); 3.258(0.4); 3.221(16.0); 2.891(0.4); 2.731(0.8); 2.717(0.8); 2.706(0.5); 2.681(0.6); 2.676(1.3); 2.672(1.7); 2.667(1.3); 2.663(0.6); 2.525(5.9); 2.512(100.9); 2.507(199.0); 2.503(258.9); 2.498(185.7); 2.494(88.4); 2.338(0.6); 2.334(1.2); 2.329(1.7); 2.325(1.2); 2.320(0.6); 1.281(0.3); 1.235(2.9); 0.854(0.6); 0.506(0.8); 0.492(0.8); 0.487(0.8); 0.481(0.8); 0.467(0.9); 0.450(0.8); 0.436(0.5); 0.146(0.5); 0.008(4.2); 0.000(109.4); −0.009(3.4); −0.150(0.5)

### Example 168

¹H-NMR (601.6 MHz. DMSO): δ=9.074(2.0); 9.070(2.1); 8.982(2.2); 8.979(2.2); 8.959(0.3); 7.669(2.7); 7.666(2.8); 7.577(1.9); 7.574(0.6); 7.563(3.2); 7.536(1.9); 7.532(1.7); 7.522(1.0); 7.518(0.9); 5.040(0.9); 5.030(0.9); 5.020(0.9); 3.671(1.8); 3.665(1.3); 3.658(1.1); 3.316(36.9); 3.217(16.0); 3.095(2.1); 2.732(0.4); 2.726(0.5); 2.720(0.6); 2.714(1.0); 2.708(0.6); 2.703(0.5); 2.613(0.4); 2.522(0.8); 2.519(1.1); 2.516(1.2); 2.507(21.9); 2.505(45.3); 2.501(61.5); 2.498(44.7); 2.495(20.8); 2.386(0.4); 0.639(0.4); 0.536(0.4); 0.532(0.3); 0.528(0.4); 0.524(0.5); 0.521(0.6); 0.512(0.5); 0.450(0.5); 0.442(0.6); 0.431(0.4); 0.426(0.4); 0.415(0.4); 0.401(0.4); 0.000(0.6)

### Example 169

¹H-NMR (601.6 MHz. DMSO): δ=7.645(3.6); 7.642(3.4); 7.519(1.2); 7.516(1.1); 7.505(2.9); 7.502(2.7); 7.487(2.2); 7.473(0.9); 7.375(0.4); 4.921(0.7); 4.277(0.4); 4.272(0.6); 4.259(1.0); 4.252(1.3); 4.244(0.9); 4.225(1.1); 3.632(0.5); 3.382(0.5); 3.365(0.5); 3.315(68.2); 3.154(16.0); 2.999(2.1); 2.891(0.5); 2.770(0.4); 2.731(0.5); 2.616(0.6); 2.613(0.7); 2.610(0.5); 2.522(1.4); 2.519(1.9); 2.516(2.1); 2.507(43.3); 2.504(87.1); 2.501(116.3); 2.498(84.0); 2.495(39.1); 2.388(0.5); 2.385(0.7); 2.382(0.5); 1.800(4.6); 0.823(0.6); 0.814(1.4); 0.809(1.4); 0.802(1.8); 0.792(1.4); 0.781(1.0); 0.741(0.4); 0.000(1.0)

### Example 170

¹H-NMR (601.6 MHz. DMSO): =8.410(3.9); 8.407(3.8); 8.402(3.7); 8.399(4.1); 7.953(0.9); 7.643(3.6); 7.528(2.8); 7.514(3.6); 7.458(5.2); 7.450(5.2); 7.445(3.9); 7.437(3.9); 5.084(3.6); 5.071(3.6); 4.355(0.8); 3.316(105.9); 3.156(16.0); 3.116(0.7); 3.104(0.8); 2.890(7.3); 2.731(5.9); 2.619(0.3); 2.616(0.7); 2.613(1.1); 2.610(0.8); 2.607(0.3); 2.522(1.9); 2.519(2.4); 2.516(2.3); 2.507(55.4); 2.504(120.5); 2.501(167.3); 2.498(122.4); 2.495(57.3); 2.391(0.6); 2.388(0.9); 2.385(1.2); 2.382(0.9); 2.379(0.5); 2.305(0.7); 1.455(4.6); 1.401(0.3); 1.235(0.4); 0.455(0.9); 0.424(0.9); 0.310(2.1); 0.303(1.9); 0.000(1.6)

### Example 171

¹H-NMR (601.6 MHz. DMSO): δ=8.691(1.3); 8.688(1.3); 8.683(1.3); 8.680(1.3); 7.676(1.5); 7.674(1.6); 7.561(1.0); 7.553(1.4); 7.548(1.3); 7.540(2.0); 7.531(1.7); 7.528(1.6); 7.517(0.5); 7.514(0.5); 7.376(0.3); 6.609(0.6); 5.061(0.8); 5.048(0.8); 3.316(37.8); 3.160(16.0); 3.129(0.5); 3.104(0.4); 2.890(0.5); 2.731(0.4); 2.731(0.4); 2.613(0.4); 2.522(0.7); 2.519(0.9); 2.516(1.0); 2.507(19.6); 2.504(41.5); 2.501(56.4); 2.498(40.9); 2.495(18.7); 2.385(0.3); 2.282(0.5); 1.476(2.2); 1.465(2.2); 0.450(0.4); 0.000(0.6)

### Example 172

¹H-NMR (601.6 MHz. DMSO): δ=7.586(2.3); 7.583(2.4); 7.461(0.6); 7.457(0.6); 7.447(1.6); 7.444(1.6); 7.430(1.1); 7.416(0.5); 4.984(0.8); 4.970(0.8); 4.171(1.5); 4.163(2.6); 4.156(1.3); 4.154(1.3); 3.315(28.5); 3.108(16.0); 2.902(0.9); 2.896(1.5); 2.890(2.4); 2.888(1.4); 2.882(0.7); 2.731(1.1); 2.731(1.0); 2.522(0.6); 2.519(0.8); 2.516(0.9); 2.507(15.8); 2.504(33.1); 2.501(45.2); 2.498(32.5); 2.495(14.8); 2.388(0.3); 2.385(0.4); 2.382(0.4); 2.379(0.5); 2.376(0.4); 2.368(0.7); 2.361(0.4); 2.358(0.4); 1.458(2.0); 1.360(3.7); 1.348(3.7); 0.741(0.6); 0.735(0.5); 0.729(0.7); 0.723(0.7); 0.711(0.7); 0.700(0.6); 0.000(0.5)

### Example 173

¹H-NMR (601.6 MHz. DMSO): δ=8.760(5.8); 8.753(5.3); 7.723(3.3); 7.715(3.8); 7.675(13.4); 7.674(13.1); 7.672(13.7); 7.547(3.1); 7.529(5.7); 7.516(3.7); 7.489(1.3); 7.458(1.4); 5.101(2.4); 5.089(2.4); 4.967(0.8); 4.694(0.6); 4.199(1.5); 4.189(2.0); 4.177(1.4); 3.316(256.1); 3.188(3.5); 3.141(16.0); 3.104(0.8); 2.891(2.3); 2.731(1.8); 2.619(0.8); 2.616(1.7); 2.613(2.3); 2.610(1.7); 2.607(0.7); 2.522(5.5); 2.519(7.3); 2.516(8.4); 2.507(131.9); 2.504(275.0); 2.501(373.6); 2.498(272.9); 2.495(127.0); 2.392(0.8); 2.388(1.7); 2.385(2.3); 2.382(1.7); 2.379(0.8); 2.317(0.7); 2.188(2.1); 1.459(7.8); 1.448(8.0); 1.402(0.7); 1.388(1.0); 1.362(2.5); 1.281(0.4); 1.235(0.7); 1.148(0.4); 1.098(0.4); 0.685(1.6); 0.520(3.0); 0.416(0.9); 0.213(1.8); 0.068(1.7); 0.000(3.3)

### Example 174

¹H-NMR (601.6 MHz. DMSO): δ=8.946(2.1); 8.942(2.4); 8.908(2.9); 8.904(2.4); 7.642(3.5); 7.639(3.6); 7.523(0.8); 7.509(1.9); 7.493(2.4); 7.490(2.3); 7.479(1.0); 7.476(1.0); 5.041(1.7); 5.029(1.8); 3.316(47.7); 3.159(16.0); 3.095(0.5); 2.891(1.1); 2.732(0.9); 2.731(0.9); 2.616(0.3); 2.613(0.5); 2.522(0.9); 2.519(1.2); 2.516(1.3); 2.507(24.8); 2.504(53.2); 2.501(73.0); 2.498(53.0); 2.495(24.1); 2.389(0.3); 2.386(0.5); 2.318(0.5); 1.423(3.9); 1.412(3.8); 0.484(0.4); 0.000(0.8)

### Example 177

¹H-NMR (400.0 MHz. DMSO): δ=8.485(1.5); 8.480(1.6); 8.473(1.9); 8.468(2.0); 8.460(0.8); 7.777(0.5); 7.761(0.6); 7.588(0.4); 7.540(1.6); 7.528(1.5); 7.521(1.4); 7.509(1.5); 7.494(0.4); 7.476(0.9); 7.457(1.9); 7.439(4.0); 7.425(1.4); 7.421(2.2); 7.415(0.9); 7.405(0.9); 7.400(0.9); 7.396(0.6); 7.380(2.5); 7.375(2.6); 7.362(2.8); 7.150(0.6); 7.069(0.3); 7.038(0.4); 7.026(0.5); 4.612(0.7); 4.594(0.9); 4.580(0.8); 3.329(69.8); 3.211(16.0); 3.191(0.4); 3.183(0.3); 3.157(2.7); 3.150(1.8); 3.135(1.4); 3.089(8.1); 2.891(0.5); 2.780(1.2); 2.733(0.6); 2.525(0.9); 2.521(1.3); 2.512(16.0); 2.508(32.0); 2.503(42.0); 2.498(30.3); 2.494(14.4); 1.235(0.4); 0.000(5.1)

### Example 178

¹H-NMR (400.0 MHz. DMSO): δ=7.438(0.5); 7.419(1.0); 7.400(1.4); 7.391(1.5); 7.361(2.7); 7.301(0.9); 7.285(1.0); 4.504(0.4); 4.488(0.7); 4.473(0.6); 4.237(1.8); 4.228(2.4); 4.218(1.8); 3.556(0.5); 3.537(0.6); 3.494(0.6); 3.482(0.6); 3.325(22.2); 3.149(14.0); 2.991(5.6); 2.892(1.4); 2.525(0.4); 2.507(17.4); 2.502(22.5); 2.498(16.8); 1.655(16.0); 1.235(0.3); 0.000(4.2)

### Example 179

¹H-NMR (400.0 MHz. DMSO): δ=9.041(1.8); 9.035(2.0); 8.999(1.1); 8.993(1.4); 8.961(2.1); 8.955(2.9); 8.948(1.1); 7.478(0.6); 7.459(1.7); 7.440(3.8); 7.436(2.2); 7.429(1.4); 7.425(2.2); 7.420(0.9); 7.410(0.7); 7.404(0.6); 7.401(0.4); 7.378(1.0); 7.374(1.6); 7.371(0.9); 7.360(0.8); 7.355(2.5); 7.345(1.1); 7.341(2.0); 7.199(1.2); 7.083(0.5); 7.080(0.5); 7.069(0.7); 7.061(0.4); 7.058(0.4); 4.596(0.8); 4.584(0.9); 4.575(1.0); 4.564(0.8); 4.497(0.5); 4.488(0.6); 4.476(0.6); 4.467(0.5); 3.728(0.4); 3.717(0.5); 3.694(1.6); 3.682(1.5); 3.673(1.5); 3.652(1.4); 3.639(0.5); 3.618(0.5); 3.508(0.5); 3.487(0.4); 3.471(0.8); 3.449(0.8); 3.394(0.9); 3.385(0.9); 3.356(0.9); 3.347(1.2); 3.330(27.5); 3.217(0.4); 3.214(0.6); 3.202(16.0); 3.173(0.5); 3.093(9.0); 3.084(0.5); 3.055(9.6); 3.046(0.3); 2.904(0.4); 2.892(0.6); 2.833(14.8); 2.806(0.3); 2.732(0.4); 2.545(0.4); 2.525(0.9); 2.521(1.3); 2.512(14.3); 2.508(28.9); 2.503(38.2); 2.498(27.4); 2.494(13.0); 1.235(0.4); 0.008(0.3); 0.000(10.2)

### Example 180

¹H-NMR (400.0 MHz. DMSO): δ=8.828(2.1); 8.819(2.4); 8.813(2.4); 8.798(1.5); 7.882(0.7); 7.831(2.0); 7.819(2.6); 7.801(1.4); 7.784(2.3); 7.677(0.4); 7.666(0.4); 7.487(1.3); 7.468(3.5); 7.449(4.0); 7.440(5.2); 7.430(4.2); 7.414(1.4); 7.410(1.5); 7.379(5.0); 7.361(4.7); 7.185(1.4); 7.126(0.8); 7.056(1.2); 4.588(1.6); 4.577(1.5); 4.568(2.0); 4.557(1.5); 4.481(1.0); 4.474(0.9); 4.461(1.4); 4.451(1.0); 3.729(0.7); 3.719(1.0); 3.695(1.6); 3.685(1.9); 3.622(0.7); 3.413(0.7); 3.350(379.8); 3.290(0.8); 3.261(0.9); 3.254(0.8); 3.207(16.0); 3.177(6.3); 3.155(1.6); 3.137(0.8); 3.114(5.7); 3.084(6.0); 3.054(0.4); 3.043(0.3); 2.892(0.9); 2.778(2.9); 2.733(1.6); 2.674(0.6); 2.527(1.3); 2.509(65.0); 2.505(83.6); 2.500(62.2); 2.336(0.4); 2.332(0.6); 1.235(0.9); 0.000(0.7)

### Example 181

¹H-NMR (400.0 MHz. DMSO): δ=8.761(1.3); 8.757(1.4); 8.748(2.2); 8.741(1.2); 8.736(1.1); 7.792(1.0); 7.772(1.4); 7.679(1.1); 7.667(1.2); 7.659(1.0); 7.647(1.0); 7.613(0.8); 7.602(0.8); 7.583(0.3); 7.485(0.7); 7.466(1.8); 7.452(2.5); 7.448(3.8); 7.434(1.2); 7.430(2.0); 7.414(0.7); 7.409(0.7); 7.406(0.4); 7.386(1.8); 7.366(3.4); 7.356(1.8); 7.353(2.9); 7.129(1.2); 7.023(0.5); 7.014(0.7); 7.004(0.5); 6.960(0.8); 6.930(1.1); 6.826(1.7); 6.795(2.3); 6.691(0.9); 6.661(1.1); 4.632(0.8); 4.616(1.4); 4.600(0.9); 4.481(0.7); 4.464(0.8); 4.449(0.7); 3.701(1.5); 3.694(1.8); 3.681(2.2); 3.330(71.7); 3.249(0.5); 3.229(1.2); 3.216(16.0); 3.197(0.4); 3.168(0.5); 3.141(8.2); 3.100(8.0); 2.891(0.6); 2.813(0.4); 2.765(13.8); 2.732(0.5); 2.525(0.7); 2.512(16.8); 2.508(33.4); 2.503(43.7); 2.498(31.7); 2.494(15.4); 1.235(0.4); 0.000(4.1)

### Example 182

¹H-NMR (400.0 MHz. DMSO): δ=7.819(3.2); 7.800(4.2); 7.774(0.9); 7.765(0.8); 7.746(1.8); 7.727(1.1); 7.652(5.6); 7.651(6.7); 7.647(6.5); 7.640(1.9); 7.623(1.8); 7.604(0.7); 7.541(0.8); 7.532(0.4); 7.519(3.9); 7.511(11.7); 7.507(6.8); 7.489(0.8); 7.485(0.9); 7.410(1.8); 7.391(2.7); 7.373(1.1); 5.983(3.0); 5.824(4.1); 3.323(105.5); 3.191(14.4); 3.075(16.0); 2.773(10.5); 2.740(13.4); 2.676(0.6); 2.672(0.8); 2.667(0.6); 2.525(2.5); 2.520(4.0); 2.512(43.0); 2.507(86.4); 2.502(114.2); 2.498(81.8); 2.493(38.4); 2.334(0.6); 2.329(0.8); 2.325(0.6); 1.431(11.6); 1.413(8.1); 1.398(4.4); 1.354(7.3); 1.336(0.9); 1.261(6.4); 1.250(1.3); 0.008(2.7); 0.000(77.0); −0.009(2.3)

### Example 183

¹H-NMR (400.0 MHz, DMSO): =8.186(0.7); 8.165(0.7); 8.131(0.6); 8.126(0.6); 8.111(1.2); 8.107(1.2); 8.092(0.6); 8.088(0.6); 8.061(0.6); 8.043(0.6); 7.881(1.2); 7.876(1.3); 7.862(2.4); 7.857(2.8); 7.842(1.5); 7.839(1.6); 7.827(0.4); 7.822(0.4); 7.657(0.6); 7.652(0.6); 7.644(0.6); 7.639(1.1); 7.636(1.0); 7.634(1.0); 7.631(0.9); 7.626(1.0); 7.624(1.0); 7.620(1.3); 7.613(0.7); 7.605(0.7); 7.600(0.7); 7.557(0.5); 7.553(0.7); 7.538(1.1); 7.534(1.5); 7.524(0.6); 7.519(1.2); 7.515(1.2); 7.504(1.6); 7.501(1.6); 7.493(0.7); 7.483(1.7); 7.481(1.7); 7.471(2.4); 7.462(0.7); 7.452(3.2); 7.433(1.0); 7.431(0.9); 7.379(0.5); 7.369(1.7); 7.361(2.0); 7.355(2.9); 7.348(3.6); 7.339(3.3); 7.333(3.4); 7.325(3.1); 7.316(2.6); 7.314(2.5); 7.301(1.8); 7.295(5.3); 7.276(3.6); 7.261(2.2); 7.251(1.2); 7.242(1.8); 7.237(1.5); 7.235(1.6); 7.229(1.2); 7.226(1.4); 7.224(1.4); 7.218(3.6); 7.205(3.0); 7.200(2.4); 7.195(3.3); 7.189(1.5); 7.183(3.5); 7.173(1.6); 7.165(0.8); 7.160(1.6); 5.757(0.6); 4.328(1.4); 4.313(1.8); 4.243(1.7); 4.227(2.4); 4.204(0.6); 4.188(0.3); 4.148(0.5); 4.131(0.8); 4.127(0.6); 4.114(0.6); 4.110(0.8); 4.093(0.4); 3.185(16.0); 3.170(13.7); 3.036(0.4); 3.017(0.4); 2.526(0.7); 2.512(17.1); 2.508(34.6); 2.504(45.4); 2.499(33.0); 2.495(16.1); 1.397(1.1); 1.174(0.7); 1.154(6.6); 1.137(6.6); 1.002(5.0); 0.985(4.9); 0.008(1.1); 0.000(30.9); −0.009(1.2)

### Example 184

¹H-NMR (400.0 MHz, DMSO): =8.592(0.9); 8.571(0.9); 8.495(0.8); 8.475(0.8); 8.453(1.3); 8.448(1.5); 8.441(1.6); 8.436(2.9); 8.431(1.8); 8.424(1.7); 8.419(1.6); 7.767(1.3); 7.762(1.3); 7.748(1.5); 7.743(1.4); 7.569(1.4); 7.564(1.4); 7.550(2.0); 7.545(1.8); 7.490(1.4); 7.478(1.4); 7.471(1.4); 7.463(2.0); 7.459(1.5); 7.451(1.9); 7.444(1.4); 7.432(1.4); 7.386(1.3); 7.368(2.4); 7.364(2.3); 7.354(2.4); 7.350(2.7); 7.347(2.5); 7.338(0.9); 7.332(1.9); 7.325(0.5); 7.303(0.5); 7.296(0.3); 7.289(0.4); 7.229(3.5); 7.207(5.9); 7.201(1.9); 7.190(1.2); 7.185(2.9); 7.178(1.2); 7.156(0.4); 5.756(3.6); 4.291(1.3); 4.277(1.7); 4.229(1.6); 4.214(2.1); 4.190(0.6); 4.172(0.6); 4.155(0.4); 4.106(0.5); 4.090(0.8); 4.085(0.6); 4.073(0.6); 4.068(0.8); 4.052(0.5); 3.321(5.4); 3.186(16.0); 3.163(13.1); 3.121(2.8); 3.095(1.9); 2.524(0.9); 2.511(19.0); 2.506(36.6); 2.502(46.8); 2.497(33.3); 2.493(15.8); 2.451(0.6); 2.433(1.6); 2.415(1.6); 2.397(0.6); 1.147(5.9); 1.131(5.8); 1.001(4.7); 0.984(4.7); 0.946(1.8); 0.929(3.8); 0.911(2.2); 0.709(0.7); 0.693(0.7); 0.008(1.5); 0.000(29.9); −0.009(1.0)

### Example 185

¹H-NMR (400.0 MHz, DMSO): =8.714(0.9); 8.693(0.9); 8.623(0.9); 8.604(0.8); 7.503(0.9); 7.498(0.6); 7.486(1.1); 7.481(1.7); 7.465(1.8); 7.461(1.1); 7.448(0.7); 7.444(1.0); 7.427(0.3); 7.360(1.5); 7.352(1.8); 7.346(2.3); 7.338(3.6); 7.330(2.8); 7.325(2.2); 7.317(2.0); 7.211(2.4); 7.206(2.6); 7.200(0.9); 7.189(4.0); 7.183(4.1); 7.171(0.8); 7.167(2.0); 7.161(1.9); 7.151(2.0); 7.131(2.7); 7.126(2.6); 7.111(2.3); 7.107(2.8); 7.086(1.7); 7.078(0.3); 4.279(1.5); 4.264(2.0); 4.230(1.7); 4.215(2.1); 4.192(0.7); 4.173(0.6); 4.158(0.4); 4.114(0.5); 4.097(0.8); 4.093(0.6); 4.081(0.6); 4.077(0.7); 4.060(0.5); 3.470(0.3); 3.321(9.2); 3.185(16.0); 3.157(14.8); 3.146(0.4); 2.670(0.4); 2.524(1.1); 2.510(25.8); 2.506(50.2); 2.501(64.7); 2.497(46.1); 2.492(21.9); 2.328(0.4); 1.162(0.4); 1.145(0.8); 1.127(0.5); 1.109(6.1); 1.092(6.0); 1.023(0.3); 1.005(0.7); 0.987(0.4); 0.970(5.4); 0.953(5.3); 0.008(1.0); 0.000(20.9); −0.008(0.7)

### Example 186

¹H-NMR (400.0 MHz, DMSO): =8.389(1.1); 8.367(1.1); 7.602(1.6); 7.600(1.6); 7.582(1.9); 7.580(1.8); 7.389(0.7); 7.387(0.8); 7.371(3.3); 7.353(3.0); 7.349(3.5); 7.334(2.2); 7.327(1.5); 7.322(1.3); 7.307(1.5); 7.303(1.5); 7.288(0.7); 7.284(0.6); 7.227(2.2); 7.205(3.7); 7.183(1.7); 7.007(1.6); 7.003(1.6); 6.989(1.5); 6.984(1.4); 4.213(1.8); 4.196(2.3); 4.092(0.6); 4.075(0.9); 4.071(0.7); 4.058(0.7); 4.054(0.9); 4.037(0.5); 3.320(7.2); 3.175(16.0); 3.157(0.8); 2.670(0.4); 2.506(45.0); 2.501(56.2); 2.497(40.8); 2.328(0.4); 1.398(0.4); 1.164(6.4); 1.147(6.4); 0.000(15.7)

### Example 188

¹H-NMR (400.0 MHz, DMSO): =12.793(1.1); 8.054(1.0); 8.034(1.0); 7.821(0.9); 7.804(0.9); 7.801(1.0); 7.460(0.4); 7.456(0.4); 7.441(0.9); 7.438(0.9); 7.422(0.7); 7.419(0.6); 7.370(0.3); 7.355(1.8); 7.350(1.1); 7.341(2.0); 7.334(2.5); 7.320(2.3); 7.303(2.0); 7.298(2.1); 7.283(2.0); 7.279(2.3); 7.253(1.3); 7.237(2.6); 7.234(2.4); 7.229(2.8); 7.223(4.2); 7.206(5.5); 7.185(2.2); 7.169(0.4); 7.164(0.5); 7.157(0.5); 7.152(0.4); 7.138(0.4); 5.756(7.5); 4.268(1.3); 4.251(2.5); 4.228(0.6); 4.212(0.9); 4.192(0.8); 4.175(0.5); 4.144(0.4); 4.135(0.5); 3.324(1.8); 3.144(16.0); 2.517(9.6); 2.511(9.7); 2.506(17.6); 2.502(22.1); 2.497(15.9); 2.291(11.2); 2.180(0.8); 2.034(2.1); 1.199(0.9); 1.184(0.8); 1.152(0.5); 0.952(5.9); 0.935(5.8); 0.008(0.4); 0.000(7.9)

### Example 189

¹H-NMR (400.0 MHz, DMSO): =14.226(2.0); 8.759(1.0); 8.738(1.0); 8.664(0.8); 8.645(0.9); 7.772(0.9); 7.749(1.0); 7.740(0.6); 7.729(1.7); 7.716(2.5); 7.695(4.6); 7.678(1.6); 7.673(1.4); 7.664(1.0); 7.657(0.9); 7.644(1.6); 7.636(1.3); 7.625(1.8); 7.605(3.9); 7.582(2.6); 7.562(0.6); 7.376(1.3); 7.362(1.9); 7.356(3.1); 7.341(3.3); 7.335(2.5); 7.321(2.0); 7.216(2.5); 7.212(2.5); 7.194(4.4); 7.189(3.8); 7.172(2.2); 7.167(1.6); 5.757(8.9); 4.243(1.7); 4.229(3.0); 4.215(2.3); 4.199(0.6); 4.182(0.7); 4.163(0.6); 4.147(0.4); 4.127(0.6); 4.110(0.8); 4.092(0.7); 4.075(0.5); 3.177(16.0); 3.151(13.3); 3.111(0.8); 2.512(13.3); 2.508(25.0); 2.504(31.1); 2.499(22.2); 1.124(0.5); 1.081(6.1); 1.064(6.1); 1.002(0.4); 0.960(4.6); 0.944(4.6); 0.008(0.5); 0.000(11.3); −0.008(0.5)

### Example 190

¹H-NMR (400.0 MHz, DMSO): =8.468(1.2); 8.446(1.2); 7.738(1.5); 7.719(2.0); 7.679(0.6); 7.661(1.6); 7.642(1.3); 7.616(1.2); 7.597(1.4); 7.578(0.5); 7.375(1.8); 7.361(2.4); 7.354(2.7); 7.339(2.3); 7.239(2.4); 7.216(3.9); 7.194(1.7); 7.079(1.6); 7.060(1.5); 4.243(0.4); 4.205(1.9); 4.189(2.4); 4.096(0.6); 4.079(1.0); 4.075(0.7); 4.058(0.9); 4.041(0.5); 3.322(12.0); 3.169(16.0); 3.157(2.6); 2.506(27.4); 2.502(32.9); 2.497(22.9); 1.398(0.9); 1.142(6.5); 1.125(6.3); 0.952(0.9); 0.935(0.9); 0.000(3.6)

### Example 191

¹H-NMR (400.0 MHz, DMSO): =8.390(0.8); 8.368(0.9); 7.544(0.6); 7.541(0.6); 7.536(0.4); 7.530(0.3); 7.443(0.8); 7.440(1.0); 7.423(2.0); 7.420(2.2); 7.413(0.4); 7.409(1.1); 7.405(1.2); 7.392(1.6); 7.387(1.4); 7.371(1.3); 7.368(1.8); 7.361(0.6); 7.355(1.9); 7.346(2.8); 7.342(1.6); 7.339(1.2); 7.333(1.9); 7.328(1.7); 7.324(1.6); 7.309(0.7); 7.306(0.7); 7.227(0.5); 7.222(2.0); 7.217(0.6); 7.205(1.1); 7.199(3.3); 7.194(0.7); 7.182(0.7); 7.177(1.5); 7.065(1.4); 7.061(1.3); 7.047(1.3); 7.042(1.2); 4.203(1.5); 4.186(2.1); 4.108(0.5); 4.091(0.8); 4.087(0.6); 4.074(0.6); 4.070(0.8); 4.053(0.4); 3.323(7.7); 3.171(16.0); 3.156(2.0); 2.524(0.4); 2.511(9.2); 2.506(18.2); 2.502(23.6); 2.497(16.5); 2.492(7.7); 1.164(5.9); 1.148(5.8); 0.964(0.6); 0.947(0.6); 0.008(0.6); 0.000(15.0); −0.009(0.5)

### Example 193

¹H-NMR (400.0 MHz, DMSO): =8.786(2.0); 8.765(2.0); 8.738(0.6); 8.716(0.6); 8.316(0.4); 7.686(0.5); 7.666(1.3); 7.652(1.6); 7.647(1.9); 7.632(2.0); 7.622(2.7); 7.612(8.5); 7.610(8.8); 7.603(3.4); 7.593(2.0); 7.583(2.7); 7.566(0.7); 7.520(0.9); 7.499(1.8); 7.487(0.4); 7.474(6.8); 7.470(12.0); 7.457(1.4); 7.451(1.4); 7.436(0.7); 7.431(0.7); 5.756(1.4); 4.720(4.3); 4.708(4.7); 4.699(1.3); 4.341(0.9); 4.329(0.9); 4.324(1.1); 4.320(1.0); 4.312(1.0); 4.308(1.1); 4.303(1.0); 4.291(0.9); 4.283(0.4); 4.279(0.4); 4.274(0.5); 4.261(0.4); 3.369(0.9); 3.363(0.9); 3.351(1.4); 3.345(3.5); 3.322(106.2); 3.309(4.6); 3.303(1.6); 3.292(1.3); 3.286(1.1); 3.268(0.3); 2.675(0.8); 2.671(1.1); 2.666(0.8); 2.662(0.4); 2.541(0.6); 2.524(3.0); 2.511(66.1); 2.506(130.6); 2.502(169.3); 2.497(119.5); 2.493(55.9); 2.338(0.4); 2.333(0.8); 2.328(1.1); 2.324(0.8); 2.319(0.4); 1.678(0.9); 1.398(1.8); 1.187(0.3); 1.170(0.6); 1.152(0.4); 1.141(6.5); 1.123(16.0); 1.106(12.9); 1.090(4.4); 1.030(10.4); 1.013(10.4); 0.008(0.6); 0.000(16.3); −0.008(0.6)

### Example 194

¹H-NMR (400.0 MHz, DMSO): =8.506(2.0); 8.484(2.0); 8.460(0.7); 8.438(0.7); 7.808(0.4); 7.789(0.4); 7.753(2.4); 7.734(3.3); 7.722(1.5); 7.713(1.3); 7.707(1.2); 7.695(2.7); 7.677(1.8); 7.633(2.5); 7.626(3.9); 7.623(6.1); 7.620(5.5); 7.614(4.6); 7.595(1.1); 7.536(0.4); 7.516(2.5); 7.511(1.9); 7.506(1.6); 7.501(0.4); 7.482(13.0); 7.479(11.0); 7.458(0.4); 7.342(0.9); 7.324(0.8); 7.271(2.6); 7.253(2.4); 5.756(4.0); 4.765(1.3); 4.751(1.4); 4.715(4.5); 4.701(4.8); 4.305(1.1); 4.288(1.5); 4.273(1.4); 4.269(1.5); 4.252(1.0); 3.372(1.0); 3.367(1.0); 3.361(0.6); 3.355(1.4); 3.349(3.1); 3.344(1.9); 3.323(43.0); 3.311(3.5); 3.305(1.5); 3.293(1.1); 3.287(1.1); 3.270(0.4); 2.676(0.3); 2.671(0.5); 2.667(0.4); 2.524(1.2); 2.511(29.7); 2.507(60.9); 2.502(80.1); 2.497(57.0); 2.493(26.8); 2.333(0.4); 2.329(0.5); 2.324(0.4); 1.989(0.5); 1.886(0.8); 1.398(5.1); 1.206(0.4); 1.175(0.4); 1.157(0.4); 1.153(0.4); 1.140(7.7); 1.136(3.7); 1.122(16.0); 1.118(7.0); 1.105(8.1); 1.100(6.9); 1.092(11.4); 1.082(4.8); 1.075(11.0); 1.000(0.7); 0.982(1.5); 0.964(0.7); 0.008(0.5); 0.000(16.1); −0.009(0.5)

### Example 195

¹H-NMR (400.0 MHz, DMSO): =8.186(1.4); 8.180(1.4); 8.165(1.4); 8.159(1.4); 8.097(0.5); 8.092(0.5); 8.075(0.5); 8.070(0.5); 7.598(6.3); 7.595(6.7); 7.530(0.7); 7.526(1.0); 7.522(0.6); 7.517(0.9); 7.512(1.9); 7.505(2.9); 7.496(3.6); 7.492(3.8); 7.483(9.8); 7.479(11.4); 7.474(9.4); 7.458(5.4); 7.449(2.1); 7.444(1.7); 7.428(0.5); 7.423(0.5); 7.296(0.5); 7.281(2.3); 7.271(2.6); 7.269(2.6); 7.260(3.2); 7.253(5.7); 7.243(1.7); 7.233(4.1); 7.225(0.9); 5.757(0.4); 4.808(1.4); 4.796(1.5); 4.744(4.4); 4.730(4.7); 4.359(1.1); 4.341(1.7); 4.323(1.7); 4.307(1.1); 3.384(2.4); 3.366(7.9); 3.349(8.0); 3.326(10.8); 2.526(0.5); 2.508(28.6); 2.504(36.9); 2.499(26.9); 1.990(0.9); 1.957(0.8); 1.397(4.3); 1.212(0.4); 1.194(0.5); 1.176(0.6); 1.161(4.3); 1.153(8.0); 1.143(7.1); 1.136(16.0); 1.125(6.2); 1.118(7.7); 1.112(2.3); 1.108(3.2); 1.097(11.8); 1.080(11.6); 0.000(4.0)

### Example 196

¹H-NMR (400.0 MHz, DMSO): =8.746(1.8); 8.725(1.9); 8.677(0.6); 8.656(0.6); 7.595(5.8); 7.513(0.6); 7.496(1.5); 7.492(2.3); 7.472(13.7); 7.458(1.8); 7.454(2.6); 7.447(3.2); 7.442(1.8); 7.426(1.1); 7.421(0.6); 7.144(3.4); 7.123(5.5); 7.104(4.3); 7.083(1.0); 4.732(5.1); 4.718(5.3); 4.504(0.5); 4.492(0.5); 4.328(0.4); 4.318(1.1); 4.300(1.4); 4.287(1.2); 4.284(1.3); 4.266(0.8); 4.038(0.6); 4.020(0.5); 3.382(0.7); 3.376(0.9); 3.364(1.7); 3.358(3.2); 3.347(3.3); 3.341(4.7); 3.322(54.7); 3.306(1.8); 3.293(0.8); 3.284(0.8); 3.261(0.3); 2.708(0.7); 2.696(0.9); 2.675(0.5); 2.671(0.6); 2.666(0.5); 2.541(0.3); 2.511(41.1); 2.506(79.9); 2.502(103.2); 2.497(74.9); 2.465(0.3); 2.333(0.5); 2.329(0.7); 2.324(0.5); 2.175(0.4); 1.989(2.4); 1.678(1.0); 1.398(12.8); 1.193(0.7); 1.187(0.3); 1.175(1.4); 1.169(0.7); 1.157(0.9); 1.142(7.7); 1.124(16.0); 1.110(7.9); 1.107(9.5); 1.093(2.5); 1.089(2.3); 1.065(10.1); 1.047(9.9); 1.020(0.4); 1.003(0.4); 0.906(1.7); 0.890(1.7); 0.839(0.6); 0.822(0.5); 0.000(9.1); −0.008(0.4)

### Example 197

¹H-NMR (400.0 MHz, DMSO): =8.381(2.2); 8.359(2.2); 8.302(0.7); 8.280(0.7); 7.849(4.0); 7.829(4.2); 7.612(4.8); 7.608(5.0); 7.505(1.0); 7.497(4.4); 7.484(10.2); 7.478(5.2); 7.461(0.6); 7.457(0.7); 7.442(0.6); 7.427(2.0); 7.409(3.5); 7.393(1.9); 7.391(1.9); 7.164(1.2); 7.156(2.2); 7.152(2.3); 7.144(1.2); 7.137(3.5); 7.133(3.5); 7.117(1.7); 7.113(1.8); 7.089(2.9); 7.085(2.7); 7.070(2.7); 7.066(2.3); 5.756(7.6); 4.786(1.4); 4.773(1.4); 4.742(4.1); 4.727(4.3); 4.303(0.5); 4.286(1.3); 4.270(1.6); 4.250(1.5); 4.234(0.9); 3.436(0.4); 3.419(1.3); 3.413(0.8); 3.401(1.5); 3.396(2.2); 3.378(2.1); 3.368(0.4); 3.360(0.8); 3.350(1.7); 3.333(4.0); 3.322(27.2); 3.316(3.9); 3.310(2.0); 3.298(1.1); 3.292(1.6); 3.275(0.5); 2.671(0.3); 2.506(41.4); 2.502(53.4); 2.497(38.5); 2.328(0.3); 1.679(0.8); 1.170(0.6); 1.160(0.4); 1.146(6.9); 1.135(7.1); 1.128(16.0); 1.123(13.0); 1.118(10.9); 1.111(9.2); 1.106(11.5); 0.007(1.5); 0.000(31.7); −0.008(1.2)

### Example 198

¹H-NMR (400.0 MHz, DMSO): =8.173(1.5); 8.151(1.5); 8.095(0.5); 8.072(0.7); 7.607(1.3); 7.588(3.3); 7.583(3.5); 7.509(1.4); 7.488(5.3); 7.475(6.3); 7.471(4.7); 7.454(0.9); 7.450(1.1); 7.291(1.1); 7.275(2.3); 7.257(1.5); 7.253(1.5); 7.206(2.6); 7.199(2.5); 7.182(3.6); 7.163(2.2); 7.118(2.1); 7.114(2.2); 7.105(0.4); 7.097(1.5); 5.756(0.5); 4.784(1.1); 4.770(1.1); 4.690(2.9); 4.674(3.1); 4.340(1.0); 4.323(1.4); 4.306(1.0); 4.302(1.2); 4.285(0.7); 3.359(0.8); 3.353(0.8); 3.336(3.3); 3.321(33.3); 3.301(3.3); 3.295(1.2); 3.284(0.9); 3.278(0.9); 2.675(0.4); 2.671(0.5); 2.666(0.3); 2.607(1.5); 2.510(28.4); 2.506(55.4); 2.501(72.0); 2.497(52.5); 2.333(0.3); 2.328(0.4); 2.324(0.3); 2.214(6.0); 2.117(16.0); 1.162(8.0); 1.145(7.9); 1.130(5.3); 1.124(2.5); 1.113(11.1); 1.106(7.2); 1.095(5.5); 1.089(4.6); 0.008(0.6); 0.000(15.9); −0.008(0.7)

### Example 199

¹H-NMR (400.0 MHz, DMSO): =8.575(2.0); 8.554(2.0); 8.535(0.7); 8.513(0.7); 7.689(0.4); 7.679(0.7); 7.671(1.5); 7.665(1.8); 7.661(1.8); 7.649(2.3); 7.622(1.8); 7.616(2.9); 7.609(1.6); 7.604(5.2); 7.598(3.6); 7.592(4.2); 7.581(4.2); 7.576(4.9); 7.571(5.0); 7.513(2.1); 7.492(7.3); 7.483(4.7); 7.478(4.0); 7.466(2.6); 7.462(2.7); 7.457(2.4); 7.429(1.7); 7.417(1.7); 7.407(1.3); 7.135(0.5); 7.073(1.6); 6.996(1.1); 6.934(3.5); 6.857(0.6); 6.794(1.8); 5.756(3.2); 4.801(1.2); 4.787(1.3); 4.712(3.7); 4.695(3.9); 4.366(0.4); 4.362(0.4); 4.352(0.4); 4.348(0.4); 4.340(1.0); 4.331(0.5); 4.323(1.5); 4.319(1.1); 4.306(1.1); 4.302(1.4); 4.285(0.9); 4.039(0.3); 4.021(0.4); 3.370(0.9); 3.364(1.1); 3.352(1.7); 3.346(3.6); 3.332(3.9); 3.328(5.1); 3.324(15.6); 3.315(3.3); 3.298(1.0); 3.291(0.8); 2.524(0.7); 2.511(14.7); 2.507(28.6); 2.502(37.3); 2.498(27.3); 2.494(13.4); 1.989(1.5); 1.679(0.7); 1.397(2.6); 1.235(0.6); 1.193(0.6); 1.176(10.5); 1.159(10.0); 1.139(9.2); 1.121(16.0); 1.104(6.6); 0.000(3.3)

### Example 200

¹H-NMR (400.0 MHz, DMSO): =8.420(1.6); 8.398(1.6); 8.350(0.5); 8.328(0.5); 7.618(2.4); 7.616(3.0); 7.606(4.5); 7.602(4.7); 7.599(3.3); 7.596(3.5); 7.505(0.9); 7.494(3.3); 7.485(6.3); 7.481(5.1); 7.477(3.8); 7.460(0.5); 7.455(0.6); 7.431(0.4); 7.428(0.4); 7.421(1.0); 7.418(1.1); 7.412(1.0); 7.409(1.0); 7.402(2.5); 7.399(2.4); 7.394(0.8); 7.391(0.8); 7.383(1.8); 7.381(1.6); 7.343(1.8); 7.339(2.0); 7.324(2.4); 7.319(2.5); 7.305(1.2); 7.300(1.1); 7.233(0.8); 7.229(0.7); 7.214(0.7); 7.210(0.6); 7.171(2.2); 7.166(2.2); 7.152(2.0); 7.148(1.8); 5.756(2.6); 4.777(1.0); 4.764(1.1); 4.723(3.1); 4.709(3.3); 4.318(0.4); 4.302(0.9); 4.285(1.2); 4.269(0.9); 4.265(1.2); 4.248(0.7); 4.038(0.5); 4.020(0.6); 3.395(0.9); 3.389(0.6); 3.377(1.1); 3.371(1.9); 3.365(0.5); 3.354(2.0); 3.348(1.4); 3.330(3.1); 3.323(15.7); 3.313(2.5); 3.308(1.3); 3.296(0.8); 3.290(1.1); 2.524(0.6); 2.511(12.9); 2.506(25.3); 2.502(33.0); 2.497(24.1); 2.493(11.8); 1.989(2.4); 1.397(5.9); 1.193(0.7); 1.175(1.3); 1.157(0.8); 1.141(5.4); 1.123(16.0); 1.115(6.2); 1.111(5.2); 1.105(11.6); 1.098(2.7); 0.000(2.9)

### Example 201

¹H-NMR (601.6 MHz, DMSO): =8.415(1.7); 8.401(1.8); 8.346(0.6); 8.332(0.6); 7.599(3.4); 7.596(3.3); 7.504(0.6); 7.499(1.7); 7.490(2.0); 7.485(5.8); 7.475(3.1); 7.472(3.0); 7.462(0.9); 7.458(1.0); 7.453(2.1); 7.440(3.8); 7.419(1.7); 7.416(1.6); 7.407(2.7); 7.404(2.4); 7.394(1.5); 7.391(1.3); 7.378(0.5); 7.376(0.5); 7.369(1.4); 7.367(1.6); 7.364(1.0); 7.357(2.3); 7.355(2.4); 7.344(1.1); 7.343(1.1); 7.272(0.7); 7.270(0.8); 7.260(0.6); 7.257(0.6); 7.216(2.0); 7.213(2.2); 7.203(1.8); 7.201(1.8); 5.755(4.2); 5.754(3.3); 4.776(0.9); 4.767(1.0); 4.717(2.8); 4.707(3.0); 4.306(0.8); 4.295(1.2); 4.281(1.2); 4.270(0.7); 3.375(0.8); 3.371(0.6); 3.363(1.0); 3.359(1.8); 3.347(2.7); 3.333(5.8); 3.329(7.8); 3.326(8.5); 3.322(9.3); 3.305(0.9); 3.301(1.1); 3.290(0.4); 2.508(7.6); 2.505(17.2); 2.502(24.7); 2.499(19.2); 2.497(10.0); 1.947(0.5); 1.910(0.3); 1.154(0.5); 1.143(0.4); 1.134(5.1); 1.122(16.0); 1.113(7.9); 1.110(12.6); 1.102(2.4); 0.987(0.4); 0.000(3.2)

### Example 202

¹H-NMR (400.0 MHz, DMSO): =8.684(2.2); 8.662(2.2); 8.606(0.7); 8.585(0.7); 8.042(2.9); 8.039(3.0); 8.021(3.3); 8.019(3.4); 8.013(1.1); 7.995(1.1); 7.992(1.1); 7.795(1.5); 7.792(1.8); 7.776(3.5); 7.773(4.2); 7.757(2.3); 7.754(2.7); 7.688(2.0); 7.684(2.1); 7.677(0.9); 7.673(1.0); 7.668(2.7); 7.665(2.7); 7.657(1.0); 7.654(1.0); 7.649(1.4); 7.645(1.4); 7.638(0.6); 7.634(0.5); 7.620(3.4); 7.618(6.1); 7.615(4.7); 7.499(0.4); 7.489(4.2); 7.486(5.2); 7.482(13.2); 7.479(12.4); 7.393(3.0); 7.389(3.1); 7.374(3.8); 7.371(3.0); 7.358(1.0); 7.354(0.9); 5.757(10.3); 4.769(1.5); 4.756(1.7); 4.748(4.7); 4.735(4.9); 4.295(0.5); 4.278(1.2); 4.265(1.3); 4.261(1.6); 4.258(1.3); 4.248(1.2); 4.244(1.5); 4.227(0.9); 3.432(0.4); 3.415(1.3); 3.409(0.8); 3.397(1.5); 3.391(2.4); 3.379(0.8); 3.374(2.6); 3.363(1.0); 3.355(1.8); 3.350(1.6); 3.346(2.6); 3.338(1.7); 3.325(43.5); 3.311(1.2); 3.305(1.4); 3.287(0.4); 2.671(0.4); 2.525(0.9); 2.511(23.6); 2.507(47.7); 2.502(62.6); 2.498(45.1); 2.493(21.7); 2.329(0.4); 1.897(0.7); 1.171(0.6); 1.150(8.5); 1.142(3.5); 1.132(16.0); 1.125(6.0); 1.115(7.0); 1.107(2.8); 1.084(11.0); 1.067(10.9); 0.000(2.1)

### Example 203

¹H-NMR (400.0 MHz, DMSO): =10.949(0.4); 8.084(0.6); 8.063(0.6); 8.046(0.5); 8.024(0.5); 7.725(2.0); 7.711(2.0); 7.457(1.3); 7.449(0.9); 7.446(0.9); 7.438(2.4); 7.430(1.2); 7.426(1.2); 7.418(0.4); 7.410(0.6); 7.407(0.6); 7.400(0.6); 7.392(0.9); 7.388(0.8); 7.382(0.8); 7.379(0.7); 7.373(0.5); 7.369(0.5); 7.363(0.5); 7.360(0.4); 7.341(1.1); 7.336(1.2); 7.329(0.4); 7.322(1.2); 7.317(1.2); 7.303(0.5); 7.298(0.5); 7.106(1.4); 7.093(1.3); 7.038(1.8); 7.024(1.7); 5.081(0.3); 4.782(0.9); 4.767(1.0); 4.682(1.1); 4.668(1.2); 4.325(0.4); 4.321(0.4); 4.317(0.4); 4.308(0.5); 4.295(0.3); 4.290(0.4); 3.323(35.7); 3.284(1.5); 3.211(8.7); 3.156(6.8); 3.127(0.5); 2.671(0.4); 2.524(0.9); 2.511(21.8); 2.506(44.8); 2.502(59.6); 2.497(42.9); 2.493(20.6); 2.328(0.4); 1.656(1.4); 1.398(16.0); 1.091(3.3); 1.074(5.6); 1.057(2.5); 0.008(0.6); 0.000(17.9); −0.009(0.6)

### Example 204

¹H-NMR (400.0 MHz, DMSO): =8.860(1.0); 8.839(1.1); 8.822(0.4); 8.800(0.4); 7.612(0.9); 7.605(2.4); 7.601(2.3); 7.489(0.5); 7.468(3.3); 7.463(5.0); 7.448(1.2); 7.441(0.4); 7.427(0.4); 5.756(1.1); 4.753(0.7); 4.740(0.7); 4.681(1.9); 4.667(2.0); 4.249(0.6); 4.232(0.7); 4.213(0.6); 4.196(0.4); 4.038(0.5); 4.021(0.5); 3.347(1.1); 3.324(42.3); 3.313(3.4); 3.295(1.1); 2.709(16.0); 2.525(0.6); 2.511(14.8); 2.507(29.7); 2.502(38.8); 2.498(28.3); 1.989(2.3); 1.398(3.2); 1.193(0.6); 1.175(1.3); 1.157(0.6); 1.140(3.1); 1.129(1.7); 1.123(6.8); 1.112(3.5); 1.105(3.4); 1.098(2.2); 1.077(4.9); 1.060(4.8); 0.008(0.8); 0.000(22.7); −0.008(0.8)

### Example 205

¹H-NMR (400.0 MHz, DMSO): =8.063(2.1); 8.041(2.1); 7.971(0.7); 7.952(1.2); 7.733(5.2); 7.726(2.0); 7.719(5.4); 7.713(1.9); 7.594(1.4); 7.589(1.5); 7.574(4.3); 7.569(4.4); 7.496(1.3); 7.476(7.2); 7.470(5.1); 7.465(4.8); 7.452(1.7); 7.448(2.0); 7.431(0.4); 7.427(0.4); 7.073(1.7); 7.059(1.7); 7.025(5.3); 7.011(5.2); 5.756(5.5); 4.842(1.2); 4.829(1.3); 4.721(3.8); 4.706(4.0); 4.319(0.4); 4.302(1.2); 4.286(1.6); 4.270(1.3); 4.265(1.3); 4.249(0.8); 3.391(1.0); 3.385(0.8); 3.374(1.3); 3.368(2.4); 3.360(1.3); 3.355(1.6); 3.350(2.7); 3.342(2.9); 3.325(32.8); 3.313(1.8); 3.307(1.5); 3.301(1.2); 3.289(0.5); 3.284(0.4); 2.891(5.2); 2.732(4.4); 2.672(0.4); 2.525(0.8); 2.507(47.4); 2.503(62.2); 2.498(45.6); 2.329(0.4); 1.679(0.8); 1.170(0.5); 1.145(6.5); 1.127(16.0); 1.122(11.6); 1.109(14.3); 1.105(12.4); 1.091(5.4); 1.004(0.4); 0.000(2.2)

### Example 206

¹H-NMR (400.0 MHz, DMSO): =8.892(1.4); 8.885(4.1); 8.884(3.9); 8.880(3.7); 8.878(3.5); 8.682(3.2); 8.678(3.3); 8.670(3.4); 8.666(3.2); 8.510(1.9); 8.488(1.9); 8.441(0.6); 8.419(0.6); 8.096(0.5); 8.091(0.8); 8.085(1.8); 8.080(2.3); 8.075(1.9); 8.072(1.1); 8.065(1.9); 8.060(2.4); 8.055(1.6); 7.599(1.5); 7.595(1.5); 7.543(4.8); 7.538(5.3); 7.513(2.7); 7.492(8.0); 7.479(2.7); 7.478(2.5); 7.472(3.0); 7.470(2.6); 7.465(4.1); 7.460(5.3); 7.452(2.5); 7.444(1.8); 7.440(2.7); 7.436(1.5); 7.419(0.4); 7.415(0.4); 5.758(5.2); 4.883(1.3); 4.870(1.3); 4.733(3.7); 4.717(3.9); 4.407(0.4); 4.403(0.3); 4.394(0.5); 4.390(0.4); 4.386(0.4); 4.378(1.0); 4.361(1.4); 4.357(1.1); 4.345(1.1); 4.340(1.4); 4.324(0.9); 4.057(1.0); 4.040(2.9); 4.022(3.0); 4.004(1.0); 3.374(0.4); 3.363(2.3); 3.356(1.3); 3.346(6.7); 3.339(1.8); 3.328(22.6); 3.311(2.2); 3.304(0.5); 2.527(0.5); 2.514(11.1); 2.509(22.6); 2.505(29.9); 2.500(21.5); 2.496(10.2); 1.991(13.0); 1.397(0.6); 1.194(4.3); 1.186(10.4); 1.176(8.3); 1.169(10.3); 1.159(3.8); 1.134(8.5); 1.124(3.7); 1.117(16.0); 1.107(5.5); 1.099(6.8); 1.089(2.3); 0.008(0.9); 0.000(24.9); −0.009(0.8)

### Example 207

¹H-NMR (400.0 MHz, DMSO): =8.912(1.4); 8.908(1.3); 8.891(1.9); 8.887(1.9); 8.681(1.1); 8.676(2.3); 8.672(1.7); 8.669(1.4); 8.664(2.4); 8.660(1.5); 8.531(1.0); 8.510(1.3); 8.493(0.7); 8.118(0.6); 8.113(0.9); 8.108(0.6); 8.098(1.4); 8.093(2.1); 8.088(1.4); 8.078(0.9); 8.073(1.3); 8.068(0.8); 7.490(0.8); 7.488(0.8); 7.484(1.2); 7.482(1.2); 7.478(0.9); 7.476(0.9); 7.470(2.6); 7.465(2.0); 7.458(1.7); 7.456(1.6); 7.450(2.7); 7.446(2.2); 7.440(1.7); 7.436(1.3); 7.427(1.0); 7.422(1.2); 7.416(1.3); 7.413(1.4); 7.394(2.5); 7.380(2.0); 7.361(1.7); 7.345(0.7); 7.341(0.6); 7.336(0.9); 7.330(1.0); 7.316(1.8); 7.312(1.9); 7.297(1.5); 7.293(1.4); 7.278(0.6); 7.274(0.5); 5.757(0.4); 4.823(1.4); 4.808(1.5); 4.682(2.0); 4.667(2.2); 4.432(0.4); 4.415(0.9); 4.398(1.1); 4.394(1.0); 4.378(1.0); 4.361(0.6); 4.038(0.9); 4.020(0.9); 3.324(26.8); 3.196(16.0); 3.165(10.9); 2.525(0.7); 2.511(17.5); 2.507(35.6); 2.502(46.7); 2.498(33.5); 2.493(15.8); 1.989(4.0); 1.398(6.4); 1.216(0.4); 1.199(0.5); 1.193(1.2); 1.175(2.3); 1.163(6.0); 1.157(1.8); 1.146(5.9); 1.111(3.9); 1.094(3.9); 0.008(1.0); 0.000(28.3); −0.009(0.9)

### Example 208

¹H-NMR (400.0 MHz, DMSO): =9.010(1.4); 9.003(1.7); 8.999(1.4); 8.993(1.5); 8.944(1.6); 8.938(1.4); 8.923(1.6); 8.917(1.3); 8.870(0.7); 8.849(0.7); 8.787(0.6); 8.766(0.7); 7.499(0.9); 7.497(0.9); 7.484(0.9); 7.480(2.0); 7.477(2.1); 7.471(0.9); 7.466(1.0); 7.461(1.4); 7.458(1.9); 7.453(1.5); 7.443(1.5); 7.437(2.2); 7.423(1.3); 7.420(1.0); 7.404(0.5); 7.401(0.4); 7.393(0.4); 7.389(0.5); 7.386(1.0); 7.379(0.9); 7.375(1.1); 7.371(1.1); 7.366(0.8); 7.360(0.9); 7.356(1.5); 7.350(1.5); 7.343(0.4); 7.337(0.9); 7.332(1.0); 7.318(0.4); 4.722(1.5); 4.712(2.0); 4.709(1.9); 4.703(1.8); 4.368(0.4); 4.359(0.4); 4.351(0.4); 4.347(0.4); 4.341(0.4); 4.338(0.4); 4.330(0.4); 4.320(0.4); 4.295(0.3); 4.282(0.4); 4.278(0.4); 4.273(0.4); 4.265(0.4); 4.260(0.4); 4.256(0.4); 4.243(0.3); 3.323(43.6); 3.222(13.2); 3.178(12.6); 2.671(0.4); 2.524(1.0); 2.520(1.7); 2.511(23.5); 2.507(48.3); 2.502(63.8); 2.497(45.1); 2.493(20.8); 2.329(0.4); 1.398(16.0); 1.154(4.3); 1.137(4.2); 1.012(4.5); 0.994(4.5); 0.008(1.4); 0.000(43.1); −0.009(1.3)

### Example 209

¹H-NMR (400.0 MHz, DMSO): =8.869(0.8); 8.857(1.8); 8.844(1.2); 8.842(1.2); 8.662(0.8); 8.641(0.9); 8.586(0.6); 8.564(0.6); 8.286(1.2); 8.283(1.2); 8.277(0.8); 8.274(0.8); 8.265(1.3); 8.263(1.3); 8.257(0.9); 8.254(0.8); 7.734(1.0); 7.732(1.0); 7.725(0.8); 7.719(1.3); 7.714(1.0); 7.707(0.6); 7.705(0.7); 7.701(0.8); 7.483(1.2); 7.480(1.7); 7.474(0.9); 7.468(1.0); 7.463(2.8); 7.461(3.0); 7.456(1.7); 7.449(1.4); 7.442(1.8); 7.437(1.8); 7.431(1.0); 7.427(0.9); 7.413(1.5); 7.410(1.2); 7.394(0.7); 7.391(0.5); 7.377(0.4); 7.372(1.3); 7.366(1.1); 7.359(1.0); 7.354(1.5); 7.347(1.0); 7.342(1.7); 7.336(1.7); 7.329(0.6); 7.323(0.8); 7.318(0.8); 4.745(1.3); 4.731(1.4); 4.718(2.0); 4.708(2.0); 4.352(0.4); 4.342(0.4); 4.334(0.5); 4.330(0.5); 4.324(0.5); 4.320(0.5); 4.313(0.5); 4.303(0.4); 4.260(0.4); 4.256(0.4); 4.246(0.3); 4.241(0.4); 4.056(0.5); 4.038(1.7); 4.020(1.7); 4.002(0.6); 3.323(45.4); 3.214(15.9); 3.167(10.6); 2.675(0.3); 2.671(0.5); 2.666(0.3); 2.524(1.3); 2.520(2.0); 2.511(26.3); 2.507(53.5); 2.502(70.6); 2.497(50.3); 2.493(23.6); 2.329(0.4); 2.324(0.3); 1.989(7.7); 1.398(16.0); 1.193(2.1); 1.175(4.2); 1.170(0.5); 1.157(2.1); 1.153(0.4); 1.115(3.7); 1.098(3.7); 1.023(5.7); 1.006(5.6); 0.008(1.7); 0.000(47.5); −0.009(1.5)

### Example 210

¹H-NMR (400.0 MHz, DMSO): =8.924(2.2); 8.659(3.1); 8.652(3.7); 8.593(3.7); 8.587(3.1); 8.317(1.5); 7.602(2.9); 7.596(3.0); 7.534(2.0); 7.513(3.3); 7.459(1.9); 7.453(1.8); 7.438(1.1); 7.432(1.0); 5.757(0.6); 5.012(4.3); 3.324(16.4); 3.301(0.5); 3.180(16.0); 2.891(0.4); 2.511(12.4); 2.507(24.6); 2.502(32.2); 2.498(23.4); 2.493(11.4); 1.051(0.4); 1.045(0.5); 1.040(0.6); 1.031(0.8); 1.020(0.6); 1.016(0.7); 1.005(0.6); 0.944(0.4); 0.926(0.8); 0.922(0.5); 0.908(1.0); 0.894(0.9); 0.881(0.6); 0.874(0.5); 0.813(0.4); 0.798(0.4); 0.789(0.8); 0.774(1.2); 0.766(2.0); 0.755(0.8); 0.744(0.7); 0.735(0.4); 0.008(1.8); 0.000(45.0); −0.009(1.7)

### Example 211

¹H-NMR (400.0 MHz, DMSO): =8.772(2.7); 8.010(1.9); 7.989(2.1); 7.844(0.4); 7.789(1.0); 7.786(0.9); 7.770(2.4); 7.768(2.0); 7.751(1.7); 7.749(1.4); 7.718(0.6); 7.711(0.5); 7.701(0.8); 7.669(1.3); 7.666(1.2); 7.649(1.9); 7.630(1.3); 7.627(1.1); 7.617(3.1); 7.612(2.9); 7.573(0.3); 7.555(0.4); 7.539(0.6); 7.523(1.6); 7.502(4.0); 7.485(2.5); 7.480(2.1); 7.464(0.8); 7.459(0.8); 7.439(2.0); 7.436(1.7); 7.420(1.8); 7.417(1.4); 5.756(0.7); 5.015(4.7); 3.326(6.3); 3.168(16.0); 3.112(0.5); 3.094(0.6); 3.075(0.4); 3.043(1.0); 3.027(1.0); 2.671(0.4); 2.506(55.0); 2.502(65.3); 2.498(45.0); 2.329(0.4); 1.181(2.6); 1.169(2.6); 1.163(4.7); 1.151(1.4); 1.145(2.3); 1.049(0.4); 1.033(1.2); 1.023(1.0); 1.015(1.8); 1.008(0.8); 0.997(1.1); 0.915(0.6); 0.899(0.8); 0.888(0.9); 0.874(0.6); 0.862(0.5); 0.803(0.5); 0.792(0.6); 0.779(1.0); 0.767(0.8); 0.763(0.8); 0.751(0.6); 0.716(0.7); 0.705(0.8); 0.689(0.8); 0.681(0.5); 0.676(0.5); 0.664(0.3); 0.008(5.4); 0.000(71.7); −0.009(2.7); −0.150(0.3)

### Example 212

¹H-NMR (400.0 MHz, DMSO): =8.913(1.0); 8.892(1.1); 7.472(1.2); 7.470(1.3); 7.461(0.4); 7.453(1.8); 7.450(1.9); 7.439(0.7); 7.432(0.7); 7.418(2.0); 7.413(3.6); 7.396(1.9); 7.393(1.6); 7.377(0.9); 7.364(1.3); 7.358(1.4); 7.344(1.1); 7.339(1.1); 7.327(0.6); 7.322(0.6); 7.298(0.3); 5.756(1.6); 4.699(0.4); 4.686(0.4); 4.629(2.2); 4.616(2.3); 4.279(0.6); 4.266(0.6); 4.262(0.8); 4.259(0.7); 4.249(0.6); 4.245(0.7); 4.241(0.6); 4.228(0.5); 4.038(0.4); 4.020(0.4); 3.324(23.8); 3.185(16.0); 3.168(3.0); 2.707(15.8); 2.691(1.2); 2.524(0.6); 2.511(15.4); 2.506(31.3); 2.502(41.3); 2.497(30.2); 2.493(14.7); 1.989(1.6); 1.398(2.9); 1.193(0.5); 1.175(0.9); 1.157(0.4); 1.108(1.4); 1.091(1.3); 1.054(6.1); 1.037(6.0); 0.008(2.0); 0.000(52.1); −0.009(2.0)

### Example 213

¹H-NMR (400.0 MHz, DMSO): =8.817(0.9); 8.796(1.1); 8.736(0.7); 8.730(0.3); 8.716(0.7); 8.690(3.2); 8.683(5.3); 8.677(2.6); 8.620(0.4); 8.614(3.8); 8.608(3.1); 8.602(2.5); 8.596(2.1); 7.489(1.3); 7.486(1.3); 7.478(0.9); 7.469(2.9); 7.467(2.8); 7.460(1.2); 7.453(3.0); 7.437(1.0); 7.434(1.0); 7.419(1.6); 7.417(1.4); 7.400(0.8); 7.397(0.7); 7.392(0.6); 7.388(0.6); 7.384(0.4); 7.378(1.3); 7.373(2.0); 7.358(1.3); 7.355(1.6); 7.350(1.4); 7.344(1.1); 7.340(0.7); 7.335(0.6); 7.331(0.9); 7.326(0.9); 7.312(0.4); 7.307(0.3); 5.757(5.0); 4.727(1.7); 4.723(2.2); 4.713(2.9); 4.376(0.5); 4.365(0.5); 4.359(0.6); 4.355(0.6); 4.348(0.5); 4.344(0.6); 4.337(0.6); 4.327(0.5); 4.319(0.4); 4.305(0.4); 4.301(0.4); 4.297(0.4); 4.288(0.4); 4.284(0.4); 4.279(0.4); 4.267(0.3); 3.325(22.6); 3.236(0.8); 3.228(16.0); 3.192(1.1); 3.185(11.0); 2.525(0.5); 2.520(0.8); 2.512(12.3); 2.507(25.1); 2.503(33.0); 2.498(23.7); 2.494(11.2); 1.989(0.3); 1.397(0.7); 1.201(0.5); 1.185(0.5); 1.173(4.1); 1.156(4.1); 1.047(5.8); 1.029(5.8); 0.008(0.7); 0.000(23.4); −0.009(0.8)

### Example 214

¹H-NMR (400.0 MHz, DMSO): =8.764(2.3); 8.743(2.4); 8.688(6.2); 8.682(7.1); 8.613(7.1); 8.607(6.0); 8.317(1.3); 7.618(6.2); 7.491(14.8); 7.489(13.1); 4.764(5.1); 4.752(5.3); 4.352(1.0); 4.339(1.1); 4.334(1.4); 4.330(1.2); 4.322(1.2); 4.318(1.4); 4.313(1.1); 4.301(1.0); 3.405(1.1); 3.399(1.0); 3.387(1.4); 3.381(3.5); 3.363(4.8); 3.344(4.2); 3.325(212.7); 3.303(1.1); 2.675(1.3); 2.671(1.7); 2.666(1.2); 2.541(1.0); 2.506(199.2); 2.502(255.2); 2.497(185.1); 2.333(1.3); 2.329(1.7); 2.324(1.2); 1.398(1.6); 1.155(7.6); 1.137(16.0); 1.120(7.5); 1.105(0.4); 1.078(12.3); 1.061(12.2); 0.146(1.1); 0.007(10.2); 0.000(239.3); −0.008(10.2); −0.150(1.2)

The following examples illustrate in a non-limiting manner the efficacy of the compounds of formula (I) according to the invention.

### BIOLOGICAL EXAMPLES

### Boophilus microplus—Injectiontest (BOOPMI Inj)

Solvent: dimethyl sulfoxide

To produce a suitable preparation of active compound, 10 mg of active compound are dissolved in 0.5 ml solvent, and the concentrate is diluted with solvent to the desired concentration.

Five adult engorged female ticks (Boophilus microplus) are injected with 1 μl compound solution into the abdomen. Ticks are transferred into replica plates and incubated in a climate chamber.

After 7 days the egg deposition of fertile eggs is monitored. Eggs where fertility is not visible are stored in a climate chamber till hatching after about 42 days. An efficacy of 100% means all eggs are infertile; 0% means all eggs are fertile.

In this test, for example, the following compounds from the preparation examples showed good activity of 80% at an application rate of 20 μg/animal: 103

Cooperia curticei—Test (COOPCU)

Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 nematode larvae (Cooperia curticei) are transferred into a test tube containing the compound solution.

After 5 days percentage of larval mortality is recorded. 100% efficacy means all larvae are killed; 0% efficacy means no larvae are killed.

In this test for example, the following compounds from the preparation examples showed good activity of 100% at an application rate of 100 ppm: 1, 30, 34, 35, 39, 58, 63, 70, 71

In this test for example, the following compounds from the preparation examples showed good activity of 90% at an application rate of 100 ppm: 6, 69

In this test for example, the following compounds from the preparation examples showed good activity of 80% at an application rate of 100 ppm: 65

In this test for example, the following compounds from the preparation examples showed good activity of 100% at an application rate of 20 ppm: 2, 99, 107, 202, 205

In this test for example, the following compounds from the preparation examples showed good activity of 90% at an application rate of 20 ppm: 73, 92, 103, 209

In this test for example, the following compounds from the preparation examples showed good activity of 80% at an application rate of 20 ppm: 8, 64, 128, 130, 203, 208

Haemonchus contortus—Test (HAEMCO)

Solvent: dimethyl sulfoxide

To produce a suitable preparation of active compound, 10 mg of active compound are dissolved in 0.5 ml solvent, and the concentrate is diluted with “Ringer's solution” to the desired concentration.

Approximately 40 larvae of the red stomach worm (Haemonchus contortus) are transferred into a test tube containing compound solution.

After 5 days percentage of larval mortality is recorded. 100% efficacy means all larvae are killed, 0% efficacy means no larvae are killed.

In this test for example, the following compounds from the preparation examples showed good activity of 100% at an application rate of 100 ppm: 30, 35, 39, 63, 65, 70

In this test for example, the following compounds from the preparation examples showed good activity of 90% at an application rate of 100 ppm: 12, 34, 59, 69, 71

In this test for example, the following compounds from the preparation examples showed good activity of 80% at an application rate of 100 ppm: 1, 58

In this test for example, the following compounds from the preparation examples showed good activity of 100% at an application rate of 20 ppm: 64, 73, 123

In this test for example, the following compounds from the preparation examples showed good activity of 90% at an application rate of 20 ppm: 2, 66, 128, 131, 205

In this test for example, the following compounds from the preparation examples showed good activity of 80% at an application rate of 20 ppm: 98, 120, 129, 130, 136, 202, 203

Meloidogyne incognita—test (MELGIN)

Solvent: 125.0 parts by weight of acetone

To produce a suitable preparation of active compound, 1 part by weight of active compound is mixed with the stated amount of solvent, and the concentrate is diluted with water to the desired concentration.

Vessels are filled with sand, a solution of the active ingredient, a suspension containing eggs and larvae of the southern root-knot nematode (Meloidogyne incognita) and salad seeds. The salad seeds germinate and the seedlings grow. Galls develop in the roots.

After 14 days the nematicidal activity is determined on the basis of the percentage of gall formation. 100% means that no galls were found; 0% means the number of galls found on the roots of the treated plants was equal to that in untreated control plants.

In this test, for example, the following compounds from the preparation examples showed good activity of 100% at an application rate of 20 ppm: 12, 30, 73, 107, 108, 210, 213

In this test, for example, the following compounds from the preparation examples showed good activity of 90% at an application rate of 20 ppm: 25, 35, 39, 92, 100, 103, 104, 105, 106, 140, 141, 142, 143, 144, 156, 191, 194, 202, 208.

1. A compound of formula (I) 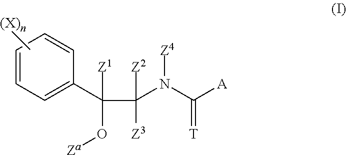
wherein
A represents phenyl of formula A^{a} 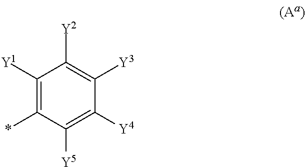
wherein
* indicates the bond which connects A^{a} to the C=T moiety of the compound of formula (I),
Y¹ represents halogen, nitro, SH, SF₅, CHO, OCHO, NHCHO, cyano, C₁-C₈-alkyl, C₁-C₈-halogenoalkyl having 1 to 5 halogen atoms, C₂-C₈-alkenyl, C₂-C₈-alkynyl, C₃-C₆-cycloalkyl, C₃-C₆-halogenocycloalkyl having 1 to 9 halogen atoms, C₁-C₈-alkylsulfanyl, —C₁-C₈-halogenoalkylsulfanyl having 1 to 5 halogen atoms, C₁-C₈-alkoxy, C₁-C₈-halogenoalkoxy having 1 to 5 halogen atoms, C₁-C₈-alkoxy-C₁-C₈-alkyl, C₂-C₈-alkenyloxy-C₁-C₈-alkyl, C₃-C₈-alkynyloxy-C₁-C₈-alkyl, C₂-C₈-alkenyloxy, C₃-C₈-alkynyloxy, C₁-C₈-alkoxycarbonyl, C₁-C₈-halogenoalkoxycarbonyl having 1 to 5 halogen atoms, C₁-C₈-alkylcarbonyloxy, C₁-C₈-halogenoalkylcarbonyloxy having 1 to 5 halogen atoms, C₁-C₈-alkylsulfinyl, C₁-C₈-halogenoalkylsulfinyl having 1 to 5 halogen atoms, C₁-C₈-alkylsulfonyl, C₁-C₈-halogenoalkylsulfonyl having 1 to 5 halogen atoms, C₁-C₈-alkylsulfonamide, substituted or unsubstituted tri-(C₁-C₈)-alkylsilyl, substituted or unsubstituted aryl and substituted or unsubstituted aryloxy;
Y², Y³, Y⁴ and Y⁵ independently from each other represent hydrogen or Y¹; or
A represents a carbo-linked heterocyclyl group selected from the group consisting of A¹ to A³³ wherein
A¹ is a heterocycle of formula (A¹) 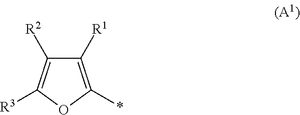
wherein
R¹ to R³ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms,
A² is a heterocycle of formula (A²) 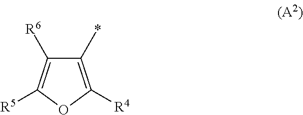
wherein
R⁴ to R⁶ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms,
A³ is a heterocycle of formula (A³) 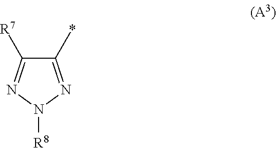
wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl,
A⁴ is a heterocycle of formula (A⁴) 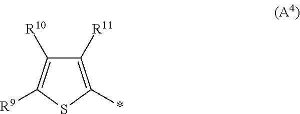
wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms,
A⁵ is a heterocycle of formula (A⁵) 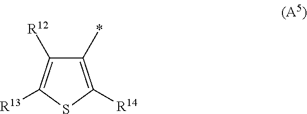
wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms,
A⁶ is a heterocycle of formula (A⁶) 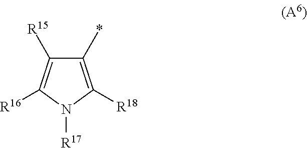
wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl,
A⁷ is a heterocycle of formula (A⁷) 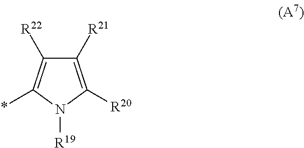
wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms,
A⁸ is a heterocycle of formula (A⁸) 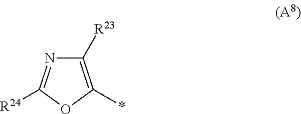
wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms,
A⁹ is a heterocycle of formula (A⁹) 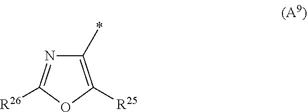
wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms,
A¹⁰ is a heterocycle of formula (A¹⁰) 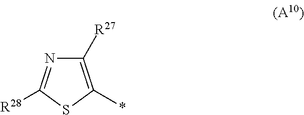
wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹¹ is a heterocycle of formula (A¹¹) 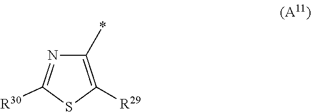
wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹² is a heterocycle of formula (A¹²) 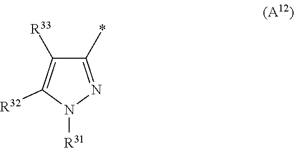
wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms,
A¹³ is a heterocycle of formula (A¹³) 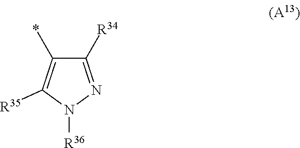
wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl,
A¹⁴ is a heterocycle of formula (A¹⁴) 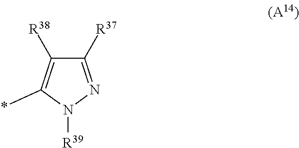
wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl,
A¹⁵ is a heterocycle of formula (A¹⁵) 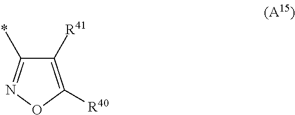
wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms,
A¹⁶ is a heterocycle of formula (A¹⁶) 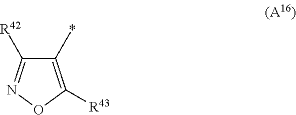
wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino,
A¹⁷ is a heterocycle of formula (A¹⁷) 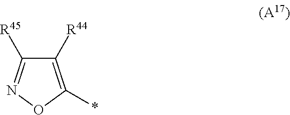
wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms,
A¹⁸ is a heterocycle of formula (A¹⁸) 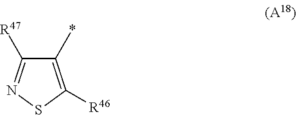
wherein
R⁴⁷ represents hydrogen, halogen, substituted or unsubstituted C₁-C₅-alkyl or C₁-C₅-halogenoalkyl comprising 1 to 9 halogen atoms, and
R⁴⁶ represents hydrogen, halogen, substituted or unsubstituted C₁-C₅-alkyl, C₁-C₅-halogenoalkyl comprising 1 to 9 halogen atoms or substituted or unsubstituted C₁-C₅-alkylsulfanyl,
A¹⁹ is a heterocycle of formula (A¹⁹) 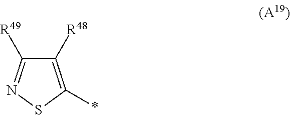
wherein
R⁴⁹ and R⁴⁸ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms,
A²⁰ is a heterocycle of formula (A²⁰) 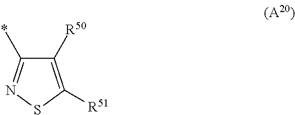
wherein
R⁵⁰ and R⁵¹ independently from each other represent hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms,
A²¹ is a heterocycle of formula (A²¹) 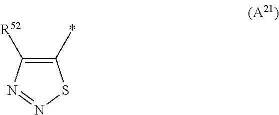
wherein
R⁵² represents hydrogen, halogen, substituted or unsubstituted C₁-C₅-alkyl or C₁-C₅-halogenoalkyl comprising 1 to 9 halogen atoms,
A²² is a heterocycle of formula (A²²) 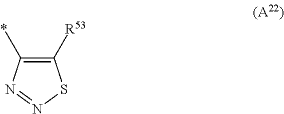
wherein
R⁵³ represents hydrogen, halogen, substituted or unsubstituted C₁-C₅-alkyl or C₁-C₅-halogenoalkyl comprising 1 to 9 halogen atoms,
A²³ is a heterocycle of formula (A²³) 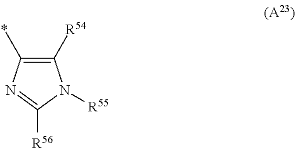
wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl,
A²⁴ is a heterocycle of formula (A²⁴) 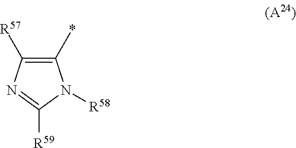
wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl,
A²⁵ is a heterocycle of formula (A²⁵) 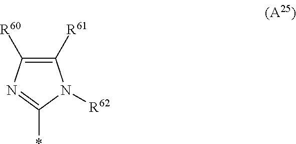
wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl,
A²⁶ is a heterocycle of formula (A²⁶) 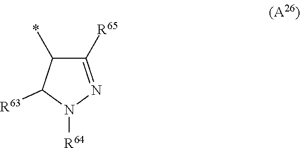
wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms,
A²⁷ is a heterocycle of formula (A²⁷) 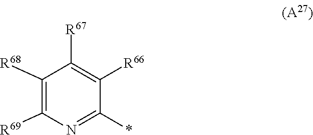
wherein
R⁶⁶ represents hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms and C₁-C₄-halogenoalkoxy having 1 to 5 halogen atoms, and
R⁶⁷, R⁶⁸ and R⁶⁹ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, SC₁-C₄-alkylsulfinyl and C₁-C₄-alkylsulfonyl,
A²⁸ is a heterocycle of formula (A²⁸) 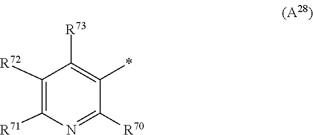
wherein
R⁷⁰ represents hydrogen, halogen, hydroxy, cyano, C₁-C₄-alkyl, C₁-C₄-halogenoalkyl having 1 to 5 halogen atoms, C₁-C₄-alkoxy, C₁-C₅-alkylsulfanyl, C₂-C₅-alkenylsulfanyl, C₁-C₄-halogenoalkylsulfanyl having 1 to 5 halogen atoms, C₁-C₄-halogenoalkoxy having 1 to 5 halogen atoms, phenyloxy (optionally substituted by halogen or C₁-C₄-alkyl) and phenylsulfanyl (optionally substituted by halogen or C₁-C₄-alkyl), and
R⁷¹, R⁷² and R⁷³ independently from each other represent hydrogen, halogen, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkoxy having 1 to 5 halogen atoms, C₁-C₄-alkylsulfinyl, C₁-C₄-alkylsulfonyl, N-morpholine optionally substituted by halogen or C₁-C₄-alkyl, and thienyl (optionally substituted by halogen or C₁-C₄-alkyl),
A²⁹ is a heterocycle of formula (A²⁹) 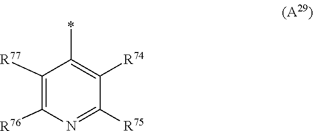
wherein
R⁷⁴, R⁷⁵, R⁷⁶ and R⁷⁷ independently from each other represent hydrogen, halogen, hydroxy, cyano, C₁-C₄-alkyl, C₁-C₄-halogenoalkyl having 1 to 5 halogen atoms, C₁-C₄-alkoxy, C₁-C₄-alkylsulfanyl, C₁-C₄-halogenoalkylsulfanyl having 1 to 5 halogen atoms, C₁-C₄-halogenoalkoxy having 1 to 5 halogen atoms, C₁-C₄-alkylsulfinyl and —C₄-alkylsulfonyl,
A³⁰ is a heterocycle of formula (A³⁰) 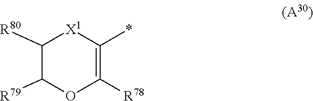
wherein
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl,
A³¹ is a heterocycle of formula (A³¹) 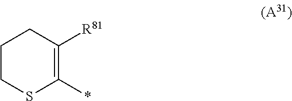
wherein
R⁸¹ represents C₁-C₄-alkyl and C₁-C₄-halogenoalkyl having 1 to 5 halogen atoms,
A³² is a heterocycle of formula (A³²) 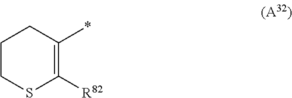
wherein
R⁸² represents C₁-C₄-alkyl and C₁-C₄-halogenoalkyl having 1 to 5 halogen atoms, and
A³³ is a heterocycle of formula (A³³) 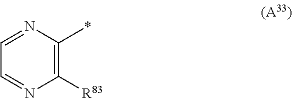
wherein
R⁸³ represents hydrogen, halogen, C₁-C₄-alkyl and C₁-C₄-halogenoalkyl having 1 to 5 halogen atoms,
T represents oxygen or sulphur,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkoxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ represents hydrogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl
Z² and Z³ independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Z⁴ represents hydrogen, cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino,
Z^{a} represents substituted or unsubstituted C₁-C₈-alkyl, substituted or unsubstituted C₃-C₈-alkenyl, substituted or unsubstituted C₃-C₈-alkynyl, substituted or unsubstituted C₃-C₇-cycloalkyl, substituted or unsubstituted C₁-C₆-alkylideneamino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
And/or an N-oxide, metallic complex, metalloidic complex and/or an optically active isomer of a compound of formula (I),
Capable of being used for controlling one or more nematodes which damage plants.

2. Compound of formula (I-1) 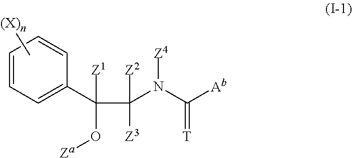
in which,
Z² and Z³ independently represent halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Z⁴ represents a hydrogen atom
A^{b} represents A³ to A¹⁷, A²¹ to A²⁶ and A³⁰ to A³³ provided that when A^{b} represents A¹³, R³⁴ does not represent a halogenomethyl group.
A³ is a heterocycle of formula (A³) 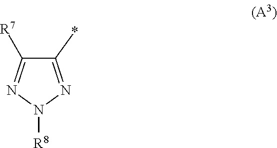
wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl,
A⁴ is a heterocycle of formula (A⁴) 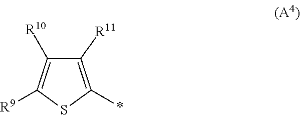
wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms,
A⁵ is a heterocycle of formula (A⁵) 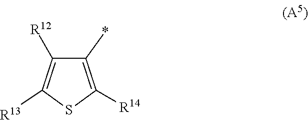
wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms,
A⁶ is a heterocycle of formula (A⁶) 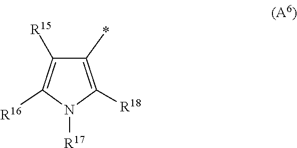
wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl,
A⁷ is a heterocycle of formula (A⁷) 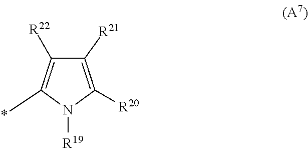
wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms,
A⁸ is a heterocycle of formula (A⁸) 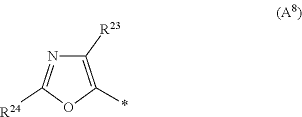
wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms,
A⁹ is a heterocycle of formula (A⁹) 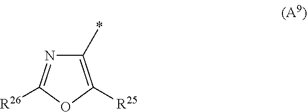
wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms,
A¹⁰ is a heterocycle of formula (A¹⁰) 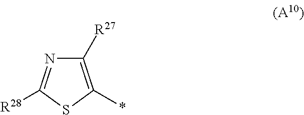
wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹¹ is a heterocycle of formula (A¹¹) 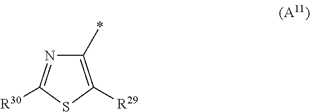
wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹² is a heterocycle of formula (A¹²) 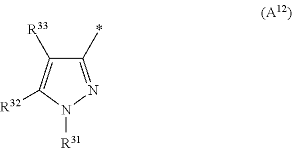
wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms,
A¹³ is a heterocycle of formula (A¹³) 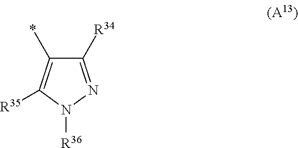
wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl,
A¹⁴ is a heterocycle of formula (A¹⁴) 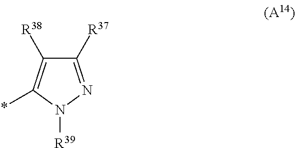
wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl,
A¹⁵ is a heterocycle of formula (A¹⁵) 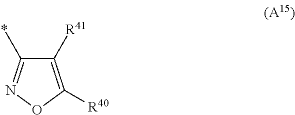
wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms,
A¹⁶ is a heterocycle of formula (A¹⁶) 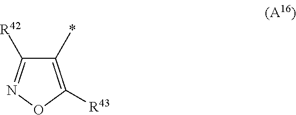
wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino,
A¹⁷ is a heterocycle of formula (A¹⁷) 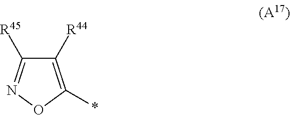
wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms,
A²¹ is a heterocycle of formula (A²¹) 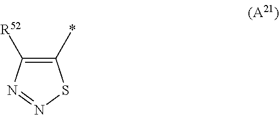
wherein
R⁵² represents hydrogen, halogen, substituted or unsubstituted C₁-C₅-alkyl or C₁-C₅-halogenoalkyl comprising 1 to 9 halogen atoms,
A²² is a heterocycle of formula (A²²) 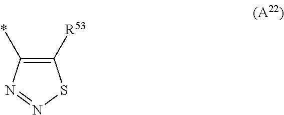
wherein
R⁵³ represents hydrogen, halogen, substituted or unsubstituted C₁-C₅-alkyl or C₁-C₅-halogenoalkyl comprising 1 to 9 halogen atoms,
A²³ is a heterocycle of formula (A²³) 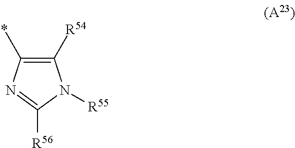
wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl,
A²⁴ is a heterocycle of formula (A²⁴) 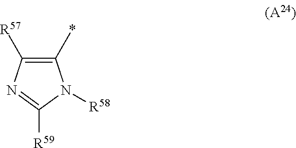
wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl,
A²⁵ is a heterocycle of formula (A²⁵) 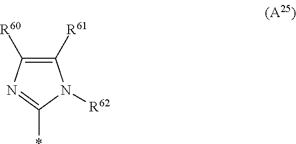
wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl,
A²⁶ is a heterocycle of formula (A²⁶) 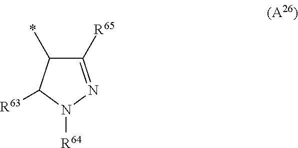
wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms,
A³⁰ is a heterocycle of formula (A³⁰) 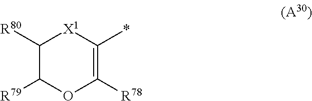
wherein
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl,
A³¹ is a heterocycle of formula (A³¹) 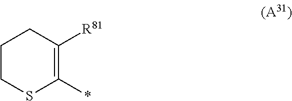
wherein
R⁸¹ represents C₁-C₄-alkyl and C₁-C₄-halogenoalkyl having 1 to 5 halogen atoms,
A³² is a heterocycle of formula (A³²) 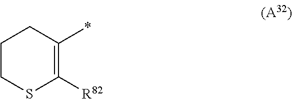
wherein
R⁸² represents C₁-C₄-alkyl and C₁-C₄-halogenoalkyl having 1 to 5 halogen atoms, and
A³³ is a heterocycle of formula (A³³) 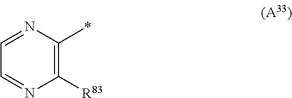
wherein
R⁸³ represents hydrogen, halogen, C₁-C₄-alkyl and C₁-C₄-halogenoalkyl having 1 to 5 halogen atoms,
T represents oxygen or sulphur,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkoxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ represents hydrogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl
Z^{a} represents substituted or unsubstituted C₁-C₈-alkyl, substituted or unsubstituted C₃-C₈-alkenyl, substituted or unsubstituted C₃-C₈-alkynyl, substituted or unsubstituted C₃-C₇-cycloalkyl, substituted or unsubstituted C₁-C₆-alkylideneamino,
represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
And/or an N-oxide, metallic complex, metalloidic complex and/or optically active isomer of a compound of formula (I-1),

3. Compounds of formula (I-2) 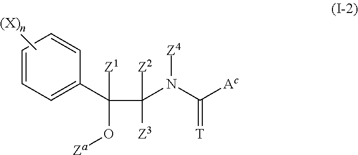
in which,
Z² and Z³ independently represent hydrogen, halogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl, or
Z² and Z³ form together with the carbon atom to which they are attached a 3- to 6-membered, saturated carbocycle or saturated heterocycle, which is optionally substituted by 1 to 6 groups Q which can be the same or different,
Z⁴ represents cyano, unsubstituted C₃-C₇-cycloalkyl or C₃-C₇-cycloalkyl substituted by 1 to 10 substituents that can be the same or different, selected from the list consisting of halogen, cyano, C₁-C₈-alkyl, C₁-C₈-halogenoalkyl comprising 1 to 9 halogen atoms, C₁-C₈-alkoxycarbonyl, C₁-C₈-halogenoalkoxycarbonyl comprising 1 to 9 halogen atoms, C₁-C₈-alkylaminocarbonyl and di-(C₁-C₈-alkyl)-amino, and
A^{c} represents A³ to A¹⁷, A²¹ to A²⁶ and A³⁰ to A³³, provided that when
A^{c} represents A¹³, R³⁴ does not represent a halogenomethyl group.
A³ is a heterocycle of formula (A³) 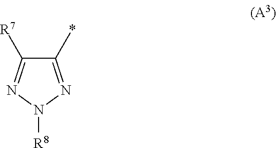
wherein
R⁷ represents hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or C₁-C₅-halogenoalkoxy comprising 1 to 9 halogen atoms, and
R⁸ represents hydrogen or substituted or unsubstituted C₁-C₅-alkyl,
A⁴ is a heterocycle of formula (A⁴) 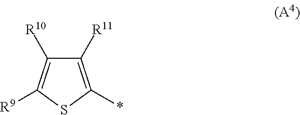
wherein
R⁹ to R¹¹ independently from each other represent hydrogen, halogen, substituted or unsubstituted C₁-C₅-alkyl, amino, substituted or unsubstituted C₁-C₅-alkoxy, substituted or unsubstituted C₁-C₅-alkylsulphanyl, C₁-C₅-halogenoalkyl comprising 1 to 9 halogen atoms or C₁-C₅-halogenoalkoxy comprising 1 to 9 halogen atoms,
A⁵ is a heterocycle of formula (A⁵) 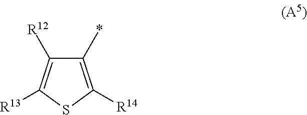
wherein
R¹² and R¹³ independently from each other represent hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms, and
R¹⁴ represents hydrogen, halogen, substituted or unsubstituted C₁-C₅-alkyl, substituted or unsubstituted C₁-C₅-alkoxy, amino, C₁-C₅-halogenoalkyl comprising 1 to 9 halogen atoms or C₁-C₅-halogenoalkoxy comprising 1 to 9 halogen atoms,
A⁶ is a heterocycle of formula (A⁶) 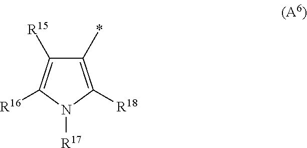
wherein
R¹⁵ represents hydrogen, halogen, cyano, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R¹⁶ and R¹⁸ independently from each other represent hydrogen, halogen, substituted or unsubstituted C₁-C₅-alkoxycarbonyl, substituted or unsubstituted C₁-C₅-alkyl, C₁-C₅-halogenoalkoxy comprising 1 to 9 halogen atoms or C₁-C₅-halogenoalkyl comprising 1 to 9 halogen atoms, and
R¹⁷ represents hydrogen or substituted or unsubstituted C₁-C₅-alkyl,
A⁷ is a heterocycle of formula (A⁷) 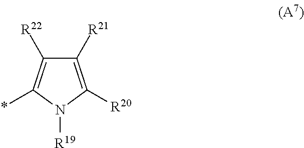
wherein
R¹⁹ represents hydrogen or substituted or unsubstituted C₁-C₅-alkyl, and
R²⁰ to R²² independently from each other represent hydrogen, halogen, substituted or unsubstituted C₁-C₅-alkyl or C₁-C₅-halogenoalkyl comprising 1 to 9 halogen atoms,
A⁸ is a heterocycle of formula (A⁸) 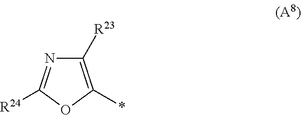
wherein
R²³ represents hydrogen, halogen, substituted or unsubstituted C₁-C₅-alkyl or C₁-C₅-halogenoalkyl comprising 1 to 9 halogen atoms, and
R²⁴ represents hydrogen or substituted or unsubstituted C₁-C₅-alkyl or C₁-C₅-halogenoalkyl comprising 1 to 9 halogen atoms,
A⁹ is a heterocycle of formula (A⁹) 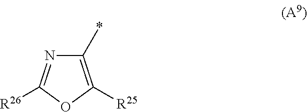
wherein
R²⁵ represents hydrogen, halogen, substituted or unsubstituted C₁-C₅-alkyl or C₁-C₅-halogenoalkyl comprising 1 to 9 halogen atoms, and
R²⁶ represents hydrogen, substituted or unsubstituted C₁-C₅-alkyl or C₁-C₅-halogenoalkyl comprising 1 to 9 halogen atoms,
A¹⁰ is a heterocycle of formula (A¹⁰) 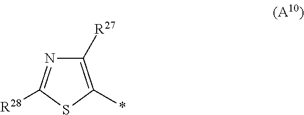
wherein
R²⁷ represents hydrogen, halogen, substituted or unsubstituted C₁-C₅-alkyl or C₁-C₅-halogenoalkyl comprising 1 to 9 halogen atoms, and
R²⁸ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹¹ is a heterocycle of formula (A¹¹) 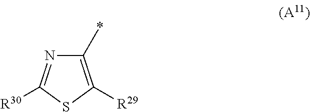
wherein
R²⁹ represents hydrogen, halogen, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms, and
R³⁰ represents hydrogen, halogen, substituted or unsubstituted C₁-C₅-alkyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino,
A¹² is a heterocycle of formula (A¹²) 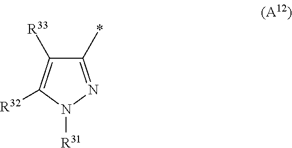
wherein
R³¹ represents hydrogen or substituted or unsubstituted C₁-C₅-alkyl, and
R³² represents hydrogen, halogen, substituted or unsubstituted C₁-C₅-alkyl or C₁-C₅-halogenoalkyl comprising 1 to 9 halogen atoms, and
R³³ represents hydrogen, halogen, nitro, substituted or unsubstituted C₁-C₅-alkyl, substituted or unsubstituted C₁-C₅-alkoxy, C₁-C₅-halogenoalkoxy comprising 1 to 9 halogen atoms or C₁-C₅-halogenoalkyl comprising 1 to 9 halogen atoms,
A¹³ is a heterocycle of formula (A¹³) 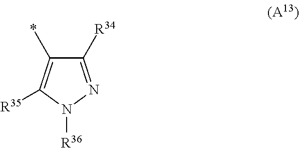
wherein
R³⁴ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms, and
R³⁵ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or substituted or unsubstituted di-(C₁-C₅-alkyl)-amino, and
R³⁶ represents a hydrogen atom or substituted or unsubstituted C₁-C₅-alkyl,
A¹⁴ is a heterocycle of formula (A¹⁴) 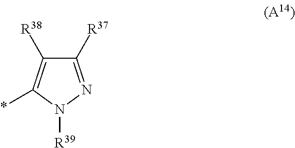
wherein
R³⁷ and R³⁸ independently from each other represent hydrogen, halogen, substituted or unsubstituted C₁-C₅-alkyl, C₁-C₅-halogenoalkyl comprising 1 to 9 halogen atoms, substituted or unsubstituted C₁-C₅-alkoxy or a substituted or unsubstituted C₁-C₅-alkylsulphanyl, and
R³⁹ represents hydrogen or substituted or unsubstituted C₁-C₅-alkyl,
A¹⁵ is a heterocycle of formula (A¹⁵) 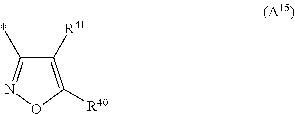
wherein
R⁴⁰ and R⁴¹ independently from each other represent hydrogen, halogen, substituted or unsubstituted C₁-C₅-alkyl or C₁-C₅-halogenoalkyl comprising 1 to 9 halogen atoms, A¹⁶ is a heterocycle of formula (A¹⁶) 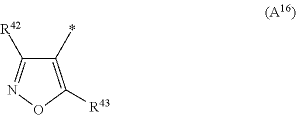
wherein
R⁴² and R⁴³ independently from each other represent hydrogen, halogen, substituted or unsubstituted C₁-C₅-alkyl, C₁-C₅-halogenoalkyl comprising 1 to 9 halogen atoms or amino,
A¹⁷ is a heterocycle of formula (A¹⁷) 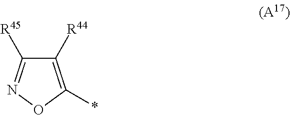
wherein
R⁴⁴ and R⁴⁵ independently from each other represent hydrogen, halogen, substituted or unsubstituted C₁-C₅-alkyl or C₁-C₅-halogenoalkyl comprising 1 to 9 halogen atoms,
A²¹ is a heterocycle of formula (A²¹) 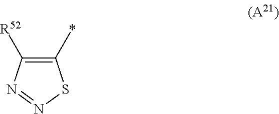
wherein
R⁵² represents hydrogen, halogen, substituted or unsubstituted C₁-C₅-alkyl or C₁-C₅-halogenoalkyl comprising 1 to 9 halogen atoms,
A²² is a heterocycle of formula (A²²) 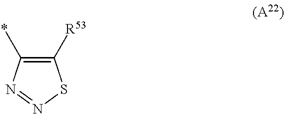
wherein
R⁵³ represents hydrogen, halogen, substituted or unsubstituted C₁-C₅-alkyl or C₁-C₅-halogenoalkyl comprising 1 to 9 halogen atoms,
A²³ is a heterocycle of formula (A²³) 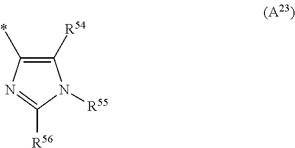
wherein
R⁵⁴ and R⁵⁶ independently from each other represent hydrogen, halogen, substituted or unsubstituted C₁-C₅-alkyl or C₁-C₅-halogenoalkyl comprising 1 to 9 halogen atoms, and
R⁵⁵ represents hydrogen or substituted or unsubstituted C₁-C₅-alkyl,
A²⁴ is a heterocycle of formula (A²⁴) 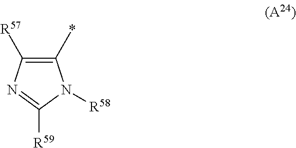
wherein
R⁵⁷ and R⁵⁹ independently from each other represent hydrogen, halogen, substituted or unsubstituted C₁-C₅-alkyl or C₁-C₅-halogenoalkyl comprising 1 to 9 halogen atoms, and
R⁵⁸ represents hydrogen or substituted or unsubstituted C₁-C₅-alkyl,
A²⁵ is a heterocycle of formula (A²⁵) 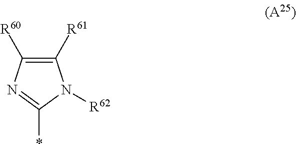
wherein
R⁶⁰ and R⁶¹ independently from each other represent hydrogen, halogen, substituted or unsubstituted C₁-C₅-alkyl or C₁-C₅-halogenoalkyl comprising 1 to 9 halogen atoms, and
R⁶² represents a hydrogen atom or substituted or unsubstituted C₁-C₅-alkyl,
A²⁶ is a heterocycle of formula (A²⁶) 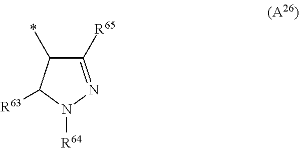
wherein
R⁶³ represents hydrogen, halogen, substituted or unsubstituted C₁-C₅-alkyl, cyano, substituted or unsubstituted C₁-C₅-alkoxy, substituted or unsubstituted C₁-C₅-alkylsulphanyl, C₁-C₅-halogenoalkyl comprising 1 to 9 halogen atoms, C₁-C₅-halogenoalkoxy comprising 1 to 9 halogen atoms, amino, substituted or unsubstituted C₁-C₅-alkylamino or di(C₁-C₅-alkyl)amino, and
R⁶⁴ represents hydrogen or substituted or unsubstituted C₁-C₅-alkyl, and
R⁶⁵ represents hydrogen, halogen, substituted or unsubstituted C₁-C₅-alkyl, substituted or unsubstituted C₃-C₅-cycloalkyl, C₁-C₅-halogenoalkyl comprising 1 to 9 halogen atoms, substituted or unsubstituted C₁-C₅-alkoxy, substituted or unsubstituted C₃-C₅-alkynyloxy or C₁-C₅-halogenoalkoxy comprising 1 to 9 halogen atoms,
A³⁰ is a heterocycle of formula (A³⁰) 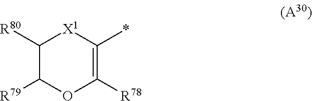
wherein
X¹ represents —S—, —SO—, —SO₂— and —CH₂—, and
R⁷⁸ represents C₁-C₄-alkyl and C₁-C₄-halogenoalkyl having 1 to 5 halogen atoms, and
R⁷⁹ and R⁸⁰ independently from each other represent hydrogen and C₁-C₄-alkyl,
A³¹ is a heterocycle of formula (A³¹) 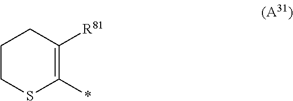
wherein
R⁸¹ represents C₁-C₄-alkyl and C₁-C₄-halogenoalkyl having 1 to 5 halogen atoms,
A³² is a heterocycle of formula (A³²) 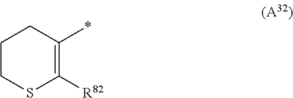
wherein
R⁸² represents C₁-C₄-alkyl and C₁-C₄-halogenoalkyl having 1 to 5 halogen atoms, and
A³³ is a heterocycle of formula (A³³) 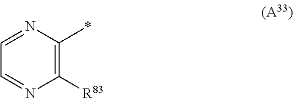
wherein
R⁸³ represents hydrogen, halogen, C₁-C₄-alkyl and C₁-C₄-halogenoalkyl having 1 to 5 halogen atoms,
T represents oxygen or sulphur,
n represents 0, 1, 2, 3, 4 or 5,
X independently from each other X represents halogen, nitro, cyano, isonitrile, hydroxy, amino, sulfanyl, pentafluoro-λ⁶-sulfanyl, formyl, formyloxy, formylamino, substituted or unsubstituted (hydroxyimino)-C₁-C₈-alkyl, substituted or unsubstituted (C₁-C₈-alkoxyimino)-C₁-C₈-alkyl, substituted or unsubstituted (C₂-C₈-alkenyloxyimino)-C₁-C₈-alkyl, substituted or unsubstituted (C₃-C₈-alkynyloxyimino)-C₁-C₈-alkyl, substituted or unsubstituted (benzyloxyimino)-C₁-C₈-alkyl, carboxy, carbamoyl, thiocarbamoyl, N-hydroxycarbamoyl, carbamate, substituted or unsubstituted C₁-C₈-alkyl, C₁-C₈-halogenoalkyl having 1 to 9 halogen atoms, substituted or unsubstituted C₂-C₈-alkenyl, C₂-C₈-halogenoalkenyl having 1 to 9 halogen atoms, substituted or unsubstituted C₂-C₈-alkynyl, C₂-C₈-halogenoalkynyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted C₁-C₈-alkylsulfinyl, C₁-C₈-halogenoalkylsulfinyl having 1 to 9 halogen atoms, substituted or unsubstituted C₁-C₈-alkylsulfonyl, C₁-C₈-halogenoalkylsulfonyl having 1 to 9 halogen atoms, substituted or unsubstituted C₁-C₈-alkylamino, substituted or unsubstituted di-(C₁-C₈-alkyl)-amino, substituted or unsubstituted C₂-C₈-alkenyloxy, C₂-C₈-halogenoalkenyloxy having 1 to 9 halogen atoms, substituted or unsubstituted C₃-C₈-alkynyloxy, C₃-C₈-halogenoalkynyloxy having 1 to 9 halogen atoms, substituted or unsubstituted C₃-C₇-cycloalkyl, C₃-C₇-halogenocycloalkyl having 1 to 9 halogen atoms, substituted or unsubstituted C₃-C₇-cycloalkoxy, substituted or unsubstituted C₄-C₇-cycloalkenyl, C₄-C₇-halogenocycloalkenyl having 1 to 9 halogen atoms, substituted or unsubstituted (C₃-C₇-cycloalkyl)-C₁-C₈-alkyl, substituted or unsubstituted (C₃-C₇-cycloalkyl)-C₂-C₈-alkenyl, substituted or unsubstituted (C₃-C₇-cycloalkyl)-C₂-C₈-alkynyl, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkylcarbonyl, C₁-C₈-halogenoalkylcarbonyl having 1 to 9 halogen atoms, substituted or unsubstituted C₁-C₈-alkylcarbonyloxy, C₁-C₈-halogenoalkylcarbonyloxy having 1 to 9 halogen atoms, substituted or unsubstituted C₁-C₈-alkylcarbonylamino, C₁-C₈-halogenoalkylcarbonylamino having 1 to halogen atoms, substituted or unsubstituted C₁-C₈-alkoxycarbonyl, C₁-C₈-halogenoalkoxycarbonyl having 1 to 9 halogen atoms, substituted or unsubstituted C₁-C₈-alkyloxycarbonyloxy, C₁-C₈-halogenoalkoxycarbonyloxy having 1 to 9 halogen atoms, substituted or unsubstituted C₁-C₈-alkylcarbamoyl, substituted or unsubstituted di-C₁-C₈-alkylcarbamoyl, substituted or unsubstituted C₁-C₈-alkylaminocarbonyloxy, substituted or unsubstituted di-C₁-C₈-alkylaminocarbonyloxy, substituted or unsubstituted N—(C₁-C₈-alkyl)-hydroxycarbamoyl, substituted or unsubstituted C₁-C₈-alkoxycarbamoyl, substituted or unsubstituted N—(C₁-C₈-alkyl)-C₁-C₈-alkoxycarbamoyl, aryl optionally substituted by 1 to 6 groups Q which can be the same or different, aryl-C₁-C₈-alkyl optionally substituted by 1 to 6 groups Q which can be the same or different, aryl-C₂-C₈-alkenyl optionally substituted by 1 to 6 groups Q which can be the same or different, aryl-C₂-C₈-alkynyl optionally substituted by 1 to 6 groups Q which can be the same or different, aryloxy optionally substituted by 1 to 6 groups Q which can be the same or different, arylsulfanyl optionally substituted by 1 to 6 groups Q which can be the same or different, arylamino optionally substituted by 1 to 6 groups Q which can be the same or different, aryl-C₁-C₈-alkyloxy optionally substituted by 1 to 6 groups Q which can be the same or different, aryl-C₁-C₈-alkylsulfanyl optionally substituted by 1 to 6 groups Q which can be the same or different, aryl-C₁-C₈-alkylamino optionally substituted by 1 to 6 groups Q which can be the same or different, pyridinyl which can be substituted by 1 to 4 groups Q, pyridinyloxy which is optionally substituted by 1 to 4 groups Q, or
two substituents X together with the carbon atoms to which they are attached form a 5- or 6-membered, saturated carbocycle or saturated heterocycle, which is optionally substituted by 1 to 4 groups Q which can be the same or different,
Z¹ represents hydrogen, cyano, substituted or unsubstituted C₁-C₈-alkyl, C₁-C₈-halogenoalkyl having 1 to 5 halogen atoms, substituted or unsubstituted C₁-C₈-alkoxy, substituted or unsubstituted C₁-C₈-alkylsulfanyl, or substituted or unsubstituted C₁-C₈-alkoxycarbonyl
Z^{a} represents substituted or unsubstituted C₁-C₈-alkyl, substituted or unsubstituted C₃-C₈-alkenyl, substituted or unsubstituted C₃-C₈-alkynyl, substituted or unsubstituted C₃-C₇-cycloalkyl, substituted or unsubstituted C₁-C₆-alkylideneamino,
Q represents halogen, cyano, nitro, substituted or unsubstituted C₁-C₈-alkyl, C₁-C₈-halogenoalkyl having 1 to 9 halogen atoms, substituted or unsubstituted C₁-C₈-alkoxy, C₁-C₈-halogenoalkoxy having 1 to 9 halogen atoms, substituted or unsubstituted C₁-C₈-alkylsulfanyl, C₁-C₈-halogenoalkylsulfanyl having 1 to 9 halogen atoms, substituted or unsubstituted tri-(C₁-C₈-alkyl)-silyl, substituted or unsubstituted tri-(C₁-C₈-alkyl)-silyl-C₁-C₈-alkyl, substituted or unsubstituted C₁-C₈-alkoxyimino-C₁-C₈-alkyl, substituted or unsubstituted (benzyloxyimino)-C₁-C₈-alkyl,
And/or an N-oxide, metallic complex, metalloidic complex and/or optically active isomer of the compounds of formula (I-2).

4. Composition, comprising an effective amount of at least one compound according to claim 2 and at least one surfactant, solid or liquid diluent.

5. A compounds as described in claim 1 capable of being used for preparation of a medicament for control of one or more animal parasites.

6. A compounds as described in claim 1 capable of being used for preparation of a medicament for control of helminths.

7. A compound according to claim 2 capable of being used for preparation of a medicament for control of one or more animal parasites.

8. A compound as described in claim 2 capable of being used for preparation of a medicament for control of helminths.

9. A compound according to claim 3 capable of being used for preparation of a medicament for control of one or more animal parasites.

10. A compounds as described in claim 3 capable of being used for preparation of a medicament for control of helminths.

11. Composition, comprising an effective amount of at least one compound according to claim 3 and at least one surfactant, solid or liquid diluent
